(19) United States
(12) Patent Application Publication
(10) Pub. No.: US 2024/0108747 A1
(43) Pub. Date: Apr. 4, 2024

(54) EXTRACELLULAR VESICLE CONJUGATES AND USES THEREOF

(71) Applicant: LONZA SALES AG, BASEL (CH)

(72) Inventors: Russell E. MCCONNELL, Cambridge, MA (US); Sriram SATHYANARAYANAN, Lexington, MA (US)

(73) Assignee: LONZA SALES AG, BASEL (CH)

(21) Appl. No.: 17/441,162

(22) PCT Filed: Mar. 20, 2020
(86) PCT No.: PCT/US2020/024057

§ 371 Date: Jun. 15, 2022

Related U.S. Application Data
(60) Provisional application No. 62/822,014, filed on Mar. 21, 2019.
(60) Provisional application No. 62/835,439, filed on Apr. 17, 2019.

(51) Int. Cl. A61K 47/69; A61K 47/65

(52) U.S. Cl. CPC A61K 47/6901; A61K 47/65

(57) ABSTRACT

The present disclosure relates to extracellular vesicles (e.g., exosomes) comprising a biologically active molecule covalently linked to the extracellular vesicle via a maleimide moiety, which may be useful as an agent for the prophylaxis or treatment of cancer and other diseases. Also provided herein are methods for producing the extracellular vesicles and methods for using the extracellular vesicles to treat diseases or disorders.

### CROSS-REFERENCE TO RELATED APPLICATIONS

This application is a 35 U.S.C. § 371 National Phase Application of International Application No. PCT/US2020/024057, filed Mar. 20, 2020, which claims the priority benefit of U.S. Provisional Application Nos. 62/822,014, filed Mar. 21, 2019, and 62/835,439, filed Apr. 17, 2019, each of which is hereby incorporated by reference in its entirety.

### REFERENCE TO SEQUENCE LISTING SUBMITTED ELECTRONICALLY VIA EFS-WEB

The content of the electronically submitted sequence listing (Name: 4000_037PC03_SL_ST25.txt, Size: 235,683 bytes; and Date of Creation: May 10, 2022) submitted in this application is incorporated herein by reference in its entirety.

### TECHNICAL FIELD

The present disclosure provides extracellular vesicles (EVs), e.g., exosomes, comprising at least one biologically active molecule covalently linked to the extracellular vesicle, e.g., exosome, via a maleimide moiety, which can be useful as an agent for the prophylaxis or treatment of cancer and other diseases.

### BACKGROUND

Many bioactive compounds have potent biological activity that is of therapeutic interest. However, these compounds often exhibit toxicity in non-target organs. One way to limit exposure of non-target tissues is to chemically conjugate small molecules to affinity-based reagents such as antibodies, which can direct the therapeutic compound to specific cell types (Dosio, F. et al., Toxins (Basel) 3(7):848-883 (2011)), but this approach is limited by the number of molecules of the compound of interest that can be attached to an antibody (typically 2-6 molecules per antibody), and by the availability/existence of antibodies that specifically bind to targeted, relevant diseased/effector cells without binding to non-target cells. These two issues limit the use of antibody-drug conjugates (ADC) by decreasing potency and increasing systemic toxicity, respectively. Accordingly, there is a need for delivery systems with a higher payload than ADCs that can selectively target specific tissues or organs while at the same time limiting overall systemic exposure to the therapeutic compound.

EVs, e.g., exosomes, are important mediators of intercellular communication. They are also important biomarkers in the diagnosis and prognosis of many diseases, such as cancer. As drug delivery vehicles, EVs, e.g., exosomes, offer many advantages over traditional drug delivery methods (e.g., peptide immunization, DNA vaccines) as a new treatment modality in many therapeutic areas. However, despite its advantages, many EVs, e.g., exosomes, have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Accordingly, new and more effective engineered-EVs, e.g., exosomes, are necessary to better enable therapeutic use and other applications of EV-based technologies.

### BRIEF SUMMARY

The present disclosure provides an extracellular vesicle, e.g., exosome, comprising a biologically active molecule covalently linked to the EV, e.g., exosome, via a maleimide moiety. In some aspects, the maleimide moiety has the formula (I):

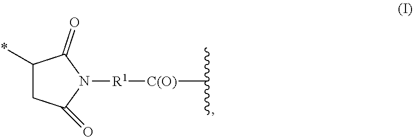

wherein
R¹ is selected from the group consisting of —C₁₋₁₀ alkylene-, —C₃₋₈ carbocyclo-, —O—(C₁₋₈ alkylene)-, -arylene-, —C₁₋₁₀ alkylene-arylene-, -arylene-C₁₋₁₀ alkylene-, —C₁₋₁₀ alkylene-(C₃₋₈ carbocyclo)-, —(C₃₋₈ carbocyclo)-C₁₋₁₀ alkylene-, —C₃₋₈ heterocyclo-, —C₁₋₁₀ alkylene-(C₃₋₈ heterocyclo)-, —(C₃₋₈ heterocyclo)-C₁₋₁₀ alkylene-, —(CH₂CH₂O)ᵣ—, and —(CH₂CH₂O)ᵣ—CH₂—;
r is an integer from 1 to 10;
* indicates the covalent attachment site of the maleimide moiety to the EV, e.g., exosome; and,
the wavy line indicates the attachment site of the maleimide moiety to the biologically active molecule.

In some aspects, R¹ is —(CH₂)ₛ—, wherein s is 4, 5, or 6. In some aspects, the maleimide moiety has the formula (II), where R¹ is —(CH₂)₅—:

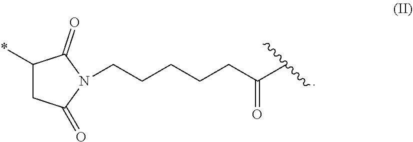 [SMILES: *C(=O)CCCCCN1C(=O)CC(*)C1=O]

In some aspects, the maleimide moiety has the formula (III), where R¹ is —(CH₂CH₂O)ᵣ—CH₂—, where r is 2:

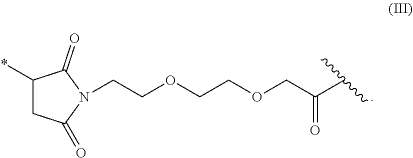 [SMILES: *C(=O)COCCOCCN1C(=O)CC(*)C1=O]

In some aspects, the maleimide moiety is covalently linked to a functional group present on the EV, e.g., exosome, wherein the functional group is a sulfhydryl group. In some aspects, the sulfhydryl group is on a protein on the surface of the EV, e.g., exosome. In some aspects, the maleimide moiety is linked to the biologically active molecule by a linker. In some aspects, the linker comprises a cleavable linker. In some aspects, the cleavable linker is cleaved by a protease. In some aspects, the protease is a cathepsin. In some aspects, the linker is a reduction-sensitive linker, or an acid labile linker.

In some aspects, the linker has the formula (IV):

-Aₐ-Y_{y}— (IV),

wherein each -A- is independently an amino acid unit, a is independently an integer from 1 to 12; —Y— is a spacer unit, and y is 0, 1, or 2.

In some aspects, -Aₐ- is a dipeptide, a tripeptide, a tetrapeptide, a pentapeptide, or a hexapeptide. In some aspects, a is 2 and -Aₐ- is selected from the group consisting of valine-alanine, valine-citrulline, phenylalanine-lysine, N-methylvaline-citrulline, cyclohexylalanine-lysine, and beta-alanine-lysine. In some aspects, -Aₐ- is valine-alanine or valine-citrulline. In some aspects, y is 1.

In some aspects, —Y— is a self-immolative spacer. In some aspects, —Y_{y}— has the formula (V):

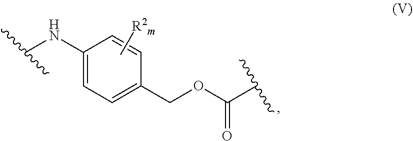

wherein each R² is independently C₁₋₈ alkyl, —O—(C₁₋₈ alkyl), halogen, nitro, or cyano; and m is an integer from 0 to 4.

In some aspects, m is 0, 1, or 2. In some aspects, m is 0. In some aspects, the cleavable linker is valine-alanine-p-aminobenzylcarbamate or valine-citrulline-p-aminobenzylcarbamate. In some aspects, —Y— is a non self-immolative spacer. In some aspects, the non self-immolative spacer is -Gly- or -Gly-Gly-.

In some aspects, the linker is an acid labile linker. In some aspects, the acid labile linker comprises a cis-aconitic linker, a hydrazide linker, a thiocarbamoyl linker, or any combination thereof. In some aspects, the acid labile linker comprises a spacer unit to link the biologically active molecule to the acid labile linker.

In some aspects, the spacer unit has the formula (V):

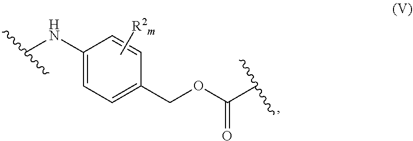

wherein each R² is independently C₁₋₈ alkyl, —O—(C₁₋₈ alkyl), halogen, nitro, or cyano; and m is an integer from 0 to 4.

In some aspects, the linker is a non-cleavable linker. In some aspects, the non-cleavable linker comprises tetraethylene glycol (TEG), polyethylene glycol (PEG), succinimide, or any combination thereof. In some aspects, the non-cleavable linker comprises a spacer unit to link the biologically active molecule to the non-cleavable linker. In some aspects, the spacer unit has the formula (V):

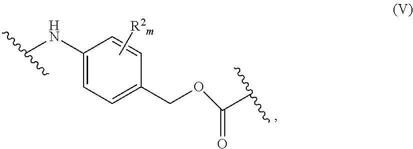

wherein each R² is independently C₁₋₈ alkyl, —O—(C₁₋₈ alkyl), halogen, nitro, or cyano; and m is an integer from 0 to 4.

The present disclosure also provides an EV, e.g., exosome, comprising a biologically active molecule and a cleavable linker, wherein the cleavable linker connects the EV, e.g., exosome, to the biologically active molecule, and the cleavable linker comprises valine-alanine-p-aminobenzylcarbamate or valine-citrulline-p-aminobenzylcarbamate. In some aspects, the EV, e.g., exosome, further comprises a maleimide moiety, which links the EV, e.g., exosome, to the cleavable linker via a functional group present on the EV, e.g., exosome.

In some aspects, the maleimide moiety has the formula (I):

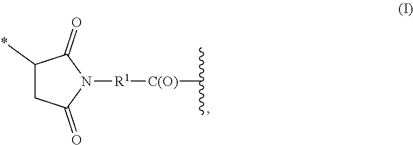

wherein
R¹ is selected from the group consisting of —C₁₋₁₀ alkylene-, —C₃₋₈ carbocyclo-, —O—(C₁₋₈ alkylene)-, -arylene-, —C₁₋₁₀ alkylene-arylene-, -arylene-C₁₋₁₀ alkylene-, —C₁₋₁₀ alkylene-(C₃₋₈ carbocyclo)-, —(C₃₋₈ carbocyclo)-C₁₋₁₀ alkylene-, —C₃₋₈ heterocyclo-, —C₁₋₁₀ alkylene-(C₃₋₈ heterocyclo)-, —(C₃₋₈ heterocyclo)-C₁₋₁₀ alkylene-, —(CH₂CH₂O)ᵣ—, and —(CH₂CH₂O)ᵣ—CH₂—;
r is an integer from 1 to 10; and
* indicates the covalent attachment site of the maleimide moiety to the EV, e.g., exosome; and, the wavy line indicates the attachment site of the maleimide moiety to the biologically active molecule.

In some aspects, R¹ is —(CH₂)ₛ—, wherein s is 4, 5, or 6. In some aspects, the maleimide moiety has the formula (II), where R¹ is —(CH₂)₅—:

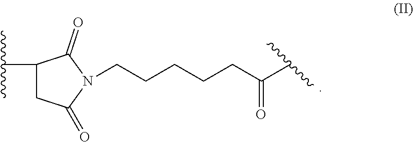 [SMILES: *C(=O)CCCCCN1C(=O)CC(*)C1=O]

In some aspects, the maleimide moiety has the formula (III), where R¹ is —(CH₂CH₂O)ᵣ—CH₂—, where r is 2:

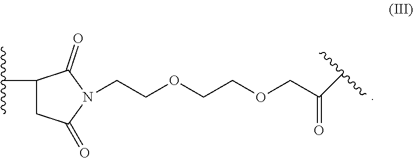 [SMILES: *C(=O)COCCOCCN1C(=O)CC(*)C1=O]

In some aspects, the maleimide moiety is covalently linked to a functional group present on the EV, e.g., exosome. In some aspects, the functional group is on a glycan on the EV, e.g., exosome. In some aspects, the functional group is sulfhydryl (thiol). In some aspects, the functional group is on a protein on the surface of the EV, e.g., exosome. In some aspects, the protein is a scaffold moiety. In some aspects, the protein is a PTGFRN polypeptide, a BSG polypeptide, a IGSF2 polypeptide, a IGSF3 polypeptide, a IGSF8 polypeptide, a ITGB1 polypeptide, a ITGA4 polypeptide, a SLC3A2 polypeptide, a ATP transporter polypeptide, or a fragment thereof.

The present disclosure also provides an EV, e.g., exosome, comprising a maleimide moiety, a cleavable linker, and a biologically active molecule, wherein the maleimide moiety links the EV, e.g., exosome, to the cleavable linker, and the cleavable linker connects the maleimide moiety to the biologically active molecule.

In some aspects, the biologically active molecule is a polypeptide, a peptide, a polynucleotide (DNA and/or RNA), a chemical compound, or any combination thereof. In some aspects, the biologically active molecule is a chemical compound. In some aspects, the chemical compound is a small molecule. In some aspects, the small molecule is a proteolysis-targeting chimera (PROTAC).

In some aspects, the biologically active molecule is nucleotide, wherein the nucleotide is a stimulator of interferon genes protein (STING) agonist. In some aspects, the STING agonist comprises a cyclic dinucleotide STING agonist or a non-cyclic dinucleotide STING agonist.

In some aspects, the EV comprises a (maleimide moiety)-(cleavable linker)-(biologically active molecule) having the formula (VI) or (VII):

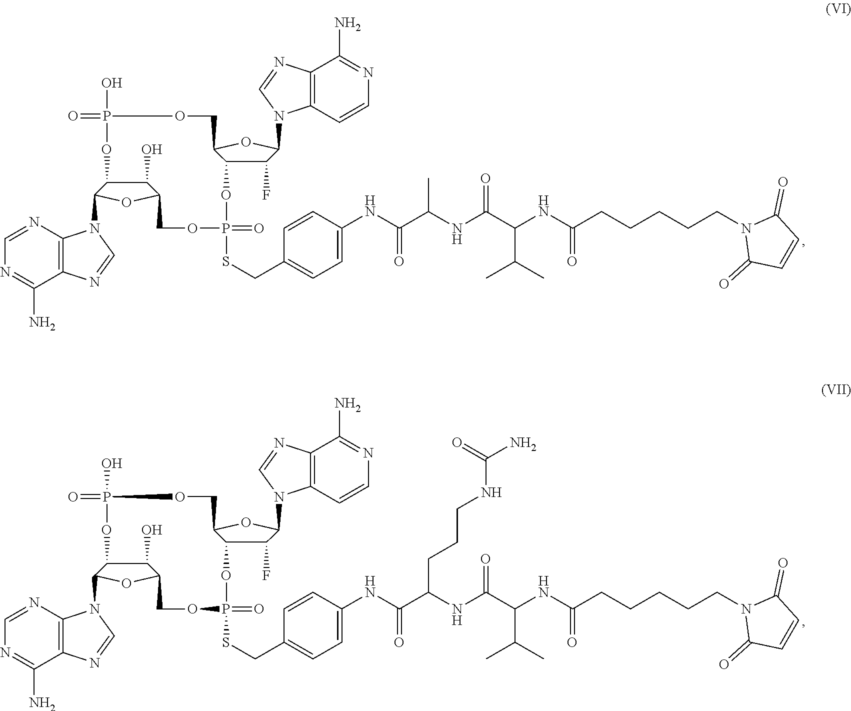

or a pharmaceutically salt thereof.

In some aspects, the EV comprises a (maleimide moiety)-(cleavable linker)-(biologically active molecule) having the formula (VIII), (IX), (X), or (XI):

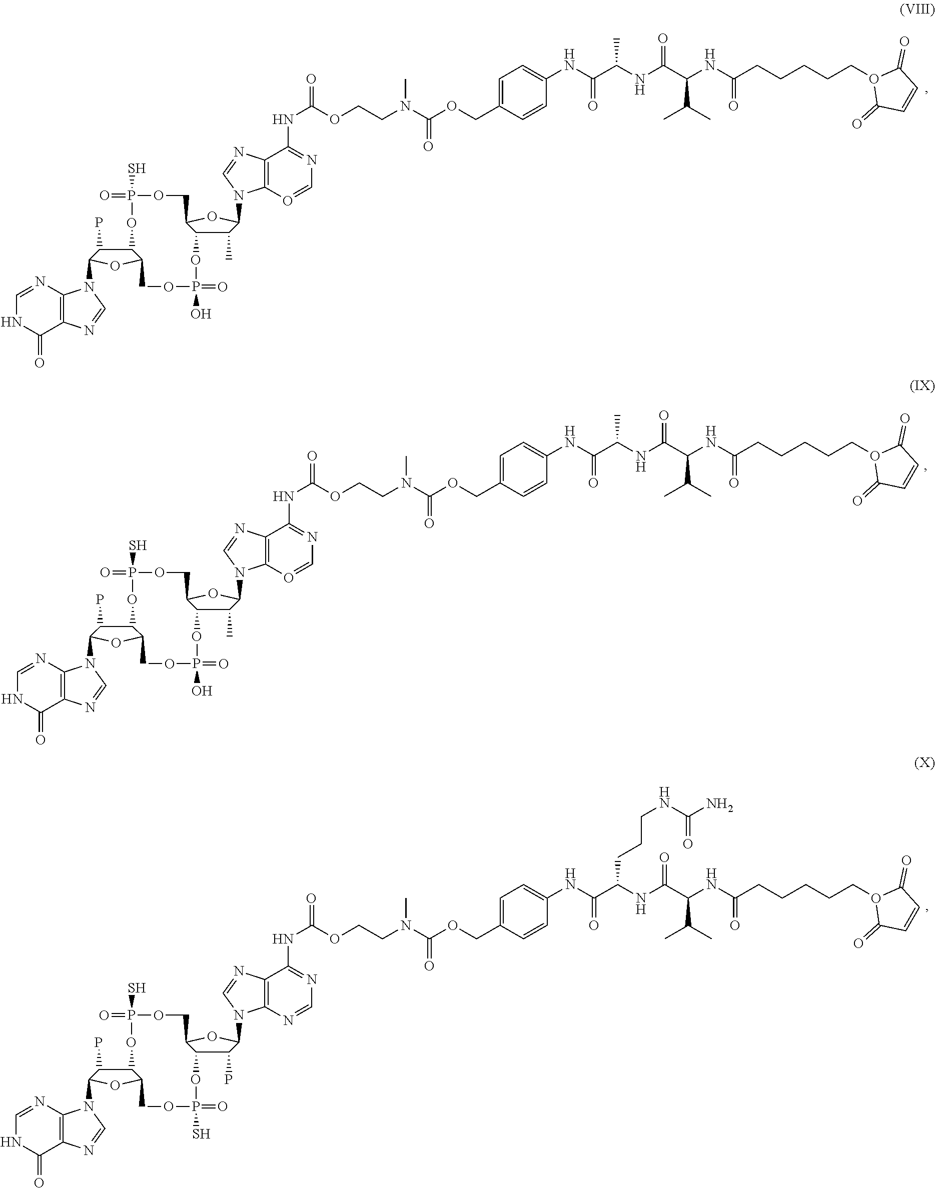

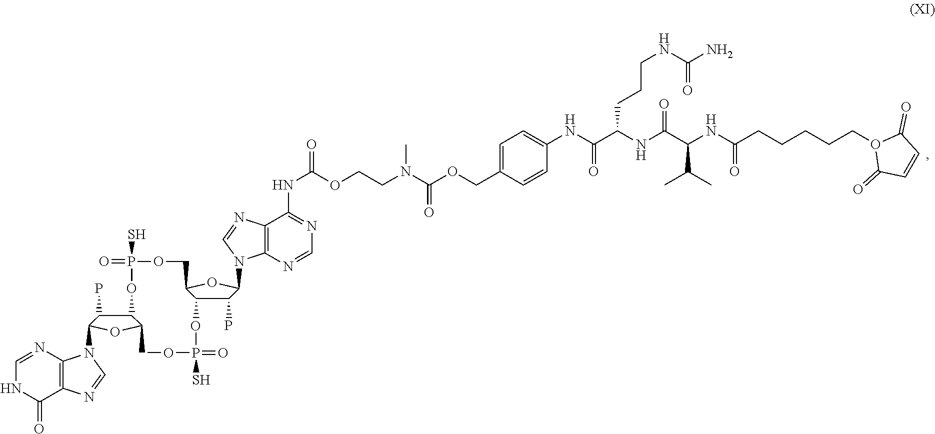

or a pharmaceutical salt thereof.

In some aspects, the EV, e.g., exosome, is modified to expose a functional group on the surface to covalently link the maleimide moiety. In some aspects, the functional group is a sulfhydryl group. In some aspects, the functional group is exposed by treating the EV, e.g., exosome, with a reducing agent. In some aspects, the reducing agent comprises TCEP (Tris(2-carboxyethyl)phosphine), DTT (dithiothreitol), BME (2-mercaptoethanol), a thiolating agent, or any combination thereof. In some aspects, the thiolating agent comprises Traut's reagent (2-iminothiolanel.

In aspects, the EVs of the present disclosure comprise exosomes.

The present disclosure also provides a pharmaceutical composition comprising an EV, e.g., exosome, disclosed herein and a pharmaceutically acceptable carrier.

The present disclosure also provides a method of conjugating a biologically active molecule to an EV, e.g., exosome, comprising linking a maleimide moiety to the EV, e.g., exosome. In some aspects, the linking comprises treating the EV, e.g., exosome, with a reducing agent. In some aspects, the reducing agent is comprises TCEP (Tris(2-carboxyethyl)phosphine), DTT (dithiothreitol), BME (2-mercaptoethanol), a thiolating agent, or any combination thereof. In some aspects, the thiolating agent comprises Traut's reagent (2-iminothiolane). In some aspects, the linking further comprises bringing the reduced EV, e.g., exosome, in contact with the maleimide moiety. In some aspects, the maleimide moiety is chemically linked to a biologically active molecule prior to the linking to the EV, e.g., exosome. In some aspects, the maleimide moiety is chemically linked to a linker to connect the maleimide moiety to the biologically active molecule.

The present disclosure also provides a kit comprising a EV, e.g., exosome, disclosed herein and instructions for use. Also provided is a kit comprising reagents to conjugate a biologically active molecule to an EV, e.g., exosome, and instructions to conduct the conjugation, thereby making an EV, e.g., exosome, of the present disclosure.

The present disclosure also provides a method of treating or preventing a disease or disorder in a subject in need thereof comprising administering an EV, e.g., exosome, of the present disclosure to the subject. In some aspects, the disease or disorder is a cancer, an inflammatory disorder, a neurodegenerative disorder, a central nervous diseases, or a metabolic disease. In some aspects, the EV, e.g., exosome, is administered intravenously, intraperitoneally, nasally, orally, intramuscularly, subcutaneously, parenterally, intrathecally, intraocularly, or intratumorally.

In some aspects, the present disclosure provide an extracellular vesicle (EV) comprising at least one biologically active molecule covalently linked to a scaffold moiety via a maleimide moiety. In some aspects, the maleimide moiety is a bifunctional molecule. In some aspects, the maleimide moiety comprises at least one linker or spacer. In some aspects, the linker is a cleavable linker. In some aspects, the scaffold moiety is a scaffold protein or a scaffold lipid. In some aspects, the scaffold protein is a Scaffold X protein. In some aspects, the Scaffold X protein is a PTGFRN polypeptide, a BSG polypeptide, a IGSF2 polypeptide, a IGSF3 polypeptide, a IGSF8 polypeptide, a ITGB1 polypeptide, a ITGA4 polypeptide, a SLC3A2 polypeptide, a ATP transporter polypeptide, or a fragment thereof. In some aspects, the biologically active molecule comprises a vaccine antigen, a vaccine adjuvant, or any combination thereof. In some aspects, the biologically active molecule comprises a STING, an ASO, a synthetic antineoplastic agent (e.g., MMAE), a cytokine release inhibitor (e.g., MCC950), an mTOR inhibitor (e.g., Rapamycin), an autotaxin inhibitor (e.g., PAT409), an LPA1 antagonist (e.g., AM152), or any combination thereof. In some aspects, the extracellular vesicle further comprises a targeting moiety, a tropism moiety, an anti-phagocytic signal, or any combination thereof. In some aspects, the targeting moiety, tropism moiety, anti-phagocytic signal, or combination thereof is linked to the extracellular vesicle via a maleimide moiety.

### BRIEF DESCRIPTION OF THE DRAWINGS/FIGURES

FIG. 1A is a schematic representation showing how maleimide chemistry can be used to chemically link a biologically active molecule (BAM) to an EV (e.g., an exosome), e.g., via a scaffold moiety described herein (e.g., a Scaffold X protein or fragment thereof or a lipid). The linkers depicted in the drawing are optional and when present can comprise a linker (e.g., a cleavable linker) or a combination thereof.

FIG. 1B shows examples of STING agonist compounds that can be linked to EVs: CP227 (Val-Ala linked to a maleimide moiety), CP229 (Val-Cit linked to a maleimide moiety), CP238 (Val-Ala linked to cholesterol), CP246 (Val-Ala linked to succinimide), CP240 (no linker), CP249 (Val-Ala linked to a maleimide moiety), CP250 (Val-Ala linked to a maleimide moiety), CP260 (Val-Cit linked to a maleimide moiety), and CP261 (Val-Cit linked to a maleimide moiety).

FIG. 2 shows results of PBMC assays assessing STING agonism (IFN-0) of sulfhydryl- or amine-reactive compounds. The activity of free STING agonist compounds (closed circles) and STING agonist compounds loaded on exosomes (open circles) was tested. Exo CP227 is CP227 conjugated to EVs via Val-Ala linked to a maleimide moiety; Exo-CP229 is CP229 conjugated to EVs via Val-Cit linked to a maleimide moiety; Exo-CP232 is CP232 conjugated to EVs without any linker; Exo-CP246 is CP246 conjugated to EVs via Val-Cit linked to succinimide; and Exo-CP250 is CP250 conjugated to EVs via Val-Cit linked to a maleimide moiety. 500,000 PBMCs/well were incubated overnight with exosomes. Interferon beta (IFNβ) release into the cell culture supernatant was measured using an ELISA.

FIGS. 3A-3C show results of PBMC assays comparing sulfhydryl-reactive and lipid-associating chemistries for loading STING agonists (center graphics). FIG. 3A shows the IFNβ release and EC₅₀ comparison of CP227 and ExoCP227, which is CP227 conjugated to EVs via Val-Ala linked to a maleimide moiety. FIG. 3B shows the IFNβ release and EC₅₀ comparison of CP229 and ExoCP229, which is CP229 conjugated to EVs via Val-Cit linked to a maleimide moiety. FIG. 3C shows the IFNβ release and EC₅₀ comparison of CP238 and ExoCP238, which is CP238 conjugated to EVs via Val-Ala linked to cholesterol.

FIG. 4A shows comparison data of the IFNβ release in the PBMC assays between ADUS100 and ExoADUS100, which is ADUS100 encapsulated (in the lumen) in EVs. FIG. 4B shows comparison data of the IFNβ release in the PBMC assays between CL656 and ExoCL656, which is CL656 encapsulated (in the lumen) in EVs. FIG. 4C shows EC50 of the various STING agonists and Exo-STING agonists described in FIGS. 3A-3C and 4A-4B.

FIG. 5A shows the loading efficiency of EVs: exoCP227, exoCP229, exoCP250, exoCP238, exoCP232, and exoCP246. The structure of the EVs are described above. The number of STING agonists loaded on each EV is shown for two experiments. FIG. 5B shows EC₅₀ comparison of various STING agonists and EVs: CP227, exo-CP227, CP229, exo-CP229, CP238, exo-CP238, ADUS100, exoADUS100, CL656, exoCL656, and exoCP250.

FIG. 6 shows the structure of monomethyl auristatin E (MMAE) and maleimide-Val-Cit-PABC-MMAE (vc-MMAE).

FIG. 7 shows MMAE cytotoxicity assessed on RAW264.7 (RAW) cells, a human macrophage cell line. The dose-response effects of MMAE on RAW cell growth are shown in microphotographs (top), and measurements of cells growth (bottom left), and confluence (bottom right).

FIG. 8A presents confluency data comparing of the potency of MMAE in free form (MMAE) or with a maleimide-Val-Cit-PABC linker (vc-MMAE).

FIG. 8B presents confluency data measuring the potency of MMAE after exosome cleanup following incubation of the exosomes with free MMAE. Exosomes were washed with guanidinium hydrochloride at concentrations between 0.1 M and 2 μM.

FIG. 8C presents confluency data measuring the potency of MMAE after exosome cleanup following incubation of the exosomes with Val-Cit-MMAE. Exosomes were washed with guanidinium hydrochloride at concentrations between 0.1 M and 2 μM.

FIG. 8D presents confluency data measuring the potency of MMAE following incubation of exosomes with Val-Cit-MMAE or free MMAE under reducing or non-reducing conditions. Exosomes were incubated with Val-Cit-MMAE or MMAE in the presence or absence of 5 mM TCEP.

FIG. 9A shows the effect of reducing conditions (0 mM TCEP to 50 mM TCEP), loading concentration of compound (10 μM to 100 μM vc-MMAE), and presence or absence of guanidinium hydrochloride (0M or 1M) on the potency of exosomes loaded with Val-Cit-MMAE. FIG. 9B shows the effect of reducing conditions (0 mM TCEP to 50 mM TCEP), loading concentration of compound (100 μM or 300 μM vc-MMAE), and presence or absence of guanidinium hydrochloride (0M or 1M) on the potency of exosomes loaded with Val-Cit-MMAE.

FIG. 10A shows a schematic representation of a PROTAC (proteolysis targeting chimera).

FIG. 10B shows a schematic representation of the mechanism of action of PROTACs.

FIG. 10C shows a formula corresponding to a PROTAC comprising a VHL (E3 ligase) binding ligand moiety, a linker, and a TBK1 (TANK-binding kinase 1) targeting ligand. The formula shows potential sites (indicated be stars) on the VHL (E3 ligase) binding ligand moiety that are susceptible to derivatization with a maleimide linker to chemically link the PROTAC to an extracellular vesicle, e.g., an exosome.

FIG. 11 is a schematic representation of the mechanism of action of a CLIPTAC.

FIG. 12 shows the chemical structures of AM152 (Cyclopropanecarboxylic acid, 1-[4′-[3-methyl-4-[[[(1R)-1-phenylethoxy]carbonyl]amino]-S-isoxazolyl][1,1′-biphenyl]-4-yl]-) and AM095 (1,1′-Biphenyl]-4-acetic acid, 4′-[3-methyl-4-[[[(1R)-1-phenylethoxy]carbonyl]amino]-S-isoxazolyl[ ]-). Arrows labeled 1 and 2 indicate locations (carboxylic acid and carbamate) suitable for derivation to introduce a maleimide reactive group. The corresponding sites indicated in AM152 are also present in AM095.

FIG. 13 is a schematic representation showing the conjugation of an LPA1 antagonist (AM152) to exosomes, to yield a population of exosomes containing a plurality of LPA1 antagonist molecules on their surface.

FIG. 14 shows an example of how a maleimide reactive group can be added to AM152 via its carboxylic acid group. The example shows the maleimide group as part of a reactive complex comprising an Ala-Val cleavable linker and a C₅ spacer interposed between the maleimide group and the carboxylic acid-reactive chloromethyl benzene group.

FIG. 15 shows two exemplary reagents that can be used to derivatize AM152. The top reagent comprises (i) a chloromethyl benzene group that can react with the carboxylic acid group of AM152 and (ii) a maleimide group. Interposed between (i) and (ii) are a cleavable Cit-Val dipeptide and a C₅ spacer. The bottom reagent comprises (i) a chloromethyl benzene group that can react with the carboxylic acid group of AM152 and (ii) a maleimide group, and interposed between (i) and (ii) are a cleavable Ala-Val dipeptide and a C₅ spacer.

FIG. 16 shows the product resulting from cleaving the Cit-Val or Ala-Val dipeptide (e.g., by cathepsin B) in the conjugation product. The product, an AM152 aniline ester, could be further processed by an endogenous esterase to yield the free acid AM152 product.

FIGS. 17 and 18 show several AM152 derivatives comprising a free maleimide group and different combinations of spacers.

FIG. 19 shows that after protection of the carboxylic acid group, it is possible to use the same reagents used to derivatize the carboxylic acid group to derivatize AM152 at its carbamate group. The resulting product would be subsequently deprotected to free the carboxylic acid group.

FIG. 20 shows illustrates an example in which the complex with the maleimide group is chemically linked to the carbamate group of AM152 via a linker. Suitable linkers include any of the linkers disclosed in the present specification.

FIG. 21 shows that AM152 can be chemically linked to a derivatized scaffold moiety instead of being derivatized and subsequently attached to a scaffold moiety via the reactive maleimide group.

FIG. 22 shows the structures of (i) MCC950, (ii) bifunctional reagents that can be used to derivatize MCC950 to introduce a maleimide reactive group, and (iii) MCC950 derivatives comprising a maleimide reactive group. The benzene groups of the bifunctional reagents (**) can react with the carbamate group of MCC950 (*) to yield the depicted MCC950 derivatives.

### DETAILED DESCRIPTION

The present disclosure is directed to extracellular vesicles (EVs), e.g., exosomes, comprising at least one biologically active molecule covalently linked to the EV, e.g., exosome, via a maleimide moiety and uses thereof. EVs, e.g., exosomes, comprising a biologically active molecule linked via a maleimide moiety show superior properties compared to conventional moieties, e.g., cholesterol or succinimide. Non-limiting examples of the various aspects are shown in the present disclosure.

Before the present disclosure is described in greater detail, it is to be understood that this invention is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “aryl” refers to a carbocyclic aromatic group. Examples of aryl groups include, but are not limited to, phenyl, naphthyl and anthracenyl. A carbocyclic aromatic group can be unsubstituted or substituted with one or more groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂—, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

The term “arylene” refers to an aryl group which has two covalent bonds and can be in the ortho, meta, or para configurations as shown in the following structures:

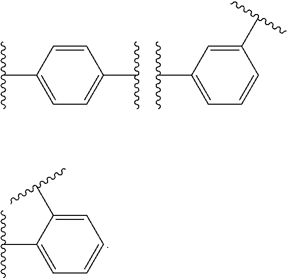

in which the phenyl group can be unsubstituted or substituted with up to four groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂—, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

The term “biologically active molecule” as use herein refers to any molecule that can be linked to an EV, e.g., exosome, for example, chemically linked via a maleimide moiety, wherein the molecule can have a therapeutic or prophylactic effect in a subject in need thereof, or be used for diagnostic purposes. Accordingly, by way of example, the term biologically active molecule includes proteins (e.g., antibodies, proteins, polypeptides, and derivatives, fragments, and variants thereof), lipids and derivatives thereof, carbohydrates (e.g., glycan portions in glycoproteins), or small molecules. In some aspects, the biologically active molecule is a radioisotope. In some aspects, the biologically active molecule is a detectable moiety, e.g., a radionuclide, a fluorescent molecule, or a contrast agent.

The term “C₁₋₈ alkyl” as used herein refers to a straight chain or branched, saturated hydrocarbon having from 1 to 8 carbon atoms. Representative “C₁₋₈ alkyl” groups include, but are not limited to, methyl, ethyl, n-propyl, n-butyl, n-pentyl, n-hexyl, n-heptyl, n-octyl, isopropyl, sec-butyl, isobutyl, tert-butyl, isopentyl, and 2-methylbutyl.

The term “C₁₋₁₀ alkylene” refers to a saturated, straight chain hydrocarbon group of the formula —(CH₂)₁₋₁₀—. Examples of C₁₋₁₀ alkylene include methylene, ethylene, propylene, butylene, pentylene, hexylene, heptylene, octylene, nonylene, and decalene.

The term “C₃₋₈ carbocycle” refers to a 3-, 4-, 5-, 6-, 7- or 8-membered saturated or unsaturated non-aromatic carbocyclic ring. Representative C₃₋₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and -cyclooctadienyl. A C₃₋₈ carbocycle group can be unsubstituted or substituted with one or more groups including, but not limited to, —C₁₋₈ alkyl, —O—(C₁₋₈ alkyl), aryl, —C(O)R′, —OC(O)R′, —C(O)OR′, —C(O)NH₂, —C(O)NHR′, —C(O)N(R′)₂—, —NHC(O)R′, —S(O)₂R′, —S(O)R′, —OH, -halogen, —N₃, —NH₂, —NH(R′), —N(R′)₂ and —CN, where each R′ is independently H, —C₁₋₈ alkyl, or aryl.

The term “C₃₋₈ carbocyclo” refers to a C₃₋₈ carbocycle group defined above wherein one or more of the carbocycle's hydrogen atoms is replaced with a bond.

The term “C₃₋₈ heterocycle” refers to an aromatic or non-aromatic C₃₋₈ carbocycle in which one to four of the ring carbon atoms are independently replaced with a heteroatom selected from the group consisting of O, S and N. Representative examples of a C₃₋₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃₋₈ heterocycle can be unsubstituted or substituted with up to seven groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂, —NHC(O)R′, —S(O)₂R′, —S(O)R′, —OH, -halogen, —N₃, —NH₂, —NH(R′), —N(R′)₂, and —CN, wherein each R′ is independently H, —C₁₋₈ alkyl, or aryl.

The term “C₃₋₈ heterocyclo” refers to a C₃₋₈ heterocycle group defined above wherein one of the heterocycle group's hydrogen atoms is replaced with a bond. A C₃₋₈ heterocyclo can be unsubstituted or substituted with up to six groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the term “conventional EV protein” means a protein previously known to be enriched in EVs.

As used herein, the term “conventional exosome protein” means a protein previously known to be enriched in exosomes, including but is not limited to CD9, CD63, CD81, PDGFR, GPI proteins, lactadherin LAMP2, and LAMP2B, a fragment thereof, or a peptide that binds thereto.

The term “derivative” as used herein refers to an EV, e.g., exosome, component (e.g., a scaffold protein, such as Scaffold X and/or Scaffold Y, a lipid, or a carbohydrate) or to a biologically active molecule (e.g., a polypeptide, polynucleotide, lipid, carbohydrate, antibody or fragment thereof, PROTAC, etc.) that has been chemically modified to either introduce a reactive maleimide group or a thiol group susceptible of reaction with a maleimide group. For example, an antibody modified with a bifunctional reagent comprising (i) a group reacting, e.g., with free amino groups, and (ii) a maleimide group, could result in antibody derivative comprising a reactive maleimide group that can react with free thiol groups in a Scaffold X protein on the EV, e.g., exosome. Conversely, a Scaffold X on the EV, e.g., exosome, could be modified with a bifunctional reagent comprising (i) a group reacting, e.g., with free amino groups, and (ii) a maleimide group, resulting in a Scaffold X derivative comprising a reactive maleimide group that can react with free thiol groups in a biologically active molecule, e.g., an antibody.

The terms “excipient” and “carrier” are used interchangeably and refer to an inert substance added to a pharmaceutical composition to further facilitate administration of a compound.

As used herein, the terms “extracellular vesicle,” “EV,” and grammatical variants thereof, are used interchangeably and refer to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise adeno-associated virus (AAV), nucleic acids (e.g., DNA or RNA, such as antisense oligonucleotides, siRNA, shRNA, or mRNA), morpholinos, proteins, carbohydrates, lipids, small molecules, antigens, vaccines, vaccine adjuvants, and/or combinations thereof. Additional payloads are described if detail below. In some aspects, the EV, e.g., exosome, can further comprise a targeting moiety, a tropism moiety, or a combination thereof. In some aspects, the term extracellular vesicle or EV refers to a population of extracellular vesicles (EVs).

In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the exosomes of the present disclosure are produced by cells that express one or more transgene products. In some aspects, the term exosome refers to a population of exosomes.

In some aspects, EVs, e.g., exosomes, e.g., nanovesicles, of the present disclosure are engineered by chemically linking at least one biologically active molecule (e.g., a protein such as an antibody or ADC, a RNA or DNA such as an antisense oligonucleotide, a small molecule drug, a toxin, a PROTAC, an AAV, or a morpholino) to the EV, e.g., exosome, e.g., nanovesicle, via a maleimide moiety. In some aspects, the maleimide moiety is part of a bifunctional reagent.

In some aspects, the EVs, e.g., exosomes or nanovesicles, of the present disclosure can comprise various macromolecular payloads either within the internal space (i.e., lumen), displayed on the external (exterior) surface or internal (luminal) surface of the EV, and/or spanning the membrane. In some aspects, the payload can comprise, e.g., nucleic acids, proteins, carbohydrates, lipids, small molecules, and/or combinations thereof. In certain aspects, an EV, e.g, an exosome, comprises a scaffold moiety, e.g., a Scaffold X protein or a fragment thereof. EVs, e.g., exosomes, can be derived from a living or dead organism, explanted tissues or organs, prokaryotic or eukaryotic cells, and/or cultured cells. In some aspects, the EVs, e.g., exosomes, are produced by cells that express one or more transgene products. In other aspects, the EVs of the present disclosure are without limitation nanovesicles, microsomes, microvesicles, extracellular bodies, or apoptotic bodies.

As used herein, the term “fragment” of a protein (e.g., a biologically active molecule such as a therapeutic protein, or a scaffold protein such as Scaffold X protein or a fragment thereof, or a Scaffold Y protein or a fragment thereof) refers to an amino acid sequence of a protein that is shorter than the naturally-occurring sequence, N- and/or C-terminally deleted or any part of the protein deleted in comparison to the naturally occurring protein.

As used herein, the term “functional fragment” refers to a protein fragment that retains protein function. Accordingly, in some aspects, a functional fragment of a Scaffold protein, e.g., a fragment of a Scaffold X protein, retains the ability to link or attach a moiety, e.g., a biologically active molecule, on the luminal surface or on the external surface of the EV, e.g., exosome, for example, via a maleimide moiety. Similarly, in certain aspects, a functional fragment of a Scaffold Y protein retains the ability to attach a moiety, e.g., a biologically active molecule, on the luminal surface of the EV, e.g., exosome, for example, via a maleimide moiety.

Whether a fragment is a functional fragment can be assessed by any art known methods to determine the protein content of EVs, e.g., exosomes, including Western Blots, FACS analysis, and fusions of the fragments with autofluorescent proteins like, e.g., GFP. In certain aspects, a functional fragment of a Scaffold X protein retains, e.g., at least about 50%, at least about 60%, at least about 70%, at least about 80%, at least about 90% or at least about 100% of the ability of the naturally occurring Scaffold X protein to attach a biologically active molecule on the luminal or on the external surface of the EV, e.g., exosome, for example, via a maleimide moiety.

As used herein, the term “linking” or “attaching” a biologically active molecule to the luminal or external surface of an EV (e.g., exosome) of the present disclosure includes both (i) “chemically linking” or “conjugating” the biologically active molecule, e.g., via a chemical linker such as a maleimide moiety, and (ii) “non-chemically linking,” also referred to as “fusing,” or “fusion” of (e.g., via a peptide bond, an amino acid linker, and/or a scaffold protein) the biologically active molecule to the EV (e.g., an exosome) or the portion of the scaffold protein located on the luminal or external surface of the EV (e.g., an exosome).

As used herein, the terms “fusing,” “fused,” “fusion,” or “non-chemically linking” a biologically active molecule on the luminal or external surface of an EV (e.g., exosome) of the present disclosure via, e.g., a scaffold protein, refers to linking the biologically active molecule to the portion of the scaffold molecule (e.g., protein) located on the luminal or external surface of the EV (e.g., exosome), respectively. In some aspects, the fusion between a biologically active molecule can be done via genetic fusion (i.e., chimeric expression).

As used herein, the terms “chemically linking” and “conjugating” are used interchangeably an each refer to the covalent attachment of two or more moieties, each one comprising, e.g., an EV, a scaffold moiety, a biologically active moiety, a linker or linkers, a targeting moiety and/or a tropism moiety, or any combination thereof, using a chemical moiety, e.g., a maleimide moiety. As a result, a first moiety (e.g., a scaffold such as a Scaffold X protein or a lipid such as cholesterol) would become “chemically linked” to a second moiety, e.g., a biologically active moiety, via a thioether linkage formed by the reaction between the maleimide group present in one moiety and an a sulfhydryl group present in the other moiety.

As used herein, the term “extracellular” can be used interchangeably with the terms “external,” “exterior,” and “extra-vesicular,” wherein each term refers to an element that is outside the membrane that encloses the EV, e.g., an exosome. As used herein, the term “intracellular” can be used interchangeably with the terms “internal,” “interior,” and “intra-vesicular,” wherein each term refers to an element that is inside the membrane that encloses the EV, e.g., an exosome. The term “lumen” refers to the space inside the membrane enclosing the EV, e.g., an exosome. Accordingly, an element that is inside the lumen of an EV, e.g., exosome, can be referred to herein as being “located in the lumen” or “luminal.”

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “immune modulator” refers to an agent that acts on a target (e.g., a target cell) that is contacted with the EV (e.g., exosome), and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA or DNA, or a small molecule. In some aspects of the present disclosure, the biologically active molecule is an immune modulator.

An “immune response”, as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects of the present disclosure, the biologically active molecule is a molecule capable of eliciting an immune response.

In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

The term “immunoconjugate” as used herein refers to a compound comprising a binding molecule (e.g., an antibody) and one or more moieties, e.g., therapeutic or diagnostic moieties, chemically conjugated to the binding molecule. In general an immunoconjugate is defined by a generic formula: A-(L-M)n wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In other aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate.

As used herein, the terms “isolated,” “purified,” “extracted,” and grammatical variants thereof are used interchangeably and refer to the state of a preparation of desired EVs (e.g., a plurality of EVs of known or unknown amount and/or concentration), that has undergone one or more processes of purification, e.g., a selection or an enrichment of the desired EV, e,g., exosome, preparation. In some aspects, isolating or purifying as used herein is the process of removing, partially removing (e.g., a fraction) of the EVs, e.g., exosomes, from a sample containing producer cells. In some aspects, an isolated EV, e.g., exosome, composition has no detectable undesired activity or, alternatively, the level or amount of the undesired activity is at or below an acceptable level or amount. In other aspects, an isolated EV, e.g., exosome, composition has an amount and/or concentration of desired EVs, e.g., exosomes, at or above an acceptable amount and/or concentration. In other aspects, the isolated EVs, e.g., exosome, composition is enriched as compared to the starting material (e.g., producer cell preparations) from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated EV, e.g. exosome, preparations are substantially free of residual biological products. In some aspects, the isolated EV, e.g., exosome, preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites. Substantially free of residual biological products can also mean that the EV, e.g., exosome, composition contains no detectable producer cells and that only EVs, e.g., exosomes, are detectable.

As used herein the term “lumen-engineered EV” refers to an EV, e.g., exosome with the luminal surface of the membrane or the lumen of the EV, e.g., exosome, modified in its composition so that the luminal surface or the lumen of the engineered EV, e.g., exosome, is different from that of the EV, e.g., exosome, prior to the modification or of the naturally occurring EV, e.g., exosome.

The engineering can be directly in the lumen (i.e., the void within the EV) or in the membrane of the EV (e.g., exosome), in particular the luminal surface of the EV, so that the lumen and/or the luminal surface of the EV, e.g., exosome is changed. For example, the membrane is modified in its composition of a protein, a lipid, a small molecule, a carbohydrate, etc. so that the luminal surface of the EV, e.g., exosome is modified. Similarly, the contents in the lumen can be modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered EV, e.g., lumen-engineered exosome, comprises an exogenous protein (i.e., a protein that the EV, e.g., exosome, does not naturally express) or a fragment or variant thereof that can be exposed on the luminal surface or lumen of the EV, e.g., exosome, or can link a moiety to the luminal surface of the EV, e.g., exosome, to the EV. In other aspects, a lumen-engineered EV, e.g., a lumen-engineered exosome, comprises a higher expression of a natural EV, e.g., exosome, protein (e.g., Scaffold X or Scaffold Y) or a fragment or variant thereof that can be exposed to the lumen of the EV, e.g., exosome, or can link a moiety to the luminal surface of the EV, e.g., exosome.

As used herein, the term “macromolecule” refers to nucleic acids, proteins, lipids, carbohydrates, metabolites, or combinations thereof.

As used herein the term “maleimide moiety” or “MM” refers to a chemical moiety linking an EV, .e.g, exosome, to a linker or a biologically active molecule and comprises the maleimide group:

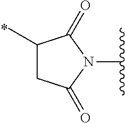 [SMILES: *N1C(=O)CC(C)C1=O]

wherein * indicates the attachment point to any thiol group on the EV, e.g., exosome, (e.g., a free thiol present in a Scaffold X protein), and the wavy line indicates the attachment site to the rest of the maleimide moiety.

In some aspects, * indicates at attachment point to any thiol group on an antibody, PROTAC, or any other biologically active molecule, and the wavy line indicates the attachment site to the rest of the maleimide moiety to the EV, e.g., exosome (e.g., a Scaffold X protein).

As used herein, the term “macromolecule” refers to nucleic acids, proteins, lipids, carbohydrates, metabolites, or combinations thereof.

The term “modified,” when used in the context of EVs, e.g., exosomes, described herein, refers to an alteration or engineering of an EV, e.g., exosome and/or its producer cell, such that the modified EV, e.g., exosome, is different from a naturally-occurring EV, e.g., exosome. In some aspects, a modified EV, e.g., exosome, described herein comprises a membrane that differs in composition of a protein, a lipid, a small molecular, a carbohydrate, etc. compared to the membrane of a naturally-occurring EV, e.g., exosome. For example, the membrane comprises higher density or number of natural EV, e.g., exosome, proteins and/or membrane comprises proteins that are not naturally found in EV, e.g., exosomes. In certain aspects, such modifications to the membrane change the exterior surface of the EV, e.g., exosome (e.g., surface-engineered EVs and exosomes described herein). In certain aspects, such modifications to the membrane change the luminal surface of the EV, e.g., exosome (e.g., lumen-engineered EV and exosomes described herein).

As used herein, the terms “modified protein” or “protein modification” refer to a protein having at least about 15% identity to the non-mutant amino acid sequence of the protein. A modification of a protein includes a fragment or a variant of the protein. A modification of a protein can further include chemical, or physical modification to a fragment or a variant of the protein.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, the term “nanovesicle” refers to an extracellular vesicle with a diameter between about 20 nm and about 250 nm (e.g., between about 30 and about 150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety, e.g., a Scaffold X protein or fragment thereof and/or a Scaffold Y protein or fragment thereof. Nanovesicles, once derived from a producer cell, can be isolated from the producer cell based on its size, density, biochemical parameters, or a combination thereof.

As used herein, the term “payload” refers to a biologically active molecule (e.g., a therapeutic agent) that acts on a target (e.g., a target cell) that is contacted with the EV, e.g., exosome, of the present disclosure. Non-limiting examples of payloads that can be introduced into an EV, e.g., exosome, include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen. As used herein, the term “antigen” refers to any agent that when introduced into a subject elicits an immune response (cellular or humoral) to itself. In some aspects, the antigen is used to elicit an immune response, i.e., as a vaccine. In other aspects, a payload comprises an adjuvant. In some aspects, the payload molecules are covalently linked to the EV, e.g., exosome, via a maleimide moiety.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as an exosome of the present disclosure, mixed or intermingled with, or suspended in one or more other chemical components, such as pharmaceutically-acceptable carriers and excipients. One purpose of a pharmaceutical composition is to facilitate administration of preparations of EVs, e.g., exosomes, to a subject in need thereof.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a maleimide moiety is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide.

In some aspects, a polynucleotide disclosed herein can be modifed to introduce a thiol group that could be used to react with a maleimide moiety. In some aspects, a polynucleotide disclosed herein can be modifed to introduce a maleimide moiety group that could be used to react with a thiol group.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV, e.g., exosome, via a maleimide moiety is a polypeptide, e.g., an antibody or a derivative thereof such as an ADC, a PROTAC, a toxin, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

In some aspects, a polypeptide disclosed herein can be modifed to introduce a thiol group that could be used to react with a maleimide moiety. In some aspects, a polypeptide disclosed herein can be modifed to introduce a maleimide moiety that could be used to react with a thiol group.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHTDF fibroblast cells, AGE.HN© neuronal precursor cells, CAP© amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, a Scaffold X protein and/or a Scaffold Y protein present in EVs, e.g., exosomes, can be recombinantly produced by overexpressing the scaffold proteins in the producer cells, so that levels of scaffold proteins in the resulting EVs, e.g., exosomes, are significantly increased with respect to the levels of scaffold proteins present in EVs, e.g., exosomes, of producer cells not overexpressing such scaffold proteins.

As used herein, the term “scaffold moiety” refers to a molecule, e.g., a protein or a fragment thereof (e.g., a functional fragment thereof), that can be used to link a payload, e.g., a biologically active molecule, or any other compound of interest (e.g., an AAV) to the EV, e.g., exosome, either on the luminal surface (such as a Scaffold Y protein) or on the external surface (such as a Scaffold X protein) of the EV, e.g., exosome. In some aspects, the scaffold protein is a polypeptide that does not naturally exist in an EV, e.g., exosome. In certain aspects, a scaffold moiety comprises a synthetic molecule. In some aspects, a scaffold moiety comprises a non-polypeptide moiety. In other aspects, a scaffold moiety comprises, e.g., a lipid, carbohydrate, protein, or combination thereof (e.g., a glycoprotein or a proteolipid) that naturally exists in the EV, e.g., exosome. In some aspects, a scaffold moiety comprises a lipid, carbohydrate, or protein that does not naturally exist in the EV, e.g., exosome. In some aspects, a scaffold moiety comprises a lipid or carbohydrate which naturally exists in the EV, e.g., exosome, but has been enriched in the EV, e.g., exosome with respect to basal/native/wild type levels. In some aspects, a scaffold moiety comprises a protein which naturally exists in the EV, e.g., exosome but has been enriched in the EV, e.g., exosome, for example, by recombinant overexpression in the producer cell, with respect to basal/native/wild type levels. In certain aspects, a scaffold moiety is a Scaffold X protein or fragment thereof. In some aspects, a scaffold moiety is a Scaffold Y protein or a fragment thereof. In further aspects, the EV comprises both a Scaffold X protein or a fragment thereof and a Scaffold Y protein or a fragment thereof.

As used herein, the term “Scaffold X” refers to EV, e.g., exosome, proteins that have been identified on the surface of EVs, e.g., exosomes, and can be engineered to be overexpressed in EVs. See, e.g., U.S. Pat. No. 10,195,290, which is incorporated herein by reference in its entirety. Non-limiting examples of Scaffold X proteins include: prostaglandin F2 receptor negative regulator (“PTGFRN”); basigin (“BSG”); immunoglobulin superfamily member 2 (“IGSF2”); immunoglobulin superfamily member 3 (“IGSF3”); immunoglobulin superfamily member 8 (“IGSF8”); integrin beta-1 (“ITGB1”); integrin alpha-4 (“ITGA4”); 4F2 cell-surface antigen heavy chain (“SLC3A2”); and a class of ATP transporter proteins (“ATP1A1,” “ATP1A2,” “ATP1A3,” “ATP1A4,” “ATP1B3,” “ATP2B1,” “ATP2B2,” “ATP2B3,” “ATP2B”), a fragment thereof, and any combination thereof. In some aspects, a Scaffold X protein can be a whole protein or a fragment thereof (e.g., functional fragment, e.g., the smallest fragment that is capable of linking another moiety on the external surface or on the luminal surface of the EV, e.g., exosome). In some aspects, a Scaffold X can link a biologically active molecule to the external surface or the lumen of the EV, e.g. an exosome. In some aspects of the present disclosure, a biologically active molecule can be chemically linked to a Scaffold X protein or fragment thereof via a maleimide moiety. In some aspects, the biologically active molecule can be chemically linked to a Scaffold X protein or fragment thereof via a maleimide moiety on the luminal surface of the EV, e.g., exosome. Non-limiting examples of other scaffold moieties that can be used with the present disclosure include: aminopeptidase N (CD13); Neprilysin (membrane metalloendopeptidase; MME); ectonucleotide pyrophosphatase/phosphodiesterase family member 1 (ENPP1); neuropilin-1 (NRP1); CD9, CD63, CD81, PDGFR, GPI proteins, lactadherin, LAMP2, and LAMP2B, a fragment thereof, and any combination thereof.

In some aspects, the scaffold moiety (e.g., EV protein described in U.S. Pat. No. 10,195,290, which is incorporated herein by reference in its entirety) forms a fusion with a binding partner (e.g., an antigen binding domain, a capsid protein, an Fc receptor, a binding partner of a chemically induced dimer, or any combination thereof) that can be used to bind another molecule (i.e., a second binding partner).

As used herein, the term “binding partner” refers to one member of at least two elements that interact with each other to form a multimer (e.g., a dimer). In some aspects, the binding partner is a first binding partner that interacts with a second binding partner. In some aspects, the binding partner is a first binding partner that interacts with a second binding partner and/or a third binding partner. Any binding partners can be used in the compositions and methods disclosed herein. In some aspects, the binding partner can be a polypeptide, a polynucleotide, a fatty acid, a small molecule, or any combination thereof. In certain aspects, the binding partner (e.g., the first binding partner and/or the second binding partner) is selected from a first and a second binding partners of a chemically induced dimer.

As used herein, the term “Scaffold Y” refers to EV, e.g., exosome, proteins that have been identified within the lumen of EV, e.g., exosomes, and can be engineered to be overexpressed in EVs. See, e.g., International Appl. No. PCT/US2018/061679, which is incorporated herein by reference in its entirety. Non-limiting examples of Scaffold Y proteins include: myristoylated alanine rich Protein Kinase C substrate (“MARCKS”); myristoylated alanine rich Protein Kinase C substrate like 1 (“MARCKSL1”); and brain acid soluble protein 1 (“BASP1”), a fragment thereof, and any combination thereof. In some aspects, a Scaffold Y protein can be a whole protein or a fragment thereof (e.g., functional fragment, e.g., the smallest fragment that is capable of linking a moiety to the luminal surface of the EV, e.g., exosome). In some aspects, a Scaffold Y protein or fragment thereof can link a moiety to the luminal surface of the EV, e.g., exosome. In some aspects of the present disclosure, a moiety, e.g., a biologically active molecule, can be linked, e.g., chemically linked, to a Scaffold Y protein or fragment thereof. In some aspects, the moiety, e.g., a biologically active molecule, can be linked, e.g., chemically linked, to a Scaffold Y protein or fragment thereof on the luminal surface of the EV, e.g., exosome.

In certain aspects, the scaffold protein comprises a fragment of an EV protein. In some aspects, the scaffold protein comprises a fragment of MARCKS, MARCKSL1, or BASP1. In some aspects, the scaffold protein comprises the amino acid sequence GGKLSKK (SEQ ID NO: 17). In some aspects, the scaffold protein comprises the amino acid sequence GGKLSKK (SEQ ID NO: 17), wherein the C-terminal Glycine residue is myristoylated.

In some aspects, the scaffold protein is a transmembrane protein. As used herein, a “transmembrane protein” refers to any protein that comprises an extracellular domain (e.g., at least one amino acid that is located external to the membrane of the EV, e.g., exosome, e.g., extra-vesicular), a transmembrane domain (e.g., at least one amino acid that is located within the membrane of an EV, e.g., within the membrane of an exosome), and an intracellular domain (e.g., at least one amino acid that is located internal to the membrane of the EV, e.g., exosome). In some aspects, a scaffold protein described herein is a type I transmembrane protein, wherein the N-terminus of the transmembrane protein is located in the extracellular space, e.g., outside the membrane the encloses the EV, e.g., exosome, e.g., extra-vesicular. In some aspects, a scaffold protein described herein is a type II transmembrane protein, wherein the N-terminus of the transmembrane protein is located in the intracellular space, e.g., inside the membrane, e.g., on the luminal side of the membrane, that encloses the EV, e.g., exosome, e.g., intra-vesicular.

The term “self-immolative spacer” as used herein refers to a spacer as defined below that will spontaneously separate from the second moiety (e.g., a biologically active molecule) if its bond to the first moiety (e.g., a cleavable linker) is cleaved.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The term “spacer” as used herein refers to a bifunctional chemical moiety which is capable of covalently linking together two spaced moieties (e.g., a cleavable linker and a biologically active molecule) into a normally stable dipartate molecule.

Unless otherwise indicated, reference to a compound that has one or more stereocenters intends each stereoisomer, and all combinations of stereoisomers, thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the term “substantially free” means that the sample comprising EVs, e.g., exosomes, comprises less than 10% of macromolecules, e.g., contaminants, by mass/volume (m/v) percentage concentration. Some fractions can contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein the term “surface-engineered EV” (e.g., Scaffold X-engineered exosome) refers to an EV with the membrane or the surface of the EV modified in its composition so that the surface of the engineered EV is different from that of the EV prior to the modification or of the naturally occurring EV.

As used herein the term “surface-engineered exosome” (e.g., Scaffold X-engineered exosome) refers to an exosome with the membrane or the surface of the exosome (external surface or luminal surface) modified in its composition so that the surface of the engineered exosome is different from that of the exosome prior to the modification or of the naturally occurring exosome.

The engineering can be on the surface of the EV, e.g., exosome or in the membrane of the EV, e.g., exosome, so that the surface of the EV, e.g., exosome is changed. For example, the membrane can be modified in its composition of, e.g., a protein, a lipid, a small molecule, a carbohydrate, or a combination thereof. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome or can link a moiety to the surface of the EV, e.g., exosome. In other aspects, a surface-engineered EV, e.g., exosome comprises a higher expression (e.g., higher number) of a natural EV, e.g., exosome protein (e.g., a Scaffold X protein) or a fragment or variant thereof that can be exposed to the surface of the EV, e.g., exosome or can link a moiety to the surface of the EV, e.g., exosome. In a specific aspect, a surface-engineered EV, e.g., exosome, comprises the modification of one or more membrane components, e.g., a protein such as a Scaffold X protein or a fragment thereof, a lipid, a small molecule, a carbohydrate, or any combination thereof, wherein at least one of the components is linked, e.g., chemically linked, to a biologically active molecule, e.g., via a maleimide moiety.

As used herein the term “therapeutically effective amount” is the amount of reagent or pharmaceutical compound comprising an EV or exosome of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refer to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition; or any combination thereof. The term also include prophylaxis or prevention of a disease or condition or its symptoms thereof. In one aspect, the term “treating” or “treatment” means inducing an immune response against an antigen in a subject in need thereof, e.g., by administering an EV, e.g., exosome, comprising an antigen (vaccine antigen) and optionally an adjuvant on the external surface of the EV, e.g., exosome.

As used herein, the term “variant” of a molecule (e.g., functional molecule, antigen, adjuvant, Scaffold X protein or fragment and/or Scaffold Y protein or fragment thereof) refers to a molecule that shares certain structural and functional identities with another molecule upon comparison by a method known in the art. For example, a variant of a protein can include a substitution, insertion, deletion, frame shift or rearrangement in another protein.

In some aspects, a variant of a Scaffold X or derivative comprises a Scaffold X variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins.

In some aspects, the variant or variant of a fragment of a Scaffold X protein disclosed herein, or derivatives thereof, retains the ability to be specifically targeted to EVs, e.g., exosomes. In some aspects, the Scaffold X or a Scaffold X derivative includes one or more mutations, for example, conservative amino acid substitutions.

In some aspects, a variant of a Scaffold Y or derivative thereof comprises a variant having at least 70% identity to MARCKS, MARCKSL1, BASP1 or a fragment of MARCKS, MARCKSL1, or BASP1.

In some aspects, the variant or variant of a fragment of a Scaffold Y protein, or derivative thereof, retains the ability to be specifically targeted to the luminal surface of EVs, e.g., exosomes. In some aspects, the Scaffold Y protein includes one or more mutations, e.g., conservative amino acid substitutions.

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, variants or derivatives include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells.

Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, covalent attachment of glycosylphosphatidylinositol (GPI), hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination.

In some aspects, a Scaffold X protein and/or Scaffold Y protein can modified at any convenient location. In some aspects, a biologically active molecule can be modified at any convenient location. In particular aspects of the present disclosure, an EV, e.g., exosome, component (e.g., a protein such as a Scaffold X protein, a Scaffold Y protein, a lipid, a glycan, or a combination thereof) and/or a biologically active molecule (e.g., an antibody or ADC, a PROTAC, a small molecule such as a cyclic dinucleotide, a toxin such as MMAE, a STING agonist, a tolerizing agent, an antisense oligonucleotide, an antigen such as a vaccine antigen, an adjuvant, a targeting moiety, a tropism moiety, or any combination thereof) can be modified to yield a derivative comprising at least one maleimide moiety.

### II. Conjugated EVs, e.g., Exosomes

Extracellular vesicles (EVs) typically are 20 nm to 1000 nm in diameter; e.g., exosomes, which are small extracellular vesicles, are typically 100 to 200 nm in diameter. EVs, e.g., exosomes, are composed of a limiting lipid bilayer and a diverse set of proteins and nucleic acids (Maas, S. L. N., et al., Trends. Cell Biol. 27(3):172-188 (2017)). EVs, e.g., exosomes, exhibit preferential uptake in discrete cell types and tissues, and their tropism can be directed by adding proteins to their surface that interact with receptors on the surface of target cells (Alvarez-Erviti, L., et al., Nat. Biotechnol. 29(4):341-345 (2011)).

Unlike antibodies, EVs, e.g., exosomes, can accommodate large numbers of molecules attached to their surface, on the order of thousands to tens of thousands of molecules per EV (e.g., exosome). EV (e.g., exosome)-drug conjugates thus represent a platform to deliver a high concentration of therapeutic compound to discrete cell types, while at the same time limiting overall systemic exposure to the compound, which in turn reduces off-target toxicity.

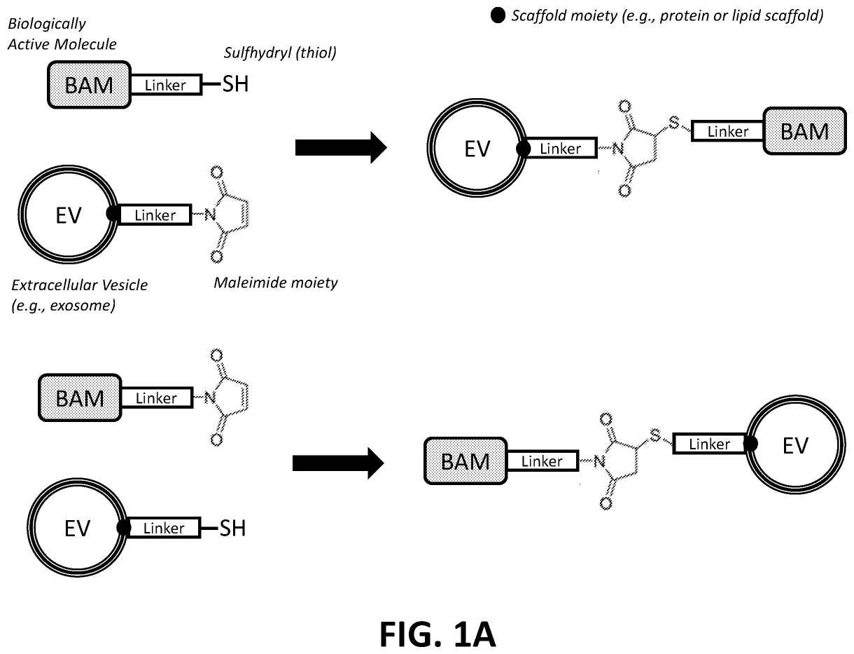

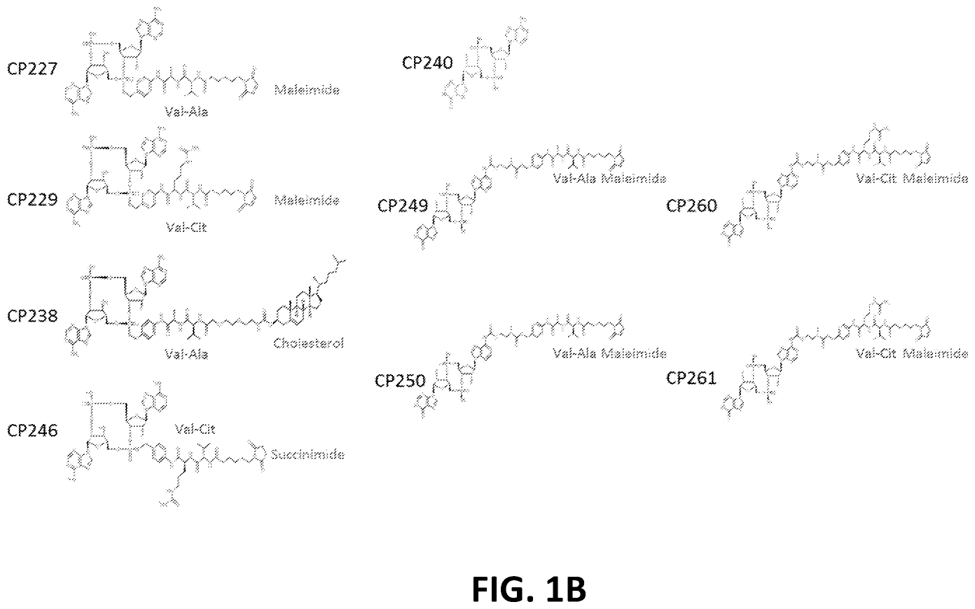

The present disclosure provide EVs, e.g., exosomes, that have been engineered by reacting a first molecular entity comprising a free thiol group with a second molecular entity comprising a maleimide group, wherein the maleimide moiety covalently links the first molecular entity (e.g., an EV, e.g., an exosome, or a component thereof such a Scaffold X protein or a lipid) with the second molecular entity (e.g., a biologically active molecule) via a maleimide moiety as presented in FIG. 1A.

Non-limiting examples of biologically active molecules that can attached to an EV (e.g., exosome) via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), morpholino, amino acids (e.g., amino acids comprising a detectable moiety or a toxin that disrupt translation), polypeptides (e.g., enzymes), lipids, carbohydrates, small molecules (e.g., small molecule drugs and toxins), antigens (e.g., vaccine antigens), adjuvants (e.g., vaccine adjuvants), etc.

In some aspects, an EV (e.g., exosome) of the present disclosure can comprise more than one type of biologically active molecule. In some aspects, biologically active molecules can be, e.g., small molecules such as cyclic dinucleotides, toxins such as auristatins (e.g., monoethyl auristatin E, MMAE), antibodies (e.g., naked antibodies or antibody-drug conjugates), STING agonists, tolerizing agents, antisense oligonucleotides, PROTACs, morpholinos, lysophosphatidic acid receptor antagonists (e.g., LPA1 antagonists) or any combinations thereof. In some aspects, an EV (e.g., exosome) of the present disclosure can comprise, e.g., a vaccine antigen and optionally a vaccine adjuvant. In some aspects, an EV (e.g., exosome) of the present disclosure can comprise a therapeutic payload (e.g., a STING or one payload disclosed below) and a targeting moiety and/or a tropism moiety.

Accordingly, the methods disclosed herein can result in molecule entities as presented in the FIG. 1A, wherein an EV (e.g., an exosome) or any molecular component thereof such as a polypeptide (e.g., a Scaffold X protein or fragment thereof), a lipid, a lipoprotein, a glycoprotein, or any variant or derivative of a naturally occurring or non-naturally occurring protein located on an EV (e.g., exosome) can be chemically linked via a maleimide moiety to a biologically active molecule, e.g., a therapeutic payload, a targeting moiety, a tropism moiety, or any combination thereof. As depicted in FIG. 1A, in some aspects, an EV (e.g., an exosome) or molecular component thereof comprising a sulfhydryl (thiol) group can react with a maleimide group attached to a biologically active moiety. In other aspects, an EV (e.g., an exosome) or molecular component thereof comprising a maleimide group can react with a sulfhydryl (thiol) group present in a biologically active moiety. In both cases, the final product is a biologically active molecule chemically attached to an EV (e.g., an exosome) via a thioether bond.

II.A. Maleimide Moiety

The maleimide moiety can be any chemical moiety comprising a maleimide group (e.g., a bifunctional chemical moiety, that connects the EV, e.g., exosome, to a linker, e.g., a peptide):

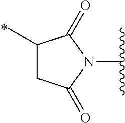 [SMILES: *N1C(=O)CC(C)C1=O]

wherein
(i) * indicates the attachment point to any available maleimide-reacting group present on the EV (e.g., exosome), e.g., a free thiol group of a Scaffold X protein; and,
(ii) the wavy line indicates the attachment site to the rest of the maleimide moiety.

In some aspects, the maleimide moiety attaches to a sulfur atom attached to the EV (e.g., exosome), e.g., a naturally occurring sulfur atom in a thiol group or a sulfur atom introduced via chemical modification or via mutation.

In some aspects, the maleimide moiety has the formula (I):

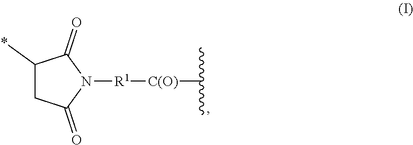

wherein
(i) R¹ is selected from the group consisting of —C₁₋₁₀ alkylene-, —C₃₋₈ carbocyclo-, —O—(C₁₋₈ alkylene)-, -arylene-, —C₁₋₁₀ alkylene-arylene-, -arylene-C₁₋₁₀ alkylene-, —C₁₋₁₀ alkylene-(C₃₋₈ carbocyclo)-, —(C₃₋₈ carbocyclo)-C₁₋₁₀ alkylene-, —C₃₋₈ heterocyclo-, —C₁₋₁₀ alkylene-(C₃₋₈ heterocyclo)-, —(C₃₋₈ heterocyclo)-C₁₋₁₀ alkylene-, —(CH₂CH₂O)ᵣ—, and —(CH₂CH₂O)ᵣ—CH₂—;
(ii) r is an integer, e.g., from 1 to 10;
(iii) * indicates the attachment point to any available reactive sulfur atom, e.g., a sulfur in a thiol group, present on the EV (e.g., exosome); and,
(iv) the wavy line indicates the attachment site of the maleimide moiety to the biologically active molecule.

In some aspects, R¹ is —C₁₋₈ alkylene-, —C₃₋₆ carbocyclo-, —O—(C₁₋₆ alkylene)-, -arylene-, —C₁₋₈ alkylene-arylene-, -arylene-C₁₋₈ alkylene-, —C₁₋₈ alkylene-(C₃₋₆ carbocyclo)-, —(C₃₋₆ carbocyclo)-C₁₋₈ alkylene-, —C₃₋₆ heterocyclo-, —C₁₋₈ alkylene-(C₃₋₆ heterocyclo)-, —(C₃₋₆ heterocyclo)-C₁₋₈ alkylene-, —(CH₂CH₂O)ᵣ—, and —(CH₂CH₂O)ᵣ—CH₂—; where r is an integer, e.g., from 1 to 10;

In some aspects, R¹ is —(CH₂)ₛ—, cyclopentyl, cyclohexyl, —O—(CH₂)ₛ—, -phenyl-, —CH₂-phenyl-, -phenyl-CH₂—, —CH₂-cyclopentyl-, -cyclopentyl- CH₂—, —CH₂-cyclohexyl-, -cyclohexyl-CH₂—, —(CH₂CH₂O)ᵣ—, and —(CH₂CH₂O)ᵣ—CH₂—; where r is an integer, e.g., from 1 to 6.

In some aspects, R¹ is —(CH₂)ₛ—, wherein s is, e.g., 4, 5, or 6.

In some aspects, the maleimide moiety has the formula (II), wherein R¹ is —(CH₂)ₛ—:

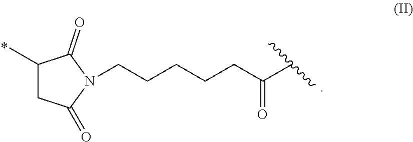 [SMILES: *C(=O)CCCCCN1C(=O)CC(C)C1=O]

In some aspects, the maleimide moiety has the formula (III), wherein R¹ is —(CH₂CH₂O)ᵣ—CH₂—, and wherein r is 2:

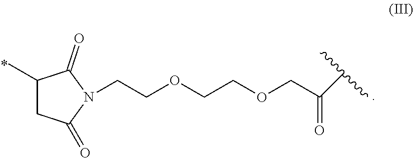 [SMILES: *C(=O)COCCOCCN1C(=O)CC(C)C1=O]

In some aspects, the maleimide moiety is covalently linked to a functional group present on the EV (e.g., exosome), wherein the functional group is a sulfhydryl (thiol) group. In one aspect, the sulfhydryl group is on a protein on the surface of the EV (e.g., exosome), e.g., a Scaffold X protein, or a fragment or variant thereof. For example, in some aspects, the sulfhydryl group can be present on a thiol lipid, e.g., cholesterol-SH, DSPE-SH, or derivatives thereof, e.g., cholesterol-PEG-SH or DSPE-PEG-SH.

In other aspects, the maleimide moiety is covalently linked to a functional group present on the EV (e.g., exosome) which has been chemically derivatized to provide a maleimide moiety. For example, in one aspect, an amine functional group present on the EV (e.g., exosome) (e.g., an amine on the side chain of a lysine or an arginine, or terminal amine group of a protein) can be derivatized with a bifunctional reagent comprising, e.g., a succinimide moiety and a maleimide moiety.

In other aspects, a carboxyl functional group present on the EV (e.g., exosome) (e.g., a carboxyl on the side chain of a glutamic acid or aspartic acid, or terminal carboxyl group of a protein) can be derivatized with a bifunctional reagent comprising, e.g., an isocyanate moiety and a maleimide moiety. In yet other aspects, a carbonyl (oxidized carbohydrate) present on the EV (e.g., exosome) can be derivatized with a bifunctional reagent comprising, e.g., a hydrazine moiety and a maleimide moiety.

In general, the methods disclosed herein can be practiced using any reagent, e.g, a bifunctional or multifunctional reagent, that upon reacting with a molecule present on the surface (external surface or luminal surface) of the EV (e.g., exosome) (e.g., a protein, lipid, sugar) will covalently or non-covalently modify the molecule to yield a modified molecule comprising at least one maleimide moiety. The molecule present on the surface (external surface or luminal surface) of the EV (e.g., exosome) can be naturally occurring, or it can be non-naturally occurring, i.e., it has been modified, e.g., via chemical modification, incubation with a composition comprising the non-naturally occurring molecule, or via mutation (e.g., by introducing one or more cysteine amino acids into a protein via mutation).

Bifunctional reagents comprising a maleimide moiety, reagents in which a number of maleimide-containing units can multimerize, or maleimide-containing reagents that can add a functional moiety (e.g, a PEG) via the maleimide group include, e.g., bifunctional reagents comprising a hydrazine moiety and a maleimide moiety, bifunctional reagents comprising an isocyanate moiety and a maleimide moiety, bifunctional reagents comprising an N-hydroxy succinimidyl ester moiety and a maleimide moiety, bifunctional reagents comprising a succinimide moiety and a maleimide moiety, biotin-maleimide, streptavidin-maleimide, N-4-maleimide butyric acid, N-(4-maleimidebutyloxy) succinimide, N-[5-(3′-maleimide propylamide)-1-carboxypentyl]iminodiacetic acid, maleimide-PEG-succinimidyl esters (e.g., maleimide-PEG₁₂-succinimidyl ester, maleimide-PEG₂-succinimidyl ester, maleimide-PEG₂₀₀₀-succinimidyl ester, maleimide-PEG₅₀₀₀-succinimidyl ester, or maleimide-PEGₙ-succinimidyl ester wherein 1<n<5000), maleimide-PEG-maleimide (e.g., e.g., maleimide-PEG₁₂-maleimide, maleimide-PEG₂-maleimide, maleimide-PEG₂₀₀₀-maleimide, maleimide-PEG₅₀₀₀-maleimide, or maleimide-PEGₙ-maleimide wherein 1<n<5000), maleimide-OH, maleimide-PEGₙ-OH wherein 1<n<5000, Maleimide-poly(ethylene glycol)-b-poly(ε-caprolactone), (S)-(−)—N-(1-phenylethyl)maleimide, N-(4-Chlorophenyl)maleimide, N-(1-Pyrenyl)maleimide, methoxypolyethylene glycol maleimide, poly(ethylene glycol) methyl ether maleimide, N-(4,4,5,5,6,6,7,7,8,8,9,9,10,10,11,11,11-Heptadecafluoroundecyl)maleimide, deferoxamine-maleimide (i.e., a chelator-maleimide), maleimide glycidyl ether, bifunctional maleimido DTPA, bifunctional NOTA-maleimide chelators, homobifunctional maleimide crosslinkers (i.e., those which have a maleimide group at each end), bis-maleimidopolyalkylene glycol, DBCO-maleimide, benzotriazole maleimide, alkyne maleimide, maleimide functionalized lipids, maleimide functionalized PEG lipid, and in general any molecule comprising at least one maleimide moiety at least one additional reactive moiety (e.g., maleimide or another reactive group) and one or more optional linkers (e.g., PEG or another polymer such as polyglycerol).

II.B. Linkers

The EVs, e.g., exosomes, of the present disclosure can comprise one or more linkers that link (i.e., connect) the maleimide moiety to the biologically active molecule or to the EV (e.g., exosome). In some aspects, the maleimide moiety is linked to the biologically active molecule by a linker. The linker can be any chemical moiety capable of, e.g., linking a maleimide moiety, e.g., of formula (II) or (III), to a biologically active molecule. In some aspects, a maleimide moiety can comprise one or more linkers. In some aspects, the linkers disclosed herein or combinations thereof can be used to connect, e.g., a maleimide moiety to a biologically active molecule, a first biologically active moiety to a second biologically active moiety, an EV (e.g., membrane lipid or a scaffold protein thereof) to a maleimide moiety, or an EV (e.g., membrane lipid or a scaffold protein thereof) to a biologically active moiety.

In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present in a maleimide moiety disclosed herein, each of the linkers can be the same or different. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker can comprise at least about 110, at least about 120, at least about 130, at least about 140, at least about 150, at least about 160, at least about 170, at least about 180, at least about 190, or at least about 200 amino acids.

In other aspects, the peptide linker can comprise at least about 200, at least about 300, at least about 400, at least about 500, at least about 600, at least about 700, at least about 800, at least about 900, or at least about 1,000 amino acids. The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

In some aspects, the linker is a glycine/serine linker. In some aspects, the peptide linker is glycine/serine linker according to the formula [(Gly)n-Ser]m (SEQ ID NO: 46) where n is any integer from 1 to 100 and m is any integer from 1 to 100. In other aspects, the glycine/serine linker is according to the formula [(Gly)x-Sery]z (SEQ ID NO: 47) wherein x in an integer from 1 to 4, y is 0 or 1, and z is an integer from 1 to 50. In some aspects, the peptide linker comprises the sequence Gn (SEQ ID NO: 48), where n can be an integer from 1 to 100. In some aspects, the peptide linker can comprise the sequence (GlyAla)n (SEQ ID NO: 49), wherein n is an integer between 1 and 100. In other aspects, the peptide linker can comprise the sequence (GlyGlySer)n (SEQ ID NO: 50), wherein n is an integer between 1 and 100.

In a specific aspect, the sequence of the peptide linker is GGGG (SEQ ID NO: 30).

In some aspects, the peptide linker can comprise the sequence (GlyAla)n, wherein n is an integer between 1 and 100. In other aspects, the peptide linker can comprise the sequence (GlyGlySer)n, wherein n is an integer between 1 and 100.

In other aspects, the peptide linker comprises the sequence (GGGS)n (SEQ ID NO:31). In still other aspects, the peptide linker comprises the sequence (GGS)n(GGGGS)n (SEQ ID NO:217). In these instances, n can be an integer from 1 to 100. In other instances, n can be an integer from one to 20, i.e., 1, 2, 3, 4, 5, 6, 7, 8, 9, 10, 11, 12, 13, 14, 15, 16, 17, 18, 19, or 20. In some aspects n is an integer from 1 to 100.

Additional examples of linkers include, but are not limited to, GGG, SGGSGGS (SEQ ID NO:218), GGSGGSGGSGGSGGG (SEQ ID NO:219), GGSGGSGGGGSGGGGS (SEQ ID NO:220), GGSGGSGGSGGSGGSGGS (SEQ ID NO:221), or GGGGSGGGGSGGGGS (SEQ ID NO:222). In some aspects, the linker is a poly-G sequence (GGGG)n (SEQ ID NO:223), where n can be an integer from 1-100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

In some aspects, the linker comprises a non-peptide linker. In other aspects, the linker consists of a non-peptide linker. In some aspects, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(n-maleimidophenyl)butyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078).

Linkers can be introduced into maleimide moieties using techniques known in the art (e.g., chemical conjugation, recombinant techniques, or peptide synthesis). In some aspects, the linkers can be introduced using recombinant techniques. In other aspects, the linkers can be introduced using solid phase peptide synthesis. In certain aspects, a maleimide moiety disclosed herein can contain simultaneously one or more linkers that have been introduced using recombinant techniques and one or more linkers that have been introduced using solid phase peptide synthesis or methods of chemical conjugation known in the art.

Linkers can be susceptible to cleavage (“cleavable linker”) thereby facilitating release of the biologically active molecule. Thus, in some aspects, a maleimide moiety disclosed herein can comprises a cleavable linker. Such cleavable linkers can be susceptible, for example, to acid-induced cleavage, photo-induced cleavage, peptidase-induced cleavage, esterase-induced cleavage, and disulfide bond cleavage, at conditions under which the biologically active molecule remains active. Alternatively, linkers can be substantially resistant to cleavage (“non-cleavable linker”).

Some cleavable linkers are cleaved by proteases (“protease cleavable linkers”). Only certain peptides are readily cleaved inside or outside cells. See, e.g., Trout et al., 79 Proc. Natl. Acad. Sci. USA, 626-629 (1982) and Umemoto et al. 43 Int. J. Cancer, 677-684 (1989). Cleavable linker can contain cleavable sites composed of α-amino acid units and peptidic bonds, which chemically are amide bonds between the carboxylate of one amino acid and the amino group of a second amino acid. Other amide bonds, such as the bond between a carboxylate and the α-amino acid group of lysine, are understood not to be peptidic bonds and are considered non-cleavable.

In some aspects, the protease-cleavable linker comprises a cleavage site for a protease, e.g., neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases, enamelysin, tumor necrosis factor α-converting enzyme, insulysin, nardilysin, mitochondrial processing peptidase, magnolysin, dactylysin-like metalloproteases, neutrophil collagenase, matrix metallopeptidases, membrane-type matrix metalloproteinases, SP2 endopeptidase, prostate specific antigen (PSA), plasmin, urokinase, human fibroblast activation protein (FAPa), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose- binding protein-associated serine protease, coagulation factors, thrombin, protein c, u and t-type plasminogen activator, cathepsin G, hepsin, prostasin, hepatocyte growth factor- activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin B, cathepsin D, cathepsin L, cathepsin M, proteinase K, pepsins, chymosyn, gastricsin, renin, yapsin and/or mapsins, Prostate-Specific antigen (PSA), or any Asp-N, Glu-C, Lys-C or Arg-C proteases in general. See, e.g., Cancer Res. 77(24):7027-7037 (2017), which is herein incorporated by reference in its entirety. In some aspects, the cleavable linker component comprises a peptide comprising one to ten amino acid residues. In these aspects, the peptide allows for cleavage of the linker by a protease, thereby facilitating release of the biologically active molecule upon exposure to intracellular proteases, such as lysosomal enzymes (Doronina et al. (2003) Nat. Biotechnol. 21:778-784). Exemplary peptides include, but are not limited to, dipeptides, tripeptides, tetrapeptides, pentapeptides, and hexapeptides. Exemplary dipeptides include, but are not limited to, valine-alanine (val-ala), valine-citrulline (val-cit), phenylalanine-lysine (phe-lys), N-methyl-valine-citrulline, cyclohexylalanine-lysine, and beta-alanine-lysine. Exemplary tripeptides include, but are not limited to, glycine-valine-citrulline (gly-val-cit) and glycine-glycine-glycine (gly-gly-gly).

A peptide can comprise naturally-occurring and/or non-natural amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno- cysteine, norleucine (“Ne”), norvaline (“Nva”), beta-alanine, L- or D-naphthalanine, ornithine (“Orn”), and the like. Peptides can be designed and optimized for enzymatic cleavage by a particular enzyme, for example, a tumor-associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

Some linkers are cleaved by esterases (“esterase cleavable linkers”). Only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable liker. Non-cleavable linkers are any chemical moiety capable of linking a maleimide moiety to a biologically active molecule in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a cyclic dinucleotide and/or the antibody does not lose its activity. In some aspects, the biologically active molecule is attached to the linker via a spacer. In one aspect, the spacer is a self-immolative spacer. In another aspect, the spacer is a non self-immolative spacer.

In some aspects, the linker comprises a non-cleavable linker comprising, e.g., tetraethylene glycol (TEG), hexaethylene glycol (HEG), polyethylene glycol (PEG), succinimide, or any combination thereof. In some aspects, the non-cleavable linker comprises a spacer unit to link the biologically active molecule to the non-cleavable linker. In some aspects, one or more non-cleavable linkers comprise smaller units (e.g., HEG, TEG, glycerol, C₂ to C₁₂ alkyl, and the like) linked together. In one aspect, the linkage is an ester linkage (e.g., phosphodiester or phosphorothioate ester) or other linkage.

II.B.1 Ethylene Glycol (HEG, TEG, PEG) Linkers

In some aspects, the linker comprises a non-cleavable linker, wherein the non-cleavable linker comprises a polyethylene glycol (PEG) characterized by a formula R³—(O—CH₂—CH₂)n- or R³-(0-CH₂—CH₂)n-O— with R³ being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

In some aspects, n has a value of 2, 3, 4, 5, 6, 7, 8, 9, 10, 11, 12, 13, 14, 15, 16, 17,18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some specific aspects, n has a value from 3 to 200, from 3 to 20, from 10 to 30, or from 9 to 45.

In some aspects, the PEG is a branched PEG. Branched PEGs have three to ten PEG chains emanating from a central core group.

In certain aspects, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group.

In some aspects, the PEG is a Comb PEGs. Comb PEGs have multiple PEG chains normally grafted onto a polymer backbone.

In certain aspects, the PEG has a molar mass between 100 g/mol and 3000 g/mol, particularly between 100 g/mol and 2500 g/mol, more particularly of approx. 100 g/mol to 2000 g/mol. In certain aspects, the PEG has a molar mass between 200 g/mol and 3000 g/mol, particularly between 300 g/mol and 2500 g/mol, more particularly of approx. 400 g/mol to 2000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₅₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₅₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₅₀₀, PEG₂₉₀₀, or PEG₃₀₀₀. In one particular aspect, the PEG is PEG₄₀₀. In another particular aspect, the PEG is PEG₂₀₀₀.

In some aspects, a linker of the present disclosure can comprise several PEG linkers, e.g., a cleavable linker flanked by PEG, HEG, or TEG linkers.

In some aspects, the linker comprises (HEG)n and/or (TEG)n, wherein n is an integer between 1 and 50, and each unit is connected, e.g., via a phosphate ester linker, a phosphorothioate ester linkage, or a combination thereof.

II.B.2 Glycerol and Polyglycerols (PG)

In some aspects, the linker comprises a non-cleavable linker comprising a glycerol unit or a polyglycerol (PG) described by the formula ((R₃—O—(CH₂—CHOH—CH₂O)ₙ—) with R3 being hydrogen, methyl or ethyl, and n having a value from 3 to 200. In some aspects, n has a value from 3 to 20. In some aspects, n has a value from 10 to 30.

In some aspects, the PG linker is a diglycerol, triglycerol, tetraglycerol (TG), pentaglycerol, or a hexaglycerol (HG) linker.

In some aspects, n has a value of 2, 3, 4, 5, 6, 7, 8, 9, 10, 11, 12, 13, 14, 15, 16, 17,18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some alternatives of these aspects, n has a value from 9 to 45. In some aspects, the heterologous moiety is a branched polyglycerol described by the formula (R³—O—(CH₂—CHOR⁵—CH₂O)ₙ) with R⁵ being hydrogen or a linear glycerol chain described by the formula (R³—O—(CH₂—CHOH—CH₂—O)ₙ—) and R³ being hydrogen, methyl or ethyl. In some aspects, the heterologous moiety is a hyperbranched polyglycerol described by the formula (R³—O—(CH₂—CHOR⁵—CH₂O)ₙ) with R⁵ being hydrogen or a glycerol chain described by the formula (R³—O—(CH₂—CHOR⁶—CH₂—O)ₙ—), with R⁶ being hydrogen or a glycerol chain described by the formula (R³—O—(CH₂—CHOR⁷—CH₂—O)ₙ—), with R⁷ being hydrogen or a linear glycerol chain described by the formula (R³—O—(CH₂—CHOH—CH₂—O)ₙ—) and R³ being hydrogen, methyl or ethyl. Hyperbranched glycerol and methods for its synthesis are described in Oudshorn et al. (2006) Biomaterials 27:5471-5479; Wilms et al. (20100 Acc. Chem. Res. 43, 129-41, and references cited therein.

In certain aspects, the PG has a molar mass between 100 g/mol and 3000 g/mol, particularly between 100 g/mol and 2500 g/mol, more particularly of approx. 100 g/mol to 2000 g/mol. In certain aspects, the PG has a molar mass between 200 g/mol and 3000 g/mol, particularly between 300 g/mol and 2500 g/mol, more particularly of approx. 400 g/mol to 2000 g/mol.

In some aspects, the PG is PG₁₀₀, PG₂₀₀, PG₃₀₀, PG₄₀₀, PG₅₀₀, PG₆₀₀, PG₇₀₀, PG₈₀₀, PG₉₀₀, PG₁₀₀₀, PG₁₁₀₀, PG₁₂₀₀, PG₁₃₀₀, PG₁₄₀₀, PG₁₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₂₇₀₀, PG₂₅₀₀, PG₂₉₀₀, or PG₃₀₀₀. In one particular aspect, the PG is PG₄₀₀. In another particular aspect, the PG is PG₂₀₀₀.

In some aspects, the linker comprises (glycerol)n, and/or (HG)n and/or (TG)n, wherein n is an integer between 1 and 50, and each unit is connected, e.g., via a phosphate ester linker, a phosphorothioate ester linkage, or a combination thereof.

II.B.3 Aliphatic (Alkyl) Linkers

In some aspects, the linker comprises at least one aliphatic (alkyl) linker, e.g., propyl, butyl, hexyl, or C2-C12 alkyl, such as C2-C10 alkyl or C2-C6 alkyl.

In some aspects, the linker comprises an alkyl chain, e.g., an unsubstituted alkyl. In some aspects, the linker combination comprises an substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenyleyl alkenyl, alkenyl aryl alkynyl, alkynyl aryl alkyl, alkynyl aryl alkenyl, alkynyl aryl alkynyl, alkyl heteroaryl alkyl, alkyl heteroaryl alkyl, alkyl heteroaryl alkenyl, alkyl heteroaryl alkynyl, alkenyl heteroaryl alkyl, alkenyl heteroaryl alkenyl, alkenyl heteroaryl alkynyl, alkynyl heteroarylalkyl, alkynylheteroarylalkenyl, alkynylheteroarylalkynyl, alkylheterocyclylalkyl, alkylheterocyclylalkenyl, alkylheterocyclylalkynyl, alkenylheterocyclylalkyl, alkenylheterocyclylalkenyl, or alkenylheterocyclylalkynyl.

Optionally these components are substituted. Substituents include alcohol, alkoxy (such as methoxy, ethoxy, and propoxy), straight or branched chain alkyl (such as C1-C12 alkyl), amine, aminoalkyl (such as amino C1-C12 alkyl), phosphoramidite, phosphate, phosphoramidate, phosphorodithioate, thiophosphate, hydrazide, hydrazine, halogen, (such as F, Cl, Br, or I), amide, alkylamide (such as amide C1-C12 alkyl), carboxylic acid, carboxylic ester, carboxylic anhydride, carboxylic acid halide, ether, sulfonyl halide, imidate ester, isocyanate, isothiocyanate, haloformate, carboduimide adduct, aldehydes, ketone, sulfhydryl, haloacetyl, alkyl halide, alkyl sulfonate, —C(═O)CH═CHC(═O) (maleimide), thioether, cyano, sugar (such as mannose, galactose, and glucose), α,β-unsaturated carbonyl, alkyl mercurial, or α,β-unsaturated sulfone.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one aspect, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-S-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁₀, or C₂-C₈) and at least one heteroatom chosen, e.g., from N, O, S, Si, B and P (in one aspect, N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃.

Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl,” “heterocyclic,” “heterocycle,” or “heterocyclyl,” by itself or in combination with other terms, represents a carbocyclic, non-aromatic ring (e.g., 3- to 8-membered ring and for example, 4-, 5-, 6- or 7-membered ring) containing at least one and up to 5 heteroatoms selected from, e.g., N, O, S, Si, B and P (for example, N, O and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule.

Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. In one aspects, the aryl group is selected from phenyl, benzo[d][1,3]dioxolyl and naphthyl. The aryl group, in yet another aspect, is phenyl.

The term “arylalkyl” or “aralkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” or “aralkyl” groups include benzyl, phenethyl, pyridylmethyl and the like.

By “aryloxy” is meant the group —O-aryl, where aryl is as defined herein. In one example, the aryl portion of the aryloxy group is phenyl or naphthyl. The aryl portion of the aryloxy group, in one aspect, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si and B (for example, N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom.

In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable aryl group substituents described below.

Examples of aliphatic linkers include the following structures: —O—CO—O—, NH—CO—O, —NH—CO—NH—, —NH—(CH₂)ₙ₁—, —S—(CH₂)ₙ₁—, —CO—(CH₂)ₙ₁—CO—, —CO—(CH₂)ₙ₁—NH—, —NH—(CH₂)ₙ₁—NH—, —CO—NH—(CH₂)ₙ₁—NH—CO—, —C(═S)—NH—(CH₂)ₙ₁—NH—CO—, —C(═S)—NH—(CH₂)ₙ₁—NH—C—(═S)—, —CO—O—(CH₂)ₙ₁—O—CO—, —C(═S)—O—(CH₂)ₙ₁—O—CO—, —C(═S)—O—(CH₂)ₙ₁—O—C(═S), —CO NH (CH₂)ₙ₁—O—CO, —C(═S) NH (CH₂)ₙ₁—O—CO—, —C(═S)—NH—(CH₂)ₙ₁—O—C(═S)—, —CO—NH—(CH₂)ₙ₁—O—CO—, —C(═S)—NH—(CH₂)ₙ₁—CO—, —C(═S)—O—(CH₂)ₙ₁—NH—CO—, —C(═S)—NH—(CH₂)ₙ₁—O—C—(═S)—, —NH—(CH₂CH₂O)ₙ₂—CH(CH₂OH)—, —NH—(CH₂CH₂O)ₙ₂—CH₂—, —NH—(CH₂CH₂O)ₙ₂—CH₂—CO—, —O—(CH₂)ₙ₃—S—S—(CH₂)ₙ₄—O—P(═O)₂, —CO—(CH₂)ₙ₃—O—CO—NH—(CH₂)ₙ₄—, —CO—(CH₂)ₙ₃—CO—NH—(CH₂)ₙ₄—, —(CH₂)ₙ₁NH—, —C(O)(CH₂)ₙ₁NH—, —C(O)—(CH₂)ₙ₁—C(O)—, —C(O)—(CH₂)ₙ₁—C(O)O—, —C(O)—O—, —C(O)—(CH₂)ₙ₁—NH C(O), —C(O)—(CH₂)ₙ₁, —C(O) NH—, —C(O)—, (CH₂)ₙ₁—C(O)—, (CH₂)ₙ₁—C(O)O—, (CH₂)ₙ₁—, (CH₂)ₙ₁—NH—C(O), wherein n1 is an integer between 1 and 40 (e.g., 2 to 20, or 2 to 12); n2 is an integer between 1 and 20 (e.g., 1 to 10, or 1 to 6); n3 and n4 can be the same or different, and are an integer between 1 and 20 (e.g., 1 to 10, or 1 to 6).

In some aspects, the linker comprises (C3)n, (C4)n, (C5)n, (C6)n, (C7)n, or (C8)n, or a combination thereof, wherein n is an integer between 1 and 50, and each unit is connected, e.g., via a phosphate ester linker, a phosphorothioate ester linkage, or a combination thereof.

II.B.4 Cleavable Linkers

In some aspects, the linker can be a cleavable linker. The term cleavable linker refers to a linker comprising at least one linkage or chemical bond that can be broken or cleaved. As used herein, the term cleave refers to the breaking of one or more chemical bonds in a relatively large molecule in a manner that produces two or more relatively smaller molecules. Cleavage can be mediated, e.g., by a nuclease, peptidase, protease, phosphatase, oxidase, or reductase, for example, or by specific physicochemical conditions, e.g., redox environment, pH, presence of reactive oxygen species, or specific wavelengths of light.

In some aspects, the term “cleavable,” as used herein, refers, e.g., to rapidly degradable linkers, such as, e.g., phosphodiester and disulfides, while the term “non-cleavable” refers, e.g., to more stable linkages, such as, e.g., nuclease-resistant phosphorothioates. In some aspects, the cleavable linker is a dinucleotide or trinucleotide linker, a disulfide, an imine, a thioketal, a val-cit dipeptide, or any combination thereof. In some aspects, the cleavable linker comprises valine-alanine-p-aminobenzylcarbamate or valine-citrulline-p-aminobenzylcarbamate.

II.B.4.a Redox Cleavable Linkers

In some aspects, the linker comprises a redox cleavable linker. As a non-limiting example, one type of cleavable linker is a redox cleavable linking group that is cleaved upon reduction or upon oxidation. In some aspects, the redox cleavable linker contains a disulfide bond, i.e., it is a disulfide cleavable linker. Redox cleavable linkers can be reduced, e.g., by intracellular mercaptans, oxidases, or reductases.

II.B.4.b Reactive Oxygen Species (ROS) Cleavable Linkers

In some aspects, the linker can comprise a cleavable linker which can be cleaved by a reactive oxygen species (ROS), such as superoxide (O₂⁻) or hydrogen peroxide (H₂O₂), generated, e.g., by inflammation processes such as activated neutrophils. In some aspects, the ROS cleavable linker is a thioketal cleavable linker. See, e.g., U.S. Pat. No. 8,354,455B2, which is herein incorporated by reference in its entirety.

II.B.4.c pH Dependent Cleavable Linkers

In some aspects, the linker is an “acid labile linker” comprising an acid cleavable linking group, which is a linking group that is selectively cleaved under acidic conditions (pH<7). As a non-limiting example, the acid cleavable linking group is cleaved in an acidic environment, e.g., about 6.0, 5.5, 5.0 or less. In some aspects, the pH is about 6.5 or less. In some aspects, the linker is cleaved by an agent such as an enzyme that can act as a general acid, e.g., a peptidase (which can be substrate specific) or a phosphatase. Within cells, certain low pH organelles, such as endosomes and lysosomes, can provide a cleaving environment to the acid cleavable linking group. Although the pH of human serum is 7.4, the average pH in cells is slightly lower, ranging from about 7.1 to 7.3. Endosomes also have an acidic pH, ranging from 5.5 to 6.0, and lysosomes are about 5.0 at an even more acidic pH. Accordingly, pH dependent cleavable linkers are sometimes called endosomically labile linkers in the art.

The acid cleavable group can have the general formula —C═NN—, C (O)—O—, or —OC (O). In another non-limiting example, when the carbon attached to the ester oxygen (alkoxy group) is attached to an aryl group, a substituted alkyl group, or a tertiary alkyl group such as dimethyl pentyl or t-butyl, for example. Examples of acid cleavable linking groups include, but are not limited to amine, imine, amino ester, benzoic imine, diortho ester, polyphosphoester, polyphosphazene, acetal, vinyl ether, hydrazone, cis-aconitate, hydrazide, thiocarbamoyl, imizine, azidomethyl-methylmaleic anhydride, thiopropionate, a masked endosomolytic agent, a citraconyl group, or any combination thereof. Disulfide linkages are also susceptible to pH.

In some aspects, the linker comprises a low pH-labile hydrazone bond. Such acid-labile bonds have been extensively used in the field of conjugates, e.g., antibody-drug conjugates. See, for example, Zhou et al. (2011) Biomacromolecules 12:1460-7; Yuan et al. (2008) Acta Biomater. 4:1024-37; Zhang et al. (2008) Acta Biomater. 6:838-50; Yang et al. (2007) J. Pharmacol. Exp. Ther. 321:462-8; Reddy et al. (2006) Cancer Chemother. Pharmacol. 58:229-36; Doronina et al. (2003) Nature Biotechnol. 21:778-84, all of which are herein incorporated by reference in their entireties.

In certain aspects, the linker comprises a low pH-labile bond selected from the following: ketals that are labile in acidic environments (e.g., pH less than 7, greater than about 4) to form a diol and a ketone; acetals that are labile in acidic environments (e.g., pH less than 7, greater than about 4) to form a diol and an aldehyde; imines or iminiums that are labile in acidic environments (e.g., pH less than 7, greater than about 4) to form an amine and an aldehyde or a ketone; silicon-oxygen-carbon linkages that are labile under acidic condition; silicon-nitrogen (silazane) linkages; silicon-carbon linkages (e.g., arylsilanes, vinylsilanes, and allylsilanes); maleamates (amide bonds synthesized from maleic anhydride derivatives and amines); ortho esters; hydrazones; activated carboxylic acid derivatives (e.g., esters, amides) designed to undergo acid catalyzed hydrolysis); or vinyl ethers.

Further examples can be found in U.S. Pat. Nos. 9,790,494B2 and 8,137,695B2, the contents of which are incorporated herein by reference in their entireties.

II.B.4.d Enzymatic Cleavable Linkers

In some aspects, the linker can comprise a linker cleavable by intracellular or extracellular enzymes, e.g., proteases, esterases, nucleases, amidases. The range of enzymes that can cleave a specific linker in a linker combination depends on the specific bonds and chemical structure of the linker. Accordingly, peptidic linkers can be cleaved, e.g., by peptidases, linkers containing ester linkages can be cleaved, e.g., by esterases; linkers containing amide linkages can be cleaved, e.g., by amidases; etc.

II.B.4.e Protease Cleavable Linkers

In some aspects, the linker comprises a protease cleavable linker, i.e., a linker that can be cleaved by an endogenous protease. Only certain peptides are readily cleaved inside or outside cells. See, e.g., Trout et al., (1982) Proc. Natl. Acad. Sci. USA 79:626-629, and Umemoto et al. (1989) Int. J. Cancer 43:677-684. Cleavable linkers can contain cleavable sites composed of α-amino acid units and peptidic bonds, which chemically are amide bonds between the carboxylate of one amino acid and the amino group of a second amino acid. Other amide bonds, such as the bond between a carboxylate and the α-amino acid group of lysine, are understood not to be peptidic bonds and are considered non-cleavable.

In some aspects, the protease-cleavable linker comprises a cleavage site for a protease, e.g., neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases, enamelysin, tumor necrosis factor α-converting enzyme, insulysin, nardilysin, mitochondrial processing peptidase, magnolysin, dactylysin-like metalloproteases, neutrophil collagenase, matrix metallopeptidases, membrane-type matrix metalloproteinases, SP2 endopeptidase, prostate specific antigen (PSA), plasmin, urokinase, human fibroblast activation protein (FAPa), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose- binding protein-associated serine protease, coagulation factors, thrombin, protein c, u and t-type plasminogen activator, cathepsin G, hepsin, prostasin, hepatocyte growth factor- activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin B, cathepsin D, cathepsin L, cathepsin M, proteinase K, pepsins, chymosyn, gastricsin, renin, yapsin and/or mapsins, Prostate-Specific antigen (PSA), or any Asp-N, Glu-C, Lys-C or Arg-C proteases in general. See, e.g., Cancer Res. 77(24):7027-7037 (2017), which is herein incorporated by reference in its entirety. In some aspects, the cleavable linker component comprises a peptide comprising one to ten amino acid residues. In these aspects, the peptide allows for cleavage of the linker by a protease, thereby facilitating release of the biologically active molecule upon exposure to intracellular proteases, such as lysosomal enzymes (Doronina et al. (2003) Nat. Biotechnol. 21:778-784). Exemplary peptides include, but are not limited to, dipeptides, tripeptides, tetrapeptides, pentapeptides, and hexapeptides.

A peptide can comprise naturally-occurring and/or non-natural amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno- cysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor-associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

Exemplary dipeptides include, but are not limited to, valine-alanine, valine-citrulline, phenylalanine-lysine, N-methyl-valine-citrulline, cyclohexylalanine-lysine, and beta-alanine-lysine. Exemplary tripeptides include, but are not limited to, glycine-valine-citrulline (gly-val-cit) and glycine-glycine-glycine (gly-gly-gly).

ILB.4.f Esterase Cleavable Linkers

Some linkers are cleaved by esterases (“esterase cleavable linkers”). Only certain esters can be cleaved by esterases and amidases present inside or outside of cells. Esters are formed by the condensation of a carboxylic acid and an alcohol. Simple esters are esters produced with simple alcohols, such as aliphatic alcohols, and small cyclic and small aromatic alcohols. Examples of ester-based cleavable linking groups include, but are not limited to, esters of alkylene, alkenylene and alkynylene groups. The ester cleavable linking group has the general formula —C(O)—O— or —OC(O)—.

II.B.4.g Phosphatase Cleavable Linkers

In some aspects, a linker combination can includes a phosphate-based cleavable linking group is cleaved by an agent that degrades or hydrolyzes phosphate groups. An example of an agent that cleaves intracellular phosphate groups is an enzyme such as intracellular phosphatase. Examples of phosphate-based linking groups are —O—P (O) (ORₖ)—O—, —O—P (S) (ORₖ)—O—, —O—P (S) (SRₖ)—O—, —S—P (O) (ORₖ)—O—, —O—P (O) (ORₖ)—S—, —S—P (O) (ORₖ)—S—, —O—P (S) (ORₖ)—S—, —SP (S) (ORₖ)—O—, —OP (O) (Rₖ)—O—, —OP (S) (Rₖ)—O—, —SP (O) (Rₖ)—O—, —SP (S) (Rₖ)—O—, —SP (O) (Rₖ)—S—, or —OP (S) (Rₖ)—S—, wherein, Rₖ is NH₂, BH₃, CH₃, C₁₋₆ alkyl, C₆₋₁₀ aryl, C₁₋₆ alkoxy or C₆₋₁₀ aryl-oxy. In some aspects, C₁₋₆ alkyl and C₆₋₁₀ aryl are unsubstituted. Further non-limiting examples are —O—P (O) (OH)—O—, —O—P (S) (OH)—O—, —O—P (S) (SH)—O—, —S—P (O) (OH)—O—, —O—P (O) (OH)—S—, —S—P (O) (OH)—S—, —O—P (S) (OH)—S—, —S—P (S) (OH)—O—, —O—P (O) (H)—O—, —O—P (S) (H)—O—, —S—P (O) (H)—O—, —SP (S) (H)—O—, —SP (O) (H)—S—, —OP (S) (H)—S—, or —O—P (O) (OH)—O—.

II.B.4.h Photoactivated Cleavable Linkers

In some aspects, the combination comprises a photoactivated cleavable linker, e.g., a nitrobenzyl linker or a linker comprising a nitrobenzyl reactive group.

II.C Self-immolative Spacer

In some aspects, the self-immolative spacer in the EV (e.g., exosome) of the present disclosure undergoes 1,4 elimination after the enzymatic cleavage of the protease-cleavable linker. In some aspects, the self-immolative spacer in the EV (e.g., exosome) of the present disclosure undergoes 1,6 elimination after the enzymatic cleavage of the protease-cleavable linker. In some aspects, the self-immolative spacer is, e.g., a p-aminobenzyl carbamate (PABC), a p-amino benzyl ether (PABE), a p-amino benzyl carbonate, or a combination thereof.

In certain aspects, the self-immolative spacer comprises an aromatic group. In some aspects, the aromatic group is selected from the group consisting of benzyl, cinnamyl, naphthyl, and biphenyl. In some aspects, the aromatic group is heterocyclic. In other aspects, the aromatic group comprises at least one substituent. In some aspects, the at least one substituent is selected from the group consisting of F, Cl, I, Br, OH, methyl, methoxy, NO₂, —NH₂, NO³⁺, NHCOCH₃, N(CH₃)₂, —NHCOCF₃, alkyl, haloalkyl, C₁-C₈ alkylhalide, carboxylate, sulfate, sulfamate, and sulfonate.

In other aspects, at least one C in the aromatic group is substituted with N, O, or C—R″, wherein R″ is independently selected from H, F, Cl, I, Br, OH, methyl, methoxy, NO₂, NH₂, NO³⁺, —NHCOCH₃, N(CH₃)₂, —NHCOCF₃, alkyl, haloalkyl, C₁-C₈ alkylhalide, carboxylate, sulfate, sulfamate, and sulfonate.

In some aspects, the self-immolative spacer comprises an aminobenzyl carbamate group, an aminobenzyl ether group, or an aminobenzyl carbonate group. In one aspect, the self-immolative spacer is p-amino benzyl carbamate (PABC). P-amino benzyl carbamate (PABC) is the most efficient and most widespread connector linkage for self-immolative site-specific prodrug activation (see, e.g., Carl et al. (1981) J. Med. Chem. 24:479-480; WO 1981/001145; Rautio et al. (2008) Nature Reviews Drug Discovery 7:255-270; Simplicio et al. (2008) Molecules 13:519-547, all of which are herein incorporated by reference in their entireties). PABC allows the release of any amine drugs, peptides, and proteins upon cleavage by a protease and 1,6 spontaneous fragmentation.

In some aspects, the self-immolative spacer connects a biologically active molecule (e.g., an antibody) to a protease-cleavable substrate. In specific aspects, the carbamate group of a PABC self-immolative spacer is connected to the N-terminus of a biologically active molecule (e.g., an antibody), and the amino group of the PABC self-immolative spacer is connected to a protease-cleavable substrate.

The aromatic ring of the aminobenzyl group can optionally be substituted with one or more (e.g., R₁ and/or R₂) substituents on the aromatic ring, which replace a hydrogen that is otherwise attached to one of the four non-substituted carbons that form the ring. As used herein, the symbol “Rₓ” (e.g., R₁, R₂, R₃, R₄) is a general abbreviation that represents a substituent group as described herein.

Substituent groups can improve the self-immolative ability of the p-aminobenzyl group. See Hay et al. (1999) J. Chem Soc., Perkin Trans. 1:2759-2770; see also, Sykes et al. J. (2000) Chem. Soc., Perkin Trans. 1:1601-1608.

Self-immolative elimination can take place, e.g., via 1,4 elimination, 1,6 elimination (e.g., PABC), 1,8 elimination (e.g., p-amino-cinnamyl alcohol), D-elimination, cyclisation-elimination (e.g., 4-aminobutanol ester and ethylenediamines), cyclization/lactonization, cyclization/lactolization, etc. See, e.g., Singh et al. (2008) Curr. Med. Chem. 15:1802-1826 and Greenwald et al. (2000) J. Med. Chem. 43:475-487.

In some aspects, the self-immolative spacer can comprise, e.g., cinnamyl, naphthyl, or biphenyl groups (see, e.g., Blencowe et al. (2011) Polym. Chem. 2:773-790). In some aspects, the self-immolative spacer comprises a heterocyclic ring (see., e.g., U.S. Pat. Nos. 7,375,078; 7,754,681). Numerous homoaromatic (see, e.g., Carl et al. (1981) J. Med. Chem. 24:479; Senter et al. (1990) J. Org. Chem. 55:2975; Taylor et al. (1978) J. Org. Chem. 43:1197; Andrianomenjanahary et al. (1992) Bioorg. Med. Chem. Lett. 2:1903), and coumarin (see, e.g., Weinstein et al. (2010) Chem. Commun. 46:553), furan, thiophene, thiazole, oxazole, isoxazole, pyrrole, pyrazole (see, e.g., Hay et al. (2003) J. Med. Chem. 46:5533), pyridine (see, e.g., Perry-Feigenbaum et al. (2009) Org. Biomol. Chem. 7:4825), imidazone (see, e.g., Nailor et al. (1999) Bioorg. Med. Chem. Lett. Z:1267; Hay and Denny (1997) Tetrahedron Lett. 38:8425), and triazole (see, e.g., Bertrand and Gesson (2007) J. Org. Chem. 72:3596) based heteroaromatic groups that are self-immolative under both aqueous and physiological conditions are known in the art. See also, U.S. Pat. Nos. 7,691,962; 7,091,186; and U.S. Pat. Publ. Nos. US2006/0269480; US2010/0092496; US2010/0145036; US2003/0130189; and US2005/0256030, all of which are herein incorporated by reference in their entireties.

In some aspects, a maleimide moiety disclosed herein comprises more than one self-immolative spacer in tandem, e.g., two or more PABC units. See, e.g., de Groot et al. (2001) J. Org. Chem. 66:8815-8830. In some aspects, a maleimide moiety disclosed herein can comprise a self-immolative spacer (e.g., a p-aminobenzylalcohol or a hemithioaminal derivative of p-carboxybenzaldehyde or glyoxilic acid) linked to a fluorigenic probe (see, e.g., Meyer et al. (2010) Org. Biomol. Chem. 8:1777-1780).

Where substituent groups in the self-immolative linkers are specified by their conventional chemical formulae, written from left to right, they equally encompass the chemically identical substituents, which would result from writing the structure from right to left. For example, “—CH₂O—” is intended to also recite “—OCH₂—”.

Substituent groups in self-immolative, for example, R₁ and/or R₂ substituents in a p-aminobenzyl self-immolative linker as discuss above can include, e.g., alkyl, alkylene, alkenyl, alkynyl, alkoxy, alkylamino, alkylthio, heteroalkyl, cycloalkyl, heterocycloalkyl, aryl, arylalkyl, aryloxy, heteroaryl, etc. When a compound of the present disclosure includes more than one substituent, then each of the substituents is independently chosen.

In some specific aspects, the linker has the formula (IV):

-Aₐ-Y_{y}— (IV),

wherein each -A- is independently an amino acid unit, a is independently an integer from 1 to 12; —Y— is a spacer unit, and y is 0, 1, or 2. In some aspects, -Aₐ- is a dipeptide, a tripeptide, a tetrapeptide, a pentapeptide, or a hexapeptide. In some aspects, -Aₐ- is selected from the group consisting of valine-alanine, valine-citrulline, phenylalanine-lysine, N-methylvaline-citrulline, cyclohexylalanine-lysine, and beta-alanine-lysine. In some aspects, -Aₐ- is valine-alanine or valine-citrulline. In some aspects, y is 1. In some aspects, —Y— is a self-immolative spacer.

In some aspects, the self-immolative spacer —Y_{y}— has the formula (V):

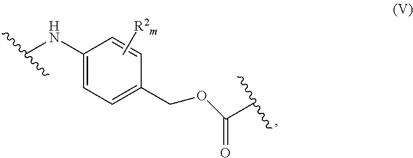

wherein each R² is independently C₁₋₈ alkyl, —O—(C₁₋₈ alkyl), halogen, nitro, or cyano; and m is an integer from 0 to 4. In some aspects, m is 0, 1, or 2. In some aspects, m is 0.

In some aspects, the cleavable linker of formula (IV) is valine-alanine-p-aminobenzylcarbamate or valine-citrulline-p-aminobenzylcarbamate. In some aspects, the spacer unit—Y— is a non self-immolative spacer, such as for example, -Gly- or -Gly-Gly-.

In some aspects, the linker is an acid labile linker. In some aspects, the acid labile linker comprises a cis-aconitic linker, a hydrazide linker, a thiocarbamoyl linker, or any combination thereof. In some aspects, the acid labile linker comprises a spacer unit to link the biologically active molecule to the acid labile linker. Suitable spacer units are those described above in connection with —Y_{y}—.

In some aspects, the linker is a non-cleavable linker comprising, e.g., tetraethylene glycol (TEG), polyethylene glycol (PEG), succinimide, or any combination thereof. In some aspects, the non-cleavable linker comprises a spacer unit to link the biologically active molecule to the non-cleavable linker.

In some aspects, the present disclosure provides an EV (e.g., exosome) comprising a biologically active molecule and a cleavable linker, wherein the cleavable linker connects the EV (e.g., exosome) to the biologically active molecule, and the cleavable linker comprises valine-alanine-p-aminobenzylcarbamate or valine-citrulline-p-aminobenzylcarbamate. In some aspects, the EV (e.g., exosome) further comprises a maleimide moiety, which links the EV (e.g., exosome) to the cleavable linker via a functional group present on the EV (e.g., exosome). Suitable maleimide moieties are those described above, such as for example, maleimide moieties of formulae (I), (II), and (III). In some aspects, the maleimide moiety is covalently linked to a functional group present on the EV (e.g., exosome), wherein the functional group is sulfhydryl (thiol), wherein the sulfhydryl group is on a protein on the surface of the EV (e.g., exosome), for example, the external surface of the EV (e.g., exosome).

The present disclosure also provides an EV (e.g., exosome) comprising a maleimide moiety, a cleavable linker, and a biologically active molecule, wherein the maleimide moiety links the EV (e.g., exosome) to the cleavable linker, and the cleavable linker connects the maleimide moiety to the biologically active molecule.

II.D Biologically Active Molecules

In some aspects, an EV (e.g., exosome) disclosed herein is capable of delivering a payload (e.g., a biologically active molecule chemically linked to the EV, e.g., exosome, via a maleimide moiety) to a target. The payload is an agent that acts on a target (e.g., a target cell) that is contacted with the EV (e.g., exosome). Contacting can occur in vitro or in a subject. Non-limiting examples of payloads that can be linked to an EV (e.g., exosome), e.g., chemically linked via a maleimide moiety, include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), morpholino, amino acids (e.g., amino acids comprising a detectable moiety or a toxin that disrupt translation), polypeptides (e.g., enzymes), lipids, carbohydrates, small molecules (e.g., small molecule drugs and toxins), antigens (e.g., vaccine antigens), adjuvants, or combinations thereof.

In some aspects, an EV (e.g., exosome) can comprise more than one payload, e.g., a first payload in solution the lumen of EV (e.g., exosome), and a second payload linked, e.g., to the external surface of the EV (e.g., exosome) via a maleimide moiety. In some aspects, the payload comprises a small molecule. In some aspects, the payload comprises a peptide. In some aspects, the payload comprises an antigen, e.g., a vaccine antigen. In some aspects, the payload comprises a vaccine adjuvant.

II.D.1 Payloads Targeting Antigens and Vaccine Antigens

In some aspects, the payload interacts with an antigen, e.g., a tumor antigen. In some aspects, the biological function of the antigen, e.g., a tumor antigen, is modulated by the interaction with the payload (e.g., if the antigen is a receptor, the payload may be a receptor agonist or a receptor antagonist). In other aspects, the payload comprises an antigen capable of inducing an immune reaction (i.e., a vaccine antigen). In some aspects, the payload can comprise an antigen capable of inducing an immune reaction (i.e., a vaccine antigen) and an adjuvant (i.e., a vaccine adjuvant). In some aspects, the vaccine antigen and vaccine adjuvant can be on the same EV, e.g., exosome. In other aspects, the vaccine and vaccine adjuvant can be in different EVs, e.g., exosomes.

Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, or combinations thereof.

Payloads interacting with and, e.g., modulating the biological function of tumor antigens comprise, e.g., antibodies and binding fragments thereof, aptamers, antibody drug conjugates (ADC), and small molecules.

In some aspects, the antigen is a universal tumor antigen. As used herein, the term “universal tumor antigen” refers to an immunogenic molecule, such as a protein, that is, generally, expressed at a higher level in tumor cells than in non-tumor cells and also is expressed in tumors of different origins. In some aspects, the universal tumor antigen is expressed in more than about 30%, about 40%, about 50%, about 60%, about 70%, about 80%, about 90% or more of cancers (e.g., human cancers). In some aspects, the universal tumor antigen can be expressed in non-tumor cells (e.g., normal cells) but at lower levels than it is expressed in tumor cells. In certain aspects, the expression level of the universal tumor antigen is greater than about 1-fold, about 2-fold, about 3-fold, about 4-fold, about 5-fold, about 6-fold, about 7-fold, about 8-fold, about 9-fold, about 10-fold or more on tumor cells compared to non-tumor cells. In certain aspects, the universal tumor antigen is not expressed in normal cells and only expressed in tumor cells. Non-limiting examples of universal tumor antigens that can be used with the present disclosure include endothelial lining antigens in tumor vasculature, survivin, tumor protein D52 (TPD52), androgen receptor epitopes, ephrin type-A receptor 2 (EphA2), human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (D1).

In further aspects, an antigen can comprise a neoantigen. As used herein, the term “neoantigen” refers to antigens encoded by tumor-specific mutated genes.

In some aspects, the antigen is derived from a bacterium, a virus, fungus, protozoa, or any combination thereof. In some aspects, the antigen is derived from an oncogenic virus (also referred to herein as cancer associated viruses (CAVs)). In further aspects, the antigen is derived from a group comprising: a Human Gamma herpes virus 4 (i.e., Epstein Barr virus (EBV)), influenza A virus, influenza B virus, cytomegalovirus, Staphylococcus aureus, Mycobacterium tuberculosis, Chlamydia trachomatis, HIV-1, HIV-2, corona viruses (e.g., COVID-19, MERS-CoV, and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B virus (HBV), Hepatitis C virus (HCV), human T-lymphotropic virus (HTLV1), human herpes virus 8 (HHV8), Merkel cell polyomavirus (MCV), herpes simplex virus 2 (HSV-2), Klebsiella sp., Pseudomonas aeruginosa, Enterococcus sp., Proteus sp., Enterobacter sp., Actinobacter sp., coagulase-negative staphylococci (CoNS), Mycoplasma sp., Adenovirus, Adeno-associated virus (AAV), or combinations thereof.

In some aspects, the antigen derived from EBV is BZLF1. BZLF1 (also known as Zta or EB1) is an immediate-early viral gene of EBV, which induces cancers and infects primarily the B-cells of 95% of the human population. This gene (along with others) produces the expression of other EBV genes in other stages of disease progression, and is involved in converting the virus from the latent to the lytic form. ZEBRA (BamHI Z Epstein-Barr virus replication activator, also known as Zta and BZLF1)) is an early lytic protein of EBV encoded by BZLF1. See Hartlage et al. (2015) Cancer Immunol. Res. 3(7): 787-94, and Rist et al. (2015) J. Virology 70:703-12, both of which are incorporated herein by reference in their entireties. EV, e.g., exosomes, disclosed herein comprising an EBV antigen, e.g., BZLF1, can be used, e.g., to treat post-transplant lymphoproliferative disorder (PTLD). Such EV can be administered to EBV negative patients receiving EBV positive transplants. BZLF1 is a dominant T cell antigen associated with durable remission in PTLD patients. The EV, e.g., exosomes, disclosed herein comprising BZLF1 can elicit a potent CD8 T-cell mediated immunity to BZLF1. Accordingly, mucosal immunity and tissue resident memory cells can protect the patient from developing PTLDF. Non-limiting exemplary antigens include, but are not limited to, the antigens disclosed in U.S. Pat. No. 8,617,564B2.

In some aspects, the antigen is derived from Mycobacterium tuberculosis to induce cellular and/or humoral immune response. In some aspects, the antigen comprises one or more epitopes of Mycobacterium tuberculosis (TB antigen). Various antigens are associated with Mycobacterium tuberculosis infection, including ESAT-6, TB10.4, CFP10, Rv2031 (hspX), Rv2654c (TB7.7), and Rv1038c (EsxJ). See, e.g., Lindestam et al., J. Immunol. 188(10):5020-31 (2012), which is incorporated herein in its entirety. In some aspects, the antigen useful for the present disclosure comprises one or more epitopes of ESAT6. In some aspects, the antigen useful for the present disclosure comprises one or more epitopes of TB10.4. In some aspects, the antigen useful for the present disclosure comprises one or more epitopes of CFP10. In some aspects, the antigen useful for the present disclosure comprises one or more epitopes of Rv2031 (hspX). In some aspects, the antigen useful for the present disclosure comprises one or more epitopes of Rv2654c (TB7.7). In some aspects, the antigen useful for the present disclosure comprises one or more epitopes of Rv1038c (EsxJ). In some aspects, the antigen useful for the present disclosure comprises an epitope selected from the group consisting of ESAT6, TB10.4 (ESAT-6-like protein EsxH; cfp7), CFP10, Rv2031 (hspX), Rv2654c (TB7.7), Rv1038c (EsxJ), and any combination thereof.

In some aspects, the TB antigen comprises a particular epitope of a TB antigen, e.g., a particular epitope of ESAT6 or TB10.4. In some aspects, the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in MTEQQWNFAGIEAAASAIQGNVTSIHSLLDEGKQSLTKLAAAWGGSGSEAYQGVQQK WDATATELNNALQNLARTISEAGQAMASTEGNVTGMFA (SEQ ID NO: 230; GenBank: AWM98862.1). In some aspects, wherein the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in

(SEQ ID NO: 231; NCBI Reference Sequence:
WP_057308237.1)
MSQIMYNYPAMLGHAGDMAGYAGTLQSLGAEIAVEQAALQSAWQGDTGI
TYQAWQAQWNQAMEDLVRAYHAMSSTHEANTMAMMARDPAEAAKWGG.

In some aspects, an antigen comprises a self-antigen. As used herein, the term “self-antigen” refers to an antigen that is expressed by a host cell or tissue. Under normal healthy state, such antigens are recognized by the body as self and do not elicit an immune response. However, under certain diseased conditions, a body's own immune system can recognize self-antigens as foreign and mount an immune response against them, resulting in autoimmunity. In certain aspects, EVs, e.g., exosomes, of the present disclosure can comprise a self-antigen (i.e., the self (germline) protein to which T cell responses have been induced and resulted in autoimmunity). Such EVs, e.g., exosomes, can be used to target the autoreactive T cells and suppress their activity. Non-limiting examples of self-antigens (including the associated disease or disorder) include: (i) beta-cell proteins, insulin, islet antigen 2 (IA-2), glutamic acid decarboxylase (GAD65), and zinc transporter 8 (ZNT8) (type I diabetes), (ii) myelin oligodendrocyte glycoprotein (MOG), myelin basic protein (MBP), proteolipid protein (PLP), and myelin-associated glycoprotein (MAG) (multiple sclerosis), (iii) citrullinated antigens and synovial proteins (rheumatoid arthritis), (iv) aquaporin-4 (AQP4) (neuromyelitis optica), (v) nicotinic acetylcholine receptors (nAChRs) (myasthenia gravis), (vi) desmoglein-1 (DSG1) and desoglein-2 (DSG2) (pemphigus vulgaris), (v) thyrotropin receptor (Graves' disease), (vi) type IV collagen (Goodpasture syndrome), (vii) thyroglobulin, thyroid peroxidase, and thyroid-stimulating hormone receptor (TSHR) (Hashimoto's thyroiditis), or (viii) combinations thereof.

II.D.2 Protacs

In some aspects, the payload comprises a proteolysis-targeting chimera (PROTAC). PROTACs are heterobifunctional molecules consisting of a ligand to a target protein, a ligand to the E3 ubiquitinating ligase, and a linker connecting the two ligands. Once the target:PROTAC:E3 ternary complex is formed, E2 ubiquitin-conjugating enzymes transfer ubiquitin to lysine residues on the surface of the target protein. In some aspects, the PROTAC target is, e.g., ERa, BCR-ABL, BRD4, PDE4, ERRa, RIPK2, c-ABL, BRD2, BRD3, BRD4, FKBP12, TBK1, BRD9, EGFR, c-Met, Sirt2, CDK9, FLT3, BTK, ALK, AR, TRIM24, SMAD3, RAR, PI3K, PCAF, METAP2, HER2, HDAC6, GCN5, ERK1/2, DHODH, CRABP-II, FLT4, or CK2. In some aspects, the PROTAC target ligand is, e.g., 4-OHT, dasatinib, JQ1, a PDE4 inhibitor, JQ1, a chloroalkane, a thizolidinedione-based ligand, a RIPK2 inhibitor, bosutinib, a JQ1 derivative, OTX015, steel factor, a TBK1 inhibitor, BI-7273, lapatinib, gefitinib, afatinib, foretinib, Sirt2 inhibitor 3b, HJB97, SNS—032, an aminopyrazole analog, AC220, RN-486, ceritinib, an AR antagonist, IACS—7e, or an ibrutinib derivative. In some aspects, the PROTAC E3 ligand is, e.g., an LCL161 derivative, VHL1, a hydroxyproline derivative, pomalidomide, thalidomide, a HIF-1α-derived (R)-hydroxyproline, VHL ligand 2, a VH032 derivative, lenalidomide, a thalidomide derivative, or VL-269. In some aspects, the E3 ligase is, e.g., IAP, VHL, or CRBN. See, for example, An & Fu (2018) EBioMedicine 36:553-562, which is herein incorporated by reference in its entirety.

PROTACS and related technologies that can be used according to the methods disclosed herein are disclosed for example in WO2018106870, US2018155322, WO2018098288, WO2018098280, WO2018098275, WO2018089736, WO2018085247, US20180125821, US20180099940, WO2018064589, WO2018053354, WO2017223452, WO2017201449, WO2017197056, WO2017197051, WO2017197046, WO2017185036, WO2017185034, WO2017185031, WO2017185023, WO2017182418, US20170305901, WO2017176708, US20170281784, WO2017117474, WO2017117473, WO2017079723, U.S. Pat. No. 9,938,264, US20170065719, WO2017024319, WO2017024318, WO2017024317, US20170037004, US20170008904, US20180147202, WO2018051107, WO2018033556, US20160272639, US20170327469, WO2017212329, WO2017211924, US20180085465, US20160045607, US20160022642, WO2017046036, US20160058872, US20180134688, US20180118733, US20180050021, U.S. Pat. No. 9,855,273, US20140255361, U.S. Pat. No. 9,115,184, US20180093990, US20150119435, US20140356322, US20140112922, U.S. Pat. No. 9,765,019, US20180100001, U.S. Pat. No. 7,390,656, or U.S. Pat. No. 7,208,157, all of which are herein incorporated by reference in their entireties.

In some aspects, when several PROTACs are present on an EV (e.g., exosome), such PROTACs can be the same or they can be different. In some aspects, when several PROTACs are present on an EV (e.g., exosome) disclosed herein, such PROTACs can be the same or they can be different. In some aspects, an EV (e.g., exosome) composition of the present disclosure can comprise two or more populations of EVs, e.g., exosomes, wherein each population of EVs, e.g., exosomes, comprises a different PROTAC or combination thereof.

In some aspects, the PROTAC comprises at least one sulfhydryl group, wherein the maleimide moiety links the EV, e.g., exosome, to the PROTAC. In some aspects, a sulfhydryl group is located on the E3 ligase ligand moiety of the PROTAC. In some aspects, a sulfhydryl group is located on the target protein ligand moiety of the PROTAC. In some aspects, a sulfhydryl group is located on the linker moiety of the PROTAC. In some aspects, the sulfhydryl group is a naturally occurring reactive group in the PROTAC.

In other aspects, the maleimide moiety is introduced in the PROTAC, for example, via chemical derivatization. In some aspects, chemical derivatization takes place via a bifunctional linker (bifunctional reagent) which comprises a moiety capable of reacting with a chemical group present in the PROTAC, a moiety comprising a moiety capable of reacting with a maleimide moiety disclosed herein.

In some aspects, the E3 ligase ligand is attached to the PROTAC via a cleavable linker, e,g, PABC. In other aspects, the target ligand is attached to the PROTAC via a cleavable linker, e,g, PABC. In other aspects, both the E3 ligase ligand and the target ligand are attached to the PROTAC via cleavable linkers. In some aspects, both cleavable linkers can be the same cleavable linker. In other aspects, both cleavable linkers are different.

The functionality of PROTACs, e.g., PROTACs linked to an EV, e.g., an exosome disclosed herein, can be assessed according to in vitro and in vivo methods known in the art. For example, since the PROTAC induces ubiquitine-mediated degradation of the target protein, PROTAC activity can be determined using assays that directly measure the degradation of the target protein (e.g., Western blots) or measure functional activities mediated by the target protein (e.g., changes in phosphorylation or phosphorylation-mediated cell signaling if the target protein is a protein kinase).

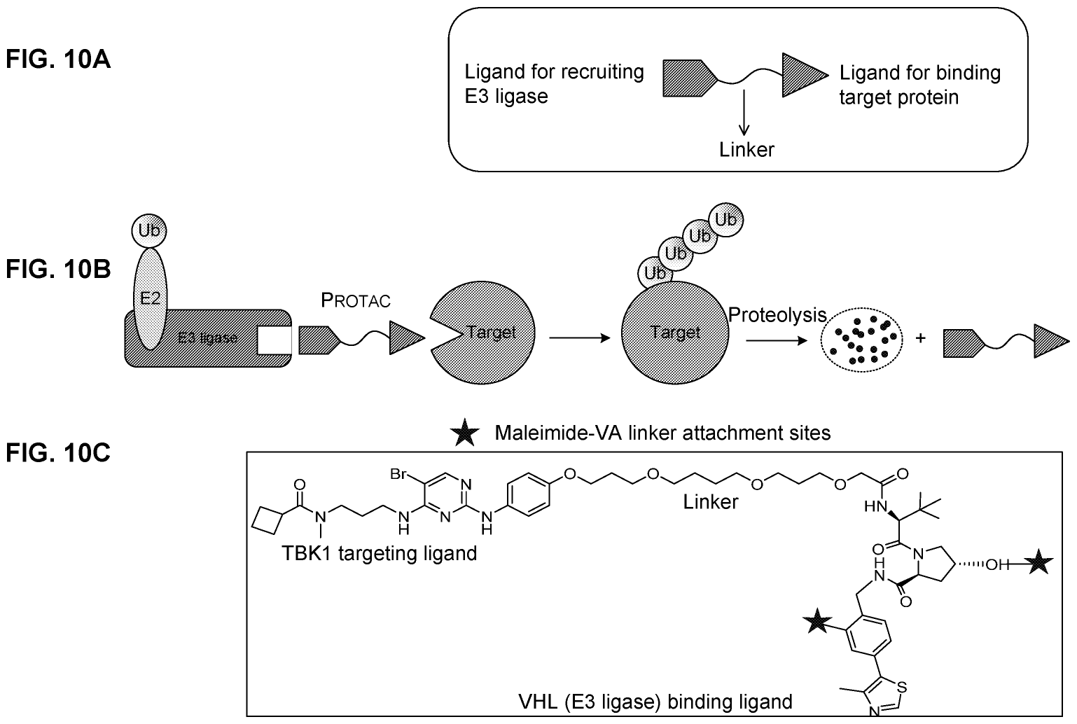

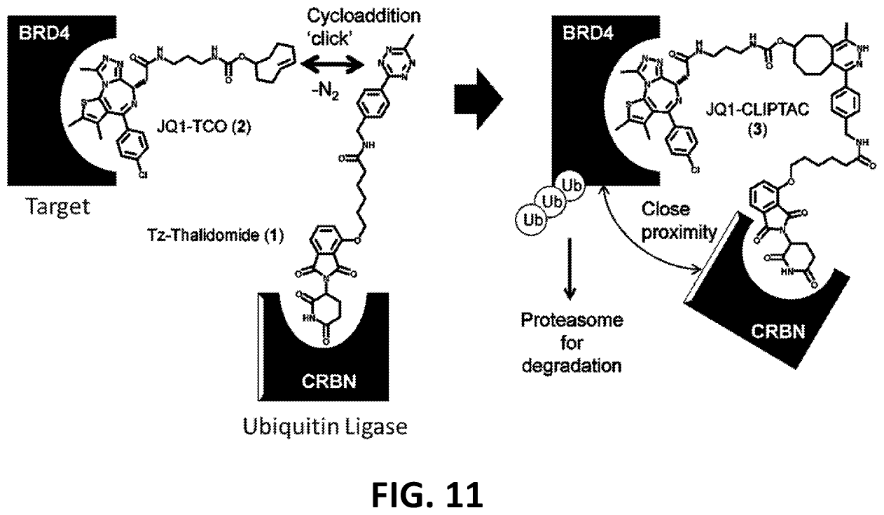

In some specific aspects, the PROTAC comprises a TBK1 targeting ligand, a linker, and a VHL (E3 ligase) binding ligands (see, e.g., FIG. 10C).

In other aspects, the EV, e.g., an exosome, comprises two precursors for the formation of a CLIPTAC (click-formed PROTAC). Accordingly, an EV, e.g., an exosome of the present disclosure can comprise two populations of CLIPTAC precursors linked to the EV via maleimide moieties. Upon binding of the UV to a target cell, the CLIPTAC precursors can be combined intracellularly by bio-orthogonal click combination to yield a heterobifunctional PROTAC. See Lebraud et al. (2016) ACS Cent. Sci. 2:927-934, which is herein incorporated by reference in its entirety.

In general, a PROTAC can be described according to the formula [ULM]-[L]-[PTM], wherein [ULM] is an Ubiquitin-L binding Moiety (a first ligand), [L] is a linker, and [PTM] is a Protein Targeting Moiety (a second ligand). Exemplary PROTACs are shown in the following table. The table indicates the ubiquitinating enzyme targeted by [ULM] and its corresponding [ULM] ligand, as well as the protein targeted by [PTM] and its corresponding [ULM] ligand.

TABLE 1
Exemplary PROTACS

| | [ULM] binding | [ULM] targeted | [PTM] target | |
|---|---|---|---|---|
| Reference | ligand | enzyme | protein | [PTM] ligand |
| US7041298B2 | IkB-alpha or an | SCF E3 ubiquitin | MetAP-2 | Ovalicin |
| | IkB-alpha peptide | ligase | (methionine | |
| | | | aminopeptidase-2) | |
| US7208157B2 | IkB-alpha or an | SCF E3 ubiquitin | Estrogen Receptor | Estradiol |
| | IkB-alpha peptide | ligase | | |
| US6638734B1 | Protein-degradation | E3 ubiquitin- | Any | Any |
| | binding domain of | protein ligase | | |
| | Siah-1 | SIAH1 | | |
| US7390656B2 | Specific ligands | BAG-1, APC, SIP- | Any | Any |
| | disclosed in | L, SIP-S or Siah-1 | | |
| | reference | E3 ubiquitin- | | |
| | | protein ligases | | |
| US9163330B2 | Stapled or stitched | Any ubiquitinating | Any | Stapled or stitched |
| | ligands disclosed in | enzyme | | ligands disclosed in |
| | reference | | | reference |
| US9765019B2 | Arg-Tag disclosed | None (Arg-Tag | Any | Any |
| | in reference | triggers | | |
| | | degradation by | | |
| | | directing complex | | |
| | | to proteasome) | | |
| US20140112922A1 | U-box motif | None (the | Any | Any (e.g., antibody) |
| | | PROTAC includes | | |
| | | a U-box motif, i.e., | | |
| | | a functional E3 | | |
| | | ligase that is | | |
| | | capable of | | |
| | | ubiquitinating the | | |
| | | target) | | |
| US20140356322A1 | Small molecule | Any E3 ubiquitine | Any | Any |
| | ubiquitin ligase | ligase | | |
| | binding moiety | | | |
| | with —OH or prolyl | | | |
| | group | | | |
| US20150119435A1 | Small molecule | None | Any | Any |
| | hydrophobic tag | | | |
| | (degradation tag or | | | |
| | degron) that | | | |
| | induces | | | |
| | proteasomal | | | |
| | degradation | | | |
| US9758522B2 | Small molecule | None | Any | Any |
| | hydrophobic tag | | | |
| | (degradation tag or | | | |
| | degron) that | | | |
| | induces | | | |
| | proteasomal | | | |
| | degradation | | | |
| US20180050021A1 | Specific ligands | Any E3 ubiquitin | Any | Any |
| | targeting E3 | ligase | | |
| | ubiquitin ligase | | | |
| | disclosed in the | | | |
| | reference | | | |
| US20180118733A1 | IAP binder | E3 ubiquitin | Any | Any |
| | | protein ligase IAP | | |
| US20180134688A1 | IAP binder | E3 ubiquitin | RIP2 kinase | RIP2 kinase inhibitor |
| | | protein ligase IAP | | |
| US20150291562A1 | Small molecule | Cereblon E3 | Any | Any |
| | ligands binding | Ubiquitin Ligase | | |
| | Cereblon E3 | | | |
| | Ubiquitin Ligase | | | |
| | (e.g., thalidomide, | | | |
| | lenalidomide, | | | |
| | pomalidomide, | | | |
| | analogs thereof, | | | |
| | isosteres thereof, or | | | |
| | derivatives thereof) | | | |
| US20160058872A1 | Small molecule | Cereblon E3 | Bromodomain- | Ligand binding to a |
| | ligands binding | Ubiquitin Ligase | containing protein or | bromodomain- |
| | Cereblon E3 | | polypeptide | containing protein or |
| | Ubiquitin Ligase | | | polypeptide |
| | (e.g., thalidomide, | | | |
| | lenalidomide, | | | |
| | pomalidomide, | | | |
| | analogs thereof, | | | |
| | isosteres thereof, or | | | |
| | derivatives thereof) | | | |
| WO2017046036A1 | Small molecule | Cereblon E3 | RIP2 kinase | RIP2 kinase small |
| | ligands binding | Ubiquitin Ligase | | molecule ligand |
| | Cereblon E3 | | | |
| | Ubiquitin Ligase | | | |
| US20160022642A1 | Any | Any | Androgen receptor | Androgen receptor |
| | | | | small molecule ligand |
| US20160045607A1 | Small molecule | Any | Estrogen Related | Ligand targeting |
| | ubiquitin ligase | | Receptor Alpha | Estrogen Related |
| | binding moiety | | | Receptor Alpha |
| | with a functional | | | |
| | group that can be | | | |
| | metabolized to | | | |
| | —OH | | | |
| US9694084B2 | Small molecule | E3 Ubiquitin | SMARCA2 or RAS | Small molecule with |
| | ligands binding E3 | Ligase (preferred | | specific formula, |
| | Ubiquitin Ligase | ligase is cereblon) | | which binf to |
| | with specific | | | SMARCA2 or RAS |
| | formulas disclosed | | | |
| | in reference | | | |
| US9821068B2 | Small molecule | E3 Ubiquitin | CREBBP, TRIM24, | Small molecule with |
| | ligands binding E3 | Ligase (preferred | BRPF1, | specific formula |
| | Ubiquitin Ligase | ligase is cereblon) | glucocorticoid | disclosed in reference |
| | with specific | | receptor, estrogen | |
| | formulas disclosed | | receptor, androgen | |
| | in reference | | receptor, DOT1L, | |
| | | | BRAF, HER3, Bcl- | |
| | | | 2, Bcl-XL, HDAC, | |
| | | | orPPAR-gamma | |
| US9770512B2 | Small molecule | E3 Ubiquitin | SMARCA2 or RAS | Small molecule with |
| | ligands binding E3 | Ligase (preferred | | specific formula, |
| | Ubiquitin Ligase | ligase is cereblon) | | which bind to |
| | with specific | | | SMARCA2 or RAS |
| | formulas disclosed | | | |
| | in reference | | | |
| US9750816B2 | Small molecule | E3 Ubiquitin | CREBBP, TRIM24, | Small molecules with |
| | ligands binding E3 | Ligase (preferred | BRPF1, | specific formula |
| | Ubiquitin Ligase | ligase is cereblon) | glucocorticoid | disclosed in reference |
| | with specific | | receptor, estrogen | |
| | formulas disclosed | | receptor, androgen | |
| | in reference | | receptor, DOT1L, | |
| | | | BRAF, HER3, Bcl- | |
| | | | 2, Bcl-XL, HDAC, | |
| | | | or ASPECTPPAR- | |
| | | | gamma | |
| US20180009779A1 | Small molecule | E3 Ubiquitin | BET bromodomain- | Specific small |
| | ligands binding E3 | Ligase (preferred | containing protein | molecule ligands |
| | Ubiquitin Ligase | ligase is cereblon) | (preferred aspect is | binding to |
| | with specific | | BRD4) | bromodomain |
| | formulas disclosed | | | disclosed in reference |
| | in reference | | | |
| US20180085465A1 | Small molecule | E3 Ubiquitin | FKBP12 (12-kDa | Specific small |
| | ligands binding E3 | Ligase (preferred | FK506-binding | molecule ligands |
| | Ubiquitin Ligase | ligase is cereblon) | protein; a cell cycle | binding to FKBP12 |
| | with specific | | regulator) | disclosed in reference |
| | formulas disclosed | | | |
| | in reference | | | |
| US20180134684A1 | Small molecule | E3 Ubiquitin | FKBP12 (12-kDa | Specific small |
| | ligands binding E3 | Ligase (preferred | FK506-binding | molecule ligands |
| | Ubiquitin Ligase | ligase is cereblon) | protein; a cell cycle | binding to FKBP12 |
| | with specific | | regulator) | disclosed in reference |
| | formulas disclosed | | | |
| | in reference | | | |
| WO2017211924A1 | IAP binder | E3 ubiquitin | IRAK3, GAK, TEC, | Ligand binding to |
| US20190152946A1 | | protein ligase IAP | PTK2B(PYK2), | IRAK3, GAK, TEC, |
| | | | AURKA, | PTK2B(PYK2), |
| | | | RPS6KA1(RSK3), | AURKA, |
| | | | MAPK9(JNK2), | RPS6KA1(RSK3), |
| | | | BTK, PTK2 or | MAPK9(JNK2), BTK, |
| | | | AKT2 | PTK2 or AKT2 |
| WO2017212329A1 | Broad, ligand | E3 ubiquitin | Any | Any |
| | binding to E3 | protein ligase (e.g., | | |
| | ubiquitin protein | cereblon) | | |
| | ligase (ligand can | | | |
| | be peptide, | | | |
| | antibody, small | | | |
| | molecule, etc) | | | |
| US20160214972A1 | Ligand binding to | E3 ubiquitin | Androgen receptor | Specific androgen |
| | E3 ubiquitin protein | protein ligase, e.g., | | receptor small |
| | ligase | von Hippel Lindau | | molecule ligands |
| | | E3 ubiquitin | | disclosed in reference |
| | | protein ligase | | |
| US20160272639A1 | Ligand binding to | Von Hippel Lindau | Any | Any |
| | Von Hippel Lindau | E3 ubiquitin | | |
| | E3 ubiquitin protein | protein ligase | | |
| | ligase | | | |
| WO2018033556A1 | Ligand binding to | Cereblon E3 | Kinases AAK1, | Ligands binding to |
| | Cereblon E3 | ubiquitin protein | ABL1, AURKA, | kinases AAK1, ABL1, |
| | ubiquitin protein | ligase | AURKB, BTK, | AURKA, AURKB, |
| | ligase | | GAK, IRAK3, | BTK, GAK, IRAK3, |
| | | | LATSl, MAPK9, | LATSl, MAPK9, |
| | | | PRKAA1, PTK2, | PRKAA1, PTK2, |
| | | | PTK2B, RPS6KA1, | PTK2B, RPS6KA1, |
| | | | RPS6KA3, or TEC | RPS6KA3, or TEC |
| | | | | disclosed in the |
| | | | | reference |
| WO2018051107A1 | Specific ligands | E3 ubiquitin | Any | Any |
| | comprising | protein ligase | | |
| | fluorohydroxy | | | |
| | proline derivatives | | | |
| | that bind to E3 | | | |
| | ubiquitin protein | | | |
| | ligases disclosed in | | | |
| | the reference | | | |
| US20180147202A1 | Ligand binding to | E3 ubiquitin | TANK-binding | TBK 1 binding ligand |
| | E3 ubiquitin protein | protein ligase, e.g., | kinase 1 | |
| | ligase | Von Hippel-Lindau | | |
| | | (VHL) E3 ubiquitin | | |
| | | ligase, IAP, | | |
| | | cereblon, or | | |
| | | MDM2. | | |
| US20170008904A1 | Ligand binding to | MDM2 E3 | Any | Any |
| | MDM2 E3 | ubiquitin protein | | |
| | ubiquitin protein | ligase | | |
| | ligase | | | |
| US20170037004A1 | Ligand binding to | IAP E3 ubiquitin | Any | Any |
| | IAP E3 ubiquitin | protein ligase | | |
| | protein ligase | | | |
| US20170065719A1 | Ligand binding to | E3 ubiquitin | Bromodomain and | Small molecule |
| | E3 ubiquitin protein | protein ligase | extra-terminal | bromodomain and |
| | ligase selected from | selected from the | domain (BET) | extra-terminal domain |
| | the group | group consisting of | | (BET)-containing |
| | consisting of VHL, | VHL, IAP, | | protein targeting |
| | IAP, Cereblon, and | Cereblon, and | | moiety |
| | MDM2 | MDM2 | | |
| US9938264B2 | Ligand binding to | Von Hippel Lindau | Tyrosine kinase, | Tyrosine kinase |
| | E3 ubiquitin protein | (VHL) E3 ubiquitin | e.g., c-ABL or BCR- | inhibitor |
| | ligase | ligase or Cereblon | ABL | |
| | | (CRBN) E3 ligase | | |
| WO2017117473A1 | Small molecule | E3 ubiquitin | Her3 | Her3 small molecule |
| | binding to E3 | protein ligase | | ligands with specific |
| | ubiquitin protein | | | structures disclosed in |
| | ligase (degron | | | reference |
| WO2017117474A1 | Small molecule | E3 ubiquitin | Her3 | Her3 small molecule |
| US20190016703A1 | binding to E3 | protein ligase | | ligands with specific |
| | ubiquitin protein | | | structures disclosed in |
| | ligase (degron | | | reference |
| WO2017182418A1 | Specific IAP | E3 ubiquitin | RIPK2 kinase | RIPK2 kinase |
| US20190119271A1 | inhibitors disclosed | protein ligase IAP | | inhibitor |
| | in reference | | | |
| WO2017185023A1 | Molecule (degron) | E3 ubiquitin | CDK9 kinase | Specific ligands |
| US20190111143A1 | binding to E3 | protein ligase | | binding CDK9 |
| | ubiquitin protein | | | disclosed in the |
| | ligase | | | reference |
| WO2017185031A1 | Molecule (degron) | E3 ubiquitin | CDK4 or CDK6 | Specific ligands |
| US20190092768A1 | binding to E3 | protein ligase | kinase | binding CDK4 or |
| | ubiquitin protein | | | CDK6 kinase |
| | ligase | | | disclosed in the |
| | | | | reference |
| WO2017185034A1 | Molecule (degron) | E3 ubiquitin | CDK8 kinase | Specific ligands |
| US20190112307A1 | binding to E3 | protein ligase | | binding CDK8 |
| | ubiquitin protein | | | disclosed in the |
| | ligase | | | reference |
| WO2017185036A1 | Molecule (degron) | E3 ubiquitin | EGFR | Specific ligands |
| US20190106417A1 | binding to E3 | protein ligase | | binding EGFR |
| | ubiquitin protein | | | disclosed in reference |
| | ligase | | | |
| WO2017197046A1 | C3-carbon | E3 Ubiquitin | Any | Any |
| US20190076542A1 | substituted- | protein ligase | | |
| | glutarimides | | | |
| | degrons binding to | | | |
| | E3 Ubiquitin | | | |
| | protein ligase | | | |
| | disclosed in | | | |
| | reference | | | |
| WO2017197051A1 | Amine-linker C3- | E3 Ubiquitin | Any | Any |
| US20190076539A1 | glutaramide | protein ligase | | |
| | degrons binding to | | | |
| | E3 Ubiquitin | | | |
| | protein ligase | | | |
| | disclosed in | | | |
| | reference | | | |
| WO2017197055A1 | Heterocyclic | E3 Ubiquitin | Any | Any |
| US20190076541A1 | degrons binding to | protein ligase | | |
| | E3 Ubiquitin | | | |
| | protein ligase | | | |
| | disclosed in | | | |
| | reference | | | |
| WO2017197056A1 | Degron binding to | E3 Ubiquitin | Bromodomain | Ligand binding to |
| | E3 Ubiquitin | protein ligase | containing protein | bromodomain |
| | protein ligase | | | |
| WO2017201449A1 | Ligand binding to | E3 Ubiquitin | Any | Any |
| US20190175612A1 | E3 Ubiquitin | protein ligase | | |
| | protein ligase | | | |
| WO2017223415A1 | Ligand binding to | E3 Ubiquitin | TRIM24 | Ligand binding to |
| | E3 Ubiquitin | protein ligase | | TRIM24 |
| | protein ligase | | | |
| WO2017223452A1 | Ligand binding to | E3 Ubiquitin | BRD9 | Ligand binding to |
| | E3 Ubiquitin | protein ligase | | BRD9 |
| | protein ligase | | | |
| WO2018053354A1 | Indole derivative | E3 Ubiquitin | Estrogen Receptor | Ligand binding to |
| US20180072711A1 | degrons disclosed | protein ligase | | Estrogen Receptor |
| | in reference | | | disclosed in reference |
| WO2018064589A1 | Small molecule | Mutant E3 | Any | Any |
| US10239888B2 | degrons capable of | Ubiquitin protein | | |
| | binding to mutant | ligase (cereblon | | |
| | cereblon E3 | mutant) | | |
| | ubiquitin protein | | | |
| | ligase | | | |
| WO2018071606A1 | Degrons binding to | Cereblon E3 | Androgen receptor | Specific ligand |
| US20180099940A1 | cereblon E3 | Ubiquitin protein | | binding to androgen |
| | ubiquitin protein | ligase | | receptor disclosed in |
| | ligase | | | reference |
| WO2018102067A2 | Ligand binding to | E3 Ubiquitin | Tau-protein | Specific ligand |
| US20180125821A1 | E3 Ubiquitin | protein ligase, e,g., | | binding to Tau-protein |
| | protein ligase, e,g., | (Von Hippel | | disclosed in reference |
| | (Von Hippel | Lindau (VHL) E3 | | |
| | Lindau (VHL) E3 | ubiquitin ligase or | | |
| | ubiquitin ligase or | Cereblon (CRBN) | | |
| | Cereblon (CRBN) | E3 ligase | | |
| | E3 ligase | | | |
| WO2018085247A1 | Ligand binding to | E3 ubiquitin ligase | MALT1 (Mucosa- | Specific ligand |
| | E3 ubiquitin ligase | | associated lymphoid | binding to MALT1 |
| | | | tissue lymphoma | disclosed in reference |
| | | | translocation protein | |
| | | | 1) | |
| WO2018089736A1 | Ligand binding to | E3 ubiquitin ligase | Protein Kinase | Specific ligands |
| | E3 ubiquitin ligase | | disclosed in | binding to protein |
| | | | reference | kinases disclosed in |
| | | | | reference |
| WO2018098275A1 | Ligand binding to | E3 ubiquitin ligase | Bruton's Tyrosine | Specific ligands |
| | E3 ubiquitin ligase | | Kinase (BTK) | binding to BTK |
| | | | | disclosed in reference |
| WO2018098280A1 | Ligand binding to | E3 ubiquitin ligase | Protein Kinases | Specific ligands |
| | E3 ubiquitin ligase | | disclosed in | binding to protein |
| | | | reference | kinases disclosed in |
| | | | | reference |
| WO2018098288A1 | Ligand binding to | E3 ubiquitin ligase | Bruton's Tyrosine | Specific ligands |
| | E3 ubiquitin ligase | | Kinase (BTK) | binding to BTK |
| | | | | disclosed in reference |
| WO2018102725A1 | Ligand binding to | E3 ubiquitin ligase | Estrogen receptor | Tetrahydronaphthalene |
| US20180155322A1 | E3 ubiquitin ligase | | | or |
| | | | | tetrahydroisoquinoline |
| | | | | ligands binding to |
| | | | | estrogen receptor |
| WO2018106870A1 | Pomalidomide, | E3 ubiquitin ligase | CDK4/6 kinase | Abemaciclib, |
| | thalidomide, | | | palbociclib, ribociclib, |
| | lenalidomide, | | | trilaciclib, G1T38, |
| | VHL-1, | | | SHR6390, or analogs |
| | adamantane, or | | | thereof |
| | analogs thereof | | | |

All other patent application and patent disclosed in the table above are incorporated by reference in their entireties.

Specific linkers that can be used in PROTACs are disclosed, for example, U.S. Pat. Appl. Publs, US20180050021A1, US20180118733A1, US20180009779A1, US20180085465A1, US20180134684A1, and US20180134688A1, U.S. Pat. Nos. U.S. Pat. No. 96,940,841B2, U.S. Pat. No. 98,210,681B2, U.S. Pat. No. 97,705,121B2, and U.S. Pat. No. 97,508,161B2, and, Int'l. Appl. Publ. WO2018085247A1 which are herein incorporated by reference in their entireties. WO2017212329A1 discloses the formation of a PROTAC comprising a linker generated via click reaction.

II.D.3 Stimulator of Interferon Gene (STING) Agonists

In some aspects, the payload comprises a nucleotide, wherein the nucleotide is a stimulator of interferon genes protein (STING) agonist. STING is a cytosolic sensor of cyclic dinucleotides that is typically produced by bacteria. Upon activation, it leads to the production of type I interferons and initiates an immune response

In some aspects, the EV (e.g., exosome) of the present disclosure comprises one or more STING agonists linked to the EV (e.g., exosome), e.g., chemically linked via a maleimide moiety. In some aspects, the STING agonist comprises a cyclic nucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

It is contemplated that any STING agonist can be used. Among the STING agonists are DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMIP, cAIM(PS)2, 3′3′-cAIMIP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In a specific aspect, the STING agonist is 3′3′-cAIMPdFSH, alternatively named 3-3 cAIMPdFSH. Additional STING agonists known in the art can also be used.

In some aspects, the STING agonist useful for the present disclosure comprises a compound selected from the group consisting of:

In some aspects, the STING agonist useful for the present disclosure comprises a compound having the following formula:

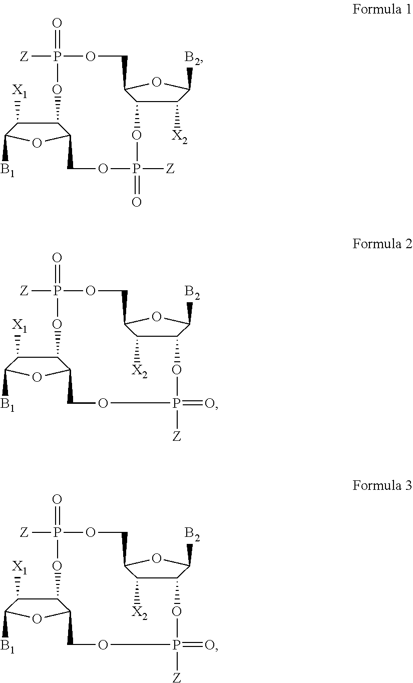

wherein:
X₁ is H, OH, or F;
X₂ is H, OH, or F;
Z is OH, OR₁, SH or SR₁, wherein:
R₁ is Na or NH₄, or
R₁ is an enzyme-labile group which provides OH or SH in vivo such as pivaloyloxymethyl;

B1 and B2 are bases chosen from:

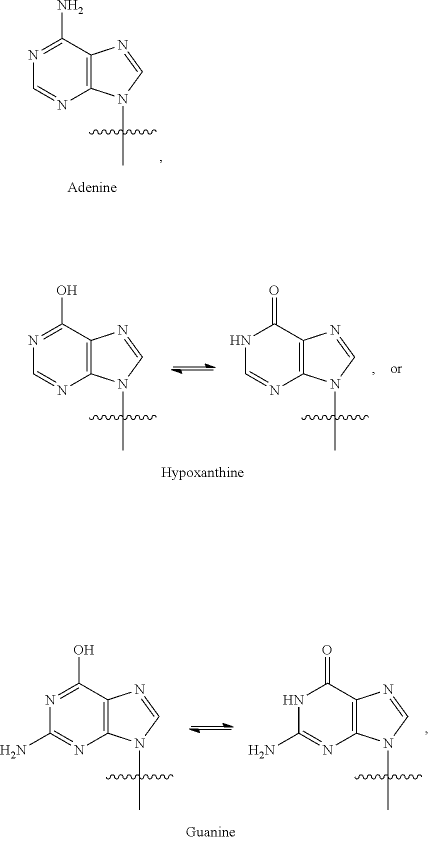 [SMILES: *n1cnc2c(N)ncnc21 ; *n1cnc2c(O)ncnc21 ; *n1cnc2c(=O)[nH]cnc21 ; *n1cnc2c(O)nc(N)nc21 ; *n1cnc2c(=O)[nH]c(N)nc21]

with proviso that:
in Formula 1: X₁ and X₂ are not OH,
in Formula 2: when X₁ and X₂ are OH, B₁ is not Adenine and B₂ is not Guanine, and
in Formula 3: when X₁ and X₂ are OH, B₁ is not Adenine, B₂ is not Guanine and Z is not OH. See WO 2016/096174, the content of which is incorporated herein by reference in its entirety.

In some aspects, the STING agonist useful for the present disclosure comprises:

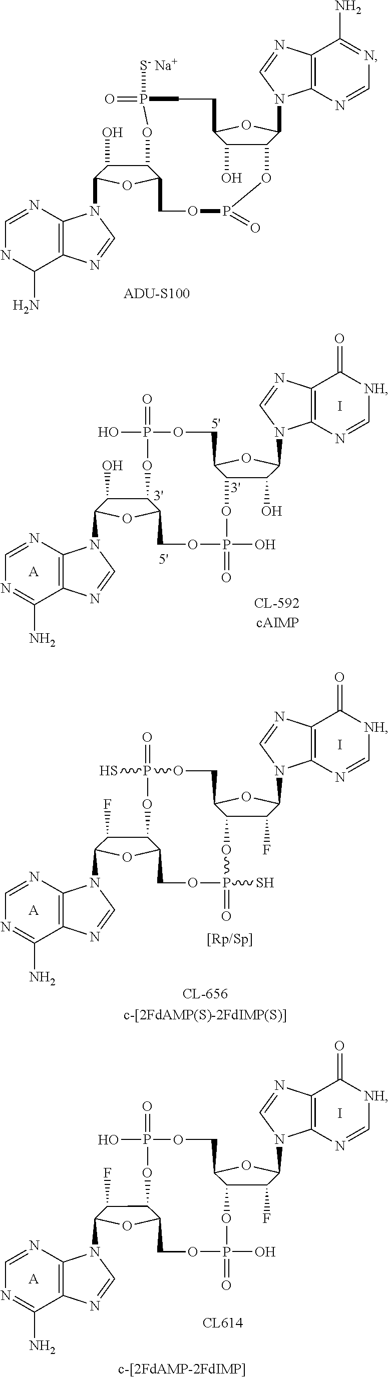

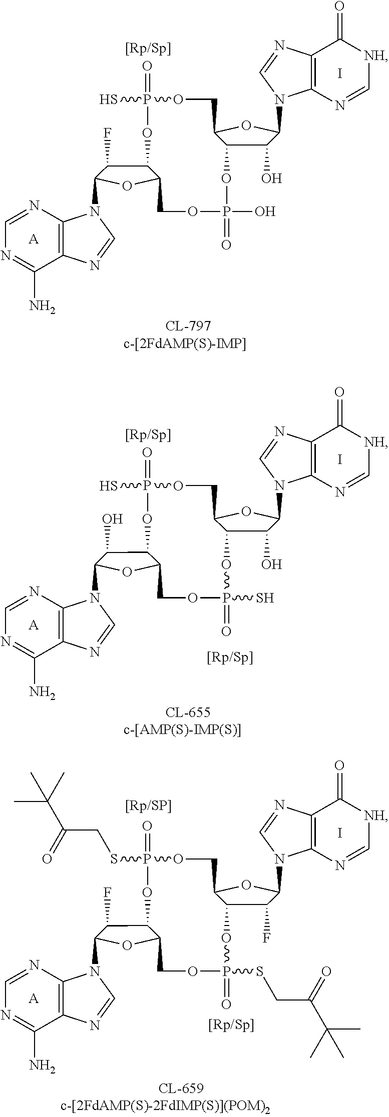

and
a pharmaceutically acceptable salt thereof. See WO 2016/096174A1.

In other aspects, the STING agonist useful for the present disclosure comprises a compound having the following formula:

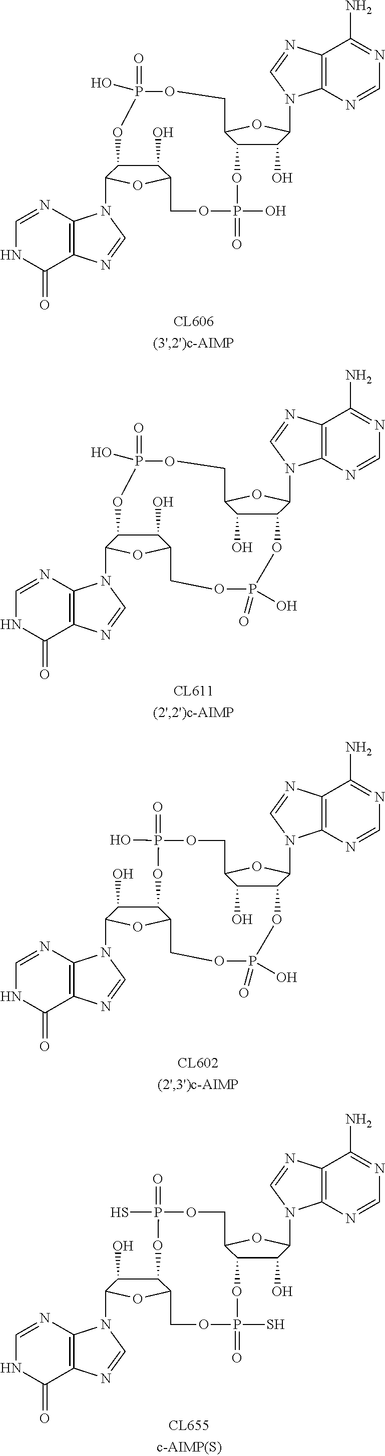 [SMILES: Nc1ncnc2c1ncn2C1OC2COP(=O)(O)O[C@H]3C(n4cnc5c(=O)[nH]cnc54)OC(COP(=O)(O)O[C@H]2[C@H]1O)[C@H]3O ; Nc1ncnc2c1ncn2C1OC2COP(=O)(O)O[C@H]3C(n4cnc5c(=O)[nH]cnc54)OC(COP(=O)(O)O[C@@H]1[C@@H]2O)[C@H]3O ; Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3O ; Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3O]

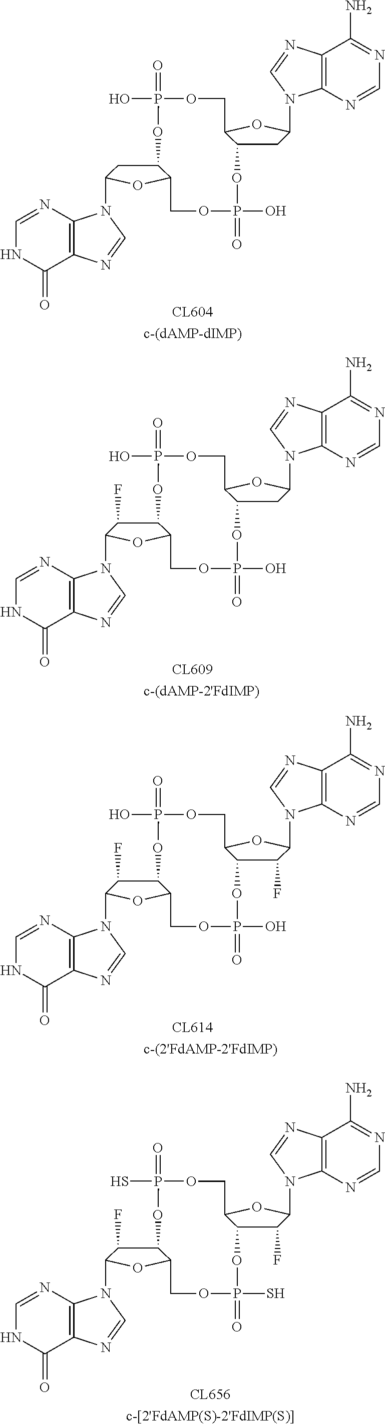 [SMILES: Nc1ncnc2c1ncn2C1C[C@@H]2OP(=O)(O)OCC3OC(n4cnc5c(=O)[nH]cnc54)C[C@@H]3OP(=O)(O)OCC2O1 ; Nc1ncnc2c1ncn2C1C[C@@H]2OP(=O)(O)OCC3OC(n4cnc5c(=O)[nH]cnc54)[C@H](F)[C@@H]3OP(=O)(O)OCC2O1 ; Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F]

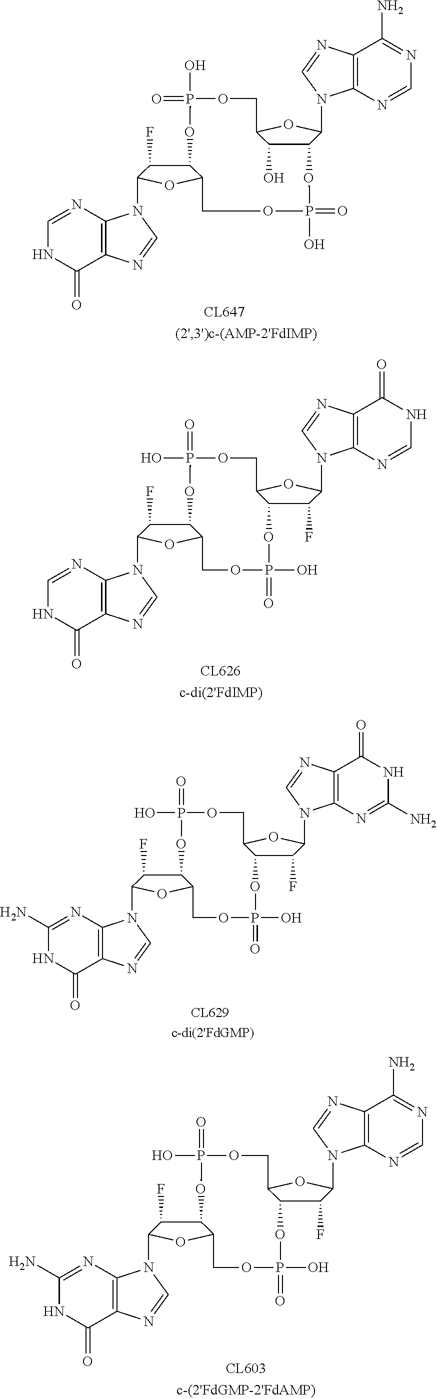 [SMILES: Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3F ; O=c1[nH]cnc2c1ncn2C1OC2COP(=O)(O)O[C@@H]3C(COP(=O)(O)O[C@H]2[C@H]1F)OC(n1cnc2c(=O)[nH]cnc21)[C@@H]3F ; Nc1nc2c(ncn2C2OC3COP(=O)(O)O[C@@H]4C(COP(=O)(O)O[C@H]3[C@H]2F)OC(n2cnc3c(=O)[nH]c(N)nc32)[C@@H]4F)c(=O)[nH]1 ; Nc1nc2c(ncn2C2OC3COP(=O)(O)O[C@@H]4C(COP(=O)(O)O[C@H]3[C@H]2F)OC(n2cnc3c(N)ncnc32)[C@@H]4F)c(=O)[nH]1]

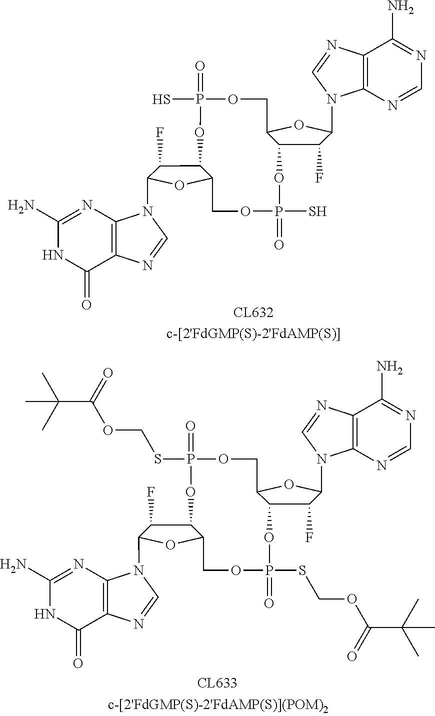 [SMILES: Nc1nc2c(ncn2C2OC3COP(=O)(S)O[C@@H]4C(COP(=O)(S)O[C@H]3[C@H]2F)OC(n2cnc3c(N)ncnc32)[C@@H]4F)c(=O)[nH]1 ; CC(C)(C)C(=O)OCSP1(=O)OCC2OC(n3cnc4c(=O)[nH]c(N)nc43)[C@H](F)[C@@H]2OP(=O)(SCOC(=O)C(C)(C)C)OCC2OC(n3cnc4c(N)ncnc43)[C@H](F)[C@@H]2O1]

or any pharmaceutically acceptable salts thereof.

In some aspects, the STING agonist useful for the present disclosure comprises a compound having the following formula:

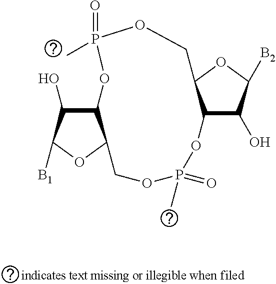

wherein each symbol is defined in WO 2014/093936, the content of which is incorporated herein by reference in its entirety.

In some aspects, the STING agonist useful for the present disclosure comprises a compound having the following formula:

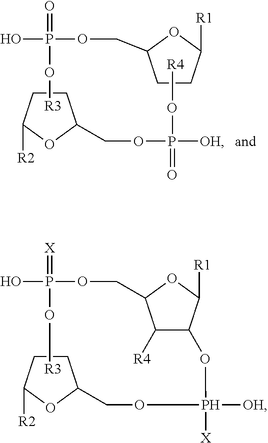

wherein each substituent is defined in WO 2014/189805, the content of which is incorporated herein by reference in its entirety.

In some aspects, the STING agonist useful for the present disclosure comprises a compound having the following formula:

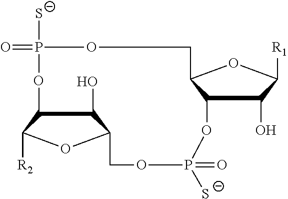

wherein each substituent is defined in WO 2015/077354, the content of which is incorporated herein by reference in its entirety. See also Cell reports 11, 1018-1030 (20151.

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound having a formula selected from

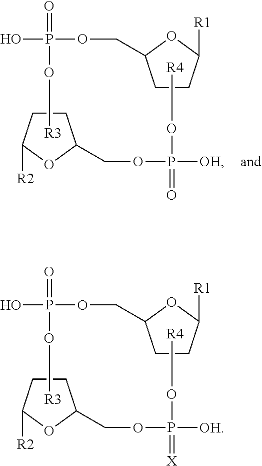

wherein each substituent (i.e., R1, R2, R3, R4, and X) is defined in WO 2014/189806, the content of which is incorporated herein by reference in its entirety.

In some aspects, the STING agonist useful for the present disclosure comprises a compound having the following formula:

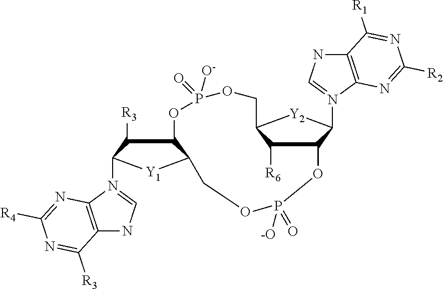

wherein each substituent (i.e., R1, R2, R3, R4, R5, R6, Y1 and Y2) is defined in WO 2015/185565, the content of which is incorporated herein by reference in its entirety.

In some aspects, the STING agonist useful for the present disclosure comprises a compound selected from the following formulas:

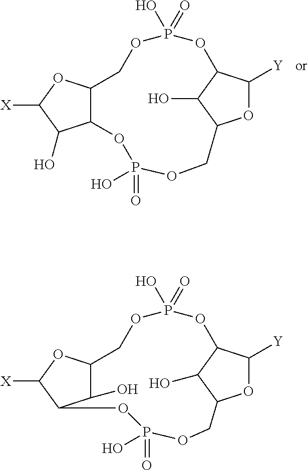

wherein each substituent (i.e., X and Y) is defined in WO 2014/179760, the content of which is incorporated herein by reference in its entirety.

In some aspects, the STING agonist useful for the present disclosure comprises a compound having one of the following formulas:

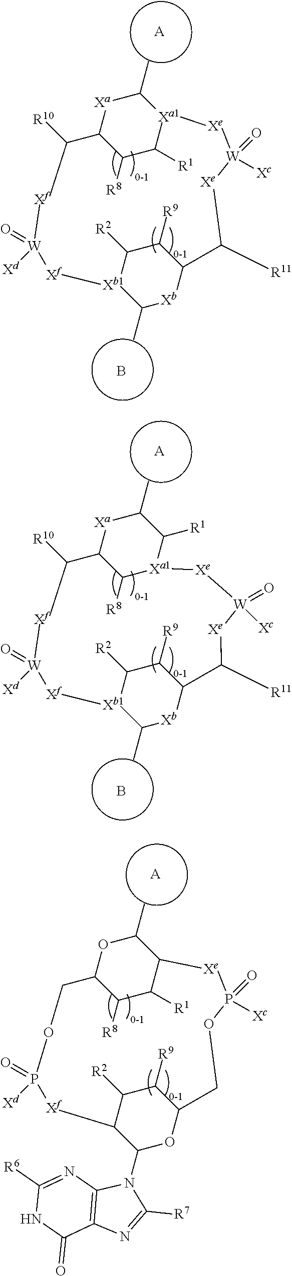

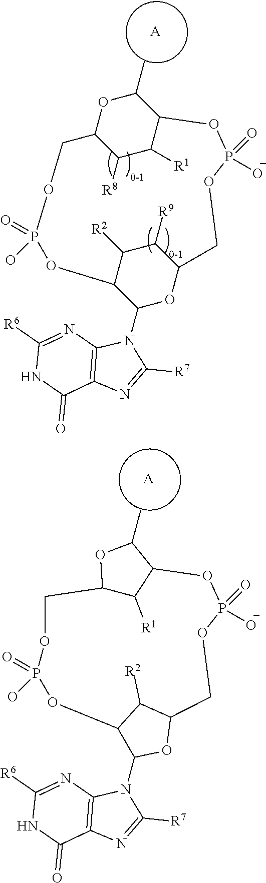

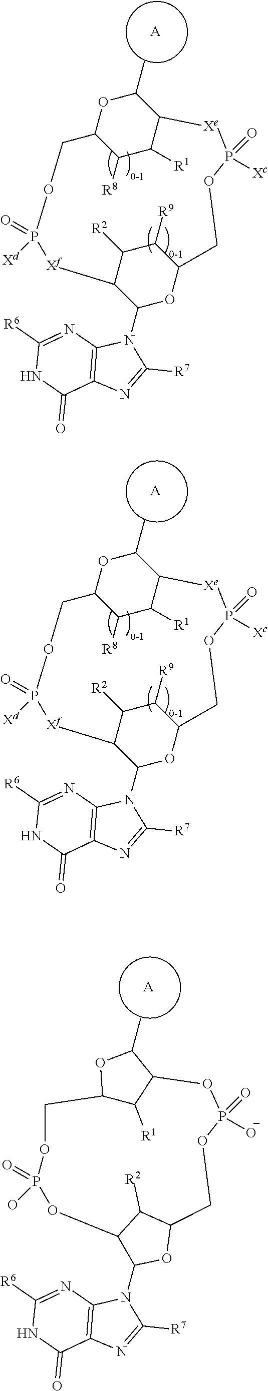

wherein each substituent (i.e., R1, R2, R3, R4, R5, R6, R7, R8, R9, R10, R11, Xa, Xa1, Xb, Xb1, Xc. Xd, Xe, and Xf) is defined in WO 2014/179335, the content of which is incorporated herein by reference in its entirety.

In some aspects, the STING agonist useful for the present disclosure comprises a compound having the following formula:

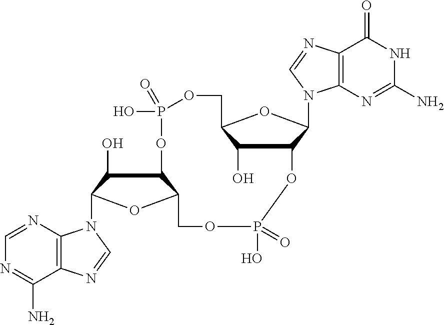

described in WO 2015/017652, the content of which is incorporated herein by reference in its entirety.

In some aspects, the STING agonist useful for the present disclosure comprises a compound having the following formula:

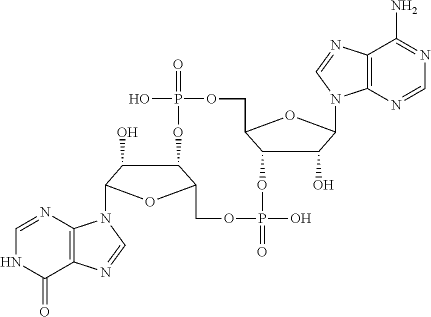 [SMILES: Nc1ncnc2c1ncn2C1OC(COP(=O)(O)O[C@@H]2C(COP(=O)(O)O)OC(n3cnc4c(=O)[nH]cnc43)[C@@H]2O)[C@@H](O)[C@H]1O]

described in WO 2016/096577, the content of which is incorporated herein by reference in its entirety.

In some aspects, the STING agonist useful for the present disclosure comprises a compound having the following formula:

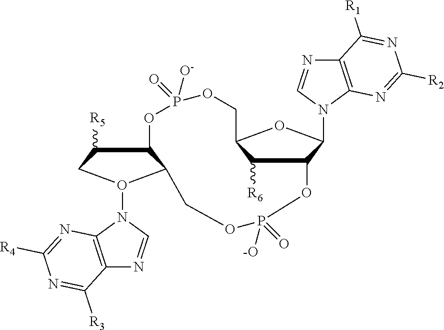

wherein every substituent (i.e., R1, R2, R3, R4, R5, and R6) is described in WO 2011/003025, the content of which is incorporated herein by reference in its entirety.

In some aspects, the STING agonist useful for the present disclosure comprises a compound having the following formula:

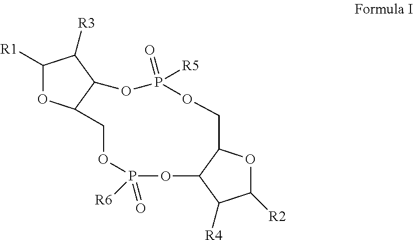

wherein each substituent (i.e., R1, R2, R3, R4, R5 and R6) is defined in WO 2016/145102, the content of which is incorporated herein by reference in its entirety.

In some aspects, the STING agonist useful for the present disclosure comprises a compound having the following formula:

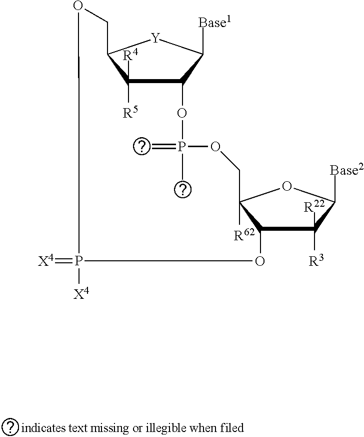

wherein every substituent is defined in WO 2017/027646, the content of which is incorporated herein by reference in its entirety.

In some aspects, the STING agonist useful for the present disclosure comprises a compound having the following formula:

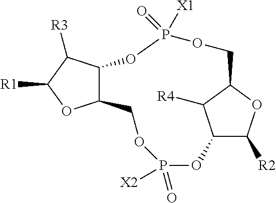

wherein every substituent is defined in WO 2017/075477, the content of which is incorporated herein by reference in its entirety.

In some aspects, the STING agonist useful for the present disclosure comprises a compound having the following formula:

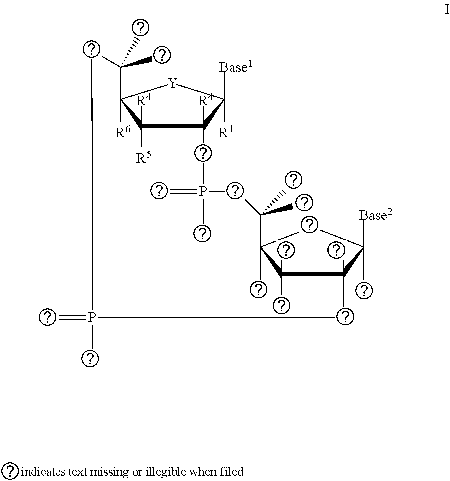

wherein every substituent is defined in WO 2017/027645, the content of which is incorporated herein by reference in its entirety.

In some aspects, the STING agonist useful for the present disclosure comprises a compound having the following formula:

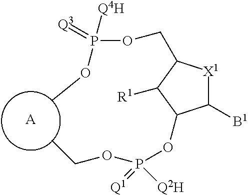

wherein every substituent is defined in WO 2018/100558, the content of which is incorporated herein by reference in its entirety.

In some aspects, the STING agonist useful for the present disclosure comprises a compound having the following formula:

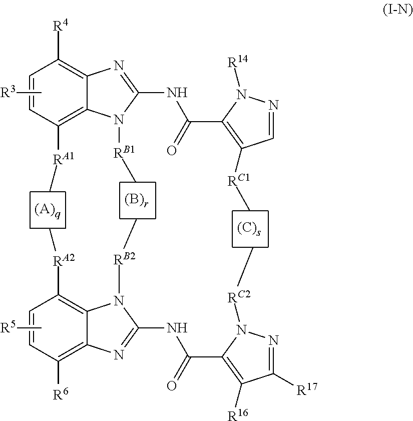

wherein every substituent is defined in WO 2017/175147, the content of which is incorporated herein by reference in its entirety.

In some aspects, the STING agonist useful for the present disclosure comprises a compound having the formula:

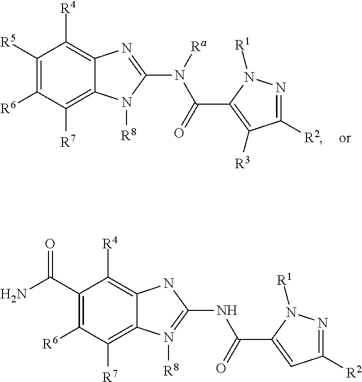

wherein every substituent is defined in WO 2017/175156, the content of which is incorporated herein by reference in its entirety.

In some aspects, the EV (e.g., exosome) comprises a cyclic dinucleotide STING agonist and/or a non-cyclic dinucleotide STING agonist. In some aspects, when several cyclic dinucleotide STING agonist are present on an EV (e.g., exosome) disclosed herein, such STING agonists can be the same or they can be different. In some aspects, when several non-cyclic dinucleotide STING agonist are present, such STING agonists can be the same or they can be different. In some aspects, an EV (e.g., exosome) composition of the present disclosure can comprise two or more populations of EVs, e.g., exosomes, wherein each population of EVs, e.g., exosomes, comprises a different STING agonist or combination thereof.

In some specific aspects, the EV, e.g., exosome, of the present disclosure comprises a (MM)-(Linker)-(biologically active molecule) having the formula (IV):

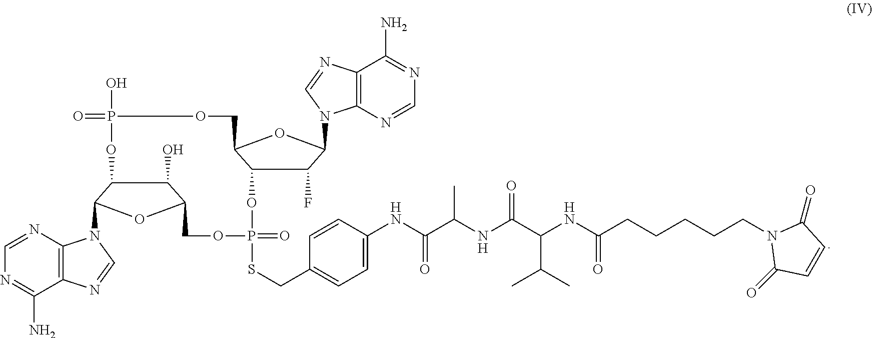 [SMILES: CC(NC(=O)C(NC(=O)CCCCCN1C(=O)C=CC1=O)C(C)C)C(=O)Nc1ccc(CSP2(=O)OC[C@H]3O[C@@H](n4cnc5c(N)ncnc54)[C@H](OP(=O)(O)OC[C@H]4O[C@@H](n5cnc6c(N)ncnc65)[C@H](F)[C@@H]4O2)[C@@H]3O)cc1]

In some specific aspects, the EV (e.g., exosome) of the present disclosure comprises a (MM)-(Linker)-(biologically active molecule) having the formula (V):

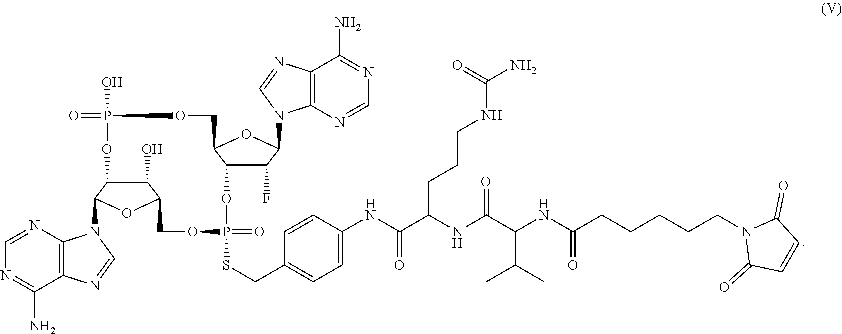

In some specific aspects, the EV (e.g., exosome) of the present disclosure comprises a compound having the formula (VI) (CP249):

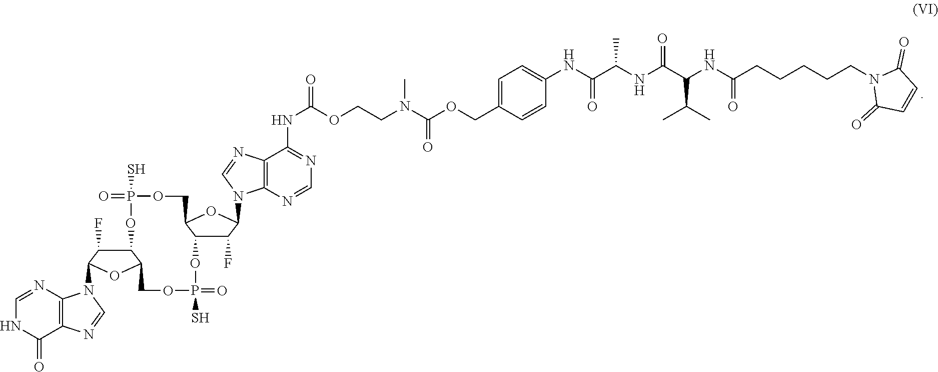

In some specific aspects, the EV (e.g., exosome) of the present disclosure comprises a compound having the formula (VII) (CP250):

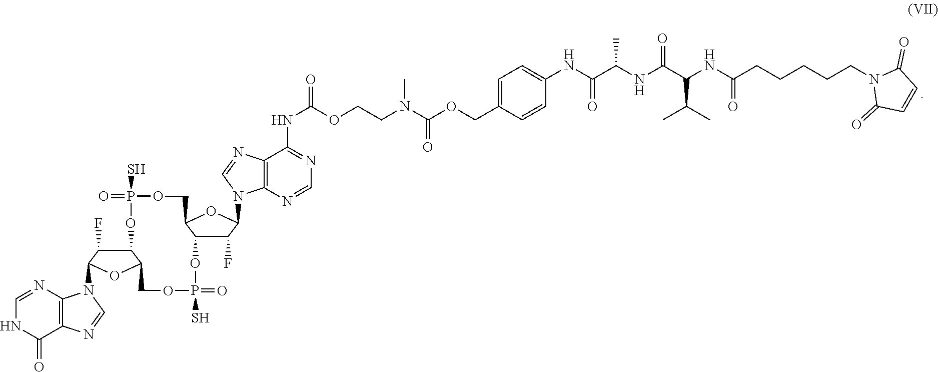

In some specific aspects, the EV (e.g., exosome) of the present disclosure comprises a compound having the formula (VIII) (CP260):

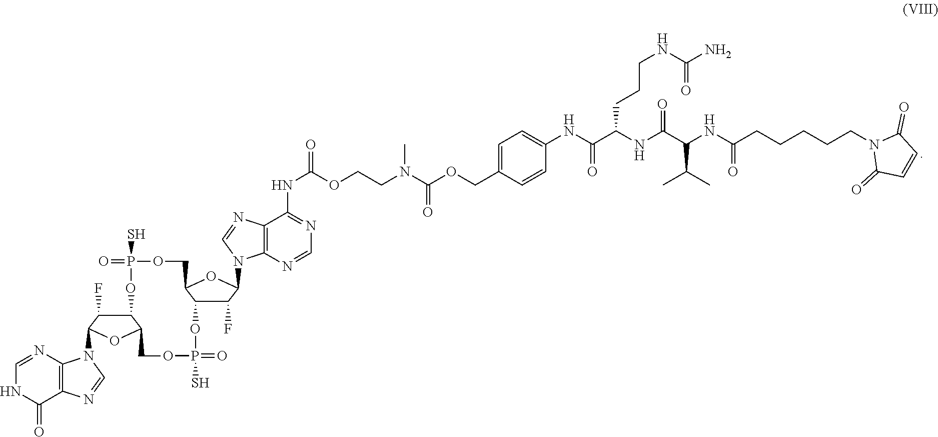

In some specific aspects, the EV (e.g., exosome) of the present disclosure comprises a compound having the formula (IX) (CP261).

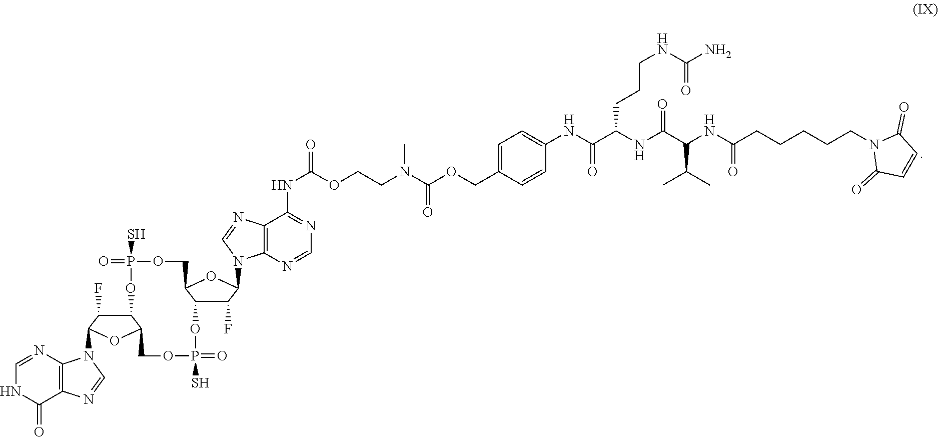

In some aspects, the STING agonist useful for the present EV conjugates includes, but are not limited to, CP247, CP250, CP260, CP261, or a pharmaceutically acceptable salt thereof. In some aspects, the STING agonist useful for the present EV conjugates includes CP247 or a pharmaceutically acceptable salt thereof. In some aspects, the STING agonist useful for the present EV conjugates includes CP250 or a pharmaceutically acceptable salt thereof. In some aspects, the STING agonist useful for the present EV conjugates includes CP260 or a pharmaceutically acceptable salt thereof. In some aspects, the STING agonist useful for the present EV conjugates includes CP261 or a pharmaceutically acceptable salt thereof.

In other aspects, the STING agonist useful for the present EV conjugates includes, but are not limited to, CP227, CP229, or a pharmaceutically acceptable salt thereof. In other aspects, the STING agonist useful for the present EV conjugates includes CP227 or a pharmaceutically acceptable salt thereof. In other aspects, the STING agonist useful for the present EV conjugates includes, but are not limited to, CP229 or a pharmaceutically acceptable salt thereof.

II.D.4 TLR Agonists

In some aspects, the payload comprises a TLR agonist. Non-limiting examples of TLR agonists include: TLR2 agonist (e.g., lipoteichoic acid, atypical LPS, MALP-2 and MALP-404, OspA, porin, LcrV, lipomannan, GPI, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

II.D.5 Antibodies

In some aspects, the payload comprises an antibody or antigen binding fragment thereof. In some aspects, the payload comprises an ADC. In some aspects, the payload comprises a small molecule comprising a synthetic antineoplastic agent (e.g., monomethyl auristatin E (MMAE) (vedotin)), a cytokine release inhibitor (e.g., MCC950), an mTOR inhibitor (e.g., Rapamycin and its analogs (Rapalogs)), an autotaxin inhibitor (e.g., PAT409), a lysophosphatidic acid receptor antagonist (e.g., AM152, also known as BMS-986020), or any combination thereof.

II.D.6 Macrophage-Targeting Biologically Active Molecules

In some aspects, the payload comprises a biologically active molecule that targets macrophages. In other aspects, the payload comprises a biologically active molecule that induces macrophage polarization. Macrophage polarization is a process by which macrophages adopt different functional programs in response to the signals from their microenvironment. This ability is connected to their multiple roles in the organism: they are powerful effector cells of innate immune system, but also important in removal of cellular debris, embryonic development and tissue repair.

By simplified classification, macrophage phenotype has been divided into 2 groups: M1 (classically activated macrophages) and M2 (alternatively activated macrophages). This broad classification was based on in vitro studies, in which cultured macrophages were treated with molecules that stimulated their phenotype switching to particular state. In addition to chemical stimulation, it has been shown that the stiffness of the underlying substrate a macrophage is grown on can direct polarization state, functional roles and migration mode. M1 macrophages were described as the pro-inflammatory type, important in direct host-defense against pathogens, such as phagocytosis and secretion of pro-inflammatory cytokines and microbicidal molecules. M2 macrophages were described to have quite the opposite function: regulation of the resolution phase of inflammation and the repair of damaged tissues. Later, more extensive in vitro and ex vivo studies have shown that macrophage phenotypes are much more diverse, overlapping with each other in terms of gene expression and function, revealing that these many hybrid states form a continuum of activation states which depend on the microenvironment. Moreover, in vivo, there is a high diversity in gene expression profile between different populations of tissue macrophages. Macrophage activation spectrum is thus considered to be wider, involving complex regulatory pathway to response to plethora of different signals from the environment. The diversity of macrophage phenotypes still remain to be fully characterized in vivo.

The imbalance of the macrophage types is related to a number of immunity-related diseases. For example, increased M1/M2 ratio can correlate with development of inflammatory bowel disease, as well as obesity in mice. On the other side, in vitro experiments implicated M2 macrophages as the primary mediators of tissue fibrosis. Several studies have associated the fibrotic profile of M2 macrophages with the pathogenesis of systemic sclerosis. Non-limiting examples of the macrophage targeting biologically active molecules are: PI3Kγ (phosphatidylinositol-4,5-bisphosphate 3-kinase catalytic subunit gamma), RIP1 (Receptor Interacting Protein (RIP) kinase 1, RIPK1), HIF-1α (Hypoxia-inducible factor 1-alpha), AHR1 (Adhesion and hyphal regulator 1), miR146a, miR155, IRF4 (Interferon regulatory factor 4), PPARγ (Peroxisome proliferator-activated receptor gamma), IL-4RA (Interleukin-4 receptor subunit alpha), TLR8 (Toll-like receptor 8), and TGF-β1 (Transforming growth factor beta-1 proprotein).

In some aspects, the payload comprises a biologically active molecule that targets PI3Kγ protein or transcript (PI3Kγ antagonist). In some aspects, the PI3Kγ antagonist is an antisense oligonucleotide. In other aspects, the PI3Kγ antagonist is a small molecule. In some aspects, the ASO targets a transcript, e.g., mRNA, encoding PI3Kγ. The sequence for the PI3Kγ gene can be found at chromosomal location 7q22.3 and under publicly available GenBank Accession Number NC_000007.14 (106865282 . . . 106908980), which is incorporated by reference in its entirety. The sequence for human PI3Kγ protein can be found under publicly available UniProt Accession Number P48736, which is incorporated by reference herein in its entirety.

In some aspects, the payload comprises a biologically active molecule that targets RIP1 protein or transcript (RIP1 antagonist). In some aspects, the RIP1 antagonist is an antisense oligonucleotide. In other aspects, the RIP1 antagonist is a small molecule. In some aspects, the ASO targets a transcript, e.g., mRNA, encoding RIP1. The sequence for the RIP1 gene can be found at chromosomal location 6p25.2 and under publicly available GenBank Accession Number NC_000006.12 (3063967 . . . 3115187), which is incorporated by reference in its entirety. The sequence for human RIP1 protein can be found under publicly available UniProt Accession Number Q13546, which is incorporated by reference herein in its entirety.

In some aspects, the payload comprises a biologically active molecule that targets HIF-1α protein or transcript (HIF-1α antagonist). In some aspects, the HIF-1α antagonist is an antisense oligonucleotide. In other aspects, the HIF-1α antagonist is a small molecule. In some aspects, the ASO targets a transcript, e.g., mRNA, encoding HIF-1α. The sequence for the HIF-1α gene can be found at chromosomal location 14q23.2 and under publicly available GenBank Accession Number NC_000014.9 (61695513 . . . 61748259), which is incorporated by reference in its entirety. The sequence for human HIF-1α protein can be found under publicly available UniProt Accession Number Q16665, which is incorporated by reference herein in its entirety. In some aspects, the ASO targets a mRNA encoding HIF-2α. The sequence for the HIF-2α gene can be found at chromosomal location 2p21 and under publicly available GenBank Accession Number NC_000002.12 (46297407 . . . 46386697), which is incorporated by reference in its entirety. The sequence for human HIF-2α protein can be found under publicly available UniProt Accession Number Q99814, which is incorporated by reference herein in its entirety.

In some aspects, the payload comprises a biologically active molecule that targets AHR1 protein or transcript (AHR1 antagonist). In other aspects, the AHR1 antagonist is a small molecule.

In some aspects, the payload comprises a biologically active molecule that targets miR146a (miR146a antagomir). In some aspects, the miR146a antagomir is an antisense oligonucleotide. In some aspects, the ASO binds to miR146a-5p (ugagaacugaauuccauggguu) (SEQ ID NO: 226). In some aspects, the ASO binds to miR146a-3p (ccucugaaauucaguucuucag) (SEQ ID NO: 227).

In some aspects, the payload comprises a biologically active molecule that mimics miR155 (miR155 mimic). In some aspects, the miR155 mimic is an RNA or DNA. In some aspects, the miR155 mimic comprises the nucleotide sequence of miR155-5p (uuaaugcuaaucgugauaggggu) (SEQ ID NO: 228). In some aspects, the miR155 mimic comprises the nucleotide sequence of miR155-3p (cuccuacauauuagcauuaaca) (SEQ ID NO: 229).

In some aspects, the payload comprises a biologically active molecule that targets IRF-4 protein or transcript (IRF4 antagonist). In some aspects, the IRF4 antagonist is an antisense oligonucleotide. In some aspects, the ASO targets a transcript, e.g., mRNA, encoding IRF-4. The sequence for the IRF-4 gene can be found at chromosomal location 6p25.3 and under publicly available GenBank Accession Number NC_000006.12 (391739 . . . 411443), which is incorporated by reference in its entirety. The sequence for human IRF-4 protein can be found under publicly available UniProt Accession Number Q15306, which is incorporated by reference herein in its entirety.

In some aspects, the payload comprises a biologically active molecule that targets PPARγ protein or transcript (PPARγ antagonist). In some aspects, the PPARγ antagonist is an antisense oligonucleotide. In other aspects, the PPARγ antagonist is a small molecule. In some aspects, the ASO targets a transcript, e.g., mRNA, encoding PPARγ. The sequence for the PPARγ gene can be found at chromosomal location 3p25.2 and under publicly available GenBank Accession Number NC_000003.12 (12287485 . . . 12434356), which is incorporated by reference in its entirety. The sequence for human PPARγ protein can be found under publicly available UniProt Accession Number P37231, which is incorporated by reference herein in its entirety.

In some aspects, the payload comprises a biologically active molecule that targets IL-4RA protein or transcript (IL-4RA antagonist). In some aspects, the IL-4RA antagonist is an antisense oligonucleotide. In some aspects, the ASO targets a transcript, e.g., mRNA, encoding IL-4RA. The sequence for the IL-4RA gene can be found at chromosomal location 16p12.1 and under publicly available GenBank Accession Number NC_000016.10 (27313668 . . . 27364778), which is incorporated by reference in its entirety. The sequence for human IL-4RA protein can be found under publicly available UniProt Accession Number P24394, which is incorporated by reference herein in its entirety.

In some aspects, the payload comprises a biologically active molecule that is an agonist of Toll-like receptor 8 (TLR8). TLR8 is also referred to as CD288. TLR8 is a key component of innate and adaptive immunity. TLRs (Toll-like receptors) control host immune response against pathogens through recognition of molecular patterns specific to microorganisms. It acts via MYD88 and TRAF6, leading to NF-kappa-B activation, cytokine secretion and the inflammatory response. The sequence for human TLR8 protein can be found under publicly available UniProt Accession Number Q9NR97, which is incorporated by reference herein in its entirety.

In some aspects, the payload comprises a biologically active molecule that targets TGF-β1 protein or transcript (TGF-β1 antagonist). In some aspects, the TGF-β1 antagonist is an antisense oligonucleotide. In some aspects, the ASO targets a transcript, e.g., mRNA, encoding TGF-β1. The sequence for the TGF-β1 gene can be found at chromosomal location 19q13.2 and under publicly available GenBank Accession Number NC_000019.10 (41330323 . . . 41353922, complement), which is incorporated by reference in its entirety. The sequence for human TGF-β1 protein can be found under publicly available UniProt Accession Number P01137, which is incorporated by reference herein in its entirety. The ASO can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids. Sugar modifications also include modifications made via altering the substituent groups on the ribose ring to groups other than hydrogen, or the 2′—OH group naturally found in RNA nucleosides. Substituents can, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′- 4′ biradical bridged) nucleosides. For example, the 2′ modified sugar can provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

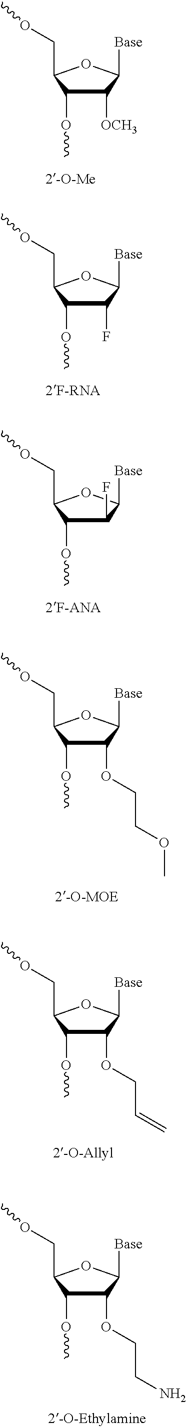

LNA nucleosides are modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., J. Org. Chem. 2010, Vol 75(5) pp. 1569-81, and Mitsuoka et al., Nucleic Acids Research 2009, 37(4), 1225-1238.

In some aspects, the modified nucleoside or the LNA nucleosides of the ASO of the disclosure has a general structure of the formula I or II:

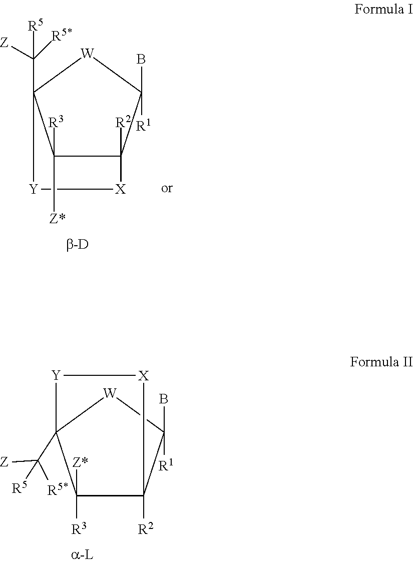

wherein
W is selected from —O—, —S—, —N(R^{a})—, —C(R^{a}R^{b})—, in particular —O—;
B is a nucleobase or a modified nucleobase moiety;
Z is an internucleoside linkage to an adjacent nucleoside or a 5′-terminal group;
Z* is an internucleoside linkage to an adjacent nucleoside or a 3′-terminal group;
R¹, R², R³, R⁵ and R^{5*} are independently selected from hydrogen, halogen, alkyl, alkenyl,
alkynyl, hydroxy, alkoxy, alkoxyalkyl, alkenyloxy, carboxyl, alkoxycarbonyl,
alkylcarbonyl, formyl, azide, heterocycle and aryl; and
X, Y, R^{a} and R^{b} are as defined herein.

II.D.7 Oligonucleotides

In some aspects, the payload comprises an antisense oligonucleotide, a phosphorodiamidate Morpholino oligomer (PMO), or a peptide-conjugated phosphorodiamidate morpholino oligomer (PPMO), an antisense oligonucleotide (ASO), a siRNA, a miRNA, a shRNA, a nucleic acid, or any combination thereof.

In some aspects, the ASO is a PI3Kγ antagonist. In some aspects, the ASO targets a transcript, e.g., mRNA, encoding PI3Kγ. The sequence for the PI3Kγ gene can be found at chromosomal location 7q22.3 and under publicly available GenBank Accession Number NC_000007.14 (106865282 . . . 106908980), which is incorporated by reference in its entirety. The sequence for human PI3Kγ protein can be found under publicly available UniProt Accession Number P48736, which is incorporated by reference herein in its entirety.

In some aspects, the ASO is a RIP1 antagonist. In some aspects, the ASO targets a transcript, e.g., mRNA, encoding RIP1. The sequence for the RIP1 gene can be found at chromosomal location 6p25.2 and under publicly available GenBank Accession Number NC_000006.12 (3063967 . . . 3115187), which is incorporated by reference in its entirety. The sequence for human RIP1 protein can be found under publicly available UniProt Accession Number Q13546, which is incorporated by reference herein in its entirety

In some aspects, the ASO is a HIF-1α antagonist. In some aspects, the ASO targets a transcript, e.g., mRNA, encoding HIF-1α. The sequence for the HIF-1α gene can be found at chromosomal location 14q23.2 and under publicly available GenBank Accession Number NC_000014.9 (61695513 . . . 61748259), which is incorporated by reference in its entirety. The sequence for human HIF-1α protein can be found under publicly available UniProt Accession Number Q16665, which is incorporated by reference herein in its entirety. In some aspects, the ASO targets a mRNA encoding HIF-2α. The sequence for the HIF-2α gene can be found at chromosomal location 2p21 and under publicly available GenBank Accession Number NC_000002.12 (46297407 . . . 46386697), which is incorporated by reference in its entirety. The sequence for human HIF-2α protein can be found under publicly available UniProt Accession Number Q99814, which is incorporated by reference herein in its entirety.

In some aspects, the ASO is a IRF4 antagonist. In some aspects, the ASO targets a transcript, e.g., mRNA, encoding IRF-4. The sequence for the IRF-4 gene can be found at chromosomal location 6p25.3 and under publicly available GenBank Accession Number NC_000006.12 (391739 . . . 411443), which is incorporated by reference in its entirety. The sequence for human IRF-4 protein can be found under publicly available UniProt Accession Number Q15306, which is incorporated by reference herein in its entirety.

In some aspects, the ASO is a PPARγ antagonist. In some aspects, the ASO targets a transcript, e.g., mRNA, encoding PPARγ. The sequence for the PPARγ gene can be found at chromosomal location 3p25.2 and under publicly available GenBank Accession Number NC_000003.12 (12287485 . . . 12434356), which is incorporated by reference in its entirety. The sequence for human PPARγ protein can be found under publicly available UniProt Accession Number P37231, which is incorporated by reference herein in its entirety.

In some aspects, the ASO is a IL-4RA antagonist. In some aspects, the ASO targets a transcript, e.g., mRNA, encoding IL-4RA. The sequence for the IL-4RA gene can be found at chromosomal location 16p12.1 and under publicly available GenBank Accession Number NC_000016.10 (27313668 . . . 27364778), which is incorporated by reference in its entirety. The sequence for human IL-4RA protein can be found under publicly available UniProt Accession Number P24394, which is incorporated by reference herein in its entirety.

In some aspects, the ASO is a TGF-β1 antagonist. In some aspects, the ASO targets a transcript, e.g., mRNA, encoding TGF-β1. The sequence for the TGF-β1 gene can be found at chromosomal location 19q13.2 and under publicly available GenBank Accession Number NC_000019.10 (41330323 . . . 41353922, complement), which is incorporated by reference in its entirety. The sequence for human TGF-β1 protein can be found under publicly available UniProt Accession Number P01137, which is incorporated by reference herein in its entirety.

In some aspects, the ASO targets a transcript, which is a STAT6 transcript, a CEBP/β transcript, a STAT3 transcript, a KRAS transcript, a NRAS transcript, an NLPR3 transcript, or any combination thereof.

STAT6 (STAT6) is also known as signal transducer and activator of transcription 6. Synonyms of STAT6/STAT6 are known and include IL-4 STAT; STAT, Interleukin4-Induced; Transcription Factor IL-4 STAT; STAT6B; STAT6C; and D12S1644. The sequence for the human STAT6 gene can be found under publicly available GenBank Accession Number NC_000012.12:c57111413-57095404. The human STAT6 gene is found at chromosome location 12q13.3 at 57111413-57095404, complement.

CEBP/β (CEBP/β) is also known as CCAAT/enhancer-binding protein beta.

Synonyms of CEBP/I/CEBP/β are known and include C/EBP beta; Liver activator protein; LAP; Liver-enriched inhibitory protein; LIP; Nuclear factor NF-IL6; transcription factor 5; TCF-5; CEBPB; CEBPb; CEBPft; CEBP B; and TCF5. The sequence for the human CEBP/β gene can be found under publicly available GenBank Accession Number NC_000020.11 (50190583 . . . 50192690). The human CEBPfp gene is found at chromosome location 20q13.13 at 50190583-50192690.

NRas is an oncogene encoding a membrane protein that shuttles between the Golgi apparatus and the plasma membrane. NRas-encoding genomic DNA can be found at Chromosomal position 1p13.2 (i.e., nucleotides 5001 to 17438 of GenBank Accession No. NG_007572). N-ras mutations have been described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Oncogenic N-Ras can induce acute myeloid leukemia (AML)- or chronic myelomonocytic leukemia (CMML)-like disease in mice. Neuroblastoma RAS viral oncogene (NRas) is known in the art by various names. Such names include: GTPase NRas, N-ras protein part 4, neuroblastoma RAS viral (v-ras) oncogene homolog neuroblastoma RAS viral oncogene homolog, transforming protein N-Ras, and v-ras neuroblastoma RAS viral oncogene homolog.

Signal Transducer and Activator of Transcription 3 (STAT3) is a signal transducer and activator of transcription that transmits signals from cell surface receptors to the nucleus. STAT3 is frequently hyperactivated in many human cancers. STAT3-encoding genomic DNA can be found at Chromosomal position 17q21.2 (i.e., nucleotides 5,001 to 80,171 of GenBank Accession No. NG_007370.1).

NLRP3 (NLRP3) is also known as NLR family pyrin domain containing 3.

Synonyms of NLRP3/NLRP3 are known and include NLRP3; Clorf7; CIASI; NALP3; PYPAF1; nucleotide-binding oligomerization domain, leucine rich repeat and pyrin domain containing 3; cold-induced autoinflammatory syndrome 1 protein; cryopyin; NACHT, LRR and PYD domains-containing protein 3; angiotensin/vasopressin receptor AII/AVP-like; caterpillar protein 1.1; CLR1.1; cold-induced autoinflammatory syndrome 1 protein; and PYRIN-containing APAF1-like protein 1. The sequence for the human NLRP3 gene can be found under publicly available GenBank Accession Number NC_000001.11:247416156-247449108. The human NLRP3 gene is found at chromosome location 1q44 at 247,416,156-247,449,108.

KRAS is known in the art by various names. Such names include: KRAS Proto-Oncogene, GTPase; V-Ki-Ras2 Kirsten Rat Sarcoma 2 Viral Oncogene Homolog; GTPase KRas; C-Ki-Ras; K-Ras 2; KRAS2; RASK2; V-Ki-Ras2 Kirsten Rat Sarcoma Viral Oncogene Homolog; Kirsten Rat Sarcoma Viral Proto-Oncogene; Cellular Transforming Proto-Oncogene; Cellular C-Ki-Ras2 Proto-Oncogene; Transforming Protein P21; PR310 C—K-Ras Oncogene; C-Kirsten-Ras Protein; K-Ras P21 Protein; and Oncogene KRAS2. The sequence for the human KRAS gene can be found at chromosomal location 12p12.1 and under publicly available GenBank Accession Number NC_000012 (25,204,789-25,250,936). The genomic sequence for human wild-type KRAS transcript corresponds to the reverse complement of residues 25,204,789-25,250,936 of NC_000012

II.D.8 Transport Peptides

In some aspects, the payload comprises a cell transport, cell penetrating, or fusogenic peptide. As used herein, the term “transport peptide” refers to any peptide sequence that facilitates movement of any attached cargo within a cell or cells, including facilitating cargo movement across a cell membrane of a cell, secretion of cargo from a cell or EV, and release of cargo from a cell or EV, as well as other means of cellular movement. In specific, but non-limiting examples, the transport peptide can be a sequence derived from a cell penetrating peptide, a non-classical secretory sequence, an endosomal release domain, a receptor binding domain, and a fusogenic peptide.

As used herein, the term “cell penetrating peptide” refers to any peptide sequence that facilitates movement of any attached cargo across a lipid bilayer, such as the membrane of a cell or the membrane of an EV. As used herein, the term “non-classical secretory sequence” refers to any protein or peptide sequence that provides for secretion of any attached cargo from a cell via an ER-Golgi independent pathway. As used herein, the term “endosomal release domain” is meant to refer to any peptide sequence that facilitates release of any attached cargo from the endosome of a cell or an EV. As used herein, the term “receptor binding domain” is meant to refer to any RNA or protein domain capable of interacting with a surface bound cellular receptor. As used herein, the ten “fusogenic peptide” is meant to refer to any peptide sequence that facilitates cargo exit from an EV or a cell.

In some aspects, an EV, e.g., an exosome, of the present disclosure comprises a transport peptide and second payload, e.g., another biologically active molecule such as a polynucleotide (e.g., an antisense oligonucleotide or an interference RNA).

II.D.9 Adeno-Associated Virus (AAV)

In some aspects, the payload comprises an adeno-associated virus (AAV). In some aspects, the AAV is linked, e.g., chemically liked via a maleimide moiety, to the luminal surface of the EV. In some aspects, the AAV is linked, e.g., chemically liked via a maleimide moiety, to the external surface of the EV. In some aspects, the AAV is linked, e.g., chemically liked via a maleimide moiety, to a scaffold, e.g., a protein scaffold such as a Protein X scaffold or a fragment thereof, or to a lipid scaffold (e.g., cholesterol). In some aspects, the AAV is chemically linked to a scaffold moiety via reaction between a maleimide group present on a AAV capsid protein and a sulfhydryl group present on the scaffold (e.g., either natively present or introduced through a linker or bifunctional reagent). In some aspects, the AAV is chemically linked to a scaffold moiety via reaction between a sulfhydryl group present on an AAV capsid protein (e.g., either natively present or introduced through a linker or bifunctional reagent) and a maleimide group present on the scaffold moiety.

In some aspects, the AAV comprises a genetic cassette. In some aspects, the genetic cassette encodes a protein selected from the group consisting of a secreted protein, a receptor, a structural protein, a signaling protein, a sensory protein, a regulatory protein, a transport protein, a storage protein, a defense protein, a motor protein, a clotting factor, a growth factor, an antioxidant, a cytokine, a chemokine, an enzyme, a tumor suppressor gene, a DNA repair protein, a structural protein, a low-density lipoprotein receptor, an alpha glucosidase, a cystic fibrosis transmembrane conductance regulator, or any combination thereof. In some aspects, the genetic cassette encodes a factor VIII protein or a factor IX protein. In some aspects, the factor VIII protein is a wild-type factor VIII, a B-domain deleted factor VIII, a factor VIII fusion protein, or any combination thereof.

In some aspects, the AAV is selected from the group consisting of AAV type 1, AAV type 2, AAV type 3A, AAV type 3B, AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, a synthetic AAV, an any combination thereof.

II.D.10 Immune Modulators

In some aspects, the payload comprises an immune modulator. In certain aspects, the immune modulator is linked, e.g., chemically linked via a maleimide moiety, to a scaffold moiety, e.g., a Scaffold X protein or a fragment thereof, on the exterior surface of the EV, e.g., exosome or on the luminal surface of the EV, e.g., exosome. In some aspects, the immune modulator is linked, e.g., chemically linked via a maleimide moiety, to a scaffold moiety, e.g., a Scaffold Y protein or a fragment thereof, on the luminal surface of the EV, e.g., exosome. In further aspects, the immune modulator is in the lumen of the EV, e.g., exosome.

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, an immune modulator comprises an activator for a positive co-stimulatory molecule or an activator for a binding partner of a positive co-stimulatory molecule. In certain aspects, the positive co-stimulatory molecule is a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2). In further aspects, the positive co-stimulatory molecule is a CD28-superfamily co-stimulatory molecule (e.g., ICOS or CD28). In some aspects, the activator for a positive co-stimulatory molecule is ICOSL, CD80, or CD86.

In some aspects, an immune modulator comprises a cytokine or a binding partner of a cytokine. In certain aspects, the cytokine comprises IL-2, IL-4, IL-7, IL-1β, IL-12, IL-15, IL-21, or combinations thereof.

In some aspects, an immune modulator comprises a protein that supports intracellular interactions required for germinal center responses. In certain aspects, the protein that supports intracellular interactions required for germinal center responses comprises a signaling lymphocyte activation molecule (SLAM) family member or a SLAM-associated protein (SAP). In some aspects, the SLAM family member comprises SLAM family member 1, CD48, CD229 (Ly9), Ly108, 2B4, CD84, NTB-A, CRACC, BLAME, CD2F-10, or combinations thereof.

IL.D.10 Lysophosphatidic Acid (LPA) Inhibitors

In some aspects, the payload comprises an inhibitor of lysophosphatidic acid (LPA), e.g., an LPA-1 inhibitor. In certain aspects, the LPA-1 inhibitor is linked, e.g., chemically linked via a maleimide moiety, to a scaffold moiety, e.g., a Scaffold X protein or a fragment thereof, on the exterior surface of the EV, e.g., exosome or on the luminal surface of the EV, e.g., exosome. In some aspects, the LPA-1 inhibitor is linked, e.g., chemically linked via a maleimide moiety, to a scaffold moiety, e.g., a Scaffold Y protein or a fragment thereof, on the luminal surface of the EV, e.g., exosome. In further aspects, the LPA-1 inhibitor is in the lumen of the EV, e.g., exosome.

LPA is a highly potent endogenous lipid mediator that protects and rescues cells from programmed cell death. LPA, through its high affinity LPA-1 receptor, is an important mediator of fibrogenesis. Thus, LPA inhibitors can function as antifibrotic agents.

In some aspects, the LPA-1 inhibitor comprises AM095, which is a potent and orally bioavailable antagonist of LPA-1 with IC₅₀ values of 0.73 and 0.98 μM for mouse or recombinant human LPA-1, respectively. In vitro, AM095 has been shown to inhibit LPA-1-induced chemotaxis of both mouse LPA-1/CHO cells and human A2058 melanoma cells with IC₅₀ values of 0.78 μM and 0.23 μM. In vivo, AM095 can dose-dependently block LPA-induced histamine release with an ED₅₀ value of 8.3 mg/kg in mice. Additionally, AM095 has been revealed to remarkably reduce the BALF collagen and protein with an ED₅₀ value of 10 mg/kg in lungs. AM095 has also been shown to decrease both macrophage and lymphocyte infiltration induced by bleomycin in mice. See Swaney et al. (2018) Mol. Can. Res. 16:1601-1613, which is herein incorporated by reference in its entirety.

In some aspects, the LPA-1 inhibitor comprises AM152 (also known as BMS-986020). AM152 is a high-affinity LPA-1 antagonist which inhibits bile acid and phospholipid transporters with IC₅₀s of 4.8 μM, 6.2 μM, and 7.5 μM for BSEP, MRP4, and MDR3, respectively. AM152 can be used for the treatment of idiopathic pulmonary fibrosis (IPF). See Kihara et al. (2015) Exp. Cell Res. 333:171-7; Rosen et al. (2017) European Respiratory Journal 50:PA1038; and, Palmer et al. (2018) Chest 154:1061-1069, which are herein incorporated by reference in their entireties. The Phase 2 study of AM152 (described in Palmer 2018) was terminated early due to gall bladder toxicity and early signs of liver toxicity liver transporter (2 specific transporters).

Additional disclosures relating to EVs (e.g., exosomes) comprising an LPA-1 inhibitor are provided elsewhere in the present disclosure (see, e.g., Example 5).

II.D.11 NLRP3 Inhibitors

In some aspects, the payload comprises an inflammasome inhibitor, e.g., an NLRP3 inhibitor. In certain aspects, the NLRP3 inhibitor is linked, e.g., chemically linked via a maleimide moiety, to a scaffold moiety, e.g., a Scaffold X protein or a fragment thereof, on the exterior surface of the EV, e.g., exosome or on the luminal surface of the EV, e.g., exosome. In some aspects, the NLRP3 inhibitor is linked, e.g., chemically linked via a maleimide moiety, to a scaffold moiety, e.g., a Scaffold Y protein or a fragment thereof, on the luminal surface of the EV, e.g., exosome. In further aspects, the NLRP3 inhibitor is in the lumen of the EV, e.g., exosome.

NLRP3, also known as a NALP3, NACHT, or cryopyrin is a protein that in human is encoded by the NLRP3 gene. NLRP3 is expressed predominantly in macrophages and is a component of the inflammasome. NLRP3 senses pathogen-derived, environmental, and host-derived factors and initiates the formation of inflammasomes, complexes involved in many inflammatory diseases. The NLRP3 inflammasome is an innate immune sensor that upon assembly activates caspase-1 and mediates the processing and release of IL-1β. Amelioration of mouse models of many diseases has been shown to occur by deletion of the NLRP3 inflammasome, including gout, type 2 diabetes, multiple sclerosis, Alzheimer's disease, and atherosclerosis. NLRP3 inflammasome has a role in the pathogenesis of gout and neuroinflammation occurring, e.g., in protein-misfolding diseases, such as Alzheimer's, Parkinson's, and prion diseases. Liu-Bryan (2010) Immunology and Cell Biology 88:20-23; Heneka et al. (2013) Nature 493:674-678; Shi et al. (2015) Life Sciences 135:9-14; Levy et al. (2015) Nature Medicine 21:213-215.

In some aspects, the NLPR3 inhibitor is a diarylsulfonylurea-containing compound. In some aspects, the diarylsulfonylurea-containing compound is MCC950 or a derivative thereof.

MCC950 (N-[[(1,2,3,5,6,7-hexahydro-s-indacen-4-yl)amino]carbonyl]-4-(1-hydroxy-1-methylethyl)-2-furansulfonamide), also known as CP-456773, is a potent and selective inhibitor of the NLRP3 (NOD-like receptor (NLR) pyrin domain-containing protein 3) inflammasome. MCC950 blocks the release of IL-1β induced by NLRP3 activators, such as ATP, MSU and nigericin, by preventing oligomerization of the inflammasome adaptor protein ASC (Apoptosis-associated Speck-like protein containing CARD). Coll et al. (2015) Nature Med. 21:248-255. MCC950 blocks the release of IL-1β in macrophages primed with LPS and activated with ATP or nigericin with an IC50 of approximately 7.5 nM. Although MCC950 blocks the release of IL-1β induced by NLRP3, MCC950 does not inhibit the NLRC4, AIM2, or NLRP1 inflammasomes. Furthermore, MCC950 does not inhibit TLR2 signaling, or priming of NLRP3.

MCC950 is active in vivo, blocking the production of IL-1β and enhancing survival in mouse models of multiple sclerosis. MCC950 also inhibits NLRP3-induced IL-1β production in models for myocardial infarction. van Hout et al (2015) Eur. Heart J. ehw247. MCC950 is also active in ex vivo samples from individuals with Muckle-Wells syndrome. MCC950 is a potential therapeutic agent for the treatment of NLRP3-associated syndromes, including auto-inflammatory and auto-immune diseases.

II.E Bio-Distribution Modifying Agents

In some aspects, the EV, e.g., exosome, comprises a bio-distribution modifying agent. As used herein, the term a “bio-distribution modifying agent,” which refers to an agent (i.e., payload) that can modify the distribution of extracellular vesicles (e.g., exosomes, nanovesicles) in vivo or in vitro (e.g., in a mixed culture of cells of different varieties). In some aspects, the term “targeting moiety” can be used interchangeably with the term bio-distribution modifying agent. In some aspects, the targeting moiety alters the tropism of the EV (e.g., exosome) (“tropism moiety”). As used herein, the term “tropism moiety” refers to a targeting moiety that when expressed on an EV (e.g., exosome) alters and/or enhances the natural movement of the EV. For example, in some aspects, a tropism moiety can promote the EV to be taken up by a particular cell, tissue, or organ. Non-limiting examples of tropism moieties that can be used with the present disclosure include those that can bind to a marker expressed specifically on a dendritic cell (e.g., Clec9A or DEC205) or T cells (e.g., CD3). Unless indicated otherwise, the term “targeting moiety,” as used herein, encompasses tropism moieties.

In some aspects, the EV, e.g., exosome, comprises a targeting moiety, i.e., a biologically active molecule directing an EV, e.g., exosome, of the present disclosure to a specific cell type or tissue comprising, a target (e.g., a target protein such as receptor), wherein another payload (e.g., another biologically active molecule) can have a therapeutic, prophylactic, or diagnostic effect. In certain aspects, the targeting moiety is linked, e.g., chemically linked via a maleimide moiety, to a scaffold moiety, e.g., a Scaffold X protein or a fragment thereof, on the exterior surface of the EV, e.g., exosome.

In some aspects, the targeting moiety is an exogenous targeting moiety is, e.g., an antibody or an antigen binding portion thereof, a protein or peptide that specifically binds to a protein (e.g., a receptor) present on the surface of a target cell or tissue.

In some aspects, the targeting moiety specifically binds to a marker for a dendritic cell. In certain aspects, the marker is present only on the dendritic cell. In some aspects, the dendritic cell comprises a plasmacytoid dendritic cell (pDC), a myeloid/conventional dendritic cell 1 (cDC1), a myeloid/conventional dendritic cell 2 (cDC2), or any combination thereof. In certain aspects, the dendritic cell is cDC1. In further aspects, the marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or any combination thereof. In certain aspects, the marker is Clec9a protein.

In some aspects, the EV, e.g., exosome, of the present disclosure can comprise a tissue or cell-specific ligand which increases EV, e.g., exosome, tropism to a specific tissue or cell, i.e., a tropism moiety. Thus, in some aspects, delivery to the EV, e.g., exosome, to a particular tissue or cell type can be improved by linking to the EV, e.g., exosome, a moiety for cell type-directed tropism (e.g., an immuno-affinity ligand targeting an antigen present on the surface of a certain neural cell type). In certain aspects, the tropism moiety is linked, e.g., chemically linked via a maleimide moiety, to a scaffold moiety, e.g., a Scaffold X protein or a fragment thereof, on the exterior surface of the EV, e.g., exosome.

Tropism can be further improved by the attachment of an anti-phagocytic signal (e.g., CD47 and/or CD24), a half-life extension moiety (e.g., albumin or PEG), or any combination thereof to the external surface of an EV, e.g., exosome of the present disclosure. In certain aspects, the anti-phagocytic signal is linked, e.g., chemically linked via a maleimide moiety, to a scaffold moiety, e.g., a Scaffold X protein or a fragment thereof, on the exterior surface of the EV, e.g., exosome.

Pharmacokinetics, biodistribution, and in particular tropism and retention in the desired tissue or anatomical location can also be accomplish by selecting the appropriate administration route (e.g., intrathecal administration or intraocular administration to improve tropism to the central nervous system).

In some aspects, when tropism to the central nervous system is desired, an EV, e.g., exosome, of the present disclosure can comprises a tissue or cell-specific target ligand which increases EV, e.g., exosome, tropism to a specific central nervous system tissue or cell. In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell-specific target ligand which increases EV, e.g., exosome, tropism to a Schwann cells binds to a Schwann cell surface marker such as Myelin Basic Protein (MBP), Myelin Protein Zero (PO), P75NTR, NCAM, PMP22, and any combination thereof. In some aspects, the cell-specific tropism moiety comprises an antibody or an antigen-binding portion thereof, an aptamer, or an agonist or antagonist of a receptor expressed on the surface of the Schwann cell.

In principle, the EVs, e.g., exosomes of the present disclosure comprising at least one tropism moiety that can direct the EV, e.g., exosome, to a specific target cell or tissue (e.g., Schwann cells in peripheral nerves) can be administered using any suitable administration method known in the art (e.g., intravenous injection or infusion) since the presence of the tropism moiety (alone or in combination with the presence of an antiphagocytic signal and the use of a specific administration route) will induce a tropism of the EVs, e.g., exosomes, towards the desired target cell or tissue.

In some aspects, a targeting moiety and/or tropism moiety disclosed herein can be linked to an EV, e.g., exosome, of the present disclosure via a scaffold moiety (e.g., a Scaffold X protein moiety or a fragment thereof, or a lipid moiety), wherein the targeting and/or tropism moiety is chemically linked to the scaffold moiety via a maleimide moiety and, optionally, one or more linkers (e.g., a cleavable linker).

II.F EVs, e.g., Exosomes

The EVs, e.g., exosomes, of the present disclosure can have a diameter between about 20 and about 300 nm. In certain aspects, an EV, e.g., exosome, of the present disclosure has a diameter between about 20 and about 290 nm, between about 20 and about 280 nm, between about 20 and about 270 nm, between about 20 and about 260 nm, between about 20 and about 250 nm, between about 20 and about 240 nm, between about 20 and about 230 nm, between about 20 and about 220 nm, between about 20 and about 210 nm, between about 20 and about 200 nm, between about 20 and about 190 nm, between about 20 and about 180 nm, between about 20 and about 170 nm, between about 20 and about 160 nm, between about 20 and about 150 nm, between about 20 and about 140 nm, between about 20 and about 130 nm, between about 20 and about 120 nm, between about 20 and about 110 nm, between about 20 and about 100 nm, between about 20 and about 90 nm, between about 20 and about 80 nm, between about 20 and about 70 nm, between about 20 and about 60 nm, between about 20 and about 50 nm, between about 20 and about 40 nm, between about 20 and about 30 nm, between about 30 and about 300 nm, between about 30 and about 290 nm, between about 30 and about 280 nm, between about 30 and about 270 nm, between about 30 and about 260 nm, between about 30 and about 250 nm, between about 30 and about 240 nm, between about 30 and about 230 nm, between about 30 and about 220 nm, between about 30 and about 210 nm, between about 30 and about 200 nm, between about 30 and about 190 nm, between about 30 and about 180 nm, between about 30 and about 170 nm, between about 30 and about 160 nm, between about 30 and about 150 nm, between about 30 and about 140 nm, between about 30 and about 130 nm, between about 30 and about 120 nm, between about 30 and about 110 nm, between about 30 and about 100 nm, between about 30 and about 90 nm, between about 30 and about 80 nm, between about 30 and about 70 nm, between about 30 and about 60 nm, between about 30 and about 50 nm, between about 30 and about 40 nm, between about 40 and about 300 nm, between about 40 and about 290 nm, between about 40 and about 280 nm, between about 40 and about 270 nm, between about 40 and about 260 nm, between about 40 and about 250 nm, between about 40 and about 240 nm, between about 40 and about 230 nm, between about 40 and about 220 nm, between about 40 and about 210 nm, between about 40 and about 200 nm, between about 40 and about 190 nm, between about 40 and about 180 nm, between about 40 and about 170 nm, between about 40 and about 160 nm, between about 40 and about 150 nm, between about 40 and about 140 nm, between about 40 and about 130 nm, between about 40 and about 120 nm, between about 40 and about 110 nm, between about 40 and about 100 nm, between about 40 and about 90 nm, between about 40 and about 80 nm, between about 40 and about 70 nm, between about 40 and about 60 nm, between about 40 and about 50 nm, between about 50 and about 300 nm, between about 50 and about 290 nm, between about 50 and about 280 nm, between about 50 and about 270 nm, between about 50 and about 260 nm, between about 50 and about 250 nm, between about 50 and about 240 nm, between about 50 and about 230 nm, between about 50 and about 220 nm, between about 50 and about 210 nm, between about 50 and about 200 nm, between about 50 and about 190 nm, between about 50 and about 180 nm, between about 50 and about 170 nm, between about 50 and about 160 nm, between about 50 and about 150 nm, between about 50 and about 140 nm, between about 50 and about 130 nm, between about 50 and about 120 nm, between about 50 and about 110 nm, between about 50 and about 100 nm, between about 50 and about 90 nm, between about 50 and about 80 nm, between about 50 and about 70 nm, between about 50 and about 60 nm, between about 60 and about 300 nm, between about 60 and about 290 nm, between about 60 and about 280 nm, between about 60 and about 270 nm, between about 60 and about 260 nm, between about 60 and about 250 nm, between about 60 and about 240 nm, between about 60 and about 230 nm, between about 60 and about 220 nm, between about 60 and about 210 nm, between about 60 and about 200 nm, between about 60 and about 190 nm, between about 60 and about 180 nm, between about 60 and about 170 nm, between about 60 and about 160 nm, between about 60 and about 150 nm, between about 60 and about 140 nm, between about 60 and about 130 nm, between about 60 and about 120 nm, between about 60 and about 110 nm, between about 60 and about 100 nm, between about 60 and about 90 nm, between about 60 and about 80 nm, between about 60 and about 70 nm, between about 70 and about 300 nm, between about 70 and about 290 nm, between about 70 and about 280 nm, between about 70 and about 270 nm, between about 70 and about 260 nm, between about 70 and about 250 nm, between about 70 and about 240 nm, between about 70 and about 230 nm, between about 70 and about 220 nm, between about 70 and about 210 nm, between about 70 and about 200 nm, between about 70 and about 190 nm, between about 70 and about 180 nm, between about 70 and about 170 nm, between about 70 and about 160 nm, between about 70 and about 150 nm, between about 70 and about 140 nm, between about 70 and about 130 nm, between about 70 and about 120 nm, between about 70 and about 110 nm, between about 70 and about 100 nm, between about 70 and about 90 nm, between about 70 and about 80 nm, between about 80 and about 300 nm, between about 80 and about 290 nm, between about 80 and about 280 nm, between about 80 and about 270 nm, between about 80 and about 260 nm, between about 80 and about 250 nm, between about 80 and about 240 nm, between about 80 and about 230 nm, between about 80 and about 220 nm, between about 80 and about 210 nm, between about 80 and about 200 nm, between about 80 and about 190 nm, between about 80 and about 180 nm, between about 80 and about 170 nm, between about 80 and about 160 nm, between about 80 and about 150 nm, between about 80 and about 140 nm, between about 80 and about 130 nm, between about 80 and about 120 nm, between about 80 and about 110 nm, between about 80 and about 100 nm, between about 80 and about 90 nm, between about 90 and about 300 nm, between about 90 and about 290 nm, between about 90 and about 280 nm, between about 90 and about 270 nm, between about 90 and about 260 nm, between about 90 and about 250 nm, between about 90 and about 240 nm, between about 90 and about 230 nm, between about 90 and about 220 nm, between about 90 and about 210 nm, between about 90 and about 200 nm, between about 90 and about 190 nm, between about 90 and about 180 nm, between about 90 and about 170 nm, between about 90 and about 160 nm, between about 90 and about 150 nm, between about 90 and about 140 nm, between about 90 and about 130 nm, between about 90 and about 120 nm, between about 90 and about 110 nm, between about 90 and about 100 nm, between about 100 and about 300 nm, between about 110 and about 290 nm, between about 120 and about 280 nm, between about 130 and about 270 nm, between about 140 and about 260 nm, between about 150 and about 250 nm, between about 160 and about 240 nm, between about 170 and about 230 nm, between about 180 and about 220 nm, or between about 190 and about 210 nm. The size of the EV (e.g., exosome) described herein can be measured according to methods known in the art. The EVs of the present disclosure comprises exosomes, microvesicles, apoptotic bodies, or any combination thereof. In some aspects, the EVs of the present disclosure comprise a population of exosomes and/or microvesicles.

EVs, e.g., exosomes, of the present disclosure comprise a bi-lipid membrane (“exosome membrane” or “EV membrane”), comprising an interior surface (luminal surface) and an exterior surface (e.g., an extracellular surface). The interior surface faces the inner core of the EV (e.g., exosome), i.e., the lumen of the EV. In certain aspects, the external surface can be in contact with the endosome, the multivesicular bodies, or the membrane/cytoplasm of a producer cell.

In some aspects, the EV, e.g., exosome, membrane comprises a bi-lipid membrane, e.g., a lipid bilayer. In some aspects, the EV, e.g., exosome, membrane comprises lipids and fatty acids. In some aspects, the EV, e.g., exosome, membrane comprises lipids comprise phospholipids, glycolipids, fatty acids, sphingolipids, phosphoglycerides, sterols, cholesterols, and phosphatidylserines. In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170.

In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine. In some aspects, the EV or exosome membrane comprises one or more polysaccharides, such as glycan. Glycans on the surface of the EV or exosomes can serve as an attachment to a maleimide moiety or a linker that connect the glycan and a maleimide moiety. The glycan can be present on one or more proteins on the surface of an EV (e.g., exosome), for example, a Scaffold X, such as a PTGFRN polypeptide, or on the lipid membrane of the EV (e.g., exosome). Glycans can be modified to have thiofucose that can serve as a functional group for attaching a maleimide moiety to the glycans. In some aspects, the Scaffold X can be modified to express a high number of glycan to allow additional attachments on the EV (e.g., exosome).

II.G. Scaffold Moieties

In some aspects, the biologically active molecule is linked to the external surface or luminal surface of the EV (e.g., exosome). In some aspects, the biologically active molecule is linked, e.g., chemically linked via a maleimide moiety, to a scaffold moiety (e.g., Scaffold X) on the external surface or on the luminal surface of the EV (e.g., exosome). In some aspects, the biologically active molecule is linked, e.g., chemically linked via a maleimide moiety, to a scaffold moiety (e.g., a cholesterol moiety) on the external surface or on the luminal surface of the EV (e.g., exosome) via a maleimide moiety.

For example, full length mature PTGRN comprises 16 cysteines, i.e., it contains 16 sulfhydryl groups, 14 of them located on the protein's extracellular portion and 2 of them on its intracellular portion. PTGRN has 6 disulfide bridges, all extracellular. Accordingly, PTGRN has 2 extra cellular sulfhydryl groups and 2 intra cellular sulfhydryl groups. Thus, in some aspects, a biologically active moiety can chemically linked via a maleimide moiety to a Scaffold X protein (e.g., PTGRN or a fragment thereof) by reaction between a maleimide reactive group present on the biologically active moiety and one of the sulfhydryl groups present on the Scaffold X protein (e.g., PTGRN or a fragment thereof). Conversely, a Scaffold X protein (e.g., PTGRN or a fragment thereof) comprising a maleimide reactive group introduce by reaction between a bifunctional reagent such as SMCC (Succinimidyl-4-(N-maleimidomethyl)cyclohexane-1-carboxylate) and lysine side chain of the Scaffold X protein could be reacted with a sulfhydryl group present in an biologically active molecule.

In certain aspects, the one or more moieties can be introduced into the EV (e.g., exosome) by transfection. In some aspects, the one or more moieties can be introduced into the EV (e.g., exosome) using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EV (e.g, exosome).

In some aspects, one or more scaffold moieties can be CD47, CD55, CD49, CD40, CD133, CD59, glypican-1, CD9, CD63, CD81, integrins, selectins, lectins, cadherins, other similar polypeptides known to those of skill in the art, or any combination thereof.

In other aspects, one or more scaffold moieties are expressed in the membrane of the EVs, e.g., exosomes, by recombinantly expressing the scaffold moieties in the producer cells. The EVs, e.g., exosomes, obtained from the producer cells can be further modified to be conjugated to a maleimide moiety or to a linker. In other aspects, the scaffold moiety, e.g., Scaffold X, is deglycosylated. In some aspects, the scaffold moiety, e.g., Scaffold X, is highly glycosylated, e.g., higher than naturally-occurring Scaffold X under the same condition.

II.G.1 Transmembrane Scaffold Moieties (e.g., Scaffold X)

Various modifications or fragments of the scaffold moiety can be used for the aspects of the present disclosure. For example, scaffold moieties modified to have enhanced affinity to a binding agent can be used for generating surface-engineered EVs, e.g., exosomes, that can be purified using the binding agent. Scaffold moieties modified to be more effectively targeted to EVs, e.g., exosomes, and/or membranes can be used. Scaffold moieties modified to comprise a minimal fragment required for specific and effective targeting to EV (e.g., exosome) membranes can be also used. In some aspects, scaffold moieties can be linked to the maleimide moiety as described herein. In other aspects, scaffold moieties are not linked to the maleimide moiety.

Scaffold moieties can be engineered synthetically or recombinantly, e.g., to be expressed as a fusion protein, e.g., fusion protein of Scaffold X to another moiety which can react with a maleimide on another molecule (e.g., a protein linker, a protein sequence comprising a reactive group, e.g., a sulfhydryl group, or a combination thereof). For example, the fusion protein can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to another moiety. In case of the fusion protein, the second moiety can be a natural peptide, a recombinant peptide, a synthetic peptide, or any combination thereof. In other aspects, the scaffold moieties can be CD9, CD63, CD81, PDGFR, GPI proteins, lactadherin, LAMP2, or LAMP2B, or any combination thereof. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); or any combination thereof.

In some aspects, the fusion molecule can comprise a scaffold protein disclosed herein (e.g., PTGFRN, BSG, IGSF2, IGSF3, IGSF8, ITGB1, ITGA4, SLC3A2, ATP transporter, or a fragment or a variant thereof) linked, e.g., chemically linked via a maleimide moiety, to a biologically active molecule either directly or through an intermediate (e.g., a chemically inducible dimer, an antigen binding domain, or a receptor). In some aspects, the fusion molecule can be chemically linked, e.g., to a targeting moiety or to a tropism moiety via a maleimide moiety.

In some aspects, the surface (e.g., Scaffold X)-engineered EVs, e.g., exosomes, described herein demonstrate superior characteristics compared to EVs, e.g., exosomes, known in the art. For example, surface (e.g., Scaffold X)-engineered contain modified proteins more highly enriched on their external surface or luminal surface of the EV (e.g., exosome) than naturally occurring EVs, e.g., exosomes, or the EVs, e.g., exosomes, produced using conventional EV (e.g., exosome) proteins. Moreover, the surface (e.g., Scaffold X)-engineered EVs, e.g., exosomes, of the present disclosure can have greater, more specific, or more controlled biological activity compared to naturally occurring EVs, e.g., exosomes, or the EVs, e.g., exosomes, produced using conventional EV (e.g., exosome) proteins.

In some aspects, the scaffold moiety, e.g., a Scaffold X protein, comprises Prostaglandin F2 receptor negative regulator (the PTGFRN polypeptide). The PTGFRN polypeptide can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full-length amino acid sequence of the human PTGFRN polypeptide (Uniprot Accession No. Q9P2B2) is shown at TABLE 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least about five, at least about 10, at least about 15, at least about 20, at least about 25, at least about 30, at least about 40, at least about 50, at least about 70, at least about 80, at least about 90, at least about 100, at least about 110, at least about 120, at least about 130, at least about 140, at least about 150 amino acids at the N terminus of the transmembrane domain, (ii) at least about five, at least about 10, at least about 15, at least about 20, or at least about 25 amino acids at the C terminus of the transmembrane domain, or both (i) and (ii).

In some aspects, the fragments of PTGFRN polypeptide lack one or more functional or structural domains, such as IgV.

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, a fragment of the PTGFRN polypeptide (corresponding to positions 687 to 878 of SEQ ID NO: 1).

In other aspects, the scaffold moiety, e.g., Scaffold X, comprises the amino acid sequence of SEQ ID NO: 2, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of SEQ ID NO: 2 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2.

In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1, amino acids 833 to 853 of SEQ ID NO: 1, SEQ ID NO: 2, or SEQ ID NO: 1. In other aspects, the Scaffold X comprises the amino acid sequence of amino acids 26 to 879 of SEQ ID NO: 1, amino acids 833 to 853 of SEQ ID NO: 1, SEQ ID NO: 2, or SEQ ID NO: 1, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof.

In some aspects, the scaffold moiety, e.g., Scaffold X, comprises the amino acid sequence of amino acids 26 to 879 of SEQ ID NO: 1, amino acids 833 to 853 of SEQ ID NO: 1, SEQ ID NO: 2, or SEQ ID NO: 1, and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of amino acids 26 to 879 of SEQ ID NO: 1, amino acids 833 to 853 of SEQ ID NO: 1, SEQ ID NO: 2, or SEQ ID NO: 1.

In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 186, 187, 188, 189, 190, or 191. In other aspects, the scaffold moiety, e.g., Scaffold X, comprises the amino acid sequence of SEQ ID NO: 186, 187, 188, 189, 190, or 191, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of SEQ ID NO: 186, 187, 188, 189, 190, or 191 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 186, 187, 188, 189, 190, or 191.

TABLE 2
Exemplary Scaffold Protein Sequences

| Protein | Sequence |
|---|---|
| PTGFRN polypeptide | MGRLASRPLLLALLSLALCRGRVVRVPTATLVRVVGTELVIPCNVSDYDGPSEQ |
| (SEQ ID NO: 1) | NFDWSFSSLGSSFVELASTWEVGFPAQLYQERLQRGEILLRRTANDAVELHIKN |
| | VQPSDQGHYKCSTPSTDATVQGNYEDTVQVKVLADSLHVGPSARPPPSLSLREG |
| | EPFELRCTAASASPLHTHLALLWEVHRGPARRSVLALTHEGRFHPGLGYEQRYH |
| | SGDVRLDTVGSDAYRLSVSRALSADQGSYRCIVSEWIAEQGNWQEIQEKAVEVA |
| | TVVIQPSVLRAAVPKNVSVAEGKELDLTCNITTDRADDVRPEVTWSFSRMPDST |
| | LPGSRVLARLDRDSLVHSSPHVALSHVDARSYHLLVRDVSKENSGYYYCHVSLW |
| | APGHNRSWHKVAEAVSSPAGVGVTWLEPDYQVYLNASKVPGFADDPTELACRVV |
| | DTKSGEANVRFTVSWYYRMNRRSDNVVTSELLAVMDGDWTLKYGERSKQRAQDG |
| | DFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVG |
| | DLSSPNETKYIISLDQDSVVKLENWTDASRVDGVVLEKVQEDEFRYRMYQTQVS |
| | DAGLYRCMVTAWSPVRGSLWREAATSLSNPIEIDFQTSGPIFNASVHSDTPSVI |
| | RGDLIKLFCIITVEGAALDPDDMAFDVSWFAVHSFGLDKAPVLLSSLDRKGIVT |
| | TSRRDWKSDLSLERVSVLEFLLQVHGSEDQDFGNYYCSVTPWVKSPTGSWQKEA |
| | EIHSKPVFITVKMDVLNAFKYPLLIGVGLSTVIGLLSCLIGYCSSHWCCKKEVQ |
| | ETRRERRRLMSMEMD |
| PTGFRN polypeptide | PSARPPPSLSLREGEPFELRCTAASASPLHTHLALLWEVHRGPARRSVLALTHE |
| Fragment 1 | GRFHPGLGYEQRYHSGDVRLDTVGSDAYRLSVSRALSADQGSYRCIVSEWIAEQ |
| (SEQ ID NO: 186) | GNWQEIQEKAVEVATVVIQPSVLRAAVPKNVSVAEGKELDLTCNITTDRADDVR |
| | PEVTWSFSRMPDSTLPGSRVLARLDRDSLVHSSPHVALSHVDARSYHLLVRDVS |
| | KENSGYYYCHVSLWAPGHNRSWHKVAEAVSSPAGVGVTWLEPDYQVYLNASKVP |
| | GFADDPTELACRVVDTKSGEANVRFTVSWYYRMNRRSDNVVTSELLAVMDGDWT |
| | LKYGERSKQRAQDGDFIFSKEHTDTFNFRIQRTTEEDRGNYYCVVSAWTKQRNN |
| | SWVKSKDVFSKPVNIFWALEDSVLVVKARQPKPFFAAGNTFEMTCKVSSKNIKS |
| | PRYSVLIMAEKPVGDLSSPNETKYIISLDQDSVVKLENWTDASRVDGVVLEKVQ |
| | EDEFRYRMYQTQVSDAGLYRCMVTAWSPVRGSLWREAATSLSNPIEIDFQTSGP |
| | IFNASVHSDTPSVIRGDLIKLFCIITVEGAALDPDDMAFDVSWFAVHSFGLDKA |
| | PVLLSSLDRKGIVTTSRRDWKSDLSLERVSVLEFLLQVHGSEDQDFGNYYCSVT |
| | PWVKSPTGSWQKEAEIHSKPVFITVKMDVLNAFKYPLLIGVGLSTVIGLLSCLI |
| | GYCSSHWCCKKEVQETRRERRRLMSMEMD |
| PTGFRN polypeptide | VATVVIQPSVLRAAVPKNVSVAEGKELDLTCNITTDRADDVRPEVTWSFSRMPD |
| Fragment 2 | STLPGSRVLARLDRDSLVHSSPHVALSHVDARSYHLLVRDVSKENSGYYYCHVS |
| (SEQ ID NO: 187) | LWAPGHNRSWHKVAEAVSSPAGVGVTWLEPDYQVYLNASKVPGFADDPTELACR |
| | VVDTKSGEANVRFTVSWYYRMNRRSDNVVTSELLAVMDGDWTLKYGERSKQRAQ |
| | DGDFIFSKEHTDTFNFRIQRTTEEDRGNYYCVVSAWTKQRNNSWVKSKDVFSKP |
| | VNIFWALEDSVLVVKARQPKPFFAAGNTFEMTCKVSSKNIKSPRYSVLIMAEKP |
| | VGDLSSPNETKYIISLDQDSVVKLENWTDASRVDGVVLEKVQEDEFRYRMYQTQ |
| | VSDAGLYRCMVTAWSPVRGSLWREAATSLSNPIEIDFQTSGPIFNASVHSDTPS |
| | VIRGDLIKLFCIITVEGAALDPDDMAFDVSWFAVHSFGLDKAPVLLSSLDRKGI |
| | VTTSRRDWKSDLSLERVSVLEFLLQVHGSEDQDFGNYYCSVTPWVKSPTGSWQK |
| | EAEIHSKPVFITVKMDVLNAFKYPLLIGVGLSTVIGLLSCLIGYCSSHWCCKKE |
| | VQETRRERRRLMSMEMD |
| PTGFRN polypeptide | SPAGVGVTWLEPDYQVYLNASKVPGFADDPTELACRVVDTKSGEANVRFTVSWY |
| Fragment 3 | YRMNRRSDNVVTSELLAVMDGDWTLKYGERSKQRAQDGDFIFSKEHTDTFNFRI |
| (SEQ ID NO: 188) | QRTTEEDRGNYYCVVSAWTKQRNNSWVKSKDVFSKPVNIFWALEDSVLVVKARQ |
| | PKPFFAAGNTFEMTCKVSSKNIKSPRYSVLIMAEKPVGDLSSPNETKYIISLDQ |
| | DSVVKLENWTDASRVDGVVLEKVQEDEFRYRMYQTQVSDAGLYRCMVTAWSPVR |
| | GSLWREAATSLSNPIEIDFQTSGPIFNASVHSDTPSVIRGDLIKLFCIITVEGA |
| | ALDPDDMAFDVSWFAVHSFGLDKAPVLLSSLDRKGIVTTSRRDWKSDLSLERVS |
| | VLEFLLQVHGSEDQDFGNYYCSVTPWVKSPTGSWQKEAEIHSKPVFITVKMDVL |
| | NAFKYPLLIGVGLSTVIGLLSCLIGYCSSHWCCKKEVQETRRERRRLMSMEMD |
| PTGFRN polypeptide | KPVNIFWALEDSVLVVKARQPKPFFAAGNTFEMTCKVSSKNIKSPRYSVLIMAE |
| Fragment 4 | KPVGDLSSPNETKYIISLDQDSVVKLENWTDASRVDGVVLEKVQEDEFRYRMYQ |
| (SEQ ID NO: 189) | TQVSDAGLYRCMVTAWSPVRGSLWREAATSLSNPIEIDFQTSGPIFNASVHSDT |
| | PSVIRGDLIKLFCIITVEGAALDPDDMAFDVSWFAVHSFGLDKAPVLLSSLDRK |
| | GIVTTSRRDWKSDLSLERVSVLEFLLQVHGSEDQDFGNYYCSVTPWVKSPTGSW |
| | QKEAEIHSKPVFITVKMDVLNAFKYPLLIGVGLSTVIGLLSCLIGYCSSHWCCK |
| | KEVQETRRERRRLMSMEMD |
| PTGFRN polypeptide | VRGSLWREAATSLSNPIEIDFQTSGPIFNASVHSDTPSVIRGDLIKLFCIITVE |
| Fragment 5 | GAALDPDDMAFDVSWFAVHSFGLDKAPVLLSSLDRKGIVTTSRRDWKSDLSLER |
| (SEQ ID NO: 190) | VSVLEFLLQVHGSEDQDFGNYYCSVTPWVKSPTGSWQKEAEIHSKPVFITVKMD |
| | VLNAFKYPLLIGVGLSTVIGLLSCLIGYCSSHWCCKKEVQETRRERRRLMSMEM |
| | D |
| PTGFRN polypeptide | SKPVFITVKMDVLNAFKYPLLIGVGLSTVIGLLSCLIGYCSSHWCCKKEVQETR |
| Fragment 6 | RERRRLMSMEMD |
| (SEQ ID NO: 191) | |
| PTGFRN polypeptide | MGRLASRPLLLALLSLALCRG |
| Signal peptide | |
| (SEQ ID NO: 192) | |
| PTGFRN polypeptide | GPIFNASVHSDTPSVIRGDLIKLFCIITVEGAALDPDDMAFDVSWFAVHSFGLD |
| Fragment | KAPVLLSSLDRKGIVTTSRRDWKSDLSLERVSVLEFLLQVHGSEDQDFGNYYCS |
| (SEQ ID NO: 2) | VTPWVKSPTGSWQKEAEIHSKPVFITVKMDVLNAFKYPLLIGVGLSTVIGLLSC |
| | LIGYCSSHWCCKKEVQETRRERRRLMSMEM |
| | 687-878 of SEQ ID NO: 1 |
| BSG polypeptide | MAAALFVLLGFALLGTHGASGAAGFVQAPLSQQRWVGGSVELHCEAVGSPVPEI |
| (SEQ ID NO: 3) | QWWFEGQGPNDTCSQLWDGARLDRVHIHATYHQHAASTISIDTLVEEDTGTYEC |
| | RASNDPDRNHLTRAPRVKWVRAQAVVLVLEPGTVFTTVEDLGSKILLTCSLNDS |
| | ATEVTGHRWLKGGVVLKEDALPGQKTEFKVDSDDQWGEYSCVFLPEPMGTANIQ |
| | LHGPPRVKAVKSSEHINEGETAMLVCKSESVPPVTDWAWYKITDSEDKALMNGS |
| | ESRFFVSSSQGRSELHIENLNMEADPGQYRCNGTSSKGSDQAIITLRVRSHLAA |
| | LWPFLGIVAEVLVLVTIIFIYEKRRKPEDVLDDDDAGSAPLKSSGQHQNDKGKN |
| | VRQRNSS |
| IGSF8 polypeptide | MGALRPTLLPPSLPLLLLLMLGMGCWAREVLVPEGPLYRVAGTAVSISCNVTGY |
| (SEQ ID NO: 4) | EGPAQQNFEWFLYRPEAPDTALGIVSTKDTQFSYAVFKSRVVAGEVQVQRLQGD |
| | AVVLKIARLQAQDAGIYECHTPSTDTRYLGSYSGKVELRVLPDVLQVSAAPPGP |
| | RGRQAPTSPPRMTVHEGQELALGCLARTSTQKHTHLAVSFGRSVPEAPVGRSTL |
| | QEVVGIRSDLAVEAGAPYAERLAAGELRLGKEGTDRYRMVVGGAQAGDAGTYHC |
| | TAAEWIQDPDGSWAQIAEKRAVLAHVDVQTLSSQLAVTVGPGERRIGPGEPLEL |
| | LCNVSGALPPAGRHAAYSVGWEMAPAGAPGPGRLVAQLDTEGVGSLGPGYEGRH |
| | IAMEKVASRTYRLRLEAARPGDAGTYRCLAKAYVRGSGTRLREAASARSRPLPV |
| | HVREEGVVLEAVAWLAGGTVYRGETASLLCNISVRGGPPGLRLAASWWVERPED |
| | GELSSVPAQLVGGVGQDGVAELGVRPGGGPVSVELVGPRSHRLRLHSLGPEDEG |
| | VYHCAPSAWVQHADYSWYQAGSARSGPVTVYPYMHALDTLFVPLLVGTGVALVT |
| | GATVLGTITCCFMKRLRKR |
| ITGB1 polypeptide | MNLQPIFWIGLISSVCCVFAQTDENRCLKANAKSCGECIQAGPNCGWCTNSTFL |
| (SEQ ID NO: 5) | QEGMPTSARCDDLEALKKKGCPPDDIENPRGSKDIKKNKNVTNRSKGTAEKLKP |
| | EDITQIQPQQLVLRLRSGEPQTFTLKFKRAEDYPIDLYYLMDLSYSMKDDLENV |
| | KSLGTDLMNEMRRITSDFRIGFGSFVEKTVMPYISTTPAKLRNPCTSEQNCTSP |
| | FSYKNVLSLTNKGEVENELVGKQRISGNLDSPEGGFDAIMQVAVCGSLIGWRNV |
| | TRLLVFSTDAGFHFAGDGKLGGIVLPNDGQCHLENNMYTMSHYYDYPSIAHLVQ |
| | KLSENNIQTIFAVTEEFQPVYKELKNLIPKSAVGTLSANSSNVIQLIIDAYNSL |
| | SSEVILENGKLSEGVTISYKSYCKNGVNGTGENGRKCSNISIGDEVQFEISITS |
| | NKCPKKDSDSFKIRPLGFTEEVEVILQYICECECQSEGIPESPKCHEGNGTFEC |
| | GACRCNEGRVGRHCECSTDEVNSEDMDAYCRKENSSEICSNNGECVCGQCVCRK |
| | RDNTNEIYSGASNGQICNGRGICECGVCKCTDPKFQGQTCEMCQTCLGVCAEHK |
| | ECVQCRAFNKGEKKDTCTQECSYFNITKVESRDKLPQPVQPDPVSHCKEKDVDD |
| | CWFYFTYSVNGNNEVMVHVVENPECPTGPDIIPIVAGVVAGIVLIGLALLLIWK |
| | LLMIIHDRREFAKFEKEKMNAKWDTGENPIYKSAVTTVVNPKYEGK |
| ITGA4 polypeptide | MAWEARREPGPRRAAVRETVMLLLCLGVPTGRPYNVDTESALLYQGPHNTLFGY |
| (SEQ ID NO: 6) | SVVLHSHGANRWLLVGAPTANWLANASVINPGAIYRCRIGKNPGQTCEQLQLGS |
| | PNGEPCGKTCLEERDNQWLGVTLSRQPGENGSIVTCGHRWKNIFYIKNENKLPT |
| | GGCYGVPPDLRTELSKRIAPCYQDYVKKFGENFASCQAGISSFYTKDLIVMGAP |
| | GSSYWTGSLFVYNITTNKYKAFLDKQNQVKFGSYLGYSVGAGHFRSQHTTEVVG |
| | GAPQHEQIGKAYIFSIDEKELNILHEMKGKKLGSYFGASVCAVDLNADGFSDLL |
| | VGAPMQSTIREEGRVFVYINSGSGAVMNAMETNLVGSDKYAARFGESIVNLGDI |
| | DNDGFEDVAIGAPQEDDLQGAIYIYNGRADGISSTFSQRIEGLQISKSLSMFGQ |
| | SISGQIDADNNGYVDVAVGAFRSDSAVLLRTRPVVIVDASLSHPESVNRTKFDC |
| | VENGWPSVCIDLTLCFSYKGKEVPGYIVLFYNMSLDVNRKAESPPRFYFSSNGT |
| | SDVITGSIQVSSREANCRTHQAFMRKDVRDILTPIQIEAAYHLGPHVISKRSTE |
| | EFPPLQPILQQKKEKDIMKKTINFARFCAHENCSADLQVSAKIGFLKPHENKTY |
| | LAVGSMKTLMLNVSLFNAGDDAYETTLHVKLPVGLYFIKILELEEKQINCEVTD |
| | NSGVVQLDCSIGYIYVDHLSRIDISFLLDVSSLSRAEEDLSITVHATCENEEEM |
| | DNLKHSRVTVAIPLKYEVKLTVHGFVNPTSFVYGSNDENEPETCMVEKMNLTFH |
| | VINTGNSMAPNVSVEIMVPNSFSPQTDKLFNILDVQTTTGECHFENYQRVCALE |
| | QQKSAMQTLKGIVRFLSKTDKRLLYCIKADPHCLNFLCNFGKMESGKEASVHIQ |
| | LEGRPSILEMDETSALKFEIRATGFPEPNPRVIELNKDENVAHVLLEGLHHQRP |
| | KRYFTIVIISSSLLLGLIVLLLISYVMWKAGFFKRQYKSILQEENRRDSWSYIN |
| | SKSNDD |
| SLC3A2 | MELQPPEASIAVVSIPRQLPGSHSEAGVQGLSAGDDSELGSHCVAQTGLELLAS |
| polypeptide, where | GDPLPSASQNAEMIETGSDCVTQAGLQLLASSDPPALASKNAEVTGTMSQDTEV |
| the first Met is | DMKEVELNELEPEKQPMNAASGAAMSLAGAEKNGLVKIKVAEDEAEAAAAAKFT |
| processed. | GLSKEELLKVAGSPGWVRTRWALLLLFWLGWLGMLAGAVVIIVRAPRCRELPAQ |
| (SEQ ID NO: 7) | KWWHTGALYRIGDLQAFQGHGAGNLAGLKGRLDYLSSLKVKGLVLGPIHKNQKD |
| | DVAQTDLLQIDPNFGSKEDFDSLLQSAKKKSIRVILDLTPNYRGENSWFSTQVD |
| | TVATKVKDALEFWLQAGVDGFQVRDIENLKDASSFLAEWQNITKGFSEDRLLIA |
| | GTNSSDLQQILSLLESNKDLLLTSSYLSDSGSTGEHTKSLVTQYLNATGNRWCS |
| | WSLSQARLLTSFLPAQLLRLYQLMLFTLPGTPVFSYGDEIGLDAAALPGQPMEA |
| | PVMLWDESSFPDIPGAVSANMTVKGQSEDPGSLLSLFRRLSDQRSKERSLLHGD |
| | FHAFSAGPGLFSYIRHWDQNERFLVVLNFGDVGLSAGLQASDLPASASLPAKAD |
| | LLLSTQPGREEGSPLELERLKLEPHEGLLLRFPYAA |
| BSG Protein | PGTVFTTVEDLGSKILLTCSLNDSATEVTGHRWLKGGVVLKEDALPGQKTEFKVDSDDQ |
| Fragment 1 | WGEYSCVFLPEPMGTANIQLHGPPRVKAVKSSEHINEGETAMLVCKSESVPPVTDWAWY |
| (SEQ ID NO: 193) | KITDSEDKALMNGSESRFFVSSSQGRSELHIENLNMEADPGQYRCNGTSSKGSDQAIIT |
| | LRVRSHLAALWPFLGIVAEVLVLVTIIFIYEKRRKPEDVLDDDDAGSAPLKSSGQHQND |
| | KGKNVRQRNSS |
| BSG Protein | HGPPRVKAVKSSEHINEGETAMLVCKSESVPPVTDWAWYKITDSEDKALMNGSESRFFV |
| Fragment 2 | SSSQGRSELHIENLNMEADPGQYRCNGTSSKGSDQAIITLRVRSHLAALWPFLGIVAEV |
| (SEQ ID NO: 194) | LVLVTIIFIYEKRRKPEDVLDDDDAGSAPLKSSGQHQNDKGKNVRQRNSS |
| BSG Protein | SHLAALWPFLGIVAEVLVLVTIIFIYEKRRKPEDVLDDDDAGSAPLKSSGQHQNDKGKN |
| Fragment 3 | VRQRNSS |
| (SEQ ID NO: 195) | |
| BSG Protein Signal | MAAALFVLLGFALLGTHG |
| peptide | |
| (SEQ ID NO: 196) | |
| IGSF8 Protein | APPGPRGRQAPTSPPRMTVHEGQELALGCLARTSTQKHTHLAVSFGRSVPEAPVGRSTL |
| Fragment #1 | QEVVGIRSDLAVEAGAPYAERLAAGELRLGKEGTDRYRMVVGGAQAGDAGTYHCTAAEW |
| (SEQ ID NO: 197) | IQDPDGSWAQIAEKRAVLAHVDVQTLSSQLAVTVGPGERRIGPGEPLELLCNVSGALPP |
| | AGRHAAYSVGWEMAPAGAPGPGRLVAQLDTEGVGSLGPGYEGRHIAMEKVASRTYRLRL |
| | EAARPGDAGTYRCLAKAYVRGSGTRLREAASARSRPLPVHVREEGVVLEAVAWLAGGTV |
| | YRGETASLLCNISVRGGPPGLRLAASWWVERPEDGELSSVPAQLVGGVGQDGVAELGVR |
| | PGGGPVSVELVGPRSHRLRLHSLGPEDEGVYHCAPSAWVQHADYSWYQAGSARSGPVTV |
| | YPYMHALDTLFVPLLVGTGVALVTGATVLGTITCCFMKRLRKR |
| IGSF8 Protein | AHVDVQTLSSQLAVTVGPGERRIGPGEPLELLCNVSGALPPAGRHAAYSVGWEMAPAGA |
| Fragment #2 | PGPGRLVAQLDTEGVGSLGPGYEGRHIAMEKVASRTYRLRLEAARPGDAGTYRCLAKAY |
| (SEQ ID NO: 198) | VRGSGTRLREAASARSRPLPVHVREEGVVLEAVAWLAGGTVYRGETASLLCNISVRGGP |
| | PGLRLAASWWVERPEDGELSSVPAQLVGGVGQDGVAELGVRPGGGPVSVELVGPRSHRL |
| | RLHSLGPEDEGVYHCAPSAWVQHADYSWYQAGSARSGPVTVYPYMHALDTLFVPLLVGT |
| | GVALVTGATVLGTITCCFMKRLRKR |
| IGSF8 Protein | REEGVVLEAVAWLAGGTVYRGETASLLCNISVRGGPPGLRLAASWWVERPEDGELSSVP |
| Fragment #3 | AQLVGGVGQDGVAELGVRPGGGPVSVELVGPRSHRLRLHSLGPEDEGVYHCAPSAWVQH |
| (SEQ ID NO: 199) | ADYSWYQAGSARSGPVTVYPYMHALDTLFVPLLVGTGVALVTGATVLGTITCCFMKRLR |
| | KR |
| IGSF8 Protein | VALVTGATVLGTITCCFMKRLRKR |
| Fragment #4 | |
| (SEQ ID NO: 200) | |
| IGSF8 Protein- | MGALRPTLLPPSLPLLLLLMLGMGCWA |
| Signal Peptide | |
| (SEQ ID NO: 201) | |
| IGSF2 protein | MAGISYVASFFLLLTKLSIGQREVTVQKGPLFRAEGYPVSIGCNVTGHQGPSEQHFQWS |
| (SEQ ID NO: 202) | VYLPTNPTQEVQIISTKDAAFSYAVYTQRVRSGDVYVERVQGNSVLLHISKLQMKDAGE |
| | YECHTPNTDEKYYGSYSAKTNLIVIPDTLSATMSSQTLGKEEGEPLALTCEASKATAQH |
| | THLSVTWYLTQDGGGSQATEIISLSKDFILVPGPLYTERFAASDVQLNKLGPTTFRLSI |
| | ERLQSSDQGQLFCEATEWIQDPDETWMFITKKQTDQTTLRIQPAVKDFQVNITADSLFA |
| | EGKPLELVCLVVSSGRDPQLQGIWFFNGTEIAHIDAGGVLGLKNDYKERASQGELQVSK |
| | LGPKAFSLKIFSLGPEDEGAYRCVVAEVMKTRTGSWQVLQRKQSPDSHVHLRKPAARSV |
| | VMSTKNKQQVVWEGETLAFLCKAGGAESPLSVSWWHIPRDQTQPEFVAGMGQDGIVQLG |
| | ASYGVPSYHGNTRLEKMDWATFQLEITFTAITDSGTYECRVSEKSRNQARDLSWTQKIS |
| | VTVKSLESSLQVSLMSRQPQVMLTNTFDLSCVVRAGYSDLKVPLTVTWQFQPASSHIFH |
| | QLIRITHNGTIEWGNFLSRFQKKTKVSQSLFRSQLLVHDATEEETGVYQCEVEVYDRNS |
| | LYNNRPPRASAISHPLRIAVTLPESKLKVNSRSQVQELSINSNTDIECSILSRSNGNLQ |
| | LAIIWYFSPVSTNASWLKILEMDQTNVIKTGDEFHTPQRKQKFHTEKVSQDLFQLHILN |
| | VEDSDRGKYHCAVEEWLLSTNGTWHKLGEKKSGLTELKLKPTGSKVRVSKVYWTENVTE |
| | HREVAIRCSLESVGSSATLYSVMWYWNRENSGSKLLVHLQHDGLLEYGEEGLRRHLHCY |
| | RSSSTDFVLKLHQVEMEDAGMYWCRVAEWQLHGHPSKWINQASDESQRMVLTVLPSEPT |
| | LPSRICSSAPLLYFLFICPFVLLLLLLISLLCLYWKARKLSTLRSNTRKEKALWVDLKE |
| | AGGVTTNRREDEEEDEGN |
| IGSF3 protein | MKCFFPVLSCLAVLGVVSAQRQVTVQEGPLYRTEGSHITIWCNVSGYQGPSEQNFQWSI |
| (SEQ ID NO: 203) | YLPSSPEREVQIVSTMDSSFPYAIYTQRVRGGKIFIERVQGNSTLLHITDLQARDAGEY |
| | ECHTPSTDKQYFGSYSAKMNLVVIPDSLQTTAMPQTLHRVEQDPLELTCEVASETIQHS |
| | HLSVAWLRQKVGEKPVEVISLSRDFMLHSSSEYAQRQSLGEVRLDKLGRTTFRLTIFHL |
| | QPSDQGEFYCEAAEWIQDPDGSWYAMTRKRSEGAVVNVQPTDKEFTVRLETEKRLHTVG |
| | EPVEFRCILEAQNVPDRYFAVSWAFNSSLIATMGPNAVPVLNSEFAHREARGQLKVAKE |
| | SDSVFVLKIYHLRQEDSGKYNCRVTEREKTVTGEFIDKESKRPKNIPIIVLPLKSSISV |
| | EVASNASVILEGEDLRFSCSVRTAGRPQGRFSVIWQLVDRQNRRSNIMWLDRDGTVQPG |
| | SSYWERSSFGGVQMEQVQPNSFSLGIFNSRKEDEGQYECHVTEWVRAVDGEWQIVGERR |
| | ASTPISITALEMGFAVTAISRTPGVTYSDSFDLQCIIKPHYPAWVPVSVTWRFQPVGTV |
| | EFHDLVTFTRDGGVQWGDRSSSFRTRTAIEKAESSNNVRLSISRASDTEAGKYQCVAEL |
| | WRKNYNNTWTRLAERTSNLLEIRVLQPVTKLQVSKSKRTLTLVENKPIQLNCSVKSQTS |
| | QNSHFAVLWYVHKPSDADGKLILKTTHNSAFEYGTYAEEEGLRARLQFERHVSGGLFSL |
| | TVQRAEVSDSGSYYCHVEEWLLSPNYAWYKLAEEVSGRTEVTVKQPDSRLRLSQAQGNL |
| | SVLETRQVQLECVVLNRTSITSQLMVEWFVWKPNHPERETVARLSRDATFHYGEQAAKN |
| | NLKGRLHLESPSPGVYRLFIQNVAVQDSGTYSCHVEEWLPSPSGMWYKRAEDTAGQTAL |
| | TVMRPDASLQVDTVVPNATVSEKAAFQLDCSIVSRSSQDSRFAVAWYSLRTKAGGKRSS |
| | PGLEEQEEEREEEEEEEEDDDDDDPTERTALLSVGPDAVFGPEGSPWEGRLRFQRLSPV |
| | LYRLTVLQASPQDTGNYSCHVEEWLPSPQKEWYRLTEEESAPIGIRVLDTSPTLQSIIC |
| | SNDALFYFVFFYPFPIFGILIITILLVRFKSRNSSKNSDGKNGVPLLWIKEPHLNYSPT |
| | CLEPPVLSIHPGAID |
| ATP1A1 protein | MGKGVGRDKYEPAAVSEQGDKKGKKGKKDRDMDELKKEVSMDDHKLSLDELHRKYGTDL |
| (SEQ ID NO: 204) | SRGLTSARAAEILARDGPNALTPPPTTPEWIKFCRQLFGGFSMLLWIGAILCFLAYSIQ |
| | AATEEEPQNDNLYLGVVLSAVVIITGCFSYYQEAKSSKIMESFKNMVPQQALVIRNGEK |
| | MSINAEEVVVGDLVEVKGGDRIPADLRIISANGCKVDNSSLTGESEPQTRSPDFTNENP |
| | LETRNIAFFSTNCVEGTARGIVVYTGDRTVMGRIATLASGLEGGQTPIAAEIEHFIHII |
| | TGVAVFLGVSFFILSLILEYTWLEAVIFLIGIIVANVPEGLLATVTVCLTLTAKRMARK |
| | NCLVKNLEAVETLGSTSTICSDKTGTLTQNRMTVAHMWFDNQIHEADTTENQSGVSFDK |
| | TSATWLALSRIAGLCNRAVFQANQENLPILKRAVAGDASESALLKCIELCCGSVKEMRE |
| | RYAKIVEIPFNSTNKYQLSIHKNPNTSEPQHLLVMKGAPERILDRCSSILLHGKEQPLD |
| | EELKDAFQNAYLELGGLGERVLGFCHLFLPDEQFPEGFQFDTDDVNFPIDNLCFVGLIS |
| | MIDPPRAAVPDAVGKCRSAGIKVIMVTGDHPITAKAIAKGVGIISEGNETVEDIAARLN |
| | IPVSQVNPRDAKACVVHGSDLKDMTSEQLDDILKYHTEIVFARTSPQQKLIIVEGCQRQ |
| | GAIVAVTGDGVNDSPALKKADIGVAMGIAGSDVSKQAADMILLDDNFASIVTGVEEGRL |
| | IFDNLKKSIAYTLTSNIPEITPFLIFIIANIPLPLGTVTILCIDLGTDMVPAISLAYEQ |
| | AESDIMKRQPRNPKTDKLVNERLISMAYGQIGMIQALGGFFTYFVILAENGFLPIHLLG |
| | LRVDWDDRWINDVEDSYGQQWTYEQRKIVEFTCHTAFFVSIVVVQWADLVICKTRRNSV |
| | FQQGMKNKILIFGLFEETALAAFLSYCPGMGVALRMYPLKPTWWFCAFPYSLLIFVYDE |
| | VRKLIIRRRPGGWVEKETYY |
| ATP1A2 protein | MGRGAGREYSPAATTAENGGGKKKQKEKELDELKKEVAMDDHKLSLDELGRKYQVDLSK |
| (SEQ ID NO: 205) | GLTNQRAQDVLARDGPNALTPPPTTPEWVKFCRQLFGGFSILLWIGAILCFLAYGIQAA |
| | MEDEPSNDNLYLGVVLAAVVIVTGCFSYYQEAKSSKIMDSFKNMVPQQALVIREGEKMQ |
| | INAEEVVVGDLVEVKGGDRVPADLRIISSHGCKVDNSSLTGESEPQTRSPEFTHENPLE |
| | TRNI |
| ATP1A3 protein | CFFSTNCVEGTARGIVIATGDRTVMGRIATLASGLEVGRTPIAMEIEHFIQLITGVAVF |
| (SEQ ID NO: 206) | LGVSFFVLSLILGYSWLEAVIFLIGIIVANVPEGLLATVTVCLTLTAKRMARKNCLVKN |
| | LEAVETLGSTSTICSDKTGTLTQNRMTVAHMWFDNQIHEADTTEDQSGATFDKRSPTWT |
| | ALSRIAGLCNRAVFKAGQENISVSKRDTAGDASESALLKCIELSCGSVRKMRDRNPKVA |
| | EIPFNSTNKYQLSIHEREDSPQSHVLVMKGAPERILDRCSTILVQGKEIPLDKEMQDAF |
| | QNAYMELGGLGERVLGFCQLNLPSGKFPRGFKFDTDELNFPTEKLCFVGLMSMIDPPRA |
| | AVPDAVGKCRSAGIKVIMVTGDHPITAKAIAKGVGIISEGNETVEDIAARLNIPMSQVN |
| | PREAKACVVHGSDLKDMTSEQLDEILKNHTEIVFARTSPQQKLIIVEGCQRQGAIVAVT |
| | GDGVNDSPALKKADIGIAMGISGSDVSKQAADMILLDDNFASIVTGVEEGRLIFDNLKK |
| | SIAYTLTSNIPEITPFLLFIIANIPLPLGTVTILCIDLGTDMVPAISLAYEAAESDIMK |
| | RQPRNSQTDKLVNERLISMAYGQIGMIQALGGFFTYFVILAENGFLPSRLLGIRLDWDD |
| | RTMNDLEDSYGQEWTYEQRKVVEFTCHTAFFASIVVVQWADLIICKTRRNSVFQQGMKN |
| | KILIFGLLEETALAAFLSYCPGMGVALRMYPLKVTWWFCAFPYSLLIFIYDEVRKLILR |
| | RYPGGWVEKETYY |
| ATP1A4 protein | MGSGGSDSYRIATSQDKKDDKDSPKKNKGKERRDLDDLKKEVAMTEHKMSVEEVCRKYN |
| (SEQ ID NO: 207) | TDCVQGLTHSKAQEILARDGPNALTPPPTTPEWVKFCRQLFGGFSILLWIGAILCFLAY |
| | GIQAGTEDDPSGDNLYLGIVLAAVVIITGCFSYYQEAKSSKIMESFKNMVPQQALVIRE |
| | GEKMQVNAEEVVVGDLVEIKGGDRVPADLRIISAHGCKVDNSSLTGESEPQTRSPDCTH |
| | DNPLETRNITFFSTNCVEGTARGVVVATGDRTVMGRIATLASGLEVGKTPIAIEIEHFI |
| | QLITGVAVFLGVSFFILSLILGYTWLEAVIFLIGIIVANVPEGLLATVTVCLTLTAKRM |
| | ARKNCLVKNLEAVETLGSTSTICSDKTGTLTQNRMTVAHMWFDNQIHEADTTEDQSGTS |
| | FDKSSHTWVALSHIAGLCNRAVFKGGQDNIPVLKRDVAGDASESALLKCIELSSGSVKL |
| | MRERNKKVAEIPFNSTNKYQLSIHETEDPNDNRYLLVMKGAPERILDRCSTILLQGKEQ |
| | PLDEEMKEAFQNAYLELGGLGERVLGFCHYYLPEEQFPKGFAFDCDDVNFTTDNLCFVG |
| | LMSMIDPPRAAVPDAVGKCRSAGIKVIMVTGDHPITAKAIAKGVGIISEGNETVEDIAA |
| | RLNIPVSQVNPRDAKACVIHGTDLKDFTSEQIDEILQNHTEIVFARTSPQQKLIIVEGC |
| | QRQGAIVAVTGDGVNDSPALKKADIGVAMGIAGSDVSKQAADMILLDDNFASIVTGVEE |
| | GRLIFDNLKKSIAYTLTSNIPEITPFLLFIMANIPLPLGTITILCIDLGTDMVPAISLA |
| | YEAAESDIMKRQPRNPRTDKLVNERLISMAYGQIGMIQALGGFFSYFVILAENGFLPGN |
| | LVGIRLNWDDRTVNDLEDSYGQQWTYEQRKVVEFTCHTAFFVSIVVVQWADLIICKTRR |
| | NSVFQQGMKNKILIFGLFEETALAAFLSYCPGMDVALRMYPLKPSWWFCAFPYSFLIFV |
| | YDEIRKLILRRNPGGWVEKETYY |
| ATP1B3 protein | MGLWGKKGTVAPHDQSPRRRPKKGLIKKKMVKREKQKRNMEELKKEVVMDDHKLTLEEL |
| (SEQ ID NO: 208) | STKYSVDLTKGHSHQRAKEILTRGGPNTVTPPPTTPEWVKFCKQLFGGFSLLLWTGAIL |
| | CFVAYSIQIYFNEEPTKDNLYLSIVLSVVVIVTGCFSYYQEAKSSKIMESFKNMVPQQA |
| | LVIRGGEKMQINVQEVVLGDLVEIKGGDRVPADLRLISAQGCKVDNSSLTGESEPQSRS |
| | PDFTHENPLETRNICFFSTNCVEGTARGIVIATGDSTVMGRIASLTSGLAVGQTPIAAE |
| | IEHFIHLITVVAVFLGVTFFALSLLLGYGWLEAIIFLIGIIVANVPEGLLATVTVCLTL |
| | TAKRMARKNCLVKNLEAVETLGSTSTICSDKTGTLTQNRMTVAHMWFDMTVYEADTTEE |
| | QTGKTFTKSSDTWFMLARIAGLCNRADFKANQEILPIAKRATTGDASESALLKFIEQSY |
| | SSVAEMREKNPKVAEIPFNSTNKYQMSIHLREDSSQTHVLMMKGAPERILEFCSTFLLN |
| | GQEYSMNDEMKEAFQNAYLELGGLGERVLGFCFLNLPSSFSKGFPFNTDEINFPMDNLC |
| | FVGLISMIDPPRAAVPDAVSKCRSAGIKVIMVTGDHPITAKAIAKGVGIISEGTETAEE |
| | VAARLKIPISKVDASAAKAIVVHGAELKDIQSKQLDQILQNHPEIVFARTSPQQKLIIV |
| | EGCQRLGAVVAVTGDGVNDSPALKKADIGIAMGISGSDVSKQAADMILLDDNFASIVTG |
| | VEEGRLIFDNLKKSIMYTLTSNIPEITPFLMFIILGIPLPLGTITILCIDLGTDMVPAI |
| | SLAYESAESDIMKRLPRNPKTDNLVNHRLIGMAYGQIGMIQALAGFFTYFVILAENGFR |
| | PVDLLGIRLHWEDKYLNDLEDSYGQQWTYEQRKVVEFTCQTAFFVTIVVVQWADLIISK |
| | TRRNSLFQQGMRNKVLIFGILEETLLAAFLSYTPGMDVALRMYPLKITWWLCAIPYSIL |
| | IFVYDEIRKLLIRQHPDGWVERETYY |
| ATP2B1 protein | MTKNEKKSLNQSLAEWKLFIYNPTTGEFLGRTAKSWGLILLFYLVFYGFLAALFSFTMW |
| (SEQ ID NO: 209) | VMLQTLNDEVPKYRDQIPSPGLMVFPKPVTALEYTFSRSDPTSYAGYIEDLKKFLKPYT |
| | LEEQKNLTVCPDGALFEQKGPVYVACQFPISLLQACSGMNDPDFGYSQGNPCILVKMNR |
| | IIGLKPEGVPRIDCVSKNEDIPNVAVYPHNGMIDLKYFPYYGKKLHVGYLQPLVAVQVS |
| | FAPNNTGKEVTVECKIDGSANLKSQDDRDKFLGRVMFKITARA |
| ATP2B2 protein | MGDMANNSVAYSGVKNSLKEANHDGDFGITLAELRALMELRSTDALRKIQESYGDVYGI |
| (SEQ ID NO: 210) | CTKLKTSPNEGLSGNPADLERREAVFGKNFIPPKKPKTFLQLVWEALQDVTLIILEIAA |
| | IVSLGLSFYQPPEGDNALCGEVSVGEEEGEGETGWIEGAAILLSVVCVVLVTAFNDWSK |
| | EKQFRGLQSRIEQEQKFTVIRGGQVIQIPVADITVGDIAQVKYGDLLPADGILIQGNDL |
| | KIDESSLTGESDHVKKSLDKDPLLLSGTHVMEGSGRMVVTAVGVNSQTGIIFTLLGAGG |
| | EEEEKKDEKKKEKKNKKQDGAIENRNKAKAQDGAAMEMQPLKSEEGGDGDEKDKKKANL |
| | PKKEKSVLQGKLTKLAVQIGKAGLLMSAITVIILVLYFVIDTFWVQKRPWLAECTPIYI |
| | QYFVKFFIIGVTVLVVAVPEGLPLAVTISLAYSVKKMMKDNNLVRHLDACETMGNATAI |
| | CSDKTGTLTMNRMTVVQAYINEKHYKKVPEPEAIPPNILSYLVTGISVNCAYTSKILPP |
| | EKEGGLPRHVGNKTECALLGLLLDLKRDYQDVRNEIPEEALYKVYTFNSVRKSMSTVLK |
| | NSDGSYRIFSKGASEIILKKCFKILSANGEAKVFRPRDRDDIVKTVIEPMASEGLRTIC |
| | LAFRDFPAGEPEPEWDNENDIVTGLTCIAVVGIEDPVRPEVPDAIKKCQRAGITVRMVT |
| | GDNINTARAIATKCGILHPGEDFLCLEGKDFNRRIRNEKGEIEQERIDKIWPKLRVLAR |
| | SSPTDKHTLVKGIIDSTVSDQRQVVAVTGDGTNDGPALKKADVGFAMGIAGTDVAKEAS |
| | DIILTDDNFTSIVKAVMWGRNVYDSISKFLQFQLTVNVVAVIVAFTGACITQDSPLKAV |
| | QMLWVNLIMDTLASLALATEPPTESLLLRKPYGRNKPLISRTMMKNILGHAFYQLVVVF |
| | TLLFAGEKFFDIDSGRNAPLHAPPSEHYTIVFNTFVLMQLFNEINARKIHGERNVFEGI |
| | FNNAIFCTIVLGTFVVQIIIVQFGGKPFSCSELSIEQWLWSIFLGMGTLLWGQLISTIP |
| | TSRLKFLKEAGHGTQKEEIPEEELAEDVEEIDHAERELRRGQILWFRGLNRIQTQMDVV |
| | NAFQSGSSIQGALRRQPSIASQHHDVTNISTPTHIRVVNAFRSSLYEGLEKPESRSSIH |
| | NFMTHPEFRIEDSEPHIPLIDDTDAEDDAPTKRNSSPPPSPNKNNNAVDSGIHLTIEMN |
| | KSATSSSPGSPLHSLETSL |
| ATP2B3 protein | MGDMTNSDFYSKNQRNESSHGGEFGCTMEELRSLMELRGTEAVVKIKETYGDTEAICRR |
| (SEQ ID NO: 211) | LKTSPVEGLPGTAPDLEKRKQIFGQNFIPPKKPKTFLQLVWEALQDVTLIILEIAAIIS |
| | LGLSFYHPPGEGNEGCATAQGGAEDEGEAEAGWIEGAAILLSVICVVLVTAFNDWSKEK |
| | QFRGLQSRIEQEQKFTVVRAGQVVQIPVAEIVVGDIAQVKYGDLLPADGLFIQGNDLKI |
| | DESSLTGESDQVRKSVDKDPMLLSGTHVMEGSGRMLVTAVGVNSQTGIIFTLLGAGGEE |
| | EEKKDKKGVKKGDGLQLPAADGAAASNAADSANASLVNGKMQDGNVDASQSKAKQQDGA |
| | AAMEMQPLKSAEGGDADDRKKASMHKKEKSVLQGKLTKLAVQIGKAGLVMSAITVIILV |
| | LYFTVDTFVVNKKPWLPECTPVYVQYFVKFFIIGVTVLVVAVPEGLPLAVTISLAYSVK |
| | KMMKDNNLVRHLDACETMGNATAICSDKTGTLTTNRMTVVQAYVGDVHYKEIPDPSSIN |
| | TKTMELLINAIAINSAYTTKILPPEKEGALPRQVGNKTECGLLGFVLDLKQDYEPVRSQ |
| | MPEEKLYKVYTFNSVRKSMSTVIKLPDESFRMYSKGASEIVLKKCCKILNGAGEPRVFR |
| | PRDRDEMVKKVIEPMACDGLRTICVAYRDFPSSPEPDWDNENDILNELTCICVVGIEDP |
| | VRPEVPEAIRKCQRAGITVRMVTGDNINTARAIAIKCGIIHPGEDFLCLEGKEFNRRIR |
| | NEKGEIEQERIDKIWPKLRVLARSSPTDKHTLVKGIIDSTHTEQRQVVAVTGDGTNDGP |
| | ALKKADVGFAMGIAGTDVAKEASDIILTDDNFSSIVKAVMWGRNVYDSISKFLQFQLTV |
| | NVVAVIVAFTGACITQDSPLKAVQMLWVNLIMDTFASLALATEPPTETLLLRKPYGRNK |
| | PLISRTMMKNILGHAVYQLALIFTLLFVGEKMFQIDSGRNAPLHSPPSEHYTIIFNTFV |
| | MMQLFNEINARKIHGERNVFDGIFRNPIFCTIVLGTFAIQIVIVQFGGKPFSCSPLQLD |
| | QWMWCIFIGLGELVWGQVIATIPTSRLKFLKEAGRLTQKEEIPEEELNEDVEEIDHAER |
| | ELRRGQILWFRGLNRIQTQIEVVNTFKSGASFQGALRRQSSVTSQSQDIRVVKAFRSSL |
| | YEGLEKPESRTSIHNFMAHPEFRIEDSQPHIPLIDDTDLEEDAALKQNSSPPSSLNKNN |
| | SAIDSGINLTTDTSKSATSSSPGSPIHSLETSL |
| ATP2B4 protein | MGDMANSSIEFHPKPQQQRDVPQAGGFGCTLAELRTLMELRGAEALQKIEEAYGDVSGL |
| (SEQ ID NO: 212) | CRRLKTSPTEGLADNTNDLEKRRQIYGQNFIPPKQPKTFLQLVWEALQDVTLIILEVAA |
| | IVSLGLSFYAPPGEESEACGNVSGGAEDEGEAEAGWIEGAAILLSVICVVLVTAFNDWS |
| | KEKQFRGLQSRIEQEQKFTVIRNGQLLQVPVAALVVGDIAQVKYGDLLPADGVLIQAND |
| | LKIDESSLTGESDHVRKSADKDPMLLSGTHVMEGSGRMVVTAVGVNSQTGIIFTLLGAG |
| | GEEEEKKDKKGKQQDGAMESSQTKAKKQDGAVAMEMQPLKSAEGGEMEEREKKKANAPK |
| | KEKSVLQGKLTKLAVQIGKAGLVMSAITVIILVLYFVIETFVVEGRTWLAECTPVYVQY |
| | FVKFFIIGVTVLVVAVPEGLPLAVTISLAYSVKKMMKDNNLVRHLDACETMGNATAICS |
| | DKTGTLTTNRMTVVQSYLGDTHYKEIPAPSALTPKILDLLVHAISINSAYTTKILPPEK |
| | EGALPRQVGNKTECALLGFVLDLKRDFQPVREQIPEDKLYKVYTFNSVRKSMSTVIRMP |
| | DGGFRLFSKGASEILLKKCTNILNSNGELRGFRPRDRDDMVRKIIEPMACDGLRTICIA |
| | YRDFSAGQEPDWDNENEVVGDLTCIAVVGIEDPVRPEVPEAIRKCQRAGITVRMVTGDN |
| | INTARAIAAKCGIIQPGEDFLCLEGKEFNRRIRNEKGEIEQERLDKVWPKLRVLARSSP |
| | TDKHTLVKGIIDSTTGEQRQVVAVTGDGTNDGPALKKADVGFAMGIAGTDVAKEASDII |
| | LTDDNFTSIVKAVMWGRNVYDSISKFLQFQLTVNVVAVIVAFTGACIT |

In other embodiments, the scaffold moiety, e.g., Scaffold X, comprises the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the corresponding mature BSG protein, IGSF8 protein, IGSF3 protein, ITGB1 protein, SLC3A2 protein, ITGA4 protein, ATP1A1 protein, ATP1A2 protein, ATP1A3 protein, ATP1A4 protein, ATP1A5 protein, ATP2B1 protein, ATP2B2 protein, ATP2B3 protein, ATP2B4 protein, or IGSF2 protein (without the signal peptide). In some aspects, the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein lacks one or more functional or structural domains, such as IgV.

Non-limiting examples of other Scaffold X proteins can be found at US patent No. U.S. Pat. No. 10,195,290B1, issued Feb. 5, 2019, which is incorporated by reference in its entirety, the ATP transporter proteins: ATP1A1, ATP1A2, ATP1A3, ATP1A4, ATP1B3, ATP2B1, ATP2B2, and ATP2B4), CD9, CD63, CD81, PDGFR, GPI proteins, lactadherin, LAMP2, and LAMP2B.

In some aspects, a scaffold moiety, e.g., Scaffold X, comprises Basigin (the BSG protein). The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ ID NO: 3. Amino acids 138-323 of SEQ ID NO:3 are the extracellular domain, amino acids 324 to 344 of SEQ ID NO:3 are the transmembrane domain, and amino acids 345 to 385 of SEQ ID NO:3 are the cytoplasmic domain of BSG.

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of human BSG protein (SEQ ID NO:3). In some aspects, the fragments of Basigin polypeptide lack one or more functional or structural domains, such as IgV, e.g., amino acids 221 to 315 of human BSG protein.

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 193, 194, or 195. In other aspects, the scaffold moiety, e.g., Scaffold X, comprises the amino acid sequence of SEQ ID NO: 193, 194, or 195, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of SEQ ID NO: 193, 194, or 195 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 193, 194, or 195.

In some aspects, a scaffold moiety, e.g., Scaffold X, comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 4 herein. The human IGSF8 protein has a signal peptide (amino acids 1 to 27 of human IGSF8 protein; SEQ ID NO: 4), an extracellular domain (amino acids 28 to 579 of human IGSF8 protein; SEQ ID NO: 4), a transmembrane domain (amino acids 580 to 600 of human IGSF8 protein; SEQ ID NO: 4), and a cytoplasmic domain (amino acids 601 to 613 of human IGSF8 protein; SEQ ID NO: 4).

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of human IGSF8 protein (SEQ ID NO: 4). In some aspects, the IGSF8 protein lacks one or more functional or structural domains, such as IgV. In other aspects, the scaffold moiety, e.g., Scaffold X, comprises the amino acid sequence of human IGSF8 protein (SEQ ID NO: 4), except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of human IGSF8 protein (SEQ ID NO: 4) and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of human IGSF8 protein (SEQ ID NO: 4).

In some aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 197, 198, 199, or 200. In other aspects, the scaffold moiety, e.g., Scaffold X, comprises the amino acid sequence of SEQ ID NO: 197, 198, 199, or 200, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of SEQ ID NO: 197, 198, 199, or 200 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 197, 198, 199, or 200.

In some aspects, a scaffold moiety, e.g., Scaffold X, for the present disclosure comprises Immunoglobulin superfamily member 3 (IgSF3 or the IGSF3 protein), which is also known as Glu-Trp-Ile EWI motif-containing protein 3 (EWI-3), and is shown as the amino acid sequence of SEQ ID NO: 203. The human IGSF3 protein has a signal peptide (amino acids 1 to 19 of the IGSF3 protein of SEQ ID NO: 203), an extracellular domain (amino acids 20 to 1124 of the IGSF3 protein of SEQ ID NO: 203), a transmembrane domain (amino acids 1125 to 1145 of the IGSF3 protein), and a cytoplasmic domain (amino acids 1146 to 1194 of the IGSF3 protein of SEQ ID NO: 203).

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of the IGSF3 protein (SEQ ID NO: 203). In some aspects, the IGSF3 protein lack one or more functional or structural domains, such as IgV.

In some aspects, a scaffold moiety, e.g., Scaffold X, for the present disclosure comprises Integrin beta-1 (the ITGB1 protein), which is also known as Fibronectin receptor subunit beta, Glycoprotein IIa (GPIIA), VLA-4 subunit beta, or CD29, and is shown as the amino acid sequence of SEQ ID NO: 5. The human ITGB1 protein has a signal peptide (amino acids 1 to 20 of the human ITGB1 protein of SEQ ID NO: 5), an extracellular domain (amino acids 21 to 728 of the human ITGB1 protein of SEQ ID NO: 5), a transmembrane domain (amino acids 729 to 751 of the human ITGB1 protein of SEQ ID NO: 5), and a cytoplasmic domain (amino acids 752 to 798 of the human ITGB1 protein of SEQ ID NO: 5).

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of the human ITGB1 protein (SEQ ID NO: 5). In some aspects, the ITGB1 protein lack one or more functional or structural domains, such as IgV.

In other aspects, the scaffold moiety, e.g., Scaffold X, comprises the ITGA4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the human ITGB1 protein of SEQ ID NO: 6 without the signal peptide (amino acids 1 to 33 of the human ITGB1 protein of SEQ ID NO: 6). In some aspects, the ITGA4 protein lacks one or more functional or structural domains, such as IgV.

In other aspects, the scaffold moiety, e.g., Scaffold X, comprises the SLC3A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the SLC3A2 protein of SEQ ID NO: 7 without the signal peptide. In some aspects, the SLC3A2 protein lacks one or more functional or structural domains, such as IgV.

In other aspects, the scaffold moiety, e.g., Scaffold X, comprises the ATP1A1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A1 protein of SEQ ID NO: 204 without the signal peptide. In some aspects, the ATP1A1 protein lacks one or more functional or structural domains, such as IgV.

In other aspects, the scaffold moiety, e.g., Scaffold X, comprises the ATP1A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A2 protein of SEQ ID NO:205 without the signal peptide. In some aspects, the ATP1A2 protein lacks one or more functional or structural domains, such as IgV.

In other aspects, the scaffold moiety, e.g., Scaffold X, comprises the ATP1A3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A3 protein of SEQ ID NO:206 without the signal peptide. In some aspects, the ATP1A3 protein lacks one or more functional or structural domains, such as IgV.

In other aspects, the scaffold moiety, e.g., Scaffold X, comprises the ATP1A4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A4 protein of SEQ ID NO:207 without the signal peptide. In some aspects, the ATP1A4 protein lacks one or more functional or structural domains, such as IgV.

In other aspects, the scaffold moiety, e.g., Scaffold, X comprises the ATP1B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1B3 protein of SEQ ID NO:208 without the signal peptide. In some aspects, the ATP1B3 protein lacks one or more functional or structural domains, such as IgV.

In other aspects, the scaffold moiety, e.g., Scaffold X, comprises the ATP2B1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B1 protein of SEQ ID NO:209 without the signal peptide. In some aspects, the ATP2B1 protein lacks one or more functional or structural domains, such as IgV.

In other aspects, the scaffold moiety, e.g., Scaffold X, comprises the ATP2B2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B2 protein of SEQ ID NO:210 without the signal peptide. In some aspects, the ATP2B2 protein lacks one or more functional or structural domains, such as IgV.

In other aspects, the scaffold moiety, e.g., Scaffold X, comprises the ATP2B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B3 protein of SEQ ID NO:211 without the signal peptide. In some aspects, the ATP2B3 protein lacks one or more functional or structural domains, such as IgV.

In other aspects, the scaffold moiety, e.g., Scaffold X, comprises the ATP2B4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B4 protein of SEQ ID NO:212 without the signal peptide. In some aspects, the ATP2B4 protein lacks one or more functional or structural domains, such as IgV.

In other aspects, the scaffold moiety, e.g., Scaffold X, comprises the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the IGSF2 protein (SEQ ID NO: 202) without the signal peptide. In some aspects, the IGSF2 protein lacks one or more functional or structural domains, such as IgV.

Non-limiting examples of other scaffold moieties, e.g., Scaffold X proteins, can be found at US patent No. U.S. Ser. No. 10/195,290B1, issued Feb. 5, 2019, which is incorporated by reference in its entirety.

In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from the N-terminus of the native protein. In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from the C-terminus of the native protein. In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from both the N-terminus and C-terminus of the native protein. In some aspects, the sequence encodes a fragment of the scaffold moiety lacking one or more functional or structural domains of the native protein.

In some aspects, the scaffold moiety, e.g., Scaffold X, e.g., a PTGFRN protein, is linked to one or more heterologous proteins. The one or more heterologous proteins can be linked to the N-terminus of the scaffold moiety. The one or more heterologous proteins can be linked to the C-terminus of the scaffold moiety. In some aspects, the one or more heterologous proteins are linked to both the N-terminus and the C-terminus of the scaffold moiety. In some aspects, the heterologous protein is a mammalian protein. In some aspects, the heterologous protein is a human protein.

In some aspects, the scaffold moiety, e.g., Scaffold X, can be used to link any moiety to the luminal surface and the external surface of the EV (e.g., exosome) at the same time. For example, the PTGFRN polypeptide can be used to link one or more biologically active molecules indirectly through a maleimide moiety or directly to a maleimide moiety or a linker to the luminal surface in addition to the external surface of the EV (e.g., exosome). Therefore, in certain aspects, Scaffold X can be used for dual purposes.

In other aspects, the EVs, e.g., exosomes, of the present disclosure comprise a higher number of Scaffold X proteins compared to the naturally-occurring EVs, e.g., exosomes. In some aspects, the EVs, e.g., exosomes, of the disclosure comprise at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold higher number of Scaffold X (e.g., a PTGFRN polypeptide) compared to the naturally-occurring EV (e.g., exosome).

The number of scaffold moieties, e.g., Scaffold X, such as, a PTGFRN polypeptide, on the EV (e.g., exosome) of the present disclosure is at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 4000, at least about 5000, at least about 6000, at least about 7000, at least about 8000, at least about 9000, or at least about 10000.

In some aspects, the number of scaffold moieties, e.g., Scaffold X, such as, a PTGFRN polypeptide, on the EV, e.g., exosome, of the present disclosure is from about 100 to about 100,000, from about 200 to about 9000, from about 300 to about 9000, from about 400 to about 9000, from about 500 to about 9000, from about 600 to about 8000, from about 800 to about 8000, from about 900 to about 8000, from about 1000 to about 8000, from about 1100 to about 8000, from about 1200 to about 8000, from about 1300 to about 8000, from about 1400 to about 8000, from about 1500 to about 8000, from about 1600 to about 8000, from about 1700 to about 8000, from about 1800 to about 8000, from about 1900 to about 8000, from about 2000 to about 8000, from about 2100 to about 8000, from about 2200 to about 8000, from about 2300 to about 8000, from about 2400 to about 8000, from about 2500 to about 8000, from about 2600, from about 2700 to about 8000, from about 2800 to about 8000, from about 2900 to about 8000, from about 3000 to about 8000, from about 4000 to about 8000, from about 5000 to about 8000, from about 6000 to about 8000, from about 7000 to about 8000, from about 8000, from 7000 to about 9000, or from about 6000 to about 10000.

In some aspects, the number of scaffold moieties, e.g., Scaffold X, such as, a PTGFRN polypeptide, on the EV (e.g., exosome) of the present disclosure is from about 5000 to about 8000, e.g., about 5000, about 6000, about 7000, or about 8000. In some aspects, the number of scaffold moieties, e.g., Scaffold X, such as, a PTGFRN polypeptide, on the EV (e.g., exosome) of the present disclosure is from about 6000 to about 8000, e.g., about 6000, about 7000, or about 8000. In some aspects, the number scaffold moieties, e.g., Scaffold X, such as, a PTGFRN polypeptide, on the EV (e.g., exosome) of the present disclosure is from about 4000 to about 9000, e.g., about 4000, about 5000, about 6000, about 7000, about 8000, about 9000.

II.G.2 Luminal Scaffold Moieties (e.g., Scaffold Y)

In some aspects, EVs, e.g., exosomes, of the present disclosure comprise an internal space (i.e., lumen) that is different from that of the naturally occurring EVs, e.g., exosomes. For example, the EV, e.g., exosome, can be changed such that the composition on the luminal surface of the EV, e.g., exosome, has the protein, lipid, or glycan content different from that of the naturally-occurring EVs, e.g., exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the exosome. Various modifications or fragments of the EV, e.g., exosome, protein that can be expressed on the luminal surface of the EV, e.g, exosome, can be used for the aspects of the present disclosure.

In some aspects, the EV, e.g, exosome, proteins that can change the luminal surface of the EV, e.g, exosome, include, but are not limited to the MARCKS protein, MARCKSL1 protein, BASP1 protein, or any combination thereof. In some aspects, the scaffold moiety, e.g., Scaffold Y, comprises Brain Acid Soluble Protein 1 (the BASP1 protein). The BASP1 protein is also known as 22 kDa neuronal tissue-enriched acidic protein or neuronal axonal membrane protein NAP-22. The full-length human BASP1 protein sequence (isomer 1) is shown in TABLE 3. An isomer produced by an alternative splicing is missing amino acids 88 to 141 from the BASP1 protein in TABLE 3 (isomer 1).

TABLE 3
Exemplary Scaffold Protein Sequences

| Protein | Sequence |
|---|---|
| BASP1 protein | MGGKLSKKKKGYNVNDEKAKEKDKKAEGAATEEEGTPKESEPQAAAEPAE |
| (SEQ ID NO: 10 | AKEGKEKPDQDAEGKAEEKEGEKDAAAAKEEAPKAEPEKTEGAAEAKAEP |
| | PKAPEQEQAAPGPAAGGEAPKAAEAAAAPAESAAPAAGEEPSKEEGEPKK |
| | TEAPAAPAAQETKSDGAPASDSKPGSSEAAPSSKETPAATEAPSSTPKAQ |
| | GPAASAEEPKPVEAPAANSDQTVTVKE |
| MARCKSL1 | MGSQSSKAPRGDVTAEEAAGASPAKANGQENGHVKSNGDLSPKGEGESPP |
| protein | VNGTDEAAGATGDAIEPAPPSQGAEAKGEVPPKETPKKKKKFSFKKPFKL |
| (SEQ ID NO: 9 | SGLSFKRNRKEGGGDSSASSPTEEEQEQGEIGACSDEGTAQEGKAAATPE |
| | SQEPQAKGAEASAASEEEAGPQATEPSTPSGPESGPTPASAEQNE |
| MARCKS protein | MGAQFSKTAA KGEAAAERPG EAAVASSPSK ANGQENGHVK VNGDASPAAA |
| (SEQ ID NO: 8) | ESGAKEELQA NGSAPAADKE EPAAAGSGAA SPSAAEKGEP AAAAAPEAGA |
| | SPVEKEAPAE GEAAEPGSPT AAEGEAASAA SSTSSPKAED GATPSPSNET |
| | PKKKKKRFSF KKSFKLSGFS FKKNKKEAGE GGEAEAPAAE GGKDEAAGGA |
| | AAAAAEAGAA SGEQAAAPGE EAAAGEEGAA GGDPQEAKPQ EAAVAPEKPP |
| | ASDETKAAEE PSKVEEKKAE EAGASAAACE APSAAGPGAP PEQEAAPAEE |
| | PAAAAASSAC AAPSQEAQPE CSPEAPPAEA AE |

In some aspects, the scaffold moiety, e.g., Scaffold Y, comprises a protein is selected from the group consisting of MARCKS, MARKSL1, BASP1, any functional fragment, variant, or derivative thereof, or any combination thereof. In some aspects, the scaffold moiety, e.g., Scaffold Y, comprises an Src protein or a fragment thereof. In some aspects, the scaffold moiety, e.g., Scaffold Y, comprises a sequence disclosed, e.g., in U.S. Pat. No. 9,611,481.

In some aspects, the scaffold moiety, e.g., Scaffold Y, of the present disclosure comprises the MARCKS protein, or a fragment, variant, or derivative thereof. The MARCKS protein (Uniprot accession no. P29966) is also known as protein kinase C substrate, 80 kDa protein, light chain. The full-length human MARCKS protein is 332 amino acids in length and comprises a calmodulin-binding domain at amino acid residues 152-176. In some aspects, the scaffold moiety, e.g., Scaffold Y, of the present disclosure comprises a mature MARCKS protein (i.e., without N-terminal methionine). In some aspects, the scaffold moiety, e.g., Scaffold Y, of the present disclosure is derived from a mature MARCKS protein, i.e., it is a fragment, variant, or derivate of a mature MARCKS protein and therefore it lacks the N-terminal protein present in the nonmature protein.

In some aspects, the scaffold moiety, e.g., Scaffold Y, of the present disclosure comprises the MARCKSL1 protein (Uniprot accession no. P49006), also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the scaffold moiety, e.g., Scaffold Y, of the present disclosure comprises a mature MARCKSL1 protein (i.e., without N-terminal methionine). In some aspects, the scaffold moiety, e.g., Scaffold Y, of the present disclosure is derived from a mature MARCKSL1 protein, i.e., it is a fragment, variant, or derivate of a mature MARCKSL1 protein and therefore it lacks the N-terminal protein present in the non-mature protein.

In some aspects, the scaffold moiety, e.g., Scaffold Y, of the present disclosure comprises the BASP1 protein (Uniprot accession number P80723), also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from isomer 1. In some aspects, the scaffold moiety, e.g., Scaffold Y, of the present disclosure comprises a mature BASP1 protein (i.e., without N-terminal methionine). In some aspects, the scaffold moiety, e.g., Scaffold Y, of the present disclosure is derived from a mature BASP1 protein, i.e., it is a fragment, variant, or derivate of a mature BASP1 protein and therefore it lacks the N-terminal protein present in the non-mature protein. The mature BASP1 protein sequence is missing the first Met from SEQ ID NO: 10 and thus contains amino acids 2 to 227 of SEQ ID NO: 10.

In other aspects, a scaffold moiety, e.g., Scaffold Y, useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 10, i.e., the mature form of BASP1 (i.e., without the N-terminal methionine amino acid present in SEQ ID NO: 10). In other aspects, the scaffold moiety, e.g., a Scaffold X protein, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to a functional fragment of the mature form of SEQ ID NO: 10 (BASP1), i.e., without the N-terminal methionine amino acid present in SEQ ID NO: 10. In other aspects, a scaffold moiety, e,g, Scaffold, Y useful for the present disclosure comprises the amino acid sequence of SEQ ID NO: 10 except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a scaffold moiety, e.g., Scaffold Y, useful for the present disclosure comprises the amino acid sequence of SEQ ID NO: 10 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 10.

In other aspects, a scaffold moiety, e.g., Scaffold Y, useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the mature form of SEQ ID NO:9 (MARCKSL1) i.e., without the N-terminal methionine amino acid present in SEQ ID NO: 9. In other aspects, the scaffold moiety, e.g., Scaffold Y, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to a functional fragment of the mature form of SEQ ID NO: 9 (MARCKSL1), i.e., without the N-terminal methionine amino acid present in SEQ ID NO: 9. In other aspects, a scaffold moiety, e.g., Scaffold Y, useful for the present disclosure comprises the amino acid sequence of SEQ ID NO: 9 except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a scaffold moiety, e.g., Scaffold Y, useful for the present disclosure comprises the amino acid sequence of SEQ ID NO: 9 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 9.

In other aspects, a scaffold moiety, e.g., Scaffold Y, useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the mature form of SEQ ID NO:8 (MARCKS) i.e., without the N-terminal methionine amino acid present in SEQ ID NO: 8. In other aspects, the scaffold moiety, e.g., Scaffold Y, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to a functional fragment of the mature form of SEQ ID NO: 8 (MARCKS). i.e., without the N-terminal methionine amino acid present in SEQ ID NO: 8. In other aspects, a scaffold moiety, e.g., Scaffold Y, useful for the present disclosure comprises the amino acid sequence of SEQ ID NO: 8 except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a scaffold moiety, e.g., Scaffold Y, useful for the present disclosure comprises the amino acid sequence of SEQ ID NO: 8 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 8.

In certain aspects, the protein sequence of any of SEQ ID NOs: 1-109 disclosed in PCT/US2018/061679 is sufficient to be a Scaffold Y for the present disclosure (e.g., scaffold moiety linked to a linker).

In certain aspects, a scaffold moiety, e.g., Scaffold Y, useful for the present disclosure comprises a peptide with the MGXKLSKKK (SEQ ID NO: 224) or GXKLSKKK (SEQ ID NO:225), where X is alanine or any other amino acid. In some aspects, an EV (e.g., exosome) comprises a peptide with sequence of (M)(G)(π)(ξ)(Φ/π)(S/A/G/N)(+)(+) or (G)(π)(ξ)(Φ/π)(S/A/G/N)(+)(+), wherein each parenthetical position represents an amino acid, and wherein 7L is any amino acid selected from the group consisting of (Pro, Gly, Ala, Ser), is any amino acid selected from the group consisting of (Asn, Gln, Ser, Thr, Asp, Glu, Lys, His, Arg), (is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In further aspects, an EV (e.g., exosome) described herein (e.g., engineered exosome) comprises a peptide with sequence of (M)(G)(π)(X)(Φ/π)(π)(+)(+) or (G)(π)(X)(Φ/π)(+)(+), wherein each parenthetical position represents an amino acid, and wherein π L is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). See Aasland et al., FEBS Letters 513 (2002) 141-144 for amino acid nomenclature.

In other aspects, the scaffold moiety, e.g., Scaffold Y, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of the sequences disclosed in U.S. Pat. No. 10,195,290B1, issued Feb. 5, 2019.

Scaffold Y-engineered exosomes described herein can be produced from a cell transformed with any sequence set forth in PCT/US2018/061679 (SEQ ID NO: 4-109 from PCT/US2018/061679).

In other aspects, the EVs, e.g., exosomes, of the present disclosure comprise a higher number of Scaffold Y proteins compared to the naturally-occurring EVs, e.g., exosomes. In some aspects, the EVs, e.g., exosomes, of the disclosure comprise at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold higher number of Scaffold Y (e.g., a BASP-1 polypeptide) compared to the naturally-occurring EV, e.g., exosome. The number of Scaffold Y, e.g., BASP-1 polypeptide, on the EV, e.g., exosome, of the present disclosure is at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 4000, at least about 5000, at least about 6000, at least about 7000, at least about 8000, at least about 9000, or at least about 10000. In some aspects, the number of Scaffold Y, e.g., a BASP-1 polypeptide, on the EV, e.g., exosome, of the present disclosure is from about 100 to about 100,000, from about 200 to about 9000, from about 300 to about 9000, from about 400 to about 9000, from about 500 to about 9000, from about 600 to about 8000, from about 800 to about 8000, from about 900 to about 8000, from about 1000 to about 8000, from about 1100 to about 8000, from about 1200 to about 8000, from about 1300 to about 8000, from about 1400 to about 8000, from about 1500 to about 8000, from about 1600 to about 8000, from about 1700 to about 8000, from about 1800 to about 8000, from about 1900 to about 8000, from about 2000 to about 8000, from about 2100 to about 8000, from about 2200 to about 8000, from about 2300 to about 8000, from about 2400 to about 8000, from about 2500 to about 8000, from about 2600, from about 2700 to about 8000, from about 2800 to about 8000, from about 2900 to about 8000, from about 3000 to about 8000, from about 4000 to about 8000, from about 5000 to about 8000, from about 6000 to about 8000, from about 7000 to about 8000, from about 8000, from 7000 to about 9000, or from about 6000 to about 10000. In some aspects, the number of Scaffold Y, e.g., a BASP-1 polypeptide, on the EV, e.g., exosome, of the present disclosure is from about 5000 to about 8000, e.g., about 5000, about 6000, about 7000, or about 8000. In some aspects, the number of Scaffold Y, e.g., a BASP-1 polypeptide, on the EV, e.g., exosome, of the present disclosure is from about 6000 to about 8000, e.g., about 6000, about 7000, or about 8000. In some aspects, the number of Scaffold Y, e.g., a BASP-1 polypeptide, on the EV, e.g., exosome, of the present disclosure is from about 4000 to about 9000, e.g., about 4000, about 5000, about 6000, about 7000, about 8000, about 9000.

In some aspects, the scaffold moiety, e.g., Scaffold Y, useful for the present disclosure comprises an “N-terminus domain” (ND) and an “effector domain” (ED), wherein the ND and/or the ED are associated with the luminal surface of the EV, e.g., an exosome. In some aspects, the scaffold moiety, e.g., Scaffold Y, useful for the present disclosure comprises an intracellular domain, a transmembrane domain, and an extracellular domain; wherein the intracellular domain comprises an “N-terminus domain” (ND) and an “effector domain” (ED); wherein the ND and/or the ED are associated with the luminal surface of the EV, e.g., an exosome. As used herein the term “associated with” refers to the interaction between a scaffold protein of the present disclosure with the luminal surface of the EV, e.g., and exosome, that does not involve covalent linking to a membrane component. For example, the scaffold moieties useful for the present disclosure can be associated with the luminal surface of the EV, e.g., via a lipid (e.g., myristic acid), and/or a polybasic domain that interacts electrostatically with the negatively charged head of membrane phospholipids. In other aspects, the scaffold moiety, e.g., Scaffold Y, comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the scaffold moiety, e.g., Scaffold Y, comprises an N-terminus domain (ND) and an effector domain (ED), wherein the ND is associated with the luminal surface of the EV, and the ED is associated with the luminal surface of the EV by an ionic interaction, wherein the ED comprises at least two, at least three, at least four, at least five, at least six, or at least seven contiguous lysines (Lys) in sequence.

In other aspects, the ED further comprises one or more low complexity regions, e.g., a PEST motif. A PEST sequence is a peptide sequence that is rich in proline (P), glutamic acid (E), serine (S), and threonine (T). In some aspects, the ED further comprises negatively charged residues (for example, Glu) and many Ser and Thr that undergo transient phosphorylation (thus, both adding negative charges to the areas out of ED).

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)ₙ, wherein n is an integer between 1 and 10.

In some aspects, the ED comprises (i) a lysine repeat in the ED or (ii) a lysine repeat with the ND, e.g., K at the C terminus in the ND and K at the N terminus in the ED, wherein the ND and ED are linked directly, i.e., by a peptide bond. In some aspects, the minimum number of the amino acids that are capable of linking a heterologous moiety, e.g., a biologically active molecule, in the lumen of the EV, e.g., exosome, e.g., about seven to about 15, about seven to about 14, about seven to about 13, about seven to about 12, about seven to about 11, about seven to about 10, about seven to about 9, or about seven to about 8 amino acid fragments.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids. In some aspects, the ED comprises at least two contiguous lysines (Lys) in sequence.

In some aspects, the ED comprises K, KK, KKK, KKKK (SEQ ID NO: 11), KKKKK (SEQ ID NO: 12), R, RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof. In some aspects, the ED comprises KK, KKK, KKKK (SEQ ID NO: 11), KKKKK (SEQ ID NO: 12), or any combination thereof. In some aspects, the ND comprises the amino acid sequence as set forth in G:X2:X3:X4:X5:X6, wherein G represents Gly; wherein “:” represents a peptide bond; wherein each of the X2 to the X6 independently represents an amino acid; and wherein the X6 represents a basic amino acid. In some aspects, the X6 amino acid is selected is selected from the group consisting of Lys, Arg, and His. In some aspects, the X5 amino acid is selected from the group consisting of Pro, Gly, Ala, and Ser. In some aspects, the X2 amino acid is selected from the group consisting of Pro, Gly, Ala, and Ser. In some aspects, the X4 is selected from the group consisting of Pro, Gly, Ala, Ser, Val, Ile, Leu, Phe, Trp, Tyr, Gln, and Met.

In some aspects, the scaffold moiety, e.g., Scaffold Y, comprises an N-terminus domain (ND) and an effector domain (ED), wherein the ND comprises the amino acid sequence as set forth in G:X2:X3:X4:X5:X6, wherein G represents Gly; wherein “:” represents a peptide bond; wherein each of the X2 to the X6 is independently an amino acid; wherein the X6 comprises a basic amino acid, and wherein the ED is linked to X6 by a peptide bond and comprises at least one lysine at the N terminus of the ED.

In some aspects, the ND of the scaffold moiety, e.g., Scaffold Y, comprises the amino acid sequence of G:X2:X3:X4:X5:X6, wherein G represents Gly; “:” represents a peptide bond; the X2 represents an amino acid selected from the group consisting of Pro, Gly, Ala, and Ser; the X3 represents any amino acid; the X4 represents an amino acid selected from the group consisting of Pro, Gly, Ala, Ser, Val, Ile, Leu, Phe, Trp, Tyr, Gln, and Met; the X5 represents an amino acid selected from the group consisting of Pro, Gly, Ala, and Ser; and the X6 represents an amino acid selected from the group consisting of Lys, Arg, and His.

In some aspects, the X3 amino acid is selected from the group consisting of Asn, Gln, Ser, Thr, Asp, Glu, Lys, His, and Arg.

In some aspects, the ND and ED are joined by a linker. In some aspects, the linker comprises one or more amino acids. In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence. When the ND and ED are joined by a linker, the ED comprise at least two lysines, at least three lysines, at least four lysines, at least five lysines, at least six lysines, or at least seven lysines. Linkers that can used to join ND and ED are disclosed elsewhere in the present specification.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the linker is a glycine/serine linker. In some aspects, the peptide linker is glycine/serine linker according to the formula [(Gly)n-Ser]m (SEQ ID NO: 46) where n is any integer from 1 to 100 and m is any integer from 1 to 100. In other aspects, the glycine/serine linker is according to the formula [(Gly)x-Sery]z (SEQ ID NO: 47) wherein x in an integer from 1 to 4, y is 0 or 1, and z is an integers from 1 to 50. In some aspects, the peptide linker comprises the sequence Gn (SEQ ID NO: 48), where n can be an integer from 1 to 100. In some aspects, the peptide linker can comprise the sequence (GlyAla)n (SEQ ID NO: 49), wherein n is an integer between 1 and 100. In other aspects, the peptide linker can comprise the sequence (GlyGlySer)n (SEQ ID NO:50), wherein n is an integer between 1 and 100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

In some aspects, the scaffold moiety, e.g., Scaffold Y, comprises ND-ED, wherein: ND comprises G:X2:X3:X4:X5:X6; wherein: G represents Gly; “:” represents a peptide bond; X2 represents an amino acid selected from the group consisting of Pro, Gly, Ala, and Ser; X3 represents any amino acid; X4 represents an amino acid selected from the group consisting of Pro, Gly, Ala, Ser, Val, Ile, Leu, Phe, Trp, Tyr, Glu, and Met; X5 represents an amino acid selected from the group consisting of Pro, Gly, Ala, and Ser; X6 represents an amino acid selected from the group consisting of Lys, Arg, and His; “—” represents an optional linker; and ED is an effector domain comprising (i) at least two contiguous lysines (Lys), which is linked to the X6 by a peptide bond or one or more amino acids or (ii) at least one lysine, which is directly linked to the X6 by a peptide bond.

In some aspects, the X2 amino acid is selected from the group consisting of Gly and Ala. In some aspects, the X3 amino acid is Lys. In some aspects, the X4 amino acid is Leu or Glu. In some aspects, the X5 amino acid is selected from the group consisting of Ser and Ala. In some aspects, the X6 amino acid is Lys. In some aspects, the X2 amino acid is Gly, Ala, or Ser; the X3 amino acid is Lys or Glu; the X4 amino acid is Leu, Phe, Ser, or Glu; the X5 amino acid is Ser or Ala; and X6 amino acid is Lys. In some aspects, the “—” linker comprises a peptide bond or one or more amino acids.

In some aspects, the ED in the scaffold moiety comprises Lys (K), KK, KKK, KKKK (SEQ ID NO: 11), KKKKK (SEQ ID NO: 12), Arg (R), RR, RRR, RRRR (SEQ ID NO: 13); RRRRR (SEQ ID NO: 14), KR, RK, KKR, KRK, RKK, KRR, RRK, (K/R)(K/R)(K/R)(K/R) (SEQ ID NO: 15), (K/R)(K/R)(K/R)(K/R)(K/R) (SEQ ID NO: 16), or any combination thereof.

In some aspects, the scaffold moiety, e.g., Scaffold Y, comprises an amino acid sequence selected from the group consisting of (i) GGKLSKK (SEQ ID NO: 17), (ii) GAKLSKK (SEQ ID NO: 18), (iii) GGKQSKK (SEQ ID NO: 19), (iv) GGKLAKK (SEQ ID NO: 20), or (v) any combination thereof.

In some aspects, the ND in the scaffold moiety, e.g., Scaffold Y, comprises an amino acid sequence selected from the group consisting of (i) GGKLSK (SEQ ID NO: 51), (ii) GAKLSK (SEQ ID NO: 52), (iii) GGKQSK (SEQ ID NO: 53), (iv) GGKLAK (SEQ ID NO: 54), and (v) any combination thereof, and the ED in the scaffold protein comprises an amino acid sequence selected from the group consisting of K, KK, KKK, KKKG (SEQ ID NO: 55), KKKGY (SEQ ID NO: 56), KKKGYN (SEQ ID NO: 57), KKKGYNV (SEQ ID NO: 58), KKKGYNVN (SEQ ID NO: 59), KKKGYS (SEQ ID NO: 60), KKKGYG (SEQ ID NO: 61), KKKGYGG (SEQ ID NO: 62), KKKGS (SEQ ID NO: 63), KKKGSG (SEQ ID NO: 64), KKKGSGS (SEQ ID NO: 66), KKKS (SEQ ID NO: 67), KKKSG (SEQ ID NO: 68), KKKSGG (SEQ ID NO: 69), KKKSGGS (SEQ ID NO: 70), KKKSGGSG (SEQ ID NO: 71), KKSGGSGG (SEQ ID NO: 72), KKKSGGSGGS (SEQ ID NO: 73), KRFSFKKS (SEQ ID NO: 241) and any combination thereof.

In some aspects, the polypeptide sequence of a Scaffold Y useful for the present disclosure consists of an amino acid sequence selected from the group consisting of (i) GGKLSKK (SEQ ID NO: 21), (ii) GAKLSKK (SEQ ID NO: 18), (iii) GGKQSKK (SEQ ID NO: 19), (iv) GGKLAKK (SEQ ID NO: 20), or (v) any combination thereof.

In some aspects, the scaffold moiety, e.g., Scaffold Y, comprises an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), and (ix) any combination thereof.

In some aspects, the polypeptide sequence of a scaffold moiety, e.g., Scaffold Y, useful for the present disclosure consists of an amino acid sequence selected from the group consisting of (i) GGKLSKKK (SEQ ID NO: 22), (ii) GGKLSKKS (SEQ ID NO: 23), (iii) GAKLSKKK (SEQ ID NO: 24), (iv) GAKLSKKS (SEQ ID NO: 25), (v) GGKQSKKK (SEQ ID NO: 26), (vi) GGKQSKKS (SEQ ID NO: 27), (vii) GGKLAKKK (SEQ ID NO: 28), (viii) GGKLAKKS (SEQ ID NO: 29), and (ix) any combination thereof. In some aspects, the scaffold protein of the present disclosure comprises at least two contiguous lysines (Lys) in sequence.

In some aspects, the scaffold moiety, e.g., Scaffold Y, is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 39, at least about 40, at least about 41, at least about 42, at least about 43, at least about 44, at least about 45, at least about 46, at least about 47, at least about 48, at least about 49, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 335, at least about 340, at least about 345, or at least about 350 amino acids in length.

In some aspects, the scaffold moiety, e.g., Scaffold, Y is between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250, between about 250 and about 260, between about 260 and about 270, between about 270 and about 280, between about 280 and about 290, between about 290 and about 300, between about 300 and about 310, between about 310 and about 320, between about 320 and about 330, between about 330 and about 340, or between about 340 and about 250 amino acids in length.

In some aspects, the scaffold moiety, e.g., Scaffold Y, comprises (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ ID NO: 39), (ix) GGKLSKKSGGSGGS (SEQ ID NO: 40), (x) GGKLSKSGGSGGSV (SEQ ID NO: 41), or (xi) GAKKSKKRFSFKKS (SEQ ID NO: 42).

In some aspects, the polypeptide sequence of a scaffold moiety, e.g., Scaffold Y, useful for the present disclosure consists of (i) GGKLSKKKKGYNVN (SEQ ID NO: 32), (ii) GAKLSKKKKGYNVN (SEQ ID NO: 33), (iii) GGKQSKKKKGYNVN (SEQ ID NO: 34), (iv) GGKLAKKKKGYNVN (SEQ ID NO: 35), (v) GGKLSKKKKGYSGG (SEQ ID NO: 36), (vi) GGKLSKKKKGSGGS (SEQ ID NO: 37), (vii) GGKLSKKKKSGGSG (SEQ ID NO: 38), (viii) GGKLSKKKSGGSGG (SEQ TD NO 39), (ix) GGKLSKKSGGSGGS (SEQ ID NO: 40), (x) GGKLSKSGGSGGSV (SEQ ID NO: 41), or (xi) GAKKSKKRFSFKKS (SEQ TD NO: 42).

Non-limiting examples of scaffold moieties, e.g., Scaffold Y, useful for the present disclosure are listed below. In some aspects, the scaffold moiety, e.g., Scaffold Y, comprises an amino acid sequence set forth in TABLE 4. In some aspects, the scaffold moiety, e.g., Scaffold Y, consists of an amino acid sequence set forth in TABLE 4.

TABLE 4
Exemplary Scaffold Moieties

| SEQ ID NO: | Scaffold Protein: GX2X3X4X5X6-ED |
|---|---|
| 75 | GGKLSKKKKGYNVNDEKAKEKDKKAEGAA |
| 76 | GGKLSKKKKGYNVNDEKAKEKDKKAEGA |
| 77 | GGKLSKKKKGYNVNDEKAKEKDKKAEG |
| 78 | GGKLSKKKKGYNVNDEKAKEKDKKAE |
| 79 | GGKLSKKKKGYNVNDEKAKEKDKKA |
| 80 | GGKLSKKKKGYNVNDEKAKEKDKK |
| 81 | GGKLSKKKKGYNVNDEKAKEKDK |
| 82 | GGKLSKKKKGYNVNDEKAKEKD |
| 83 | GGKLSKKKKGYNVNDEKAKEK |
| 84 | GGKLSKKKKGYNVNDEKAKE |
| 85 | GGKLSKKKKGYNVNDEKAK |
| 86 | GGKLSKKKKGYNVNDEKA |
| 87 | GGKLSKKKKGYNVNDEK |
| 88 | GGKLSKKKKGYNVNDE |
| 89 | GGKLSKKKKGYNVND |
| 32 | GGKLSKKKKGYNVN |
| 90 | GGKLSKKKKGYNV |
| 91 | GGKLSKKKKGYN |
| 92 | GGKLSKKKKGY |
| 93 | GGKLSKKKKG |
| 94 | GGKLSKKKK |
| 22 | GGKLSKKK |
| 17 | GGKLSKK |
| 95 | GAKKSKKRFSFKKSFKLSGFSFKKNKKEA |
| 96 | GAKKSKKRFSFKKSFKLSGFSFKKNKKE |
| 97 | GAKKSKKRFSFKKSFKLSGFSFKKNKK |
| 98 | GAKKSKKRFSFKKSFKLSGFSFKKNK |
| 99 | GAKKSKKRFSFKKSFKLSGFSFKKN |
| 100 | GAKKSKKRFSFKKSFKLSGFSFKK |
| 101 | GAKKSKKRFSFKKSFKLSGFSFK |
| 102 | GAKKSKKRFSFKKSFKLSGFSF |
| 103 | GAKKSKKRFSFKKSFKLSGFS |
| 104 | GAKKSKKRFSFKKSFKLSGF |
| 105 | GAKKSKKRFSFKKSFKLSG |
| 106 | GAKKSKKRFSFKKSFKLS |
| 107 | GAKKSKKRFSFKKSFKL |
| 108 | GAKKSKKRFSFKKSFK |
| 109 | GAKKSKKRFSFKKSF |
| 42 | GAKKSKKRFSFKKS |
| 110 | GAKKSKKRFSFKK |
| 111 | GAKKSKKRFSFK |
| 112 | GAKKSKKRFSF |
| 113 | GAKKSKKRFS |
| 114 | GAKKSKKRF |
| 115 | GAKKSKKR |
| 116 | GAKKSKK |
| 117 | GAKKAKKRFSFKKSFKLSGFSFKKNKKEA |
| 118 | GAKKAKKRFSFKKSFKLSGFSFKKNKKE |
| 119 | GAKKAKKRFSFKKSFKLSGFSFKKNKK |
| 120 | GAKKAKKRFSFKKSFKLSGFSFKKNK |
| 121 | GAKKAKKRFSFKKSFKLSGFSFKKN |
| 122 | GAKKAKKRFSFKKSFKLSGFSFKK |
| 123 | GAKKAKKRFSFKKSFKLSGFSFK |
| 124 | GAKKAKKRFSFKKSFKLSGFSF |
| 125 | GAKKAKKRFSFKKSFKLSGFS |
| 126 | GAKKAKKRFSFKKSFKLSGF |
| 127 | GAKKAKKRFSFKKSFKLSG |
| 128 | GAKKAKKRFSFKKSFKLS |
| 129 | GAKKAKKRFSFKKSFKL |
| 130 | GAKKAKKRFSFKKSFK |
| 131 | GAKKAKKRFSFKKSF |
| 132 | GAKKAKKRFSFKKS |
| 133 | GAKKAKKRFSFKK |
| 134 | GAKKAKKRFSFK |
| 135 | GAKKAKKRFSF |
| 136 | GAKKAKKRFS |
| 137 | GAKKAKKRF |
| 138 | GAKKAKKR |
| 139 | GAKKAKK |
| 140 | GAQESKKKKKKRFSFKKSFKLSGFSFKK |
| 141 | GAQESKKKKKKRFSFKKSFKLSGFSFK |
| 142 | GAQESKKKKKKRFSFKKSFKLSGFSF |
| 143 | GAQESKKKKKKRFSFKKSFKLSGFS |
| 144 | GAQESKKKKKKRFSFKKSFKLSGF |
| 145 | GAQESKKKKKKRFSFKKSFKLSG |
| 146 | GAQESKKKKKKRFSFKKSFKLS |
| 147 | GAQESKKKKKKRFSFKKSFKL |
| 148 | GAQESKKKKKKRFSFKKSFK |
| 149 | GAQESKKKKKKRFSFKKSF |
| 150 | GAQESKKKKKKRFSFKKS |
| 151 | GAQESKKKKKKRFSFKK |
| 152 | GAQESKKKKKKRFSFK |
| 153 | GAQESKKKKKKRFSF |
| 154 | GAQESKKKKKKRFS |
| 155 | GAQESKKKKKKRF |
| 156 | GAQESKKKKKKR |
| 157 | GAQESKKKKKK |
| 158 | GAQESKKKKK |
| 159 | GAQESKKKK |
| 160 | GAQESKKK |
| 161 | GAQESKK |
| 162 | GSQSSKKKKKKFSFKKPFKLSGLSFKRNRK |
| 163 | GSQSSKKKKKKFSFKKPFKLSGLSFKRNR |
| 164 | GSQSSKKKKKKFSFKKPFKLSGLSFKRN |
| 165 | GSQSSKKKKKKFSFKKPFKLSGLSFKR |
| 166 | GSQSSKKKKKKFSFKKPFKLSGLSFK |
| 167 | GSQSSKKKKKKFSFKKPFKLSGLSF |
| 168 | GSQSSKKKKKKFSFKKPFKLSGLS |
| 169 | GSQSSKKKKKKFSFKKPFKLSGL |
| 170 | GSQSSKKKKKKFSFKKPFKLSG |
| 171 | GSQSSKKKKKKFSFKKPFKLS |
| 172 | GSQSSKKKKKKFSFKKPFKL |
| 173 | GSQSSKKKKKKFSFKKPFK |
| 174 | GSQSSKKKKKKFSFKKPF |
| 175 | GSQSSKKKKKKFSFKKP |
| 176 | GSQSSKKKKKKFSFKK |
| 177 | GSQSSKKKKKKFSFK |
| 178 | GSQSSKKKKKKFSF |
| 179 | GSQSSKKKKKKFS |
| 180 | GSQSSKKKKKKF |
| 181 | GSQSSKKKKKK |
| 182 | GSQSSKKKKK |
| 183 | GSQSSKKKK |
| 184 | GSQSSKKK |
| 185 | GSQSSKK |

In some aspects, the scaffold moiety, e.g., Scaffold Y, useful for the present disclosure does not contain an N-terminal Met. In some aspects, the scaffold moiety, e.g., Scaffold Y, comprises a lipidated amino acid, e.g., a myristoylated amino acid, at the N-terminus of the scaffold protein, which functions as a lipid. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid can be any lipid known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane moieties that can act as a scaffold moiety known in the art are presented in the following table.

TABLE 5
Modification groups

| Modification | Modifying Group |
|---|---|
| S-Palmitoylation | 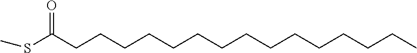 [SMILES: CCCCCCCCCCCCCCCC(=O)SC] |
| N-Palmitoylation | 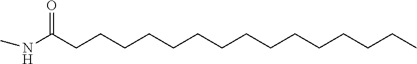 [SMILES: CCCCCCCCCCCCCCCC(=O)NC] |
| N-Myristoylation | 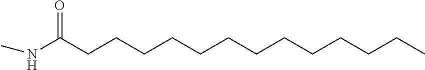 [SMILES: CCCCCCCCCCCCCC(=O)NC] |
| O-Acylation | 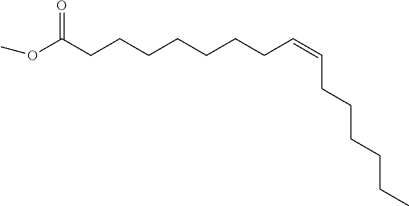 [SMILES: CCCCCC/C=C\CCCCCCCC(=O)OC] |
| Farnesylation | 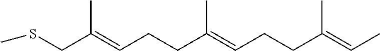 [SMILES: C/C=C(\C)CC/C=C(\C)CC/C=C(\C)CSC] |
| Geranyigeranylation | 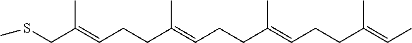 [SMILES: C/C=C(\C)CC/C=C(\C)CC/C=C(\C)CC/C=C(\C)CSC] |
| Cholesterol | 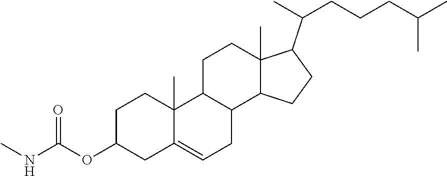 [SMILES: CNC(=O)OC1CCC2(C)C(=CCC3C2CCC2(C)C(C(C)CCCC(C)C)CCC32)C1] |

II.G.3 Scaffold Protein Fusion Constructs

In some aspects, the scaffold moiety is linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, the scaffold moiety can be used to link any moiety to the luminal surface and/or the external surface of the exosome. For example, the PTGFRN polypeptide can be used to link a biologically active molecule inside the lumen (e.g., on the luminal surface) in addition to the external surface of the EV, e.g., exosome. Therefore, in certain aspects, the scaffold moiety can be used for dual purposes, e.g., a biologically active molecule on the luminal surface and a second biologically active molecule or other payload on the external surface of the EV, e.g., exosome, or a biologically active molecule on the external surface of the exosome and a second biologically active molecule or other payload on the luminal surface of the EV, e.g., exosome.

II.G.4 Lipid

Suitable scaffold moieties capable of link a biologically active molecule to the surface of an EV, e.g., an exosome, via chemical linking with a maleimide moiety comprise for example sterols (e.g., cholesterol), phospholipid, lysophospholipids, fatty acids, or fat-soluble vitamins, as described in detail below.

In some aspects, the scaffold moiety can be a lipid. A lipid scaffold moiety can be any lipid known in the art, e.g., palmitic acid or glycosylphosphatidylinositols. In some aspects, the lipid, is a fatty acid, phosphatide, phospholipid (e.g., phosphatidyl choline, phosphatidyl serine, or phosphatidyl ethanolamine), or analogue thereof (e.g. phosphatidylcholine, lecithin, phosphatidylethanolamine, cephalin, or phosphatidylserine or analogue or portion thereof, such as a partially hydrolyzed portion thereof).

The scaffold moiety can be linked, e.g., chemically linked, to a biologically active molecule using a maleimide moiety. Such linkage can be direct or indirect via a linker or linker combination, at any chemically feasible location, e.g., at the 5′ and/or 3′ end of a nucleotide sequence, e.g., of a biologically active molecule (e.g, an ASO). In one aspect, the scaffold moiety is linked, e.g., chemically linked via a maleimide moiety, only to the 3′ end of the biologically active molecule. In one aspect, the scaffold moiety is linked, e.g., chemically linked via a maleimide moiety, only to the 5′ end of a nucleotide sequence, e.g., of a biologically active molecule (e.g, an ASO). In one aspect, the scaffold moiety is linked, e.g., chemically linked via a maleimide moiety, at a location which is not the 3′ end or 5′ end of a nucleotide sequence, e.g., of a biologically active molecule (e.g, an ASO).

In some aspects, a biologically active molecule can be linked, e.g., chemically linked via a maleimide moiety, directly or indirectly via a linker, to, e.g., any of the lipid disclosed above (for example, palmitic acid, myristic acid, fatty acid, farnesyl, geranyl-geranyl, or cholesterol). In some aspects, a scaffold moiety can comprise two or more types of scaffold moieties disclosed herein. For example, in some aspects, a scaffold moiety can comprise two lipids, e.g., a phospholipids and a fatty acid, or two phospholipids, or two fatty acids, or a lipid and a vitamin, or cholesterol and a vitamin, etc. which taken together have 6-80 carbon atoms (i.e., an equivalent carbon number (ECN) of about 6 to about 80).

In some aspects, the combination of scaffold moieties, e.g., a combination of the lipids (e.g., fatty acids) has an ECN of about 6 to about 80, about 8 to about 80, about 10 to about 80, about 12 to about 80, about 14 to about 80, about 16 to about 80, about 18 to about 80, about 20 to about 80, about 22 to about 80, about 24 to about 80, about 26 to about 80, about 28 to about 80, about 30 to about 80, about 4 to about 76, about 6 to about 76, about 8 to about 76, about 10 to about 76, about 12 to about 76, about 14 to about 76, about 16 to about 76, about 18 to about 76, about 20 to about 76, about 22 to about 76, about 24 to about 76, about 26 to about 76, about 28 to about 76, about 30 to about 76, about 6 to about 72, about 8 to about 72, about 10 to about 72, about 12 to about 72, about 14 to about 72, about 16 to about 72, about 18 to about 72, about 20 to about 72, about 22 to about 72, about 24 to about 72, about 26 to about 72, about 28 to about 72, about 30 to about 72, about 6 to about 68, about 8 to about 68, about 10 to about 68, about 12 to about 68, about 14 to about 68, about 16 to about 68, 1 about 8 to about 68, about 20 to about 68, about 22 to about 68, about 24 to about 68, about 26 to about 68, about 28 to about 68, about 30 to about 68, about 6 to about 64, about 8 to about 64, about 10 to about 64, about 12 to about 64, about 14 to about 64, about 16 to about 64, about 18 to about 64, about 20 to about 64, about 22 to about 64, about 24 to about 64, about 26 to about 64, about 28 to about 64, about 30 to about 64, about 6 to about 60, about 8 to about 60, about 10 to about 60, about 12 to about 56, about 14 to about 56, about 16 to about 56, about 18 to about 56, about 20 to about 56, about 22 to about 56, about 24 to about 56, about 26 to about 56, about 28 to about 56, about 30 to about 56, about 6 to about 52, about 8 to about 52, about 10 to about 52, about 12 to about 52, about 14 to about 52, about 16 to about 52, about 18 to about 52, about 20 to about 52, about 22 to about 52, about 24 to about 52, about 26 to about 52, about 28 to about 52, about 30 to about 52, about 6 to about 48, about 8 to about 48, about 10 to about 48, about 12 to about 48, about 14 to about 48, 1 about 6 to about 48, 1 about 8 to about 48, about 20 to about 48, 2 about 2 to about 48, about 24 to about 48, about 26 to about 48, about 28 to about 48, about 30 to about 48, about 6 to about 44, about 8 to about 44, about 10 to about 44, about 12 to about 44, about 14 to about 44, about 16 to about 44, about 18 to about 44, about 20 to about 44, about 22 to about 44, about 24 to about 44, 2 about 6 to about 44, about 28 to about 44, about 30 to about 44, about 6 to about 40, about 8 to about 40, about 10 to about 40, about 12 to about 40, about 14 to about 40, about 16 to about 40, about 18 to about 40, about 20 to about 40, 2 about 2 to about 40, about 24 to about 40, about 26 to about 40, 2 about 8 to about 40, about 30 to about 40, about 6 to about 36, about 8 to about 36, about 10 to about 36, about 12 to about 36, about 14 to about 36, about 16 to about 36, about 18 to about 36, about 20 to about 36, about 22 to about 36, about 24 to about 36, about 26 to about 36, about 28 to about 36, about 30 to about 36, about 6 to about 32, about 8 to about 32, 1 about 0 to about 32, about 12 to about 32, about 14 to about 32, 1 about 6 to about 32, 1 about 8 to about 32, about 20 to about 32, about 22 to about 32, about 24 to about 32, about 26 to about 32, 28 to about 32, or about 30 to about 32.

II.G.3.a Cholesterol and Other Sterols

In some aspects, the scaffold moiety comprises a sterol, steroid, hopanoid, hydroxysteroid, secosteroid, or analog thereof with lipophilic properties. In some aspects, the scaffold moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, or stigmasterol. Sterols can be found either as free sterols, acylated (sterol esters), alkylated (steryl alkyl ethers), sulfated (sterol sulfate), or linked to a glycoside moiety (steryl glycosides), which can be itself acylated (acylated sterol glycosides).

In some aspects, the scaffold moiety comprises a steroid. In some aspects, the steroid is selected from dihydrotestosterone, uvaol, hecigenin, diosgenin, progesterone, or cortisol.

For example, sterols can be conjugated to the biologically active molecule directly or via a linker combination at the available OH group of the sterol. Exemplary sterols have the general skeleton shown below:

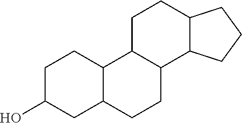 [SMILES: OC1CCC2C(CCC3C4CCCC4CCC23)C1]

As a further example, ergosterol has the structure below:

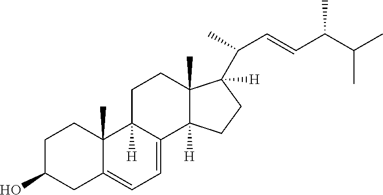

Cholesterol has the structure below:

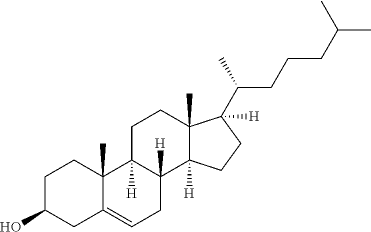

Accordingly, in some aspects, the free OH group of a sterol or steroid is used to conjugate the biologically active molecule, e.g., an ASO, directly or via a linker combination, to the sterol (e.g., cholesterol) or steroid.

II.G.3.b Fatty Acids

In some aspects, the scaffold moiety comprises a fatty acid. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an ω-3 (omega-3) or ω-6 (omega-6) fatty acid.

In some aspects, the lipid, e.g., fatty acid, has a C₂-C₆₀ chain. In some aspects, the lipid, e.g., fatty acid, has a C₂-C₂₈ chain. In some aspects, the fatty acid, has a C₂-C₄₀ chain. In some aspects, the fatty acid, has a C₂-C₁₂ or C₄-C₁₂ chain. In some aspects, the fatty acid, has a C₄-C₄₀ chain. In some aspects, the fatty acid, has a C₄-C₄₀, C₂-C₃₈, C₂-C₃₆, C₂-C₃₄, C₂-C₃₂, C₂-C₃₀, C₄-C₃₀, C₂-C₂₈, C₄-C₂₈, C₂-C₂₆, C₄-C₂₆, C₂-C₂₄, C₄-C₂₄, C₆-C₂₄, C₈-C₂₄, C₁₀-C₂₄, C₂-C₂₂, C₄-C₂₂, C₆-C₂₂, C₈-C₂₂, C₁₀-C₂₂, C₂-C₂₀, C₄-C₂₀, C₆-C₂₀, C₈-C₂₀, C₁₀-C₂₀, C₂-C₁₈, C₄-C₁₈, C₆-C₁₈, C₈-C₁₈, C₁₀-C₁₈, C₁₂-C₁₈, C₁₄-C₁₈, C₁₆-C₁₈, C₂-C₁₆, C₄-C₁₆, C₆-C₁₆, C₈-C₁₆, C₁₀-C₁₆, C₁₂-C₁₆, C₁₄-C₁₆, C₂-C₁₅, C₄-C₁₅, C₆-C₁₅, C₈-C₁₅, C₉-C₁₅, C₁₀-C₁₅, C₁₁-C₁₅, C₁₂-C₁₅, C₁₃-C₁₅, C₂-C₁₄, C₄-C₁₄, C₆-C₁₄, C₈-C₁₄, C₉-C₁₄, C₁₀-C₁₄, C₁₁-C₁₄, C₁₂-C₁₄, C₂-C₁₃, C₄-C₁₃, C₆-C₁₃, C₇-C₁₃, C₈-C₁₃, C₉-C₁₃, C₁₀-C₁₃, C₁₀-C₁₃, C₁₁-C₁₃, C₂-C₁₂, C₄-C₁₂, C₆-C₁₂, C₇-C₁₂, C₈-C₁₂, C₉-C₁₂, C₁₀-C₁₂, C₂-C₁₁, C₄-C₁₁, C₆-C₁₁, C₇-C₁₁, C₈-C₁₁, C₉-C₁₁, C₂-C₁₀, C₄-C₁₀, C₂-C₉, C₄-C₉, C₂-C₈, C₂-C₇, C₄-C₇, C₂-C₆, or C₄-C₆, chain. In some aspects, the fatty acid, has a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, or C₆₀ chain.

In some aspects, the scaffold moiety comprises two fatty acids, each of which is independently selected from a fatty acid having a chain with any one of the foregoing ranges or numbers of carbon atoms. In some aspects, one of the fatty acids is independently a fatty acid with a C6-C21 chain and one is independently a fatty acid with a C12-C36 chain. In some aspects, each fatty acid independently has a chain of about 11, about 12, about 13, about 14, about 15, about 16, about 17 or about 18 carbon atoms.

Suitable fatty acids include saturated straight-chain fatty acids, saturated branched fatty acids, unsaturated fatty acids, hydroxy fatty acids, and polycarboxylic acids. In some aspects, such fatty acids have up to about 32 carbon atoms.

Examples of useful saturated straight-chain fatty acids include those having an even number of carbon atoms, such as butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid, and heptacosanoic acid.

Examples of suitable saturated branched fatty acids include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, α-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.). Suitable saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid, and 26-methyloctacosanoic acid.

Examples of suitable unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid, 7,10,13,16,19-docosapentaenoic acid, 4,7,10,13,16,19-docosahexaenoic acid, and the like.

Examples of suitable hydroxy fatty acids include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like.

Examples of suitable polycarboxylic acids include oxalic acid, malonic acid, succinic acid, glutaric acid, adipic acid, pimelic acid, suberic acid, azelaic acid, sebacic acid, D,L-malic acid, and the like.

In some aspects, each fatty acid is independently selected from propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, or octatriacontanoic acid.

In some aspects, each fatty acid is independently selected from α-linolenic acid, stearidonic acid, eicosapemaenoic acid, docosahexaenoic acid, linoleic acid, gamma-linoleic acid, dibomo-gamma-linoleic acid, arachidonic acid, docosatetraenoic acid, palmitoleic acid, vaccenic acid, paullinic acid, oleic acid, elaidic acid, gondoic acid, eurcic acid, nervonic acid, mead acid, adrenic acid, bosseopentaenoic acid, ozubondo acid, sardine acid, herring acid, docosahexaenoic acid, or tetracosanolpentaenoic acid, or another monounsaturated or polyunsaturated fatty acid.

In some aspects, one or both of the fatty acids is an essential fatty acid. In view of the beneficial health effects of certain essential fatty acids, the therapeutic benefits of disclosed therapeutic-loaded exosonmes can be increased by including such fatty acids in the therapeutic agent. In some aspects, the essential fatty acid is an n-6 or n-3 essential fatty acid selected from the group consisting of linolenic acid, gamma-linolenic acid, dibuomno-gamma-linolenic acid, arachidonic acid, adrenic acid, docosapentaenoic n-6 acid, alpha-linolenic acid, stearidonic acid, the 20:4n-3 acid, eicosapentaenoic acid, docosapentaenoic n-3 acid, or docosahexaenoic acid.

In some aspects, each fatty acid is independently selected from all-cis-7,10,13-hexadecatrienoic acid, r-linolenic acid, stearidonic acid, eicosatrienoic acid, eicosatetraenoic acid, eicosapentaenoic acid (EPA), docosapentaenoic acid, docosahexaenoic acid (DRHA), tetracosapentaenoic acid, tetracosahexaenoic acid, or lipoic acid. In other aspects, the fatty acid is selected from eicosapentaenoic acid, docosahexaenoic acid, or lipoic acid. Other examples of fatty acids include all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-S,11,14,17-eicosatetraenoic acid), eicosapentaenloic acid (EPA), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,18,21-docosahexaenoic acid), or tetracosahexaenoic acid (nisinic acid or all-cis-6,9,12,12,15,18,21-tetracosenoic acid). In some aspects, the fatty acid is a medium-chain fatty acid such as lipoic acid.

Fatty acid chains differ greatly in the length of their chains and can be categorized according to chain length, e.g. as short to very long. Short-chain fatty acids (SCFA) are fatty acids with chains of about five or less carbons (e.g. butyric acid). In some aspects, the fatty acid is a SCFA. Medium-chain fatty acids (MCFA) include fatty acids with chains of about 6-12 carbons, which can form medium-chain triglycerides. In some aspects, the fatty acid is a MCFA. Long-chain fatty acids (LCFA) include fatty acids with chains of 13-21 carbons. In some aspects, the fatty acid is a LCFA. In some aspects, the fatty acid is a LCFA. Very long chain fatty acids (VLCFA) include fatty acids with chains of 22 or more carbons, such as between about 22 and about 60, between about 22 and about 50, or between about 22 and about 40 carbons. In some aspects, the fatty acid is a VLCFA.

II.G.3.c Phospholipids

In some aspects, the scaffold moiety comprises a phospholipid Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

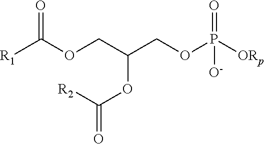

in which Rₚ represents a phospholipid moiety and R₁ and R₂ represent fatty acid moieties with or without unsaturation that can be the same or different.

A phospholipid moiety can be selected, for example, from the non-limiting group consisting of phosphatidyl choline, phosphatidyl ethanolamine, phosphatidyl glycerol, Phosphatidyl serine, phosphatidic acid, 2 lysophosphatidyl choline, and a sphingomnyelin.

Particular phospholipids can facilitate fusion to a lipid bilayer, e.g., the lipid bilayer of an exosomal membrane. For example, a cationic phospholipid can interact with one or more negatively charged phospholipids of a membrane. Fusion of a phospholipid to a membrane can allow one or more elements of a lipid-containing composition to bind to the membrane or to pass through the membrane.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosahexaenoic acid, and docosahexaenoic acid.

The phospholipids using as scaffold moieties in the present disclosure can be natural or non-natural phospholipids. Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide.

Phospholipids include, but are not limited to, glycerophospholipids such as phosphatidylcholines, phosphatidylethanolamines, phosphatidylserines, phosphatidylinositols, phosphatidyl glycerols, and phosphatidic acids.

Examples of phospholipids that can be used in the scaffold moieties disclosed herein include
Phosphatidylethanolamines: E.g., dilauroylphosphatidyl ethanolamine, dimyristoylphosphatidyl ethanolamine, dipalmitoyphosphatidyl ethanolamine, distearoylphosphatidyl ethanolamine, dioleoylphosphatidyl ethanolanrine, 1-palmitoyl-2-oleylphosphatidyl ethanolamine, 1-oleyl-2-palmitoylphosphatidyl ethanolamine, and dierucoylphosphatidyl ethanolamine;
Phosphatidyl glycerols: E g., dilauroylphosphatidyl glycerol, dinyristoylpliosphatidyl glycerol, dipalmitoylphosphatidyl glycerol, distearoylphosphatidyl glycerol, dioleoylphosphatidyl glycerol, 1-palmitoyl-2-oleyl-phosphatidyl glycerol, 1-oleyl-2-palmitoyl-phosphatidyl glycerol, and dierucoylphosphatidyl glycerol;
Phosphatidyl serines: E.g., such as dilauroylphosphatidyl serine, dimyristoylphosphatidyl serine, dipalmitoylphosphatidyl serine, distearoylphosphatidyl serine, diolcoylphosphatidyl serine, 1-palmitoyl-2-oleyl-phosphatidyl serine, 1-oleyl-2-palmitoyl-phosphatidyl serine, and dierucoylphosphatidyl serine;
Phosphatidic acids: E.g., dilauroylphosphatidic acid, dimyristoylphosphatidic acid, dipalmitoylpliosphatidic acid, distearoylphosphatidic acid, dioleoylphospliatidic acid, 1-palmitoyl-2-oleylphosphatidic acid, 1-oleyl-2-palmitoyl-phosphatidic acid, and dierucoylphosphatidic acid; and,
Phosphatidyl inositols: E.g., dilauroylphosphatidyl inositol, dimyristoylphosphatidyl inositol, dipalnitoylphosphatidyl inositol, distearoylphosphatidyl inositol, diolcoylphosphatidyl inositol, 1-palnitoyl-2-oleyl-phosphatidyl inositol, 1-oleyl-2-palmitoyl-phosphatidyl inositol, and dierucoylphosphatidyl inositol.

Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospliolipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some aspects, the scaffold moiety comprises at least one symmetric phospholipid. Symmetric phospholipids can be selected from the non-limiting group consisting of
1,2-dipropionyl-sn-glycero-3-phosphocholine (03:0 PC),
1,2-dibutyryl-sn-glycero-3-phosphocholine (04:0 PC),
1,2-dipentanoyl-sn-glycero-3-phosphocholine (05:0 PC),
1,2-dihexanoyl-sn-glycero-3-phosphocholine (06:0 PC),
1,2-diheptanoyl-sn-glycero-3-phosphocholine (07:0 PC),
1,2-dioctanoyl-sn-glycero-3-phosphocholine (08:0 PC),
1,2-dinonanoyl-sn-glycero-3-phosphocholine (09:0 PC),
1,2-didecanoyl-sn-glycero-3-phosphocholine (10:0 PC),
1,2-diundecanoyl-sn-glycero-3-phosphocholine (11:0 PC, DUPC),
1,2-dilauroyl-sn-glycero-3-phosphocholine (12:0 PC),
1,2-ditridecanoyl-sn-glycero-3-phosphocholine (13:0 PC),
1,2-dimyristoyl-sn-glycero-3-phosphocholine (14:0 PC, DMPC),
1,2-dipentadecanoyl-sn-glycero-3-phosphocholine (15:0 PC),
1,2-dipalmitoyl-sn-glycero-3-phosphocholine (16:0 PC, DPPC),
1,2-diphytanoyl-sn-glycero-3-phosphocholine (4ME 16:0 PC),
1,2-diheptadecanoyl-sn-glycero-3-phosphocholine (17:0 PC),
1,2-distearoyl-sn-glycero-3-phosphocholine (18:0 PC, DSPC),
1,2-dinonadecanoyl-sn-glycero-3-phosphocholine (19:0 PC),
1,2-diarachidoyl-sn-glycero-3-phosphocholine (20:0 PC),
1,2-dihenarachidoyl-sn-glycero-3-phosphocholine (21:0 PC),
1,2-dibehenoyl-sn-glycero-3-phosphocholine (22:0 PC),
1,2-ditricosanoyl-sn-glycero-3-phosphocholine (23:0 PC),
1,2-dilignoceroyl-sn-glycero-3-phosphocholine (24:0 PC),
1,2-dimyristoleoyl-sn-glycero-3-phosphocholine (14:1 (A9-Cis) PC),
1,2-dimyristelaidoyl-sn-glycero-3-phosphocholine (14:1 (A9-Trans) PC),
1,2-dipalmitoleoyl-sn-glycero-3-phosphocholine (16:1 (A9-Cis) PC),
1,2-dipalmitelaidoyl-sn-glycero-3-phosphocholine (16:1 (A9-Trans) PC),
1,2-dipetroselenoyl-sn-glycero-3-phosphocholine (18:1 (A6-Cis) PC),
1,2-dioleoyl-sn-glycero-3-phosphocholine (18:1 (A9-Cis) PC, DOPC),
1,2-dielaidoyl-sn-glycero-3-phosphocholine (18:1 (A9-Trans) PC),
1,2-dilinoleoyl-sn-glycero-3-phosphocholine (18:2 (Cis) PC, DLPC),
1,2-dilinolenoyl-sn-glycero-3-phosphocholine (18:3 (Cis) PC, DLnPC),
1,2-dieicosenoyl-sn-glycero-3-phosphocholine (20:1 (Cis) PC),
1,2-diarachidonoyl-sn-glycero-3-phosphocholine (20:4 (Cis) PC, DAPC),
1,2-dierucoyl-sn-glycero-3-phosphocholine (22:1 (Cis) PC),
1,2-didocosahexaenoyl-sn-glycero-3-phosphocholine (22:6 (Cis) PC, DHAPC),
1,2-dinervonoyl-sn-glycero-3-phosphocholine (24:1 (Cis) PC),
1,2-dihexanoyl-sn-glycero-3-phosphoethanolamine (06:0 PE),
1,2-dioctanoyl-sn-glycero-3-phosphoethanolamine (08:0 PE),
1,2-didecanoyl-sn-glycero-3-phosphoethanolamine (10:0 PE),
1,2-dilauroyl-sn-glycero-3-phosphoethanolamine (12:0 PE),
1,2-dimyristoyl-sn-glycero-3-phosphoethanolamine (14:0 PE),
1,2-dipentadecanoyl-sn-glycero-3-phosphoethanolamine (15:0 PE),
1,2-dipalmitoyl-sn-glycero-3-phosphoethanolamine (16:0 PE),
1,2-diphytanoyl-sn-glycero-3-phosphoethanolamine (4ME 16:0 PE),
1,2-diheptadecanoyl-sn-glycero-3-phosphoethanolamine (17:0 PE),
1,2-distearoyl-sn-glycero-3-phosphoethanolamine (18:0 PE, DSPE),
1,2-dipalmitoleoyl-sn-glycero-3-phosphoethanolamine (16:1 PE),
1,2-dioleoyl-sn-glycero-3-phosphoethanolamine (18:1 (A9-Cis) PE, DOPE),
1,2-dielaidoyl-sn-glycero-3-phosphoethanolamine (18:1 (A9-Trans) PE),
1,2-dilinoleoyl-sn-glycero-3-phosphoethanolamine (18:2 PE, DLPE),
1,2-dilinolenoyl-sn-glycero-3-phosphoethanolamine (18:3 PE, DLnPE),
1,2-diarachidonoyl-sn-glycero-3-phosphoethanolamine (20:4 PE, DAPE),
1,2-didocosahexaenoyl-sn-glycero-3-phosphoethanolamine (22:6 PE, DHAPE),
1,2-di-O-octadecenyl-sn-glycero-3-phosphocholine (18:0 Diether PC),
1,2-dioleoyl-sn-glycero-3-phospho-rac-(1-glycerol) sodium salt (DOPG), and any combination thereof.

In some aspects, the scaffold moiety comprises at least one symnmetric phospholipid selected from the non-limiting group consisting of DLPC, DMPVITC, DOPC, DPPC, DSPC, DUPC, 18:0 Diether PC, DLnPC, DAPC, DUAPC, DOPE, 4ME 16:0 PE, DSPE, DLPE, DLnPE, DAPE, DHAPE, DOPG, and any combination thereof.

In some aspects, the scaffold moiety comprises at least one asymmetric phospholipid. Asymmetric phospholipids can be selected from the non-limiting group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-acetyl-sn-glycero-3-phosphocholine (16:0-02:0 PC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-palmitoyl-2-oleoyl-sn-glycero-3-phosphocholine (16:0-18:1 PC, POPC),
1-palmitoyl-2-linoleoyl-sn-glycero-3-phosphocholine (16:0-18:2 PC, PLPC),
1-palmitoyl-2-arachidonoyl-sn-glycero-3-phosphocholine (16:0-20:4 PC),
1-palmitoyl-2-docosahexaenoyl-sn-glycero-3-phosphocholine (14:0-22:6 PC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC),
1-stearoyl-2-oleoyl-sn-glycero-3-phosphocholine (18:0-18:1 PC, SOPC),
1-stearoyl-2-linoleoyl-sn-glycero-3-phosphocholine (18:0-18:2 PC),
1-stearoyl-2-arachidonoyl-sn-glycero-3-phosphocholine (18:0-20:4 PC),
1-stearoyl-2-docosahexaenoyl-sn-glycero-3-phosphocholine (18:0-22:6 PC),
1-oleoyl-2-myristoyl-sn-glycero-3-phosphocholine (18:1-14:0 PC, OMPC),
1-oleoyl-2-palmitoyl-sn-glycero-3-phosphocholine (18:1-16:0 PC, OPPC),
1-oleoyl-2-stearoyl-sn-glycero-3-phosphocholine (18:1-18:0 PC, OSPC),
1-palmitoyl-2-oleoyl-sn-glycero-3-phosphoethanolamine (16:0-18:1 PE, POPE),
1-palmitoyl-2-linoleoyl-sn-glycero-3-phosphoethanolamine (16:0-18:2 PE),
1-palmitoyl-2-arachidonoyl-sn-glycero-3-phosphoethanolamine (16:0-20:4 PE),
1-palmitoyl-2-docosahexaenoyl-sn-glycero-3-phosphoethanolamine (16:0-22:6 PE),
1-stearoyl-2-oleoyl-sn-glycero-3-phosphoethanolamine (18:0-18:1 PE),
1-stearoyl-2-linoleoyl-sn-glycero-3-phosphoethanolamine (18:0-18:2 PE),
1-stearoyl-2-arachidonoyl-sn-glycero-3-phosphoethanolamine (18:0-20:4 PE),
1-stearoyl-2-docosahexaenoyl-sn-glycero-3-phosphoethanolamine (18:0-22:6 PE),
1-oleoyl-2-cholesterylhemisuccinoyl-sn-glycero-3-phosphocholine (OChemsPC), and
   any combination thereof.

To provide more remarkable nuclease resistance, cellular uptake efficiency, and a more remarkable RNA interference effect, phosphatidylethanolamines can be used as scaffold moieties, for example, dimyristoylphosphatidyl ethanolamine, dipalmitoylphosphatidyl ethanolamine, 1-palmitoyl-2-oleyl-phosphatidyl ethanolamine, and dioleoylphosphatidyl ethanolamine.

The binding site of lipid (e.g., a phospholipid) and a linker or biologically active molecule, e.g., an ASO, can be suitably selected according to the types of lipid and linker or biologically active molecule. Any position other than hydrophobic groups of the lipid can be linked to the linker or biologically active molecule by a chemical bond. For example, when using a phosphatidylethanolamine, the linkage can be made by forming an amide bond, etc. between the amino group of phosphatidylethanolamine and the linker or biologically active molecule.

When using a phosphatidylglycerol, the linkage can be made by forming an ester bond, an ether bond, etc. between the hydroxyl group of the glycerol residue and the linker or biologically active molecule.

When using a phosphatidylserine, the linkage can be made by forming an amide bond or an ester bond, etc. between the amino group or carboxyl group of the serine residue and the linker or biologically active molecule.

When using a phosphatidic acid, the linkage can be made by forming a phosphoester bond, etc. between the phosphate residue and the linker or biologically active molecule.

When using a phosphatidylinositol, the linkage can be made by forming an ester bond, an ether bond, etc. between the hydroxyl group of the inositol residue and the linker or biologically active molecule.

II.G.3.d Lysolipids (e.g., Lysophospholipids)

In some aspects, the scaffold moiety comprises a lysolipid, e.g., a lysophospholipid. Lysolipids are derivatives of a lipid in which one or both fatty acyl chains have been removed, generally by hydrolysis. Lysophospholipids are derivatives of a phospholipid in which one or both fatty acyl chains have been removed by hydrolysis.

In some aspects, the scaffold moiety comprises any of the phospholipids disclosed above, in which one or both acyl chains have been removed via hydrolysis, and therefore the resulting lysophospholipid comprises one or no fatty acid acyl chain.

In some aspects, the scaffold moiety comprises a lysoglycerophospholipid, a lysoglycosphingoliopid, a lysophosphitidylcholine, a lysophosphatidylethanolanine, a lysophosphatidylinositol, or a lysophosphatidylserine.

In some aspects, the scaffold moiety comprises a lysolipid selected from the non-limiting group consisting of
1-hexanoyl-2-hydroxy-sn-glycero-3-phosphocholine (06:0 Lyso PC),
1-heptanoyl-2-hydroxy-sn-glycero-3-phosphocholine (07:0 Lyso PC),
1-octanoyl-2-hydroxy-sn-glycero-3-phosphocholine (08:0 Lyso PC),
1-nonanoyl-2-hydroxy-sn-glycero-3-phosphocholine (09:0 Lyso PC),
1-decanoyl-2-hydroxy-sn-glycero-3-phosphocholine (10:0 Lyso PC),
1-undecanoyl-2-hydroxy-sn-glycero-3-phosphocholine (11:0 Lyso PC),
1-lauroyl-2-hydroxy-sn-glycero-3-phosphocholine (12:0 Lyso PC),
1-tridecanoyl-2-hydroxy-sn-glycero-3-phosphocholine (13:0 Lyso PC),
1-myristoyl-2-hydroxy-sn-glycero-3-phosphocholine (14:0 Lyso PC),
1-pentadecanoyl-2-hydroxy-sn-glycero-3-phosphocholine (15:0 Lyso PC),
1-palmitoyl-2-hydroxy-sn-glycero-3-phosphocholine (16:0 Lyso PC),
1-heptadecanoyl-2-hydroxy-sn-glycero-3-phosphocholine (17:0 Lyso PC),
1-stearoyl-2-hydroxy-sn-glycero-3-phosphocholine (18:0 Lyso PC),
1-oleoyl-2-hydroxy-sn-glycero-3-phosphocholine (18:1 Lyso PC),
1-nonadecanoyl-2-hydroxy-sn-glycero-3-phosphocholine (19:0 Lyso PC),
1-arachidoyl-2-hydroxy-sn-glycero-3-phosphocholine (20:0 Lyso PC),
1-behenoyl-2-hydroxy-sn-glycero-3-phosphocholine (22:0 Lyso PC),
1-lignoceroyl-2-hydroxy-sn-glycero-3-phosphocholine (24:0 Lyso PC),
1-hexacosanoyl-2-hydroxy-sn-glycero-3-phosphocholine (26:0 Lyso PC),
1-myristoyl-2-hydroxy-sn-glycero-3-phosphoethanolamine (14:0 Lyso PE),
1-palmitoyl-2-hydroxy-sn-glycero-3-phosphoethanolamine (16:0 Lyso PE),
1-stearoyl-2-hydroxy-sn-glycero-3-phosphoethanolamine (18:0 Lyso PE),
1-oleoyl-2-hydroxy-sn-glycero-3-phosphoethanolamine (18:1 Lyso PE),
1-hexadecyl-sn-glycero-3-phosphocholine (C16 Lyso PC), and
   any combination thereof.

I.G.3.e Vitamins

In some aspects, the scaffold moiety comprises a lipophilic vitamin, e.g., folic acid, vitamin A, vitamin E, or vitamin K.

In some aspects, the scaffold moiety comprises vitamin A. Vitamin A is a group of unsaturated nutritional organic compounds that includes retinol, retinal, retinoic acid, and several provitamin A carotenoids (most notably beta-carotene). In some aspects, the scaffold moiety comprises retinol. In some aspects, the scaffold moiety comprises a retinoid. Retinoids are a class of chemical compounds that are vitamers of vitamin A or are chemically related to it. In some aspects, the scaffold moiety comprises a first generation retinoid (e.g., retinol, tretinoin, isotreatinoin, or alitretinoin), a second-generation retinoid (e.g., etretinate or acitretin), a third-generation retinoid (e.g., adapalene, bexarotene, or tazarotene), or any combination thereof.

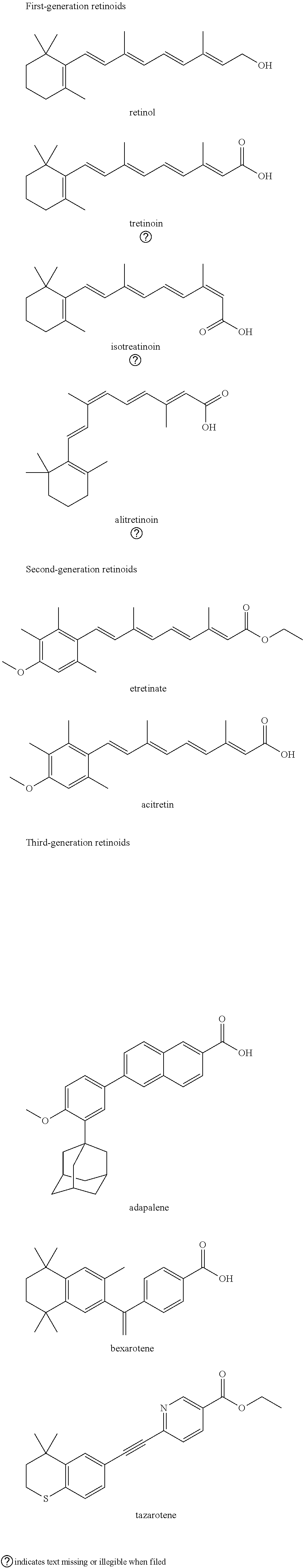

In some aspects, the scaffold moiety comprises vitamin E. Tocopherols are a class of methylated phenols many of which have vitamin E activity. Thus, in some aspects, the scaffold moiety comprises alpha-tocopherol, beta-tocopherol, gamma-tocopherol, delta-tocopherol, or a combination thereof.

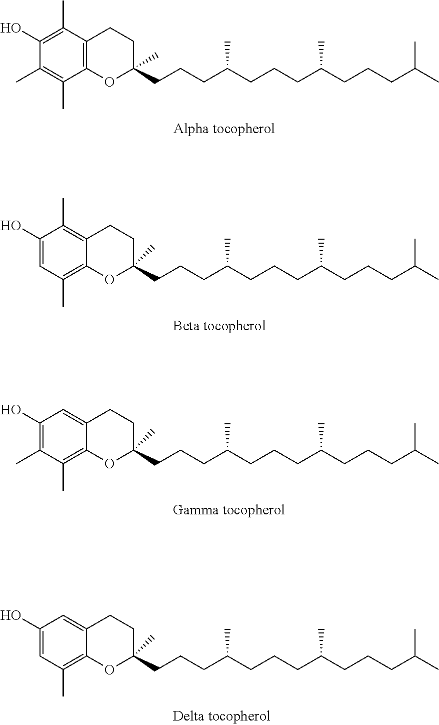 [SMILES: Cc1c(C)c2c(c(C)c1O)CC[C@@](C)(CCC[C@H](C)CCC[C@H](C)CCCC(C)C)O2 ; Cc1cc(O)c(C)c2c1O[C@](C)(CCC[C@H](C)CCC[C@H](C)CCCC(C)C)CC2 ; Cc1c(O)cc2c(c1C)O[C@](C)(CCC[C@H](C)CCC[C@H](C)CCCC(C)C)CC2 ; Cc1cc(O)cc2c1O[C@](C)(CCC[C@H](C)CCC[C@H](C)CCCC(C)C)CC2]

Tocotrienols also have vitamin E activity. The critical chemical structural difference between tocotrienols and tocopherols is that tocotrienols have unsaturated isoprenoid side chain with three carbon-carbon double bonds versus saturated side chains for tocopherols. In some aspects, the scaffold moiety comprises alpha-tocotrienol, beta-tocotrienol, gamma-tocotrienol, delta-tocotrienol, or a combination thereof. Tocotrienols can be represented by the formula below

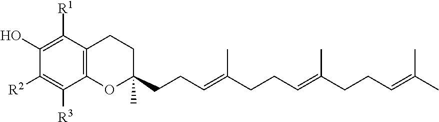

alpha(α)-Tocotrienol: R1=Me, R2=Me, R3=Me;
beta(β)-Tocotrienol: R1=Me, R2═H, R3=Me;
gamma(γ)-Tocotrienol: R1═H, R2=Me, R3=Me;
delta(δ)-Tocotrienol: R1═H, R2═H, R3=Me.

In some aspects, the scaffold moiety comprises vitamin K. Chemically, the vitamin K family comprises 2-methyl-1,4-naphthoquinone (3-) derivatives. Vitamin K includes two natural vitamers: vitamin Ki and vitamin K2. The structure of vitamin Ki (also known as phytonadione, phylloquinone, or (E)-phytonadione) is marked by the presence of a phytyl group. The structures of vitamin K2 (menaquinones) are marked by the polyisoprenyl side chain present in the molecule that can contain six to 13 isoprenyl units. Thus, vitamin K2 consists of a number of related chemical subtypes, with differing lengths of carbon side chains made of isoprenoid groups of atoms. MK-4 is the most common form of vitamin K2. Long chain forms, such as MK-7, MK-8 and MK-9 are predominant in fermented foods. Longer chain forms of vitamin K2 such as MK-10 to MK-13 are synthesized by bacteria, but they are not well absorbed and have little biological function. In addition to the natural forms of vitamin K, there is a number of synthetic forms of vitamin K such as vitamin K3 (menadione; 2-methylnaphthalene-1,4-dione), vitamin K4, and vitamin K5.

Accordingly, in some aspects, the scaffold moiety comprises vitamin Ki, K2 (e.g., MK-4, MK-5, MK-6, MK-7, MK-8, MK-9, MK-10, MK-11, MK-12, or MK-13), K3, K4, K₅, or any combination thereof.

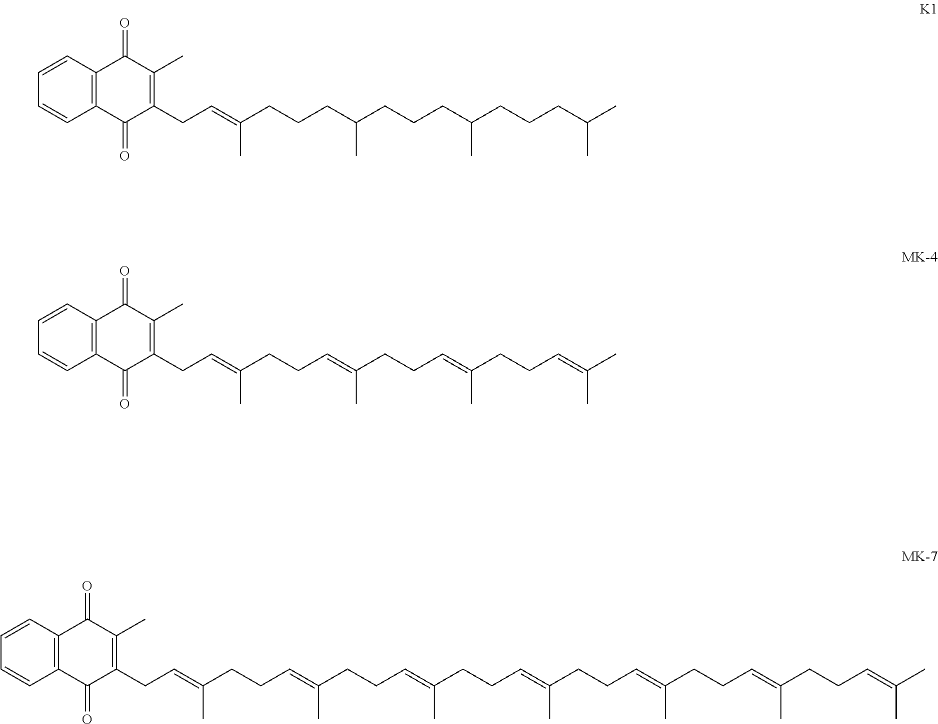 [SMILES: CC1=C(C/C=C(\C)CCCC(C)CCCC(C)CCCC(C)C)C(=O)c2ccccc2C1=O ; CC(C)=CCC/C(C)=C/CC/C(C)=C/CC/C(C)=C/CC1=C(C)C(=O)c2ccccc2C1=O ; CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O]

II.G.5 Chemically Induced Dimers

In some aspects, the scaffold moiety (e.g., a scaffold protein) is linked to a binding partner of a chemically induced dimer. In some aspects, the scaffold moiety (e.g., a scaffold protein) is linked to a binding partner of a chemically induced dimer, and the biologically active molecule is linked to a corresponding binding partner. In these aspects, the scaffold moiety (e.g., a scaffold protein) and the biologically active molecule associate with each other in the presence of the chemical that induces dimerization of the binding partners. In some aspects, the binding partner is linked to the N-terminus of the scaffold moiety. In some aspects, the binding partner is linked to the C-terminus of the scaffold moiety (e.g., a scaffold protein). In some aspects, the binding partner is linked to a luminal domain of the scaffold moiety (e.g., a scaffold protein).

In some aspects, the scaffold moiety (e.g., a scaffold protein) is linked to an affinity agent. In some aspects, the affinity agent is linked to the N-terminus of the scaffold moiety (e.g., a scaffold protein). In some aspects, the affinity agent is linked to the C-terminus of the scaffold moiety (e.g., a scaffold protein). In some aspects, the affinity agent is linked to a luminal domain of the scaffold moiety (e.g., a scaffold protein). In some aspects, the affinity agent comprises a polypeptide capable of binding to the biologically active molecule. In some aspects, the affinity agent comprises a receptor. In some aspects, the affinity agent comprises an antibody or an antigen binding domain, as disclosed herein. In some aspects, the affinity agent binds to one or more biologically active molecules.

In some aspects, the interaction between the affinity agent and the biologically active molecule is transient. In some aspects, the biologically active molecule is dissociated from the affinity agent under certain conditions. In certain aspects, the affinity of the affinity agent to the biologically active molecule is dependent on pH. In some aspects, the biologically active molecule dissociates from the affinity agent at a pH of at least about 3, at least about 4, at least about 5, at least about 6, at least about 7, at least about 8, at least about 9, at least about 10, at least about 11, or at least about 12. In some aspects, the affinity of the affinity agent for the biologically active molecule is dependent on the concentration of calcium, magnesium, sulfate, phosphate, or any combination thereof in the solution comprising the biologically active molecule and the affinity agent. In some aspects, the affinity of the affinity agent for the biologically active molecule is dependent on the salt concentration and/or ionic strength of the solution comprising the biologically active molecule and the affinity agent. In some aspects, the biologically active molecule and the affinity agent are dissociable under reducing conditions.

In some aspects, the scaffold moiety (e.g., a scaffold protein) is linked to a polypeptide that can bind to a biologically active molecule. In some aspects, the binding polypeptide is linked to the N-terminus of the scaffold moiety (e.g., a scaffold protein). In some aspects, the binding polypeptide is linked to the C-terminus of the scaffold moiety (e.g., a scaffold protein). In some aspects, the binding polypeptide is linked to a luminal domain of the scaffold moiety (e.g., a scaffold protein)

In some aspects, the binding polypeptide comprises an antigen-binding domain. In some aspects, the antigen-binding domain comprises an antigen-binding fragment of an antibody. In some aspects, the antigen-binding domain comprises a single-chain antibody or an antigen-binding fragment thereof. In some aspects, the antigen-binding domain comprises a humanized antibody or an antigen-binding fragment thereof. In some aspects, the antigen-binding domain comprises a murine antibody or an antigen-binding fragment thereof. In some aspects, the antigen-binding domain comprises a chimeric antibody (e.g., a mouse-human, a mouse-primate, or a primate-human monoclonal antibody) or an antigen binding fragment thereof. In some aspects, the antigen-binding domain comprises an antigen-binding fragment of a camelid antibody, a shark IgNAR, or an anti-idiotype antibody. In some aspects, the antigen-binding domain comprises a camelid antibody or an antigen-binding fragment thereof. In some aspects, the antigen-binding domain comprises a shark IgNAR or an antigen-binding fragment thereof. In some aspects, the antigen-binding domain comprises an anti-idiotype antibody or an antigen-binding fragment thereof.

In some aspects, the antigen-binding domain comprises a single chain antibody. In some aspects, the antigen-binding domain comprises an scFv. In some aspects, the antigen-binding domain comprises an (scFv)₂. In some aspects, the antigen-binding domain comprises an Fab. In some aspects, the antigen-binding domain comprises an Fab′. In some aspects, the antigen-binding domain comprises an F(ab′)₂. In some aspects, the antigen-binding domain comprises an F(ab1)₂. In some aspects, the antigen-binding domain comprises an Fv. In some aspects, the antigen-binding domain comprises a dAb. In some aspects, the antigen-binding domain comprises a single chain Fab. In some aspects, the antigen-binding domain comprises an Fd fragment.

In some aspects, the antigen-binding domain comprises a diabody. In some aspects, the antigen-binding domain comprises a minibody. In some aspects, the antigen-binding domain comprises an antibody-related polypeptide. In particular aspects, the antigen-binding domain comprises a nanobody.

In some aspects, the scaffold moiety (e.g., a scaffold protein) is linked to an Fc receptor, and the biologically active molecule is linked to an Fc. In certain aspects, the Fc receptor is an Fc gamma receptor selected from Fc gamma receptor I (FcγR1), FcγRIIA, FcγIIB, FcγIIIA, and FcγIIIB; and the Fc is an Fc of an IgG. In certain aspects, the Fc receptor is an FcγR1 and the Fc is an Fc of an IgG. In some aspects, the Fc receptor is an Fc alpha receptor I (FcαR1), and wherein the Fc is an Fc of an IgA. In some aspects, the Fc receptor is an Fc epsilon receptor selected from Fc epsilon receptor I (FεRI) and FcεRII, and wherein the Fc is an Fc of an IgE.

In some aspects, the scaffold moiety (e.g., a scaffold protein) is linked to a nanobody; and the biologically active molecule is linked an immunoglobulin constant region (Fc). In certain aspects, the nanobody specifically binds to the Fc.

### III. Methods of Making

EVs, e.g., exosomes, of the present disclosure can be produced by chemical synthesis, recombinant DNA technology, biochemical or enzymatic fragmentation of larger molecules, combinations of the foregoing or by any other method. In one aspect, the present disclosure provides a method of conjugating a biologically active molecule to an EV (e.g., exosome). The method comprises linking a biologically active molecule to an EV (e.g., exosome) via a maleimide moiety as described above.

Besides amine-reactive compounds, those having chemical groups that form bonds with sulfhydryls (—SH) are the most common crosslinkers and modification reagents for protein and other bioconjugate techniques. Sulfhydryls, also called thiols, exist in proteins in the side-chain of cysteine (Cys, C) amino acids. Pairs of cysteine sulfhydryl groups are often linked by disulfide bonds (—S—S—) within or between polypeptide chains as the basis of native tertiary or quaternary protein structure. Typically, only free or reduced sulfhydryl groups (—SH) [rather than sulfur atoms in disulfide bonds] are available for reaction with thiol-reactive compounds.

Sulfhydryl groups are useful targets for protein conjugation and labeling. First, sulfhydryls are present in most proteins but are not as numerous as primary amines; thus, crosslinking via sulfhydryl groups is more selective and precise. Second, sulfhydryl groups in proteins are often involved in disulfide bonds, so crosslinking at these sites typically does not significantly modify the underlying protein structure or block binding sites. Third, the number of available (i.e., free) sulfhydryl groups can be easily controlled or modified; they can be generated by reduction of native disulfide bonds, or they can be introduced into molecules through reaction with primary amines using sulfhydryl-addition reagents, such as 2-iminothiolane (Traut's Reagent), SATA, SATP, or SAT(PEG). Finally, combining sulfhydryl-reactive groups with amine-reactive groups to make heterobifunctional crosslinkers provides greater flexibility and control over crosslinking procedures. For example, using 3-Maleimido-propionic NHS ester, which contains a maleimide group and an NHS ester, the NHS ester can be used to label the primary amines (—NH₂) of proteins, amine-modified oligonucleotides, and other amine-containing molecules. The maleimide group will react with a thiol group to form a covalent bond, enabling the connection of biomolecule with a thiol.

The maleimide group reacts specifically with sulfhydryl groups when the pH of the reaction mixture is between 6.5 and 7.5; the result is formation of a stable thioether linkage that is not reversible (i.e., the bond cannot be cleaved with reducing agents). In more alkaline conditions (pH>8.5), the reaction favors primary amines and also increases the rate of hydrolysis of the maleimide group to a non-reactive maleamic acid. Maleimides do not react with tyrosines, histidines or methionines.

Thiol-containing compounds, such as dithiothreitol (DTT) and beta-mercaptoethanol (BME), must be excluded from reaction buffers used with maleimides because they will compete for coupling sites. For example, if DTT were used to reduce disulfides in a protein to make sulfhydryl groups available for conjugation, the DTT would have to be thoroughly removed using a desalting column before initiating the maleimide reaction. Interestingly, the disulfide-reducing agent TCEP does not contain thiols and does not have to be removed before reactions involving maleimide reagents.

Excess maleimides can be quenched at the end of a reaction by adding free thiols. EDTA can be included in the coupling buffer to chelate stray divalent metals that otherwise promote oxidation of sulfhydryls (non-reactive).

In one aspect, the linking comprises treating the EV (e.g., exosome) with a reducing agent. Suitable reducing agents include, for example, TCEP (Tris(2-carboxyethyl)phosphine), DTT (dithiothreitol), BME (2-mercaptoethanol), a thiolating agent, and any combination thereof. The thiolating agent can comprise, e.g., Traut's reagent (2-iminothiolane).

After the treatment with the reducing agent, the linking reaction further comprises bringing the reduced EV (e.g., exosome) in contact with the maleimide moiety. In one aspect, the maleimide moiety is linked to a biologically active molecule prior to the linking to the EV (e.g., exosome). In some aspects, the maleimide moiety is further attached to a linker to connect the maleimide moiety to the biologically active molecule. Accordingly, in some aspects, one or more linkers or spacers are interposed between the maleimide moiety and the biologically active molecule.

### IV. Therapeutic Uses

The present disclosure provides methods of treating a disease or condition is a subject in need thereof comprising administering a composition comprising EVs, e.g., exosomes, of the present disclosure to the subject. The present disclosure also provides methods of preventing or ameliorating the symptoms of a disease or condition is a subject in need thereof comprising administering a composition comprising EVs, e.g., exosomes, of the present disclosure to the subject. Also provided are methods to diagnose a disease or condition in a subject in need thereof comprising administering a composition comprising EVs, e.g., exosomes, of the present disclosure to the subject.

In one aspect, the disease or disorder is a cancer, an inflammatory disease, a neurodegenerative disorder, a central nervous disease or a metabolic disease.

Present disclosure also provides methods of preventing and/or treating a disease or disorder in a subject in need thereof, comprising administering an EV, e.g., exosome, disclosed herein to the subject. In some aspects, a disease or disorder that can be treated with the present methods comprises a cancer, graft-versus-host disease (GvHD), autoimmune disease, infectious diseases, or fibrotic diseases. In some aspects, the treatment is prophylactic. In other aspects, the EVs, e.g., exosomes, for the present disclosure are used to induce an immune response. In other aspects, the EVs, e.g., exosomes, for the present disclosure are used to vaccinate a subject.

In some aspects, the disease or disorder is a cancer. When administered to a subject with a cancer, in certain aspects, EVs, e.g., exosomes, of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, an EV, e.g., exosome, is administered in an amount and for a time sufficient to convert a “cold tumor” into a “hot tumor,” i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other The term “distal tumor” or “distant tumor” refers to a tumor that has spread from the original (or primary) tumor to distant organs or distant tissues, e.g., lymph nodes. In some aspects, the EVs, e.g., exosomes, of the disclosure treats a tumor after the metastatic spread.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikunga virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the EVs, e.g., exosomes, are administered intravenously to the circulatory system of the subject. In some aspects, the EVs, e.g., exosomes, are infused in suitable liquid and administered into a vein of the subject.

In some aspects, the EVs, e.g., exosomes, are administered intra-arterialy to the circulatory system of the subject. In some aspects, the EVs, e.g., exosomes, are infused in suitable liquid and administered into an artery of the subject.

In some aspects, the EVs, e.g., exosomes, are administered to the subject by intrathecal administration. In some aspects, the EVs, e.g., exosomes, are administered via an injection into the spinal canal, or into the subarachnoid space so that it reaches the cerebrospinal fluid (CSF).

In some aspects, the EVs, e.g., exosomes, are administered intratumorally into one or more tumors of the subject.

In some aspects, the EVs, e.g., exosomes, are administered to the subject by intranasal administration. In some aspects, the EVs, e.g., exosomes, can be insufflated through the nose in a form of either topical administration or systemic administration. In certain aspects, the EVs, e.g., exosomes, are administered as nasal spray.

In some aspects, the EVs, e.g., exosomes, are administered to the subject by intraperitoneal administration. In some aspects, the EVs, e.g., exosomes, are infused in suitable liquid and injected into the peritoneum of the subject. In some aspects, the intraperitoneal administration results in distribution of the EVs, e.g., exosomes, to the lymphatics. In some aspects, the intraperitoneal administration results in distribution of the EVs, e.g., exosomes, to the thymus, spleen, and/or bone marrow. In some aspects, the intraperitoneal administration results in distribution of the EVs, e.g., exosomes, to one or more lymph nodes. In some aspects, the intraperitoneal administration results in distribution of the EVs, e.g., exosomes, to one or more of the cervical lymph node, the inguinal lymph node, the mediastinal lymph node, or the sternal lymph node. In some aspects, the intraperitoneal administration results in distribution of the EVs, e.g., exosomes, to the pancreas.

In some aspects, the EVs, e.g., exosomes, are administered to the subject by periocular administration. In some aspects, the EVs, e.g., exosomes, are injected into the periocular tissues. Periocular drug administration includes the routes of subconjunctival, anterior sub-Tenon's, posterior sub-Tenon's, and retrobulbar administration.

In some aspects, the EVs, e.g., exosomes, are administered intraocularly. Accordingly, the present disclosure provides methods of treating an eye disease or disorder in a subject in need thereof comprising administering an effective amount of a composition comprising an extracellular vesicle (EV), e.g., exosome, of the present disclosure which comprises a payload (e.g., an AVV) to the subject, wherein the administration of the composition is intraocular.

In some aspects, the intraocular administration is selected from the group consisting of intravitreal administration, intracameral administration, subconjunctival administration, subretinal administration, subscleral administration, intrachoroidal administration, and any combination thereof. In some aspects, the intraocular administration comprises the injection of the EVs, e.g., exosomes, of the present disclosure. In some aspects, the intraocular administration is intravitreal injection.

### V. Pharmaceutical Compositions and Methods of Administration

The present disclosure also provides pharmaceutical compositions comprising EVs, e.g., exosomes, described herein that are suitable for administration to a subject. The pharmaceutical compositions generally comprise a plurality of EVs, e.g., exosomes, comprising a biologically active molecule covalently linked to the plurality of EVs, e.g., exosomes, via a maleimide moiety and a pharmaceutically-acceptable excipient or carrier in a form suitable for administration to a subject. Pharmaceutically acceptable excipients or carriers are determined in part by the particular composition being administered, as well as by the particular method used to administer the composition. Accordingly, there is a wide variety of suitable formulations of pharmaceutical compositions comprising a plurality of EVs, e.g., exosomes. See, e.g., Remington's Pharmaceutical Sciences, Mack Publishing Co., Easton, Pa. 18th ed. (1990).

The pharmaceutical compositions are generally formulated sterile and in full compliance with all Good Manufacturing Practice (GMP) regulations of the U.S. Food and Drug Administration. In some aspects, the pharmaceutical composition comprises one or more chemical compounds, such as for example, small molecules covalently linked to an EV, e.g., exosome, described herein.

In some aspects, a pharmaceutical composition comprises one or more therapeutic agents and an EV, e.g., exosome, described herein. In certain aspects, the EVs, e.g., exosomes, are co-administered with of one or more additional therapeutic agents, in a pharmaceutically acceptable carrier. In some aspects, the pharmaceutical composition comprising the EV, e.g., exosome, is administered prior to administration of the additional therapeutic agents. In other aspects, the pharmaceutical composition comprising the EV, e.g., exosome, is administered after the administration of the additional therapeutic agents. In further aspects, the pharmaceutical composition comprising the EV, e.g., exosome, is administered concurrently with the additional therapeutic agents.

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an EV, e.g., exosome, described herein. In certain aspects, the EVs, e.g., exosomes, are co-administered with of one or more additional therapeutic agents, in a pharmaceutically acceptable carrier. In some aspects, the pharmaceutical composition comprising the EVs, e.g., exosomes, is administered prior to administration of the additional therapeutic agents. In other aspects, the pharmaceutical composition comprising the EVs, e.g., exosomes, is administered after the administration of the additional therapeutic agents. In further aspects, the pharmaceutical composition comprising the EVs, e.g., exosomes, is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides (e.g., sucrose or trehalose), and other carbohydrates including glucose, mannose, or dextrins; chelating agents such as EDTA; sugars alcohols (e.g., mannitol or sorbitol); salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, of the present disclosure can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising EVs, e.g., exosomes, is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, of the present disclosure in an effective amount and in an appropriate solvent with one or a combination of ingredients enumerated herein, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EVs, e.g., exosomes.

Systemic administration of compositions comprising EVs, e.g., exosomes, of the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs, e.g., exosomes, of the present disclosure is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising EVs, e.g., exosomes, of the present disclosure is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of EVs, e.g., exosomes, of the present disclosure is subjected to radiation, e.g., X rays, gamma rays, beta particles, alpha particles, neutrons, protons, elemental nuclei, UV rays in order to damage residual replication-competent nucleic acids.

In certain aspects, the preparation of EVs, e.g., exosomes, of the present disclosure is subjected to gamma irradiation using an irradiation dose of more than about 1, about 5, about 10, about 15, about 20, about 25, about 30, about 35, about 40, about 50, about 60, about 70, about 80, about 90, about 100, or more than 100 kGy.

In certain aspects, the preparation of EVs, e.g., exosomes, of the present disclosure is subjected to X-ray irradiation using an irradiation dose of more than about 0.1, about 0.5, about 1, about 5, about 10, about 15, about 20, about 25, about 30, about 35, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, or about 10000.

The EVs, e.g., exosomes, of the present disclosure can be used concurrently with other drugs. To be specific, the EVs, e.g., exosomes, of the present disclosure can be used together with medicaments such as hormonal therapeutic agents, chemotherapeutic agents, immunotherapeutic agents, medicaments inhibiting the action of cell growth factors or cell growth factor receptors and the like.

### VI. Kits

The present disclosure also provides kits, or products of manufacture comprising one or more EVs, e.g., exosomes, of the present disclosure and optionally instructions for use. In some aspects, the kit, or product of manufacture contains a pharmaceutical composition described herein which comprises at least one EV, e.g., exosome, of the present disclosure, and instructions for use. In some aspects, the kit, or product of manufacture comprises at least one EV, e.g., exosome, of the present disclosure or a pharmaceutical composition comprising the EVs, e.g., exosomes, in one or more containers. One skilled in the art will readily recognize that the EVs, e.g., exosomes, of the present disclosure, pharmaceutical composition comprising the EVs, e.g., exosomes, of the present disclosure, or combinations thereof can be readily incorporated into one of the established kit formats which are well known in the art.

In some aspects, the kit, or product of manufacture comprises EVs, e.g., exosomes, one or more biologically active molecules, reagents to covalently attach the one or more biologically active molecules to the EVs, e.g., exosomes, via a maleimide moiety, or any combination thereof, and instructions to conduct the reaction to covalently attach the one or more biologically active molecules to the EVs, e.g., exosomes, via a maleimide moiety.

In some aspects, the kit comprises reagents to conjugate a biologically active molecule to an EV, e.g., exosome, via a maleimide moiety, and instructions to conduct the conjugation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (TRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

The following examples are provided for illustrative purposes only, and are not to be construed as limiting the scope or content of the invention in any way. The practice of the current invention will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1

### Exosome Isolation and Loading

Exosome isolation: Exosomes were collected from the supernatant of high density suspension cultures of HEK293 SF cells after 7-9 days. Cell culture medium was serially centrifuged, with the supernatant of the previous spin serving as the input for the subsequent spin: cell culture medium was centrifuged at 5,000×g for 30 minutes, the supernatant collected and the pellet discarded; the supernatant was then centrifuged at 16,000×g for 30 minutes and the supernatant collected and the pellet discarded; the supernatant was then centrifuged at 133,900×g for 3 hours, and the resulting supernatant discarded and the pellet collected and resuspended in 1 mL of PBS. The resuspended 133,900×g pellet was further purified by running in an OPTIPREP™ Iodixanol gradient: a 4-tier sterile gradient was prepared by mixing 3 mL of OPTIPREP™ (60% Iodixanol) with 1 mL of resuspended pellet to generate 4 mL of 45% Iodixanol, then overlaid serially with 3 mL 30% Iodixanol, 2 mL 22.5% Iodixanol, 2 mL 17.5% Iodixanol, and 1 mL PBS in a 12 mL Ultra-Clear (344059) tube for a SW 41 Ti rotor. The gradient was ultracentrifuged at 150,000×g for 16 hours at 4° C. Ultracentrifugation resulted in a Top Fraction known to contain exosomes, a Middle Fraction containing cell debris of moderate density, and a Bottom Fraction containing high density aggregates and cellular debris. The exosome layer was then gently collected from the top ˜2 mL of the tube.

The exosome fraction was diluted in ˜32 mL PBS in a 38.5 mL Ultra-Clear (344058) tube and centrifuged at 10,000×g for 30 minutes, the supernatant collected and ultracentrifuged at 133,900×g for 3 hours at 4° C. to pellet the purified exosomes. The pelleted exosomes were then resuspended in a minimal volume of PBS (˜200 μL) and stored at 4° C. Final purified concentration of exosomes was determined using nanoparticle tracking analysis (NTA).

Exosome Loading: To load exosomes with maleimide conjugates, exosomes were chemically reduced using TCEP (Tris(2-carboxyethyl)phosphine hydrochloride) at concentrations from 1 to 50 mM; in some cases, the reduction step includes, or is preceded by treatment with, 1-2 M Guanidine hydrochloride for one hour at room temperature. Exosomes were exchanged into PBS by diluting to 1 mL in PBS, centrifuging at 100,000×g for 20 minutes (TLA 120.2 rotor, Beckman) to pellet exosomes, the supernatant was removed and discarded, and the pellet resuspended in 1 mL PBS; this was repeated once to ensure complete buffer exchange. The final exosome pellet was resuspended in 0.1 mL PBS, to which the compound to be loaded was added to a final concentration of up to 300 μM. Exosomes were incubated overnight at 4° C., followed by washing with PBS to remove compound not conjugated to exosomes (diluting to 1 mL in PBS, centrifuging at 100,000×g for 20 minutes (TLA 120.2 rotor, Beckman) to pellet exosomes, the supernatant was removed and discarded, and the pellet resuspended in 1 mL PBS; this was repeated once to ensure complete buffer exchange).

### Example 2

### Efficacy of Free and Exosome Linked STING Agonists

FIG. 1B presents STING agonist compounds that were tested in PBMC assays.

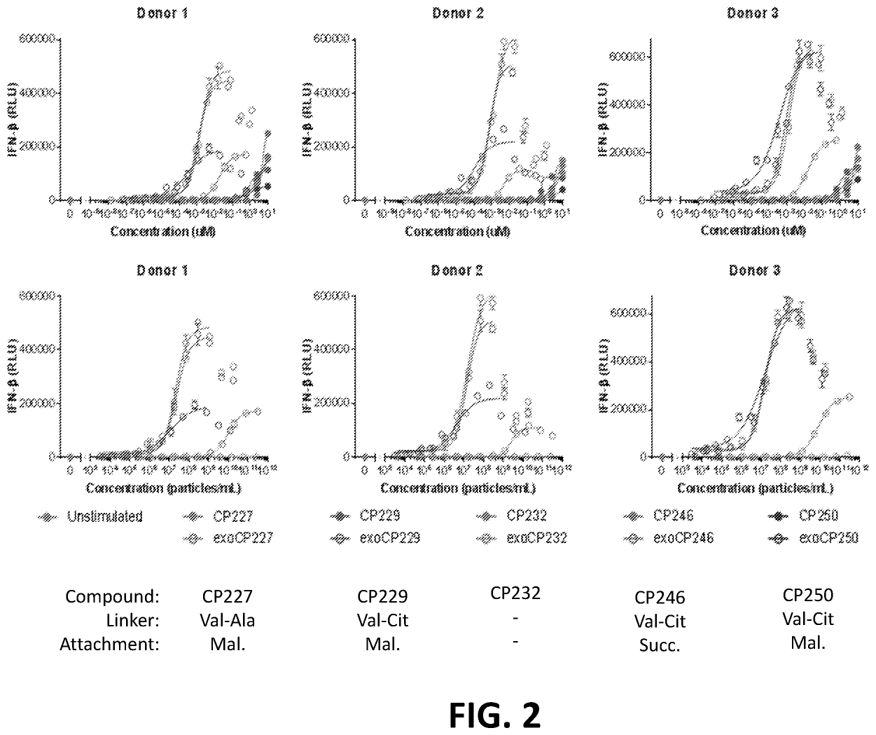

Compounds were synthesized at Sygnature. PBMCs were isolated from heparinized human blood using standard protocols employing a Ficoll-Hypaque density gradient. For each condition to be tested, 500,000 PBMCs were plated in a well of a 96-well plate and cultured overnight with the test sample. The following day, cells were spun down in the plate (500×g for 10 minutes) and the supernatant collected. Interferon beta (IFNβ) release into the cell culture supernatant was measured using an ELISA. FIG. 2.

FIG. 2 shows the STING agonism of sulfhydryl- or amine-reactive compounds assessed by PBMC assay. PBMCs derived from three different healthy human donors were used to assay the activity of either free compounds (closed circles) or compounds loaded on exosomes (open circles). All compounds with a maleimide attachment chemistry (CP227, CP229, CP250) showed a 3-4 log increase in potency when exosome-associated. Exosome association through passive loading (CP232) showed approximately a 2 log increase in potency. Notably, succinimide attachment resulted in low amounts of loading, and no induction of IFNβ release was detected in exosome-loaded samples (CP246).

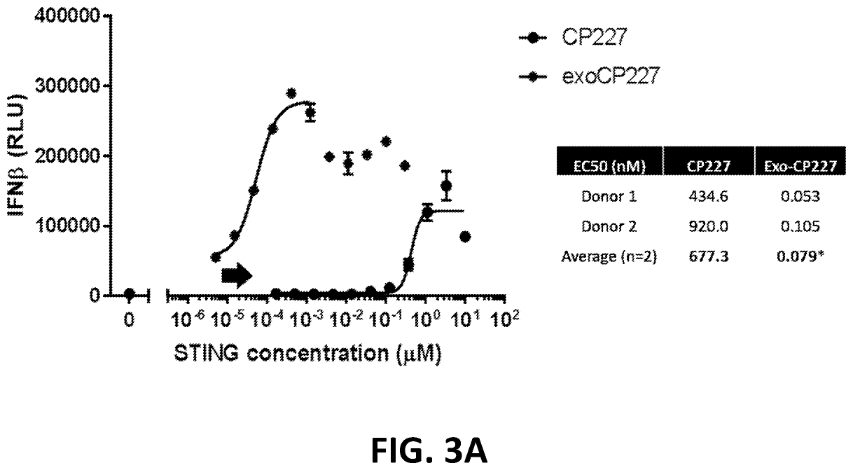

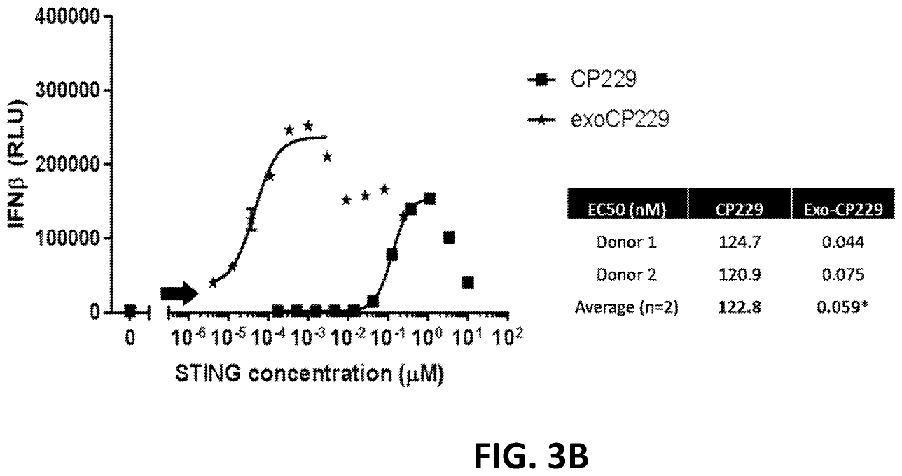

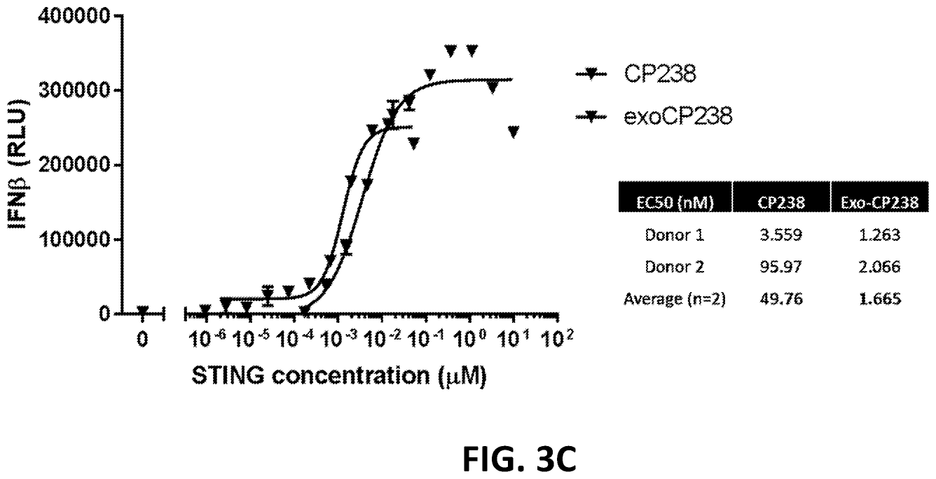

FIG. 3A-3C show a comparison of sulfhydryl-reactive and lipid-associating chemistries for loading STING agonists. PBMCs derived from two different healthy human donors were used to assay the activity of three compounds: CP227, CP229, and CP238. The compounds were tested either free (blue circles/lines) or loaded on exosomes (green circles/lines). Both of the compounds containing the sulfhydryl-reactive maleimide attachment chemistry (CP227 and CP229) demonstrate a more than 3-log increase in potency when attached to exosomes. In contrast, the compound containing a lipid-association cholesterol chemistry showed less than a 1-log shift in potency when coupled to exosomes. Thus, maleimide attachment was superior to cholesterol.

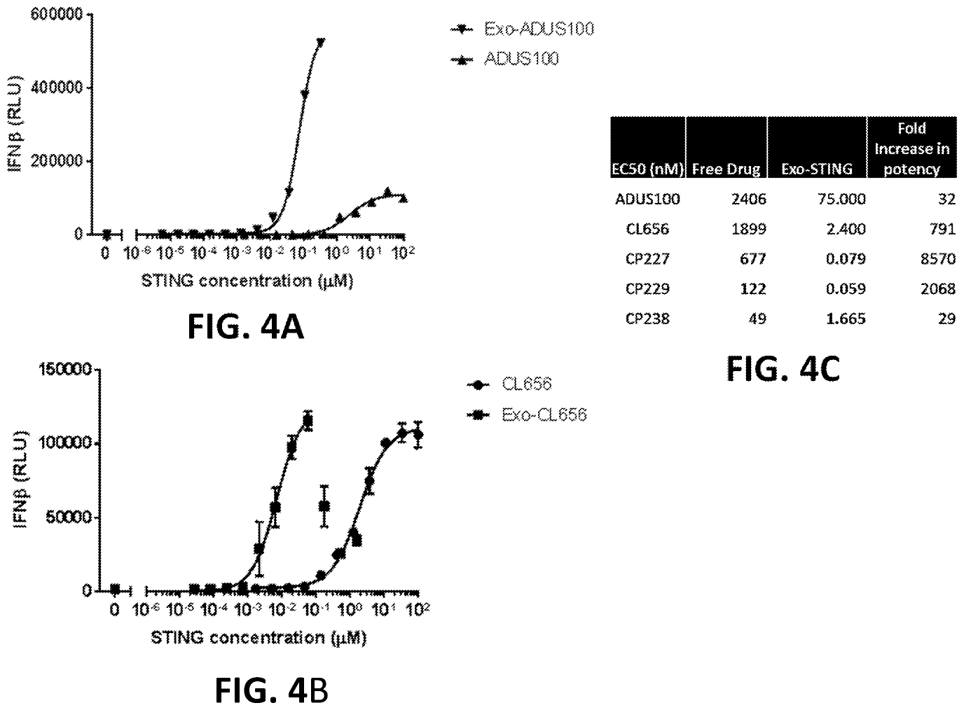

FIGS. 4A-4C show a comparison of unmodified and sulfhydryl-reactive chemistries for loading STING agonists. Exosomes passively loaded with cyclic dinucleotide STING agonists (ADUS100 and CL656) were compared with agonists chemically attached to exosomes with maleimide chemistry (same data for maleimide compounds as is in FIG. 2; i.e., the compounds tested were CP227, CP229, and CP238). The EC₅₀ values for both types of loading were compared in the table presented in FIG. 4. Generally, maleimide-conjugated compounds showed more than a 10-fold increase in the exosome-mediated potency increase when compared to unmodified compounds. Thus, maleimide attachment to the exosomes was superior to passive loading.

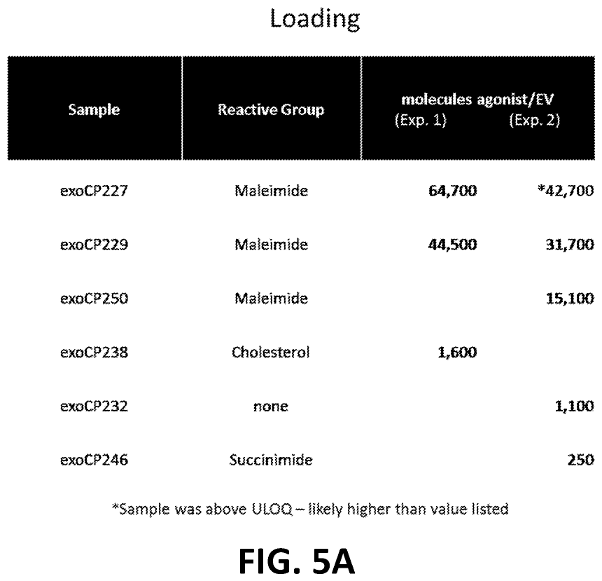

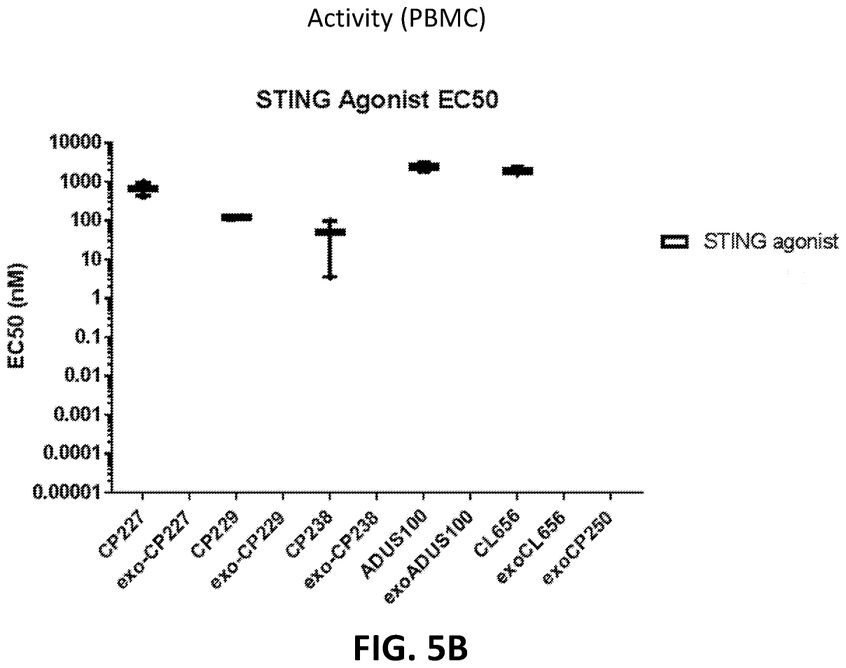

FIGS. 5A and 5B show a comparison of loading and activity of different STING agonists. The amount of STING agonist loaded on exosomes using different attachment chemistries was quantified by mass spectrometry. STING agonist EC₅₀ values were calculated from human PBMC assays. The results indicate that STING agonist loading/activity varies across methods.

### Example 3

### Efficacy of Exosome Linked MMAE

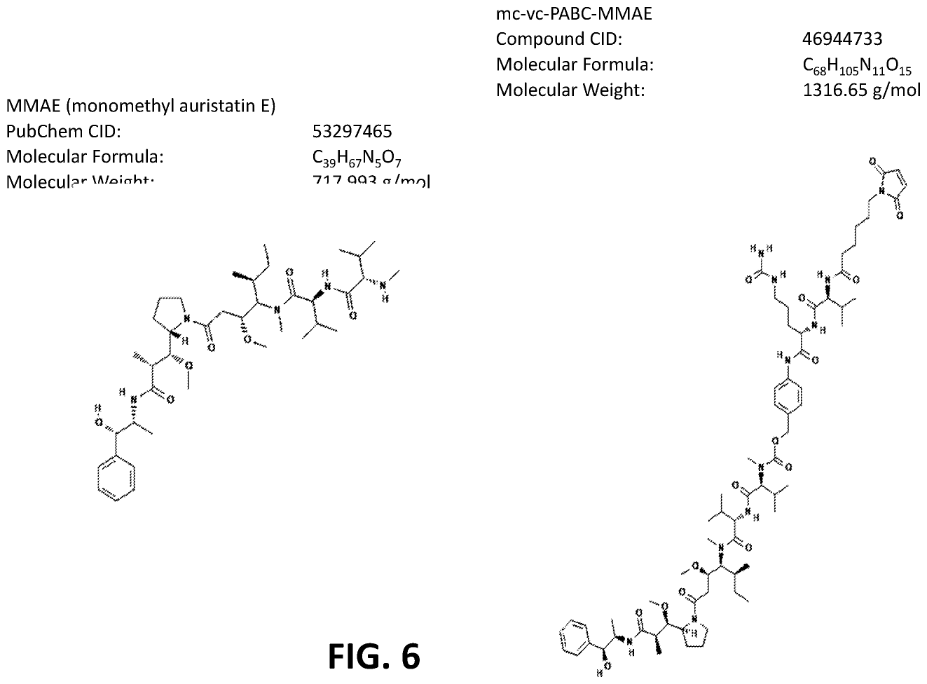

FIG. 6 shows the structure of monomethyl auristatin E (MMAE) and maleimide-vc-PABC-MMAE (vc-MMAE). These two compounds were used to test loading of a cytotoxic compound (MMAE) on exosomes. Both compounds are commercially available, and were ordered from MedChemExpress.

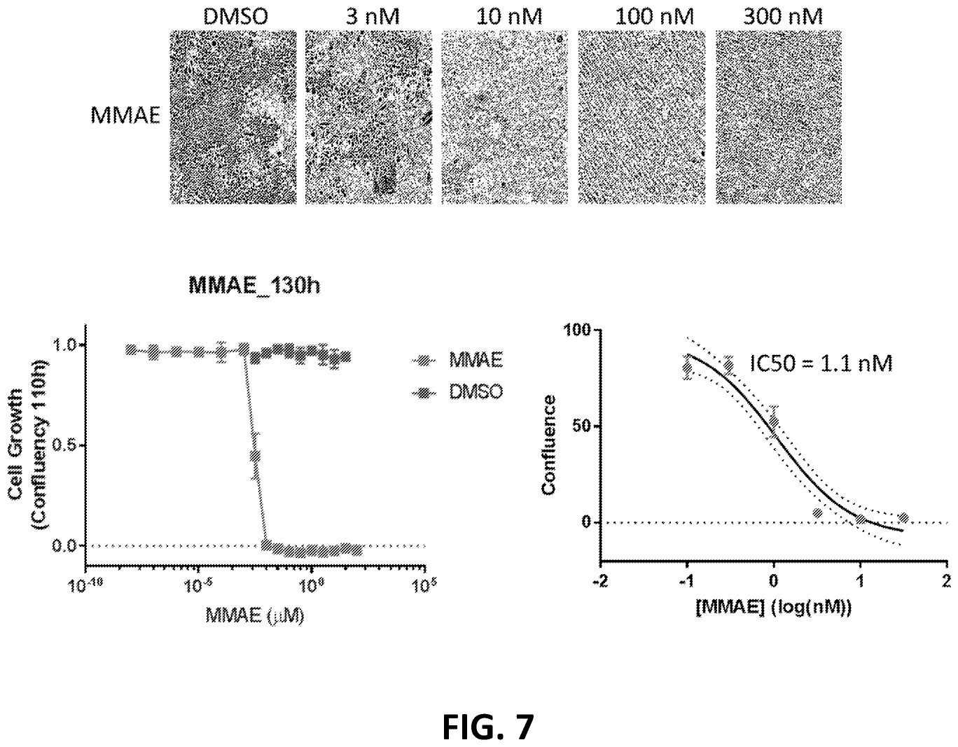

MMAE cytotoxicity was assessed on RAW264.7 (RAW) cells. See FIG. 7. RAW cells are a human macrophage cell line. 10,000 RAW cells were seeded in each well of a 96 well plate, and cell growth was monitored using an IncuCyte instrument to image cells over a period of approximately 5 days. Free MMAE or DMSO (carrier control) was added to RAW cells over a range of concentrations, and potent growth inhibition and cell death was noted for MMAE concentrations above 1.1 nM. DMSO controls showed no effect on cell viability or growth. Images show cells after 5 days of treatment with the indicated dose of MMAE or DMSO; growth inhibition is evident from the decreased number of cells after treatment with MMAE, while cell death is indicated by the rounding up of cells (evident in the 10, 100, and 300 nM images). MMAE has a steep dose-response curve.

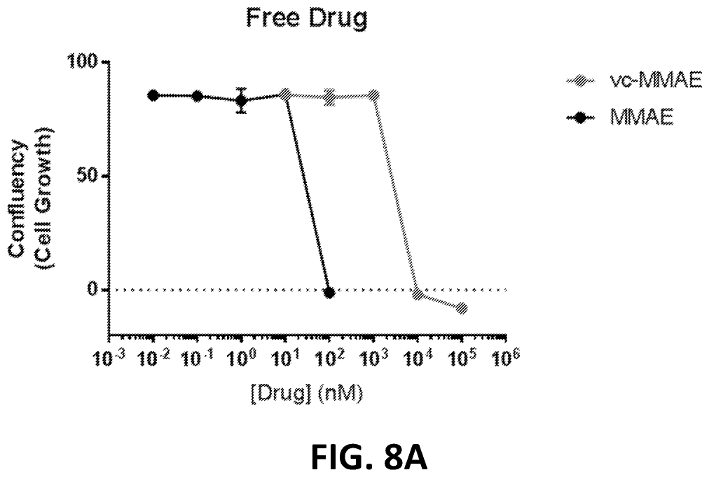

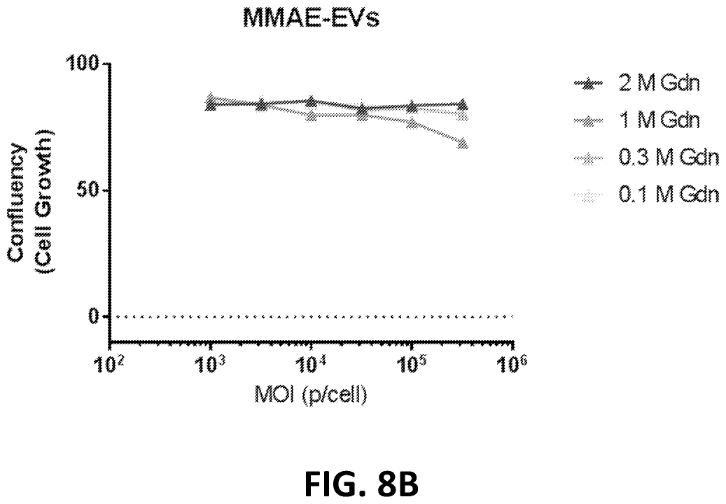

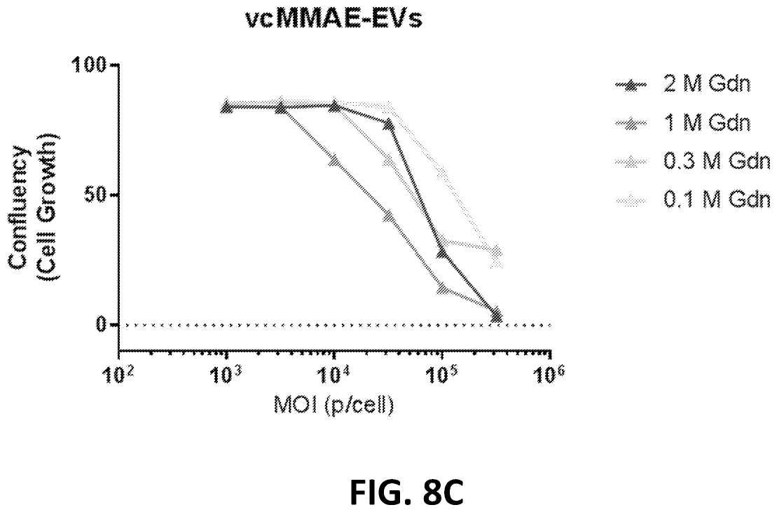

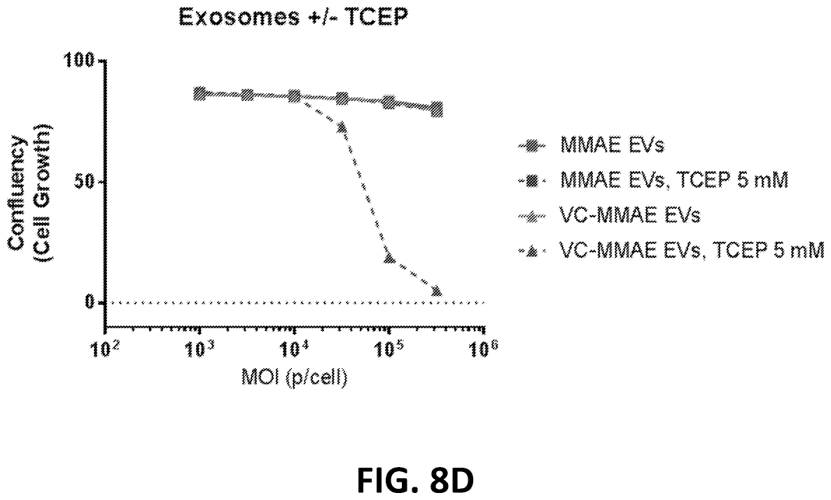

The difference in potency of MMAE with or without maleimide-Val-Cit-PABC linker is shown in FIG. 8A. RAW cells were treated with the indicated concentrations of either unmodified MMAE or vc-MMAE. vc-MMAE appears to be approximately 100-fold less toxic than unmodified MMAE when administered to cells as a free drug. Thus, the attachment of MMAE to a vc linker results in a decrease in potency.

FIG. 8B shows exosome cleanup following incubation with MMAE. Exosomes were incubated with MMAE overnight at 4C, then washed twice by ultracentrifugation (pelleted by centrifuging at 100,000×g for 20 minutes, resuspending in PBS, then pelleting a second time, followed by resuspension). Under no conditions tested were exosomes treated with MMAE found to induce any growth inhibition or toxicity when added to RAW cells. This indicates that free MMAE does not significantly bind to exosomes and that the cleanup procedure removes MMAE from exosomes.

FIG. 8C shows that exosomes loaded with vc-MMAE exhibit potent biological activity. Exosomes were chemically reduced with TCEP (5 mM) and concentrations of guanidine hydrochloride (Gdn) ranging from 0.1 to 2 μM. Exosomes were cleaned up with ultracentrifugation (pelleted by centrifuging at 100,000×g for 20 minutes, resuspending in PBS, then pelleting a second time, followed by resuspension), then added to RAW cells at the indicated MOI (number of exosomes per cell). Toxicity was assessed by measuring cell growth inhibition after 5 days of culture. RAW cells exhibited a dose-dependent decrease in proliferation following treatment with vc-MMAE loaded exosomes. Thus, vc-MMAE significantly attached to the exosomes and was not washed by the chaotropic agent and centrifugation. Furthermore, the MMAE attached to the exosome exhibited a potent inhibitory effect on cell growth.

FIG. 8D shows that chemical reduction of exosomes is required for vc-MMAE activity. Exosomes were either kept in PBS or treated with PBS with 5 mM TCEP, then incubated with either MMAE or vc-MMAE. Samples were cleaned up with ultracentrifugation, then added to RAW cells. Cell growth was notably inhibited only by TCEP-reduced exosomes incubated with vc-MMAE (dark red triangles). This indicated that the maleimide group on the vc-MMAE compound was conjugating to sulfhydryl groups created on exosomes following chemical reduction.

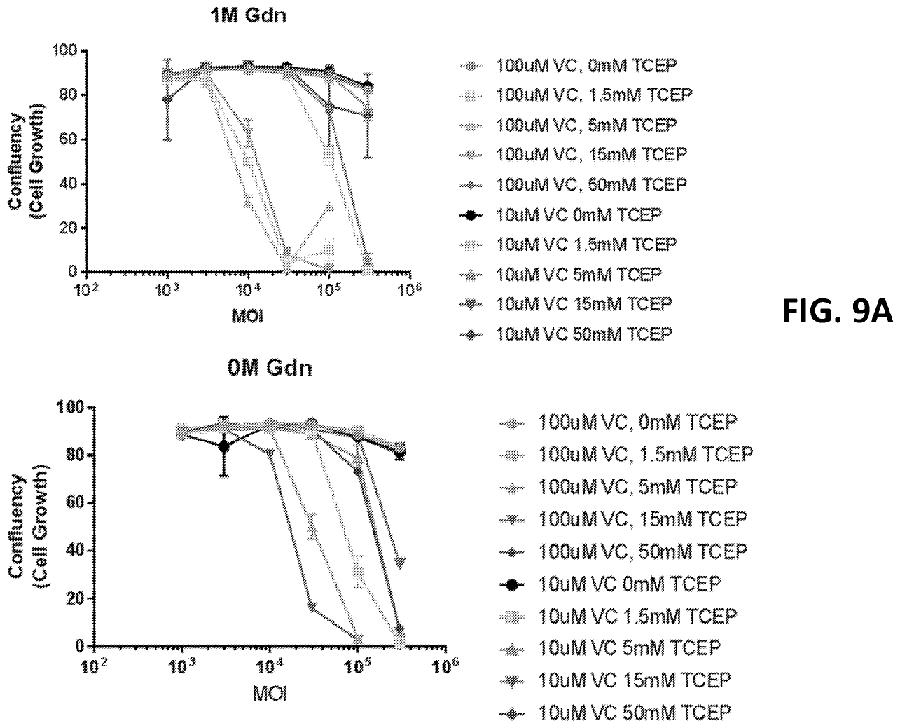

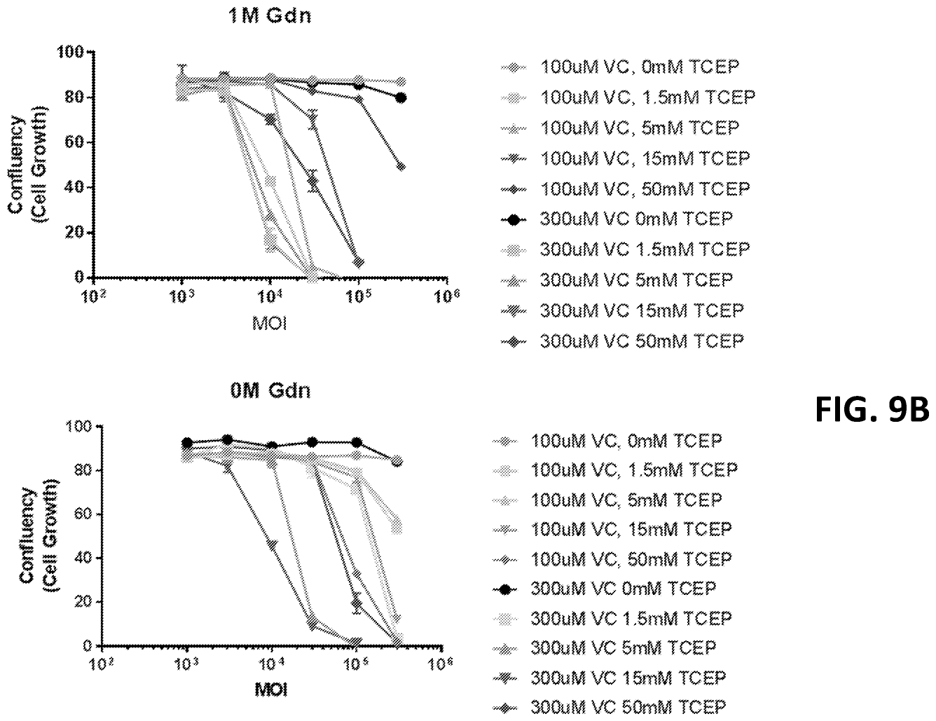

FIG. 9A shows the effect of reducing conditions and loading concentration of compound on potency of exosomes. Exosomes were treated with a range of reducing conditions (0-50 mM TCEP with or without 1 M Guanidine hydrochloride), cleaned up by ultracentrifugation, and then incubated with either 10 or 100 μM vc-MMAE overnight at 4° C. The exosomes were cleaned up by ultracentrifugation, then added to RAW cells to test their effect on cell growth. Exosomes that were not chemically reduced by treatment with TCEP showed no effect on cell growth. Comparing within reducing conditions, loading with 100 μM vc-MMAE lead to a dramatic increase in potency compared to loading with 10 μM vc-MMAE. In the presence of 1 M Gdn, all concentrations of TCEP yielded similar potency, with the exception of the highest concentration of TCEP (50 mM), which showed reduced potency. In the absence of Guanidine hydrochloride, increasing the concentration of TCEP from 1.5 to 15 mM yielded an increase in potency; 50 mM TCEP also showed reduced potency in the absence of Guanidine hydrochloride.

Similarly to FIG. 9A, FIG. 9B shows the effect of reducing conditions and loading concentration of compound on potency of exosomes but at higher vc-MMAE concentrations. The same experimental conditions used in the experiment presented in FIG. 9A were used, but using 100 and 300 μM vc-MMAE as the loading concentrations. Similar to what was observed with 10 and 100 μM vc-MMAE, increasing the loading concentration to 300 μM vc-MMAE further increased the potency. Importantly, at 300 μM vc-MMAE, comparable potency was observed between the 0 and 1M Gdn conditions at 15 mM TCEP; indicating that loading can be conducted without Guanidine hydrochloride provided that the reducing condition and loading concentration are optimized.

The MMAE experiments illustrate the complex interplay between the concentrations of TCEP, Guanidine hydrochloride, and Val-Cit-MMAE, and show many unexpected interactions (i.e. 1M Gdn is better than 2M, FIG. 8B; 15 mM TCEP is better than 50 mM, FIGS. 9A and 9B; increasing TCEP concentration from 1.5 mM to 15 mM increases potency in the absence of Gdn, but makes no difference in 1M Gdn, FIG. 9B).

### Example 4

### Exosome Linked PROTACs

A TBK1 PROTAC according to FIG. 10C is attached to an exosome according to the methods disclosed above. The PROTAC comprises a TBK1 targeting ligand, a linker, and ligand capable of binding to the VHL E3 ubiquitin ligase.

The PROTAC is attached to the exosome, e.g., the external surface and/or the luminal surface of the exosome membrane via a maleimide moiety (directly or indirectly via a linker). The PROTAC can be attached to the exosome via a maleimide-VA-PABC cleavable linker. The PROTAC can be attached to the exosome via the VHL (E3 ligase) binding ligand.

The functionality of the TBK1 PROTAC attached to a an exosome of the present disclosure can be determined using in vitro or in vivo methods. In vitro methods include Western blot to (i) directly measure TBK1 degradation in cell lines, (ii) determine inhibition of IRF3 phosphorylation following stimulation with a STING agonist (poly I:C, CL656, LPS, etc.), or (iii) determine TBK1 protein knockdown in human monocytes. Another in vitro assay can determined inhibition of STING agonism, e.g., using the B16 IRF reporter cell line (e.g., pretreat with the exosome-PROTAC conjugate, stimulate with a STING agonist, and measure report response) or human monocytes (e.g., measure IFBbeta release).

In vivo assays to determine the functionality of the exosome-PROTAC conjugates of the present disclosure include, for example, assays to determine (i) TBK1 protein knockdown in peritoneal macrophages (e.g., dose intraperitoneally with exosome-PROTAC conjugate, collect peritoneal macrophages, and measure knockdown by Western blot or flow cytometry), (ii) inhibition of STING agonism-induced serum cytokines (e.g., pretreat intraperitoneally with exosome-PROTAC conjugate, stimulate intraperitoneally with STING agonist, and measure plasma/serum cytokines at certain timepoints), or (iii) inhibition of STING agonism-induced phosphoIRF3 (for example, if knockdown is very selective and no reduction in serum cytokines is observed, determining pIRF3 levels in different cell types, e.g., using anti-pIRF antibody and flow cytometry, can help show selectivity).

### Example 5

### Exosome-Linked LPA1 Inhibitors—ExoAM152

Lysophosphatidic acid (LPA) is a highly potent endogenous lipid mediator that protects and rescues cells from programmed cell death. LPA, through its high affinity LPA1 receptor, is an important mediator of fibrogenesis.

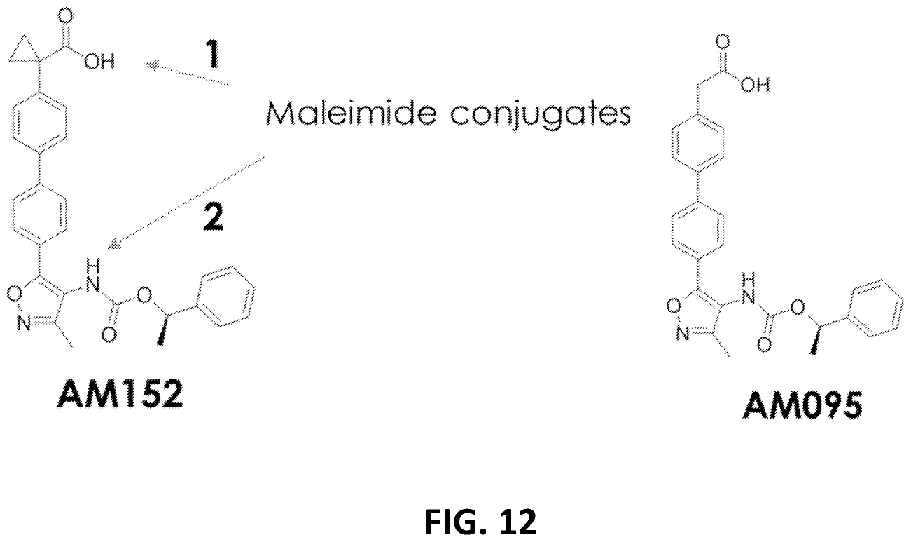

AM152 (also known as BMS-986020) is a specific LPA1 inhibitor. AM152 is a high-affinity LPA1 antagonist which inhibits bile acid and phospholipid transporters with IC₅₀s of 4.8 μM, 6.2 μM, and 7.5 μM for BSEP, MRP4, and MDR3, respectively. The chemical structures of the LPA1 inhibitors AM152 and AM095 are presented in FIG. 12. The figure shows that maleimide-containing reagents can be conjugated to the carboxylic acid and/or carbamate groups of AM152. The same approach could be used to derivatize AM095 since the same reactive groups are present in AM095.

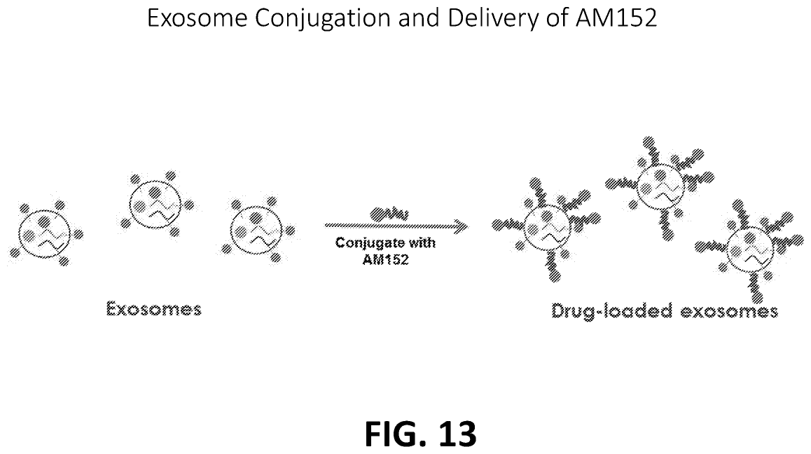

LPA1 antagonists such as AM095 and AM152 can be chemically linked to the surface of exosomes using the methods disclosed in the present specification. The results would be EV, e.g., exosomes, comprising a plurality of antagonist molecules of their surface. See FIG. 13.

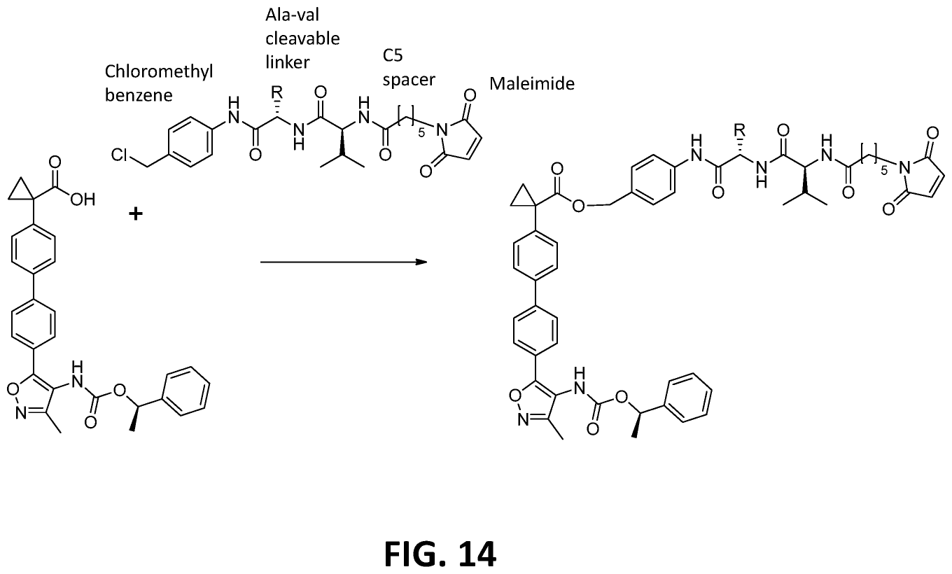

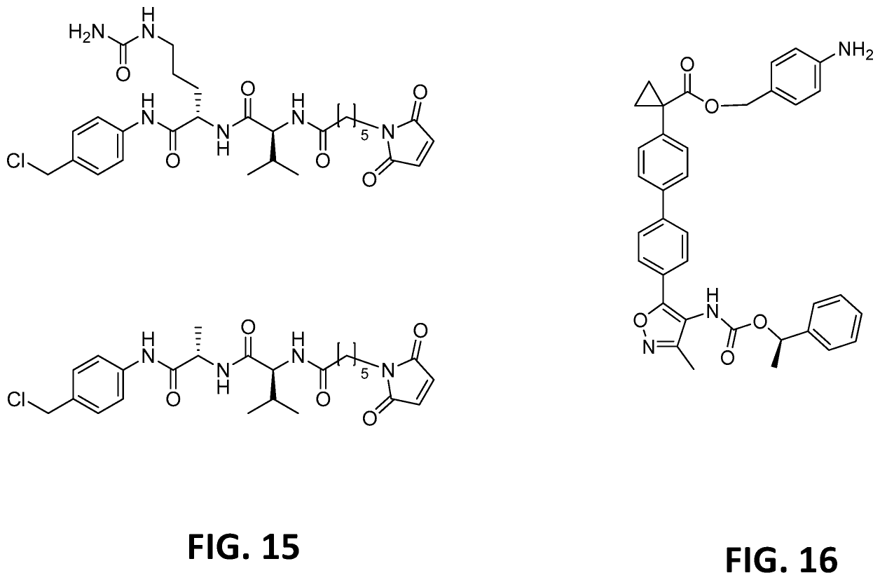

FIG. 14 shows an example of how a maleimide reactive group can be added to AM152 via the acid group. The example shows the maleimide group as part of a complex comprising an ala-val cleavable linker interposed between the maleimide group and the carboxylic acid-reactive chloromethyl benzene group. FIG. 15 shows two exemplary reagents that can be used to derivatize AM152. The top reagent comprises (i) a chloromethyl benzene group that can react with the carboxylic acid group of AM152 and (ii) a maleimide group; and interposed between them are a cleavable cit-val dipeptide and a C5 spacer. The bottom reagent comprises (i) a chloromethyl benzene group that can react with the carboxylic acid group of AM152 and (ii) a maleimide group, and interposed between them are a cleavable ala-val dipeptide and a C5 spacer. The maleimide group would be subsequently used to attach the AM152 (or AM095), e.g., to a scaffold moiety either directly or indirectly via one or more spacers or linkers.

FIG. 16 shows the product that would result from cleaving the cit-val or ala-val dipeptide (e.g., by cathepsin B) in the conjugation product. The product, an AM152 aniline ester, could be further processed by an endogenous esterase to yield the free acid AM152 product.

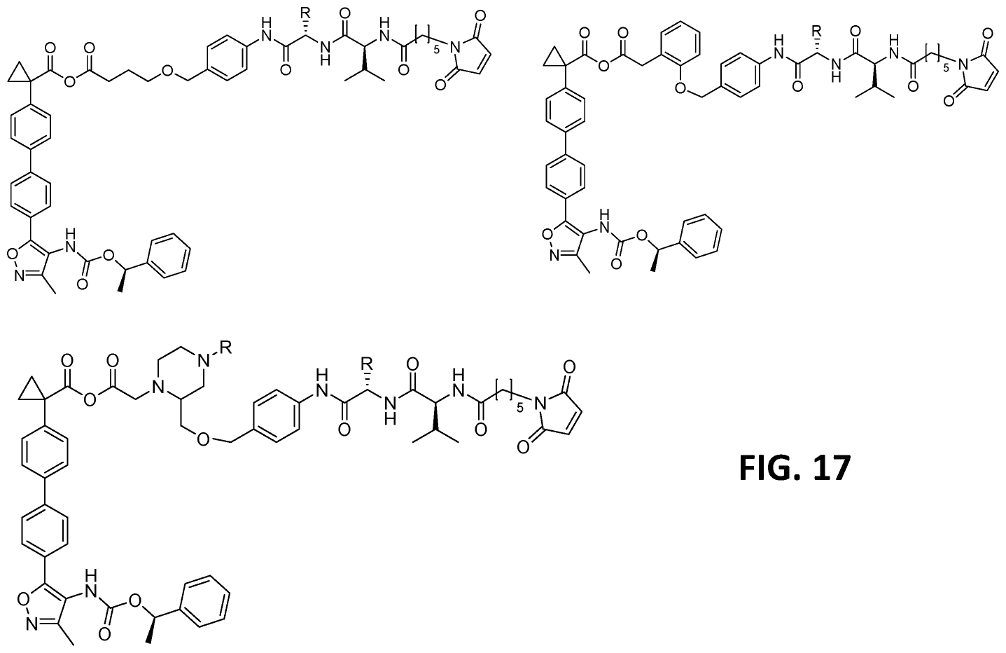

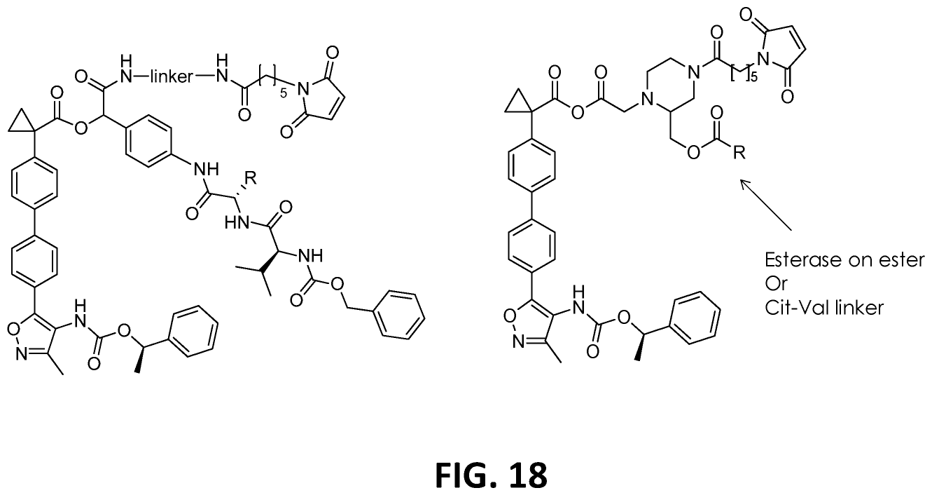

FIG. 17 shows several AM152 derivatives comprising a free maleimide group and different combinations of spacers. Additional derivatives are shown in FIG. 18.

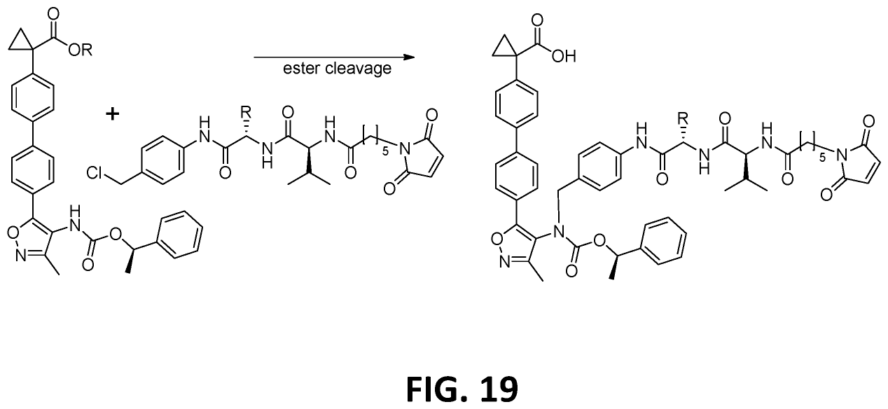

FIG. 19 shows that after protection of the carboxylic acid group, it is possible to use the same reagents used to derivatize the carboxylic acid group to derivatize AM152 at its carbamate group. The resulting product would be subsequently deprotected to free the carboxylic acid group.

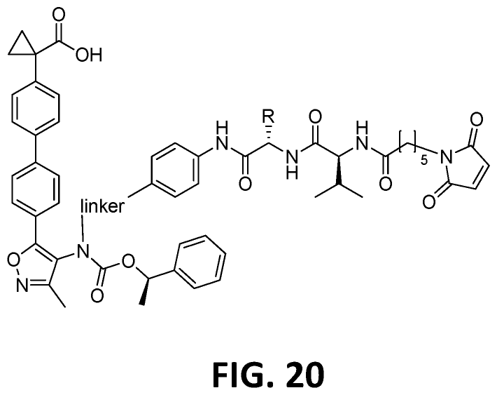

FIG. 20 illustrates an example in which the complex with the maleimide group is attached to the carbamate group of AM152 via a linker. Suitable linkers include any of the linkers disclosed in the present specification.

The processes disclosed in this example relate to the generation of an AM152 or AM095 derivative comprising a free maleimide reactive group, which could subsequently react with a scaffold moiety either directly or indirectly via one or more spacers or linkers. As a result, the AM152 or AM095 would be attached to the external surface of the EV, e.g., an exosome.

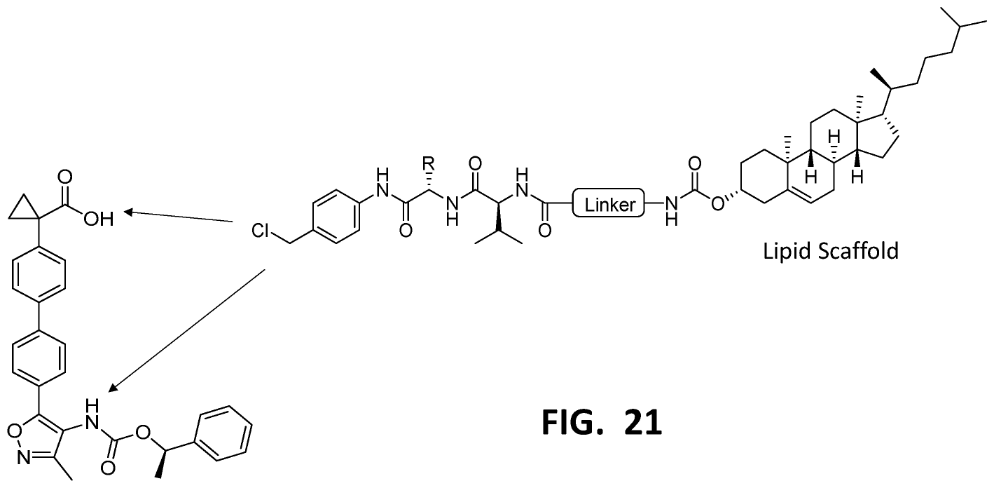

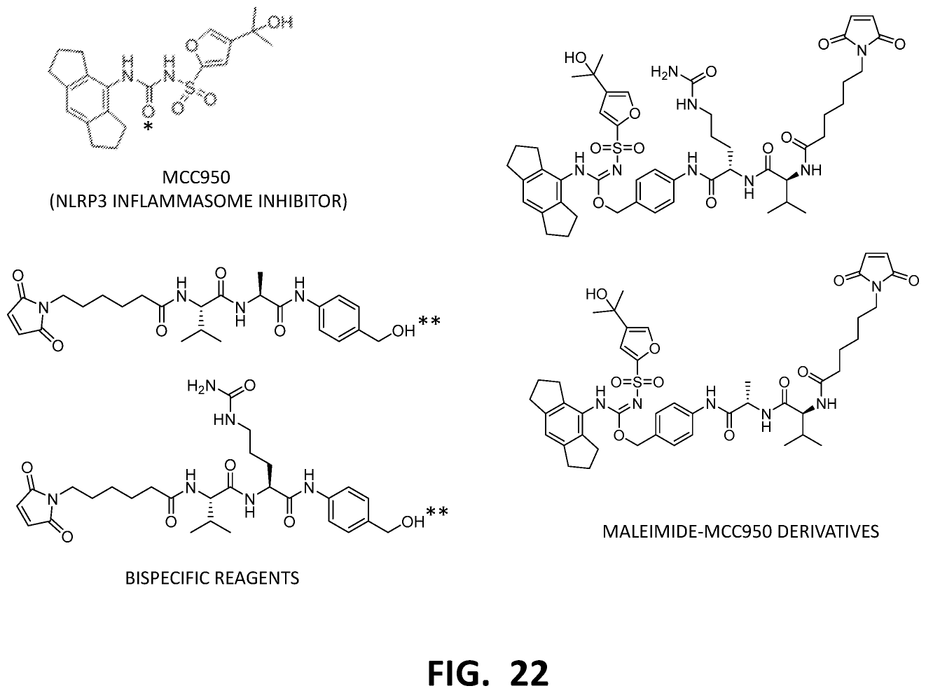

However, the invention could also be practiced by derivatizing a scaffold moiety first, e.g., with a bifunctional group comprising maleimide, and then reacting the derivatized scaffold moiety, e.g., having a free chloromethyl benzene group, with either the carboxylic acid or the carbamate group of AM152, as shown in FIG. 21.

In some aspects, chemically linking AM152 (or AM095) to the surface of EV, e.g., exosomes, via a maleimide moiety improves at least one beneficial property of unconjugated AM152 (or AM095) and/or decreases at least one deleterious property of unconjugated AM152 or AM095 (e.g., toxicity, such as gall bladder toxicity and/or liver toxicity). In some aspects, chemically linking AM152 (or AM095) to an EV, e.g., an exosome, via a maleimide moiety improves the efficacy of AM512 or AM095 (compared to free AM152 or free AM095) in the treatment of a fibrotic disease, e.g., lung fibrosis, such as IPF.

### Example 6

### Exosome-Linked NLRP3 Inhibitors—ExoMCC950

MCC950 (N-[[(1,2,3,5,6,7-hexahydro-s-indacen-4-yl)amino]carbonyl]-4-(1-hydroxy-1-methylethyl)-2-furansulfonamide) is a potent and selective inhibitor of the NLRP3 (NOD-like receptor (NLR) pyrin domain-containing protein 3) inflammasome. MCC950 blocks the release of IL-1β induced by NLRP3 activators, such as ATP, MSU and nigericin, by preventing oligomerization of the inflammasome adaptor protein ASC (Apoptosis-associated Speck-like protein containing CARD). Coll et al. (2015) Nature Med. 21:248-255. MCC950 blocks the release of IL-1β in macrophages primed with LPS and activated with ATP or nigericin with an IC50 of approximately 7.5 nM. Although MCC950 blocks the release of IL-1β induced by NLRP3, MCC950 does not inhibit the NLRC4, AIM2, or NLRP1 inflammasomes. Furthermore, MCC950 does not inhibit TLR2 signaling, or priming of NLRP3.

MCC950 is active in vivo, blocking the production of IL-1β and enhancing survival in mouse models of multiple sclerosis. MCC950 also inhibits NLRP3-induced IL-1β production in models for myocardial infarction. van Hout et al (2015) Eur. Heart J. ehw247. MCC950 is also active in ex vivo samples from individuals with Muckle-Wells syndrome. Thus, MCC950 is a potential therapeutic agent for the treatment of NLRP3-associated syndromes, including auto-inflammatory and auto-immune diseases.

FIG. 22 shows the structure of MCC950, bifunctional reagents that can be used to derivatize MCC950 to introduce a maleimide reactive group, and MCC950 derivatives comprising a maleimide reactive group. The benzene groups of the bifunctional reagents (**) can react with the carbamate group of MCC950 (*) to yield the MCC950 derivatives depicted in FIG. 22.

The processes disclosed in this example relate to the generation of an MCC950 derivative comprising a free maleimide reactive group, and optionally one of more linkers interposed between the MCC950 moiety and the maleimide group (e.g., a cleavable linker and/or one or more spacers) which could subsequently react with a scaffold moiety either directly or indirectly via one or more spacers or linkers. As a result, the MCC950 would be attached to the external surface of the EV, e.g., an exosome.

The invention could also be practiced by derivatizing a scaffold moiety first, e.g., with a bifunctional group comprising maleimide, and then reacting the derivatized scaffold moiety, e.g., having a free chloromethyl benzene group or benzene group, with the carbamate group of MCC950 or another suitable derivatizable group.

In some aspects, chemically linking MCC950 to the surface of EV, e.g., exosomes, via a maleimide moiety improves at least one beneficial property of unconjugated MCC950 and/or decreases at least one deleterious property of unconjugated MCC950 (e.g., toxicity, such as gall bladder toxicity and/or liver toxicity). In some aspects, chemically linking MCC950 to an EV, e.g., an exosome, via a maleimide moiety improves the efficacy of MCC950 (compared to free MCC950) in the treatment of an NLRP3 inflammasome-related diseases or disorders such as multiple sclerosis, type 2 diabetes, Alzheimer's disease, atherosclerosis, neuroinflammation, Parkinson's, prion diseases, cardiac injury due to myocardial infarction, gout, and in general any NLRP3-associated syndromes, including auto-inflammatory and auto-immune diseases.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

1-84. (canceled)

85. An extracellular vesicle (EV) comprising a biologically active molecule and a cleavable linker.

86. The cleavable linker of claim 85, wherein the cleavable linker comprises an enzymatic-cleavable linker.

87. The cleavable linker of claim 86, wherein the enzymatic-cleavable linker comprises a reductase-cleavable linker, a protease cleavable-linker, a nuclease-cleavable linker, an esterase-cleavable linker, a phosphatase-cleavable linker, an amidase-cleavable linker, and/or an oxidase-cleavable linker.

88. The cleavable linker of claim 85, wherein the cleavable linker comprises a redox cleavable linker.

89. The cleavable linker of claim 88, wherein the redox cleavable linker comprises a disulfide bond.

90. The cleavable linker of claim 85, wherein the cleavable linker comprise a protease-cleavable linker.

91. The EV of claim 90, wherein the protease-cleavable linker comprises valine, alanine, citrulline, phenylalanine, N-methyl-valine, lysine, cyclohexyl-alanine, beta-alanine, glycine, or any combination thereof.

92. The EV of claim 85, wherein the cleavable linker further comprises a self-immolative spacer.

93. The EV of claim 85, wherein the cleavable linker further comprises a non-cleavable-linker.

94. The EV of claim 93, wherein the non-cleavable linker comprises tetraethylene glycol (TEG), polyethylene glycol (PEG), hexaethylene glycol (HEG), succinimide, a glycine spacer, a C2 to C12 alkyl spacer, or any combination thereof.

95. The EV of claim 94, wherein the glycine spacer is glycine, or glycine-glycine.

96. The EV of claim 85, wherein the cleavable linker is attached to a scaffold protein or to a scaffold lipid.

97. The EV of claim 96, wherein the scaffold protein comprises a prostaglandin F2 receptor negative regulator (PTGFRN) polypeptide, a basigin (BSG) polypeptide, an immunoglobulin superfamily member 2 (IGSF2) polypeptide, an immunoglobulin superfamily member 3 (IGSF3) polypeptide, an immunoglobulin superfamily member 8 (IGSF8) polypeptide, an integrin beta-1 (ITGB1) polypeptide, an integrin beta-4 (ITGA4) polypeptide, a 4F2 cell-surface antigen heavy chain (SLC3A2) polypeptide, an ATP transporter polypeptide, a fragment thereof, or any combination thereof.

98. The EV of claim 96, wherein the scaffold lipid is selected from the group consisting of cholesterol and other sterols, fatty acids, phospholipids, lysolipids, vitamins, and combinations thereof.

99. The EV of claim 85, wherein the biologically active molecule comprises a vaccine antigen, a vaccine adjuvant, a small molecule, a proteolysis-targeting chimera (PROTAC), a stimulator of interferon genes protein (STING) agonist, a polynucleotide, an antisense oligonucleotide (ASO), a synthetic antineoplastic agent, a cytokine release inhibitor, a mammalian target of rapamycin (mTOR) inhibitor, an autotaxin inhibitor, a lysophosphatidic acid receptor 1 (LPA1) antagonist, or any combination thereof.

100. The EV of claim 85, further comprising a targeting moiety, a tropism moiety, an anti-phagocytic signal, or any combination thereof.

101. The EV of claim 85, wherein the EV is an exosome.

102. A pharmaceutical composition comprising the EV of claim 85 and a pharmaceutically acceptable carrier.

103. A method of treating a disease or disorder selected from the group consisting of a cancer, an inflammatory disorder, a neurodegenerative disorder, a central nervous diseases, and a metabolic disease in a subject in need thereof, comprising administering a therapeutically effective amount of the EV of claim 85 to the subject.

104. A method of attaching a biologically active molecule to an EV comprising
(i) connecting a biologically active moiety to an anchoring moiety via a cleavable linker; and,
(ii) bringing the anchoring moiety in contact with the EV.


SEQUENCE LISTING (231 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 192 aa, Homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 3 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 4 - protein, 613 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 5 - protein, 748 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 6 - protein, 1032 aa, Homo sapiens [sequence omitted]

SEQ ID NO: 7 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 8 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 9 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 10 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 11 - protein, 4 aa, Artificial Sequence
  Synthetic construct
KKKK

SEQ ID NO: 12 - protein, 5 aa, Artificial Sequence
  Synthetic construct
KKKKK

SEQ ID NO: 13 - protein, 4 aa, Artificial Sequence
  Synthetic construct
RRRR

SEQ ID NO: 14 - protein, 5 aa, Artificial Sequence
  Synthetic construct
RRRRR

SEQ ID NO: 15 - protein, 4 aa, Artificial Sequence
  Synthetic construct
  MOD_RES (1)..(4): X = Lys or Arg
XXXX

SEQ ID NO: 16 - protein, 5 aa, Artificial Sequence
  Synthetic construct
  misc_feature (1)..(5): Xaa can be any naturally occurring amino acid
XXXXX

SEQ ID NO: 17 - protein, 7 aa, Artificial Sequence
  Synthetic construct
GGKLSKK

SEQ ID NO: 18 - protein, 7 aa, Artificial Sequence
  Synthetic construct
GAKLSKK

SEQ ID NO: 19 - protein, 7 aa, Artificial Sequence
  Synthetic construct
GGKQSKK

SEQ ID NO: 20 - protein, 7 aa, Artificial Sequence
  Synthetic construct
GGKLAKK

SEQ ID NO: 21 - protein, 7 aa, Artificial Sequence
  Synthetic construct
GGKLSKK

SEQ ID NO: 22 - protein, 8 aa, Artificial Sequence
  Synthetic construct
GGKLSKKK

SEQ ID NO: 23 - protein, 8 aa, Artificial Sequence
  Synthetic construct
GGKLSKKS

SEQ ID NO: 24 - protein, 8 aa, Artificial Sequence
  Synthetic construct
GAKLSKKK

SEQ ID NO: 25 - protein, 8 aa, Artificial Sequence
  Synthetic construct
GAKLSKKS

SEQ ID NO: 26 - protein, 8 aa, Artificial Sequence
  Synthetic construct
GGKQSKKK

SEQ ID NO: 27 - protein, 8 aa, Artificial Sequence
  Synthetic construct
GGKQSKKS

SEQ ID NO: 28 - protein, 8 aa, Artificial Sequence
  Synthetic construct
GGKLAKKK

SEQ ID NO: 29 - protein, 8 aa, Artificial Sequence
  Synthetic construct
GGKLAKKS

SEQ ID NO: 30 - protein, 4 aa, Artificial Sequence
  Synthetic construct
GGGG

SEQ ID NO: 31 - protein, 4 aa, Artificial Sequence
  Synthetic construct
  misc_feature (1)..(4): Linker; can comprise 1 to 100 repeats of the sequence
GGGS

SEQ ID NO: 32 - protein, 14 aa, Artificial Sequence
  Synthetic construct
GGKLSKKKKGYNVN

SEQ ID NO: 33 - protein, 14 aa, Artificial Sequence
  Synthetic construct
GAKLSKKKKGYNVN

SEQ ID NO: 34 - protein, 14 aa, Artificial Sequence
  Synthetic construct
GGKQSKKKKGYNVN

SEQ ID NO: 35 - protein, 14 aa, Artificial Sequence
  Synthetic construct
GGKLAKKKKGYNVN

SEQ ID NO: 36 - protein, 14 aa, Artificial Sequence
  Synthetic construct
GGKLSKKKKGYSGG

SEQ ID NO: 37 - protein, 14 aa, Artificial Sequence
  Synthetic construct
GGKLSKKKKGSGGS

SEQ ID NO: 38 - protein, 14 aa, Artificial Sequence
  Synthetic construct
GGKLSKKKKSGGSG

SEQ ID NO: 39 - protein, 14 aa, Artificial Sequence
  Synthetic construct
GGKLSKKKSGGSGG

SEQ ID NO: 40 - protein, 14 aa, Artificial Sequence
  Synthetic construct
GGKLSKKSGGSGGS

SEQ ID NO: 41 - protein, 14 aa, Artificial Sequence
  Synthetic construct
GGKLSKSGGSGGSV

SEQ ID NO: 42 - protein, 14 aa, Artificial Sequence
  Synthetic construct
GAKKSKKRFSFKKS

SEQ ID NO: 43 - protein, 7 aa, Artificial Sequence
  Synthetic construct
  Amino acids 453 to 459 of SEQ ID NO44
GTTTQSR

SEQ ID NO: 44 - protein, 735 aa, Artificial Sequence [sequence omitted]

SEQ ID NO: 45 - protein, 653 aa, Artificial Sequence
  Synthetic construct
  REP1
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 46 - protein, 2 aa, Artificial Sequence
  Synthetic construct
  MISC_FEATURE (1)..(2): Shortest example of [(Gly)n-Ser]m linker, wherein n is any integer from 1 to 100 and m is any integer from 1 to 100
GS

SEQ ID NO: 47 - protein, 2 aa, Artificial Sequence
  Synthetic construct
  MISC_FEATURE (1)..(2): Shortest example of [(Gly)x-Sery]z linker, wherein x in an integer from 1 to 4, y is 0 or 1, and z is an integers from 1 to 50
GS

SEQ ID NO: 48 - protein, 1 aa, Artificial Sequence
  Synthetic construct
  MISC_FEATURE (2)..(2): Shortest example of Gn linker, wherein n can be an integer from 1 to 100
G

SEQ ID NO: 49 - protein, 2 aa, Artificial Sequence
  Synthetic construct
  MISC_FEATURE (1)..(2): Shortest example of (GlyAla)n linker, wherein n is an integer between 1 and 100
GA

SEQ ID NO: 50 - protein, 3 aa, Artificial Sequence
  Synthetic construct
  MISC_FEATURE (1)..(3): Linker; can comprise 1 to 100 repeats of the sequence
GGS

SEQ ID NO: 51 - protein, 6 aa, Artificial Sequence
  Synthetic construct
GGKLSK

SEQ ID NO: 52 - protein, 6 aa, Artificial Sequence
  Synthetic construct
GAKLSK

SEQ ID NO: 53 - protein, 6 aa, Artificial Sequence
  Synthetic construct
GGKQSK

SEQ ID NO: 54 - protein, 6 aa, Artificial Sequence
  Synthetic construct
GGKLAK

SEQ ID NO: 55 - protein, 4 aa, Artificial Sequence
  Synthetic construct
KKKG

SEQ ID NO: 56 - protein, 5 aa, Artificial Sequence
  Synthetic construct
KKKGY

SEQ ID NO: 57 - protein, 6 aa, Artificial Sequence
  Synthetic construct
KKKGYN

SEQ ID NO: 58 - protein, 7 aa, Artificial Sequence
  Synthetic construct
KKKGYNV

SEQ ID NO: 59 - protein, 8 aa, Artificial Sequence
  Synthetic construct
KKKGYNVN

SEQ ID NO: 60 - protein, 6 aa, Artificial Sequence
  Synthetic construct
KKKGYS

SEQ ID NO: 61 - protein, 6 aa, Artificial Sequence
  Synthetic construct
KKKGYG

SEQ ID NO: 62 - protein, 7 aa, Artificial Sequence
  Synthetic construct
KKKGYGG

SEQ ID NO: 63 - protein, 5 aa, Artificial Sequence
  Synthetic construct
KKKGS

SEQ ID NO: 64 - protein, 6 aa, Artificial Sequence
  Synthetic construct
KKKGSG

SEQ ID NO: 65 - protein, 6 aa, Artificial Sequence
  Synthetic construct
KKKGSG

SEQ ID NO: 66 - protein, 7 aa, Artificial Sequence
  Synthetic construct
KKKGSGS

SEQ ID NO: 67 - protein, 4 aa, Artificial Sequence
  Synthetic construct
KKKS

SEQ ID NO: 68 - protein, 5 aa, Artificial Sequence
  Synthetic construct
KKKSG

SEQ ID NO: 69 - protein, 6 aa, Artificial Sequence
  Synthetic construct
KKKSGG

SEQ ID NO: 70 - protein, 7 aa, Artificial Sequence
  Synthetic construct
KKKSGGS

SEQ ID NO: 71 - protein, 8 aa, Artificial Sequence
  Synthetic construct
KKKSGGSG

SEQ ID NO: 72 - protein, 8 aa, Artificial Sequence
  Synthetic construct
KKSGGSGG

SEQ ID NO: 73 - protein, 10 aa, Artificial Sequence
  Synthetic construct
KKKSGGSGGS

SEQ ID NO: 74 - protein, 8 aa, Artificial Sequence
  Synthetic construct
KRFSFKKS

SEQ ID NO: 75 - protein, 29 aa, Artificial Sequence
  Synthetic construct
GGKLSKKKKGYNVNDEKAKEKDKKAEGAA

SEQ ID NO: 76 - protein, 28 aa, Artificial Sequence
  Synthetic construct
GGKLSKKKKGYNVNDEKAKEKDKKAEGA

SEQ ID NO: 77 - protein, 27 aa, Artificial Sequence
  Synthetic construct
GGKLSKKKKGYNVNDEKAKEKDKKAEG

SEQ ID NO: 78 - protein, 26 aa, Artificial Sequence
  Synthetic construct
GGKLSKKKKGYNVNDEKAKEKDKKAE

SEQ ID NO: 79 - protein, 25 aa, Artificial Sequence
  Synthetic construct
GGKLSKKKKGYNVNDEKAKEKDKKA

SEQ ID NO: 80 - protein, 24 aa, Artificial Sequence
  Synthetic construct
GGKLSKKKKGYNVNDEKAKEKDKK

SEQ ID NO: 81 - protein, 23 aa, Artificial Sequence
  Synthetic construct
GGKLSKKKKGYNVNDEKAKEKDK

SEQ ID NO: 82 - protein, 22 aa, Artificial Sequence
  Synthetic construct
GGKLSKKKKGYNVNDEKAKEKD

SEQ ID NO: 83 - protein, 21 aa, Artificial Sequence
  Synthetic construct
GGKLSKKKKGYNVNDEKAKEK

SEQ ID NO: 84 - protein, 20 aa, Artificial Sequence
  Synthetic construct
GGKLSKKKKGYNVNDEKAKE

SEQ ID NO: 85 - protein, 19 aa, Artificial Sequence
  Synthetic construct
GGKLSKKKKGYNVNDEKAK

SEQ ID NO: 86 - protein, 18 aa, Artificial Sequence
  Synthetic construct
GGKLSKKKKGYNVNDEKA

SEQ ID NO: 87 - protein, 17 aa, Artificial Sequence
  Synthetic construct
GGKLSKKKKGYNVNDEK

SEQ ID NO: 88 - protein, 16 aa, Artificial Sequence
  Synthetic construct
GGKLSKKKKGYNVNDE

SEQ ID NO: 89 - protein, 15 aa, Artificial Sequence
  Synthetic construct
GGKLSKKKKGYNVND

SEQ ID NO: 90 - protein, 13 aa, Artificial Sequence
  Synthetic construct
GGKLSKKKKGYNV

SEQ ID NO: 91 - protein, 12 aa, Artificial Sequence
  Synthetic construct
GGKLSKKKKGYN

SEQ ID NO: 92 - protein, 11 aa, Artificial Sequence
  Synthetic construct
GGKLSKKKKGY

SEQ ID NO: 93 - protein, 10 aa, Artificial Sequence
  Synthetic construct
GGKLSKKKKG

SEQ ID NO: 94 - protein, 9 aa, Artificial Sequence
  Synthetic construct
GGKLSKKKK

SEQ ID NO: 95 - protein, 29 aa, Artificial Sequence
  Synthetic construct
GAKKSKKRFSFKKSFKLSGFSFKKNKKEA

SEQ ID NO: 96 - protein, 28 aa, Artificial Sequence
  Synthetic construct
GAKKSKKRFSFKKSFKLSGFSFKKNKKE

SEQ ID NO: 97 - protein, 27 aa, Artificial Sequence
  Synthetic construct
GAKKSKKRFSFKKSFKLSGFSFKKNKK

SEQ ID NO: 98 - protein, 26 aa, Artificial Sequence
  Synthetic construct
GAKKSKKRFSFKKSFKLSGFSFKKNK

SEQ ID NO: 99 - protein, 25 aa, Artificial Sequence
  Synthetic construct
GAKKSKKRFSFKKSFKLSGFSFKKN

SEQ ID NO: 100 - protein, 24 aa, Artificial Sequence
  Synthetic construct
GAKKSKKRFSFKKSFKLSGFSFKK

SEQ ID NO: 101 - protein, 23 aa, Artificial Sequence
  Synthetic construct
GAKKSKKRFSFKKSFKLSGFSFK

SEQ ID NO: 102 - protein, 22 aa, Artificial Sequence
  Synthetic construct
GAKKSKKRFSFKKSFKLSGFSF

SEQ ID NO: 103 - protein, 21 aa, Artificial Sequence
  Synthetic construct
GAKKSKKRFSFKKSFKLSGFS

SEQ ID NO: 104 - protein, 20 aa, Artificial Sequence
  Synthetic construct
GAKKSKKRFSFKKSFKLSGF

SEQ ID NO: 105 - protein, 19 aa, Artificial Sequence
  Synthetic construct
GAKKSKKRFSFKKSFKLSG

SEQ ID NO: 106 - protein, 18 aa, Artificial Sequence
  Synthetic construct
GAKKSKKRFSFKKSFKLS

SEQ ID NO: 107 - protein, 17 aa, Artificial Sequence
  Synthetic construct
GAKKSKKRFSFKKSFKL

SEQ ID NO: 108 - protein, 16 aa, Artificial Sequence
  Synthetic construct
GAKKSKKRFSFKKSFK

SEQ ID NO: 109 - protein, 15 aa, Artificial Sequence
  Synthetic construct
GAKKSKKRFSFKKSF

SEQ ID NO: 110 - protein, 13 aa, Artificial Sequence
  Synthetic construct
GAKKSKKRFSFKK

SEQ ID NO: 111 - protein, 12 aa, Artificial Sequence
  Synthetic construct
GAKKSKKRFSFK

SEQ ID NO: 112 - protein, 11 aa, Artificial Sequence
  Synthetic construct
GAKKSKKRFSF

SEQ ID NO: 113 - protein, 10 aa, Artificial Sequence
  Synthetic construct
GAKKSKKRFS

SEQ ID NO: 114 - protein, 9 aa, Artificial Sequence
  Synthetic construct
GAKKSKKRF

SEQ ID NO: 115 - protein, 8 aa, Artificial Sequence
  Synthetic construct
GAKKSKKR

SEQ ID NO: 116 - protein, 7 aa, Artificial Sequence
  Synthetic construct
GAKKSKK

SEQ ID NO: 117 - protein, 29 aa, Artificial Sequence
  Synthetic construct
GAKKAKKRFSFKKSFKLSGFSFKKNKKEA

SEQ ID NO: 118 - protein, 28 aa, Artificial Sequence
  Synthetic construct
GAKKAKKRFSFKKSFKLSGFSFKKNKKE

SEQ ID NO: 119 - protein, 27 aa, Artificial Sequence
  Synthetic construct
GAKKAKKRFSFKKSFKLSGFSFKKNKK

SEQ ID NO: 120 - protein, 26 aa, Artificial Sequence
  Synthetic construct
GAKKAKKRFSFKKSFKLSGFSFKKNK

SEQ ID NO: 121 - protein, 25 aa, Artificial Sequence
  Synthetic construct
GAKKAKKRFSFKKSFKLSGFSFKKN

SEQ ID NO: 122 - protein, 24 aa, Artificial Sequence
  Synthetic construct
GAKKAKKRFSFKKSFKLSGFSFKK

SEQ ID NO: 123 - protein, 23 aa, Artificial Sequence
  Synthetic construct
GAKKAKKRFSFKKSFKLSGFSFK

SEQ ID NO: 124 - protein, 22 aa, Artificial Sequence
  Synthetic construct
GAKKAKKRFSFKKSFKLSGFSF

SEQ ID NO: 125 - protein, 21 aa, Artificial Sequence
  Synthetic construct
GAKKAKKRFSFKKSFKLSGFS

SEQ ID NO: 126 - protein, 20 aa, Artificial Sequence
  Synthetic construct
GAKKAKKRFSFKKSFKLSGF

SEQ ID NO: 127 - protein, 19 aa, Artificial Sequence
  Synthetic construct
GAKKAKKRFSFKKSFKLSG

SEQ ID NO: 128 - protein, 18 aa, Artificial Sequence
  Synthetic construct
GAKKAKKRFSFKKSFKLS

SEQ ID NO: 129 - protein, 17 aa, Artificial Sequence
  Synthetic construct
GAKKAKKRFSFKKSFKL

SEQ ID NO: 130 - protein, 16 aa, Artificial Sequence
  Synthetic construct
GAKKAKKRFSFKKSFK

SEQ ID NO: 131 - protein, 15 aa, Artificial Sequence
  Synthetic construct
GAKKAKKRFSFKKSF

SEQ ID NO: 132 - protein, 14 aa, Artificial Sequence
  Synthetic construct
GAKKAKKRFSFKKS

SEQ ID NO: 133 - protein, 13 aa, Artificial Sequence
  Synthetic construct
GAKKAKKRFSFKK

SEQ ID NO: 134 - protein, 12 aa, Artificial Sequence
  Synthetic construct
GAKKAKKRFSFK

SEQ ID NO: 135 - protein, 11 aa, Artificial Sequence
  Synthetic construct
GAKKAKKRFSF

SEQ ID NO: 136 - protein, 10 aa, Artificial Sequence
  Synthetic construct
GAKKAKKRFS

SEQ ID NO: 137 - protein, 9 aa, Artificial Sequence
  Synthetic construct
GAKKAKKRF

SEQ ID NO: 138 - protein, 8 aa, Artificial Sequence
  Synthetic construct
GAKKAKKR

SEQ ID NO: 139 - protein, 7 aa, Artificial Sequence
  Synthetic construct
GAKKAKK

SEQ ID NO: 140 - protein, 28 aa, Artificial Sequence
  Synthetic construct
GAQESKKKKKKRFSFKKSFKLSGFSFKK

SEQ ID NO: 141 - protein, 27 aa, Artificial Sequence
  Synthetic construct
GAQESKKKKKKRFSFKKSFKLSGFSFK

SEQ ID NO: 142 - protein, 26 aa, Artificial Sequence
  Synthetic construct
GAQESKKKKKKRFSFKKSFKLSGFSF

SEQ ID NO: 143 - protein, 25 aa, Artificial Sequence
  Synthetic construct
GAQESKKKKKKRFSFKKSFKLSGFS

SEQ ID NO: 144 - protein, 24 aa, Artificial Sequence
  Synthetic construct
GAQESKKKKKKRFSFKKSFKLSGF

SEQ ID NO: 145 - protein, 23 aa, Artificial Sequence
  Synthetic construct
GAQESKKKKKKRFSFKKSFKLSG

SEQ ID NO: 146 - protein, 22 aa, Artificial Sequence
  Synthetic construct
GAQESKKKKKKRFSFKKSFKLS

SEQ ID NO: 147 - protein, 21 aa, Artificial Sequence
  Synthetic construct
GAQESKKKKKKRFSFKKSFKL

SEQ ID NO: 148 - protein, 20 aa, Artificial Sequence
  Synthetic construct
GAQESKKKKKKRFSFKKSFK

SEQ ID NO: 149 - protein, 19 aa, Artificial Sequence
  Synthetic construct
GAQESKKKKKKRFSFKKSF

SEQ ID NO: 150 - protein, 18 aa, Artificial Sequence
  Synthetic construct
GAQESKKKKKKRFSFKKS

SEQ ID NO: 151 - protein, 17 aa, Artificial Sequence
  Synthetic construct
GAQESKKKKKKRFSFKK

SEQ ID NO: 152 - protein, 16 aa, Artificial Sequence
  Synthetic construct
GAQESKKKKKKRFSFK

SEQ ID NO: 153 - protein, 15 aa, Artificial Sequence
  Synthetic construct
GAQESKKKKKKRFSF

SEQ ID NO: 154 - protein, 14 aa, Artificial Sequence
  Synthetic construct
GAQESKKKKKKRFS

SEQ ID NO: 155 - protein, 13 aa, Artificial Sequence
  Synthetic construct
GAQESKKKKKKRF

SEQ ID NO: 156 - protein, 12 aa, Artificial Sequence
  Synthetic construct
GAQESKKKKKKR

SEQ ID NO: 157 - protein, 11 aa, Artificial Sequence
  Synthetic construct
GAQESKKKKKK

SEQ ID NO: 158 - protein, 10 aa, Artificial Sequence
  Synthetic construct
GAQESKKKKK

SEQ ID NO: 159 - protein, 9 aa, Artificial Sequence
  Synthetic construct
GAQESKKKK

SEQ ID NO: 160 - protein, 8 aa, Artificial Sequence
  Synthetic construct
GAQESKKK

SEQ ID NO: 161 - protein, 7 aa, Artificial Sequence
  Synthetic construct
GAQESKK

SEQ ID NO: 162 - protein, 30 aa, Artificial Sequence
  Synthetic construct
GSQSSKKKKKKFSFKKPFKLSGLSFKRNRK

SEQ ID NO: 163 - protein, 29 aa, Artificial Sequence
  Synthetic construct
GSQSSKKKKKKFSFKKPFKLSGLSFKRNR

SEQ ID NO: 164 - protein, 28 aa, Artificial Sequence
  Synthetic construct
GSQSSKKKKKKFSFKKPFKLSGLSFKRN

SEQ ID NO: 165 - protein, 27 aa, Artificial Sequence
  Synthetic construct
GSQSSKKKKKKFSFKKPFKLSGLSFKR

SEQ ID NO: 166 - protein, 26 aa, Artificial Sequence
  Synthetic construct
GSQSSKKKKKKFSFKKPFKLSGLSFK

SEQ ID NO: 167 - protein, 25 aa, Artificial Sequence
  Synthetic construct
GSQSSKKKKKKFSFKKPFKLSGLSF

SEQ ID NO: 168 - protein, 24 aa, Artificial Sequence
  Synthetic construct
GSQSSKKKKKKFSFKKPFKLSGLS

SEQ ID NO: 169 - protein, 23 aa, Artificial Sequence
  Synthetic construct
GSQSSKKKKKKFSFKKPFKLSGL

SEQ ID NO: 170 - protein, 22 aa, Artificial Sequence
  Synthetic construct
GSQSSKKKKKKFSFKKPFKLSG

SEQ ID NO: 171 - protein, 21 aa, Artificial Sequence
  Synthetic construct
GSQSSKKKKKKFSFKKPFKLS

SEQ ID NO: 172 - protein, 20 aa, Artificial Sequence
  Synthetic construct
GSQSSKKKKKKFSFKKPFKL

SEQ ID NO: 173 - protein, 19 aa, Artificial Sequence
  Synthetic construct
GSQSSKKKKKKFSFKKPFK

SEQ ID NO: 174 - protein, 18 aa, Artificial Sequence
  Synthetic construct
GSQSSKKKKKKFSFKKPF

SEQ ID NO: 175 - protein, 17 aa, Artificial Sequence
  Synthetic construct
GSQSSKKKKKKFSFKKP

SEQ ID NO: 176 - protein, 16 aa, Artificial Sequence
  Synthetic construct
GSQSSKKKKKKFSFKK

SEQ ID NO: 177 - protein, 15 aa, Artificial Sequence
  Synthetic construct
GSQSSKKKKKKFSFK

SEQ ID NO: 178 - protein, 14 aa, Artificial Sequence
  Synthetic construct
GSQSSKKKKKKFSF

SEQ ID NO: 179 - protein, 13 aa, Artificial Sequence
  Synthetic construct
GSQSSKKKKKKFS

SEQ ID NO: 180 - protein, 12 aa, Artificial Sequence
  Synthetic construct
GSQSSKKKKKKF

SEQ ID NO: 181 - protein, 11 aa, Artificial Sequence
  Synthetic construct
GSQSSKKKKKK

SEQ ID NO: 182 - protein, 10 aa, Artificial Sequence
  Synthetic construct
GSQSSKKKKK

SEQ ID NO: 183 - protein, 9 aa, Artificial Sequence
  Synthetic construct
GSQSSKKKK

SEQ ID NO: 184 - protein, 8 aa, Artificial Sequence
  Synthetic construct
GSQSSKKK

SEQ ID NO: 185 - protein, 7 aa, Artificial Sequence
  Synthetic construct
GSQSSKK

SEQ ID NO: 186 - protein, 731 aa, Artificial Sequence
  Synthetic construct
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 187 - protein, 611 aa, Artificial Sequence
  Synthetic construct
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 188 - protein, 485 aa, Artificial Sequence
  Synthetic construct
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 189 - protein, 343 aa, Artificial Sequence
  Synthetic construct
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 190 - protein, 217 aa, Artificial Sequence
  Synthetic construct
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 191 - protein, 66 aa, Artificial Sequence
  Synthetic construct
SKPVFITVKMDVLNAFKYPLLIGVGLSTVIGLLSCLIGYCSSHWCCKKEVQETRRERRRLMSMEMD

SEQ ID NO: 192 - protein, 21 aa, Artificial Sequence
  Synthetic construct
MGRLASRPLLLALLSLALCRG

SEQ ID NO: 193 - protein, 247 aa, Artificial Sequence
  Synthetic construct
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 194 - protein, 168 aa, Artificial Sequence
  Synthetic construct
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 195 - protein, 66 aa, Artificial Sequence
  Synthetic construct
SHLAALWPFLGIVAEVLVLVTIIFIYEKRRKPEDVLDDDDAGSAPLKSSGQHQNDKGKNVRQRNSS

SEQ ID NO: 196 - protein, 18 aa, Artificial Sequence
  Synthetic construct
MAAALFVLLGFALLGTHG

SEQ ID NO: 197 - protein, 456 aa, Artificial Sequence
  Synthetic construct
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 198 - protein, 320 aa, Artificial Sequence
  Synthetic construct
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 199 - protein, 179 aa, Artificial Sequence
  Synthetic construct
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 200 - protein, 24 aa, Artificial Sequence
  Synthetic construct
VALVTGATVLGTITCCFMKRLRKR

SEQ ID NO: 201 - protein, 27 aa, Artificial Sequence
  Synthetic construct
MGALRPTLLPPSLPLLLLLMLGMGCWA

SEQ ID NO: 202 - protein, 1021 aa, Artificial Sequence
  Synthetic construct
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 203 - protein, 1195 aa, Artificial Sequence
  Synthetic construct
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 204 - protein, 1023 aa, Artificial Sequence
  Synthetic construct
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 205 - protein, 240 aa, Artificial Sequence
  Synthetic construct
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNI

SEQ ID NO: 206 - protein, 780 aa, Artificial Sequence
  Synthetic construct
CFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 207 - protein, 1026 aa, Artificial Sequence
  Synthetic construct
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 208 - protein, 1029 aa, Artificial Sequence
  Synthetic construct
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 209 - protein, 279 aa, Artificial Sequence
  Synthetic construct
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 210 - protein, 1258 aa, Artificial Sequence
  Synthetic construct
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 211 - protein, 1272 aa, Artificial Sequence
  Synthetic construct
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 212 - protein, 874 aa, Artificial Sequence
  Synthetic construct
MGDMANSSIEFHPKPQQQRDVPQAGGFGCTLAELRTLMELRGAEALQKIEEAYGDVSGLCRRLKTSPTEGLADNTNDLEKRRQIYGQNFIPPKQPKTFLQLVWEALQDVTLIILEVAAIVSLGLSFYAPPGEESEACGNVSGGAEDEGEAEAGWIEGAAILLSVICVVLVTAFNDWSKEKQFRGLQSRIEQEQKFTVIRNGQLLQVPVAALVVGDIAQVKYGDLLPADGVLIQANDLKIDESSLTGESDHVRKSADKDPMLLSGTHVMEGSGRMVVTAVGVNSQTGIIFTLLGAGGEEEEKKDKKGKQQDGAMESSQTKAKKQDGAVAMEMQPLKSAEGGEMEEREKKKANAPKKEKSVLQGKLTKLAVQIGKAGLVMSAITVIILVLYFVIETFVVEGRTWLAECTPVYVQYFVKFFIIGVTVLVVAVPEGLPLAVTISLAYSVKKMMKDNNLVRHLDACETMGNATAICSDKTGTLTTNRMTVVQSYLGDTHYKEIPAPSALTPKILDLLVHAISINSAYTTKILPPEKEGALPRQVGNKTECALLGFVLDLKRDFQPVREQIPEDKLYKVYTFNSVRKSMSTVIRMPDGGFRLFSKGASEILLKKCTNILNSNGELRGFRPRDRDDMVRKIIEPMACDGLRTICIAYRDFSAGQEPDWDNENEVVGDLTCIAVVGIEDPVRPEVPEAIRKCQRAGITVRMVTGDNINTARAIAAKCGIIQPGEDFLCLEGKEFNRRIRNEKGEIEQERLDKVWPKLRVLARSSPTDKHTLVKGIIDSTTGEQRQVVAVTGDGTNDGPALKKADVGFAMGIAGTDVAKEASDIILTDDNFTSIVKAVMWGRNVYDSISKFLQFQLTVNVVAVIVAFTGACIT

SEQ ID NO: 213 - protein, 28 aa, Artificial Sequence
  Synthetic construct
MQRVNMIMAESPGLITICLLGYLLSAEC

SEQ ID NO: 214 - protein, 18 aa, Artificial Sequence
  Synthetic construct
TVFLDHENANKILNRPKR

SEQ ID NO: 215 - protein, 415 aa, Artificial Sequence
  Synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 216 - protein, 415 aa, Artificial Sequence
  Synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 217 - protein, 8 aa, Artificial Sequence
  Synthetic construct
  MISC_FEATURE (1)..(8): Shortest example of (GGS)n(GGGGS)n linker, wherein n is an integer between 1 and 100
GGSGGGGS

SEQ ID NO: 218 - protein, 7 aa, Artificial Sequence
  Synthetic construct
SGGSGGS

SEQ ID NO: 219 - protein, 15 aa, Artificial Sequence
  Synthetic construct
GGSGGSGGSGGSGGG

SEQ ID NO: 220 - protein, 16 aa, Artificial Sequence
  Synthetic construct
GGSGGSGGGGSGGGGS

SEQ ID NO: 221 - protein, 18 aa, Artificial Sequence
  Synthetic construct
GGSGGSGGSGGSGGSGGS

SEQ ID NO: 222 - protein, 15 aa, Artificial Sequence
  Synthetic construct
GGGGSGGGGSGGGGS

SEQ ID NO: 223 - protein, 4 aa, Artificial Sequence
  Synthetic construct
  MISC_FEATURE (1)..(4): Shortest example of (GGGG)n linker, wherein n is an integer between 1 and 100
GGGG

SEQ ID NO: 224 - protein, 9 aa, Artificial Sequence
  Synthetic construct
  MISC_FEATURE (3)..(3): Xaa = any amino acid
MGXKLSKKK

SEQ ID NO: 225 - protein, 8 aa, Artificial Sequence
  Synthetic construct
  MISC_FEATURE (2)..(2): X= is any amino acid
GXKLSKKK

SEQ ID NO: 226 - RNA, 22 nt, Artificial Sequence
  Synthetic construct
ugagaacugaauuccauggguu

SEQ ID NO: 227 - RNA, 22 nt, Artificial Sequence
  Synthetic construct
ccucugaaauucaguucuucag

SEQ ID NO: 228 - RNA, 23 nt, Artificial Sequence
  Synthetic construct
uuaaugcuaaucgugauaggggu

SEQ ID NO: 229 - RNA, 22 nt, Artificial Sequence
  Synthetic construct
cuccuacauauuagcauuaaca

SEQ ID NO: 230 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 231 - protein, 96 aa, Mycobacterium tuberculosis
MSQIMYNYPAMLGHAGDMAGYAGTLQSLGAEIAVEQAALQSAWQGDTGITYQAWQAQWNQAMEDLVRAYHAMSSTHEANTMAMMARDPAEAAKWGG